(19) United States
(12) Patent Application Publication
(10) Pub. No.: US 2026/0132183 A1
(43) Pub. Date: May 14, 2026

(54) T CELL RECEPTORS DIRECTED AGAINST MELANOMA-ASSOCIATED ANTIGEN AND USES THEREOF

(71) Applicant: ACADEMISCH ZIEKENHUIS LEIDEN (H.O.D.N. LUMC), Leiden (NL)

(72) Inventors: Mirjam H. M. HEEMSKERK, Leiden (NL); J.H. Frederik FALKENBURG, Leiden (NL)

(21) Appl. No.: 18/873,567

(22) PCT Filed: Jun. 12, 2023
(86) PCT No.: PCT/NL2023/050325

§ 371 Date: Dec. 10, 2024

(30) Foreign Application Priority Data
Jun. 10, 2022 (NL) 2032130

(51) Int. Cl. C07K 14/725; A61K 9/00; A61K 31/7088; A61K 38/00; A61P 35/00; C07K 16/30; C12N 15/10; C12N 15/11

(52) U.S. Cl. CPC C07K 14/7051; A61K 9/0019; A61K 31/7088; A61K 38/00; A61P 35/00; C07K 16/3053; C12N 15/10; C12N 15/11; C07K 2317/565; C07K 2319/00; C12N 2510/00

(57) ABSTRACT

Novel nucleic acid compositions, vector systems, modified cells, and pharmaceutical compositions that encode or express T cell receptor components directed against melanoma-associated antigen (MAGE) are provided herein. These novel components may be used to enhance an immune response in a subject diagnosed with a MAGE associated disease or condition, such as a hematological malignancy or a solid tumor. Associated methods for treating such subjects are also provided herein.

### REFERENCE TO A SEQUENCE LISTING XML

This application contains a Sequence Listing which has been submitted electronically in XML format. The Sequence Listing XML is incorporated herein by reference. Said XML file, created on Jan. 23, 2026, is named 2970877-000010-US1_SL.xml and is 191,052 bytes in size.

Novel nucleic acid compositions, vector systems, modified cells, and pharmaceutical compositions that encode or express T cell receptor components directed against melanoma-associated antigen (MAGE) are provided herein. These novel components may be used to enhance an immune response in a subject diagnosed with a MAGE associated disease or condition, such as a hematological malignancy or a solid tumor. Associated methods for treating such subjects are also provided herein.

### CROSS-REFERENCE TO RELATED APPLICATIONS

This application is a National Stage Application under 35 U.S.C. 371 of PCT Application No. PCT/NL2023/050325, filed on Jun. 12, 2023, which is an International Application that claims priority from a NL Patent Application No. 2032130, filed on Jun. 10, 2022, the contents of each of the above applications are incorporated herein by reference in their entireties.

### BACKGROUND

Cancer testis (CT) genes are potential targets for cancer immunotherapy since they are highly expressed in multiple different tumor types as demonstrated by the TCGA Research network (https://www.cancer.gov/tcga). Expression in normal tissue is limited to the immunological privileged testis and placenta, which makes CT antigens ideal targets for T cells [1-4]. T cells with high-affinity T cell receptors (TCRs) targeting self-antigens, such as CT genes, are deleted in the self-repertoire during negative selection in the thymus [5, 6]. Therefore, CT-specific T cells present in the self-repertoire may only display limited antitumor efficacy, as illustrated by the limited antitumor responses observed in patients after vaccination aimed at harnessing CT-specific T cells [7, 8]. To overcome negative selection of high-affinity TCRs in the thymus, high-affinity TCRs can be identified from the human allo-HLA repertoire and included in TCR gene therapy [5, 6, 9]. Previous studies revealed that self-antigens can provoke strong immune responses when presented in the context of allo-HLA molecules [10]. Recently various potent allogeneic TCRs have been identified, of which several are in the process of clinical development [10-13]. However, the use of high-affinity TCRs for TCR gene therapy also comes with a toxicity risk. Two clinical trials targeting a melanoma-associated antigen (MAGE)-A3 peptide had to be terminated because of unexpected deaths of patients [14, 15]. In the first clinical trial the affinity-optimized TCR was not only recognizing the KVAELVHFL (SEQ ID NO: 179) peptide of MAGE-A3 but also, among others, the KMAELVHFL (SEQ ID NO: 180) peptide of MAGE-A12. The expression of MAGE-A12 in the brain most likely resulted in the death of 2 patients. In a second study, besides the EVDPIGHLY (SEQ ID NO: 133) peptide of MAGE-A3, the ESDPIVAQY (SEQ ID NO: 181) peptide derived from titin was recognized leading to cardiotoxicity and a fatal outcome in 2 patients. This underlines the importance of proper safety screenings during the selection process of tumor specific TCRs to exclude TCR cross-reactivity against look-a-like peptides [14-16].

Accordingly, there is a need for improved means for treating a MAGE associated disease or condition, such as hematological malignancy or a solid tumor.

### BRIEF SUMMARY OF THE DISCLOSURE

As described herein, the inventors sought to expand the library of potent CT-specific TCRs. Advantageously, such a library may eventually be used to select a personalized TCR gene therapy, based on HLA typing of the patient and gene expression of the tumor, for each individual patient. To increase numbers of (cancer) patients that can be treated with TCR gene therapy, the inventors sought to identify a novel set of high-affinity cancer specific TCRs targeting different cancer testis (CT) antigens in prevalent HLA class I alleles.

To identify novel CT-specific TCRs, the inventors selected the most promising CT genes to target using bioinformatics tools. From these selected genes, the inventors identified the naturally processed and presented HLA class I peptides using HLA peptidomics. Peptide-HLA tetramers were subsequently generated, and used for single cell sorting and expansion of CT specific CD8⁺ T cells from the allo-HLA repertoire of healthy donors. Using several functional assays, the inventors were able to identify high avidity CT-specific T cell clones with a safe recognition pattern. To evaluate the potential for clinical application in TCR gene therapy, TCRs were sequenced, transferred into peripheral blood derived CD8⁺ T cells, and tested for anti-tumor efficacy in vitro and in an orthotopic xenograft mice model for established multiple myeloma. Advantageously, the inventors identified several novel CT-specific TCRs that effectively target melanoma-associated antigen (MAGE)-A1, MAGE-A3, MAGE-A6 and MAGE-A9 in the context of human leukocyte antigen (HLA)-A1, -A2, -A3, -B7, -B35 and -C7 which are described herein. Notably, MAGE-A1, MAGE-A3, MAGE-A6 and MAGE-A9 are expressed in a variety of different tumor types including in melanomas, non-small cell lung carcinomas, breast carcinomas, ovarian carcinomas, colon carcinomas and multiple myelomas.

TCR gene transfer into CD8⁺ T cells resulted in efficient cytokine production and cytotoxicity of variety of different tumor types without detectable cross-reactivity. In addition, major in vivo antitumor effects of MAGE-A1 specific TCR engineered CD8⁺ T cells was observed in the orthotopic xenograft model for established multiple myeloma, in which bone marrow located tumor cells were completely eradicated after T cell injection.

The inventors have therefore been able to identify high affinity T cell clones (and corresponding TCRs) that are both potent and safe (see Table 1). Advantageously, the identification of several novel CT-specific TCRs, reactive against CT antigens presented in a variety of different HLA class I alleles, allow TCR gene therapy for an increased number of cancer patients, and thus, aid the development of personalized TCR gene therapy.

TABLE 1
list of potent and safe TCRs.

| | | | | HLA- |
|---|---|---|---|---|
| Name | Full Name | Target Peptide | Gene | restriction |
| 4F7 | CT27.4F7 | KVLEYVIKV (SEQ ID NO: 127) | MAGE-A1 | HLA-A*02:01 |
| 10C1 | CT31.10C1 | VRFFFPSL (SEQ ID NO: 128) | MAGE-A1 | HLA-C*07:02 |
| 2D8 | CT43.2D8 | YVGKEHMFY (SEQ ID NO: 129) | MAGE-A9 | HLA-A*01:01 |
| 6G4 | CT44.6G4 | LTQDLVQEKYLEY (SEQ ID NO: | MAGE-A1 | HLA-A*01:01 |
| | | 130) | | |
| 3H4 | CT23.3H4 | SLFRAVITK (SEQ ID NO: 131) | MAGE-A1 | HLA-A*03:01 |
| 3B2 | MRM23.3B2 | SLFRAVITK (SEQ ID NO: 131) | MAGE-A1 | HLA-A*03:01 |
| 3G2 | CT12.3G2 | RVRFFFPSL (SEQ ID NO: 132) | MAGE-A1 | HLA-B*07:02 |
| 2C2 | CT4.20.2C2 | RVRFFFPSL (SEQ ID NO: 132) | MAGE-A1 | HLA-B*07:02 |
| 2H9 | CT23.2H9 | EVDPIGHXY (SEQ ID NO: 183) | MAGE- | HLA-B*35:01 |
| | | | A3/A6 | |

Accordingly, the invention provides an isolated nucleic acid composition that encodes a melanoma-associated antigen (MAGE) antigen-specific binding protein having a TCR α chain variable (Vα) domain and a TCR β chain variable (Vβ) domain, the composition comprising:
(i) a nucleic acid sequence that encodes a TCR Vα domain comprising a CDR3 amino acid sequence having at least 80% sequence identity to SEQ ID NO:3, or a functional fragment thereof; and a nucleic acid sequence that encodes a TCR Vβ domain comprising a CDR3 amino acid sequence having at least 80% sequence identity to SEQ ID NO:6, or a functional fragment thereof, wherein the CDR3 sequences together specifically bind to MAGE-A1; or
(ii) a nucleic acid sequence that encodes a TCR Vα domain comprising a CDR3 amino acid sequence having at least 80% sequence identity to SEQ ID NO:17, or a functional fragment thereof; and a nucleic acid sequence that encodes a TCR Vβ domain comprising a CDR3 amino acid sequence having at least 80% sequence identity to SEQ ID NO: 20, or a functional fragment thereof, wherein the CDR3 sequences together specifically bind to MAGE-A1; or
(iii) a nucleic acid sequence that encodes a TCR Vα domain comprising a CDR3 amino acid sequence having at least 80% sequence identity to SEQ ID NO:31, or a functional fragment thereof; and a nucleic acid sequence that encodes a TCR Vβ domain comprising a CDR3 amino acid sequence having at least 80% sequence identity to SEQ ID NO:34, or a functional fragment thereof, wherein the CDR3 sequences together specifically bind to MAGE-A9; or
(iv) a nucleic acid sequence that encodes a TCR Vα domain comprising a CDR3 amino acid sequence having at least 80% sequence identity to SEQ ID NO:45, or a functional fragment thereof; and a nucleic acid sequence that encodes a TCR Vβ domain comprising a CDR3 amino acid sequence having at least 80% sequence identity to SEQ ID NO:48, or a functional fragment thereof, wherein the CDR3 sequences together specifically bind to MAGE-A1; or
(v) a nucleic acid sequence that encodes a TCR Vα domain comprising a CDR3 amino acid sequence having at least 80% sequence identity to SEQ ID NO:59, or a functional fragment thereof; and a nucleic acid sequence that encodes a TCR Vβ domain comprising a CDR3 amino acid sequence having at least 80% sequence identity to SEQ ID NO: 62, or a functional fragment thereof, wherein the CDR3 sequences together specifically bind to MAGE-A1; or
(vi) a nucleic acid sequence that encodes a TCR Vα domain comprising a CDR3 amino acid sequence having at least 80% sequence identity to SEQ ID NO:73, or a functional fragment thereof; and a nucleic acid sequence that encodes a TCR Vβ domain comprising a CDR3 amino acid sequence having at least 80% sequence identity to SEQ ID NO: 76, or a functional fragment thereof, wherein the CDR3 sequences together specifically bind to MAGE-A1; or
(vii) a nucleic acid sequence that encodes a TCR Vα domain comprising a CDR3 amino acid sequence having at least 80% sequence identity to SEQ ID NO:87, or a functional fragment thereof; and a nucleic acid sequence that encodes a TCR Vβ domain comprising a CDR3 amino acid sequence having at least 80% sequence identity to SEQ ID NO: 90, or a functional fragment thereof, wherein the CDR3 sequences together specifically bind to MAGE-A1; or
(viii) a nucleic acid sequence that encodes a TCR Vα domain comprising a CDR3 amino acid sequence having at least 80% sequence identity to SEQ ID NO: 101, or a functional fragment thereof; and a nucleic acid sequence that encodes a TCR Vβ domain comprising a CDR3 amino acid sequence having at least 80% sequence identity to SEQ ID NO: 104, or a functional fragment thereof, wherein the CDR3 sequences together specifically bind to MAGE-A1; or
(ix) a nucleic acid sequence that encodes a TCR Vα domain comprising a CDR3 amino acid sequence having at least 80% sequence identity to SEQ ID NO: 115, or a functional fragment thereof; and a nucleic acid sequence that encodes a TCR Vβ domain comprising a CDR3 amino acid sequence having at least 80% sequence identity to SEQ ID NO: 118, or a functional fragment thereof, wherein the CDR3 sequences together specifically bind to MAGE-A3 and/or MAGE-A6.

Suitably:
(i) the CDR3 of the Vα domain may comprise or consist of the amino acid sequence of SEQ ID NO: 3, and the CDR3 of the Vβ domain may comprise or consist of the amino acid sequence of SEQ ID NO:6; or
(ii) the CDR3 of the Vα domain may comprise or consist of the amino acid sequence of SEQ ID NO: 17, and the CDR3 of the Vβ domain may comprise or consist of the amino acid sequence of SEQ ID NO:20; or
(iii) the CDR3 of the Vα domain may comprise or consist of the amino acid sequence of SEQ ID NO: 31, and the CDR3 of the Vβ domain may comprise or consist of the amino acid sequence of SEQ ID NO:34; or
(iv) the CDR3 of the Vα domain may comprise or consist of the amino acid sequence of SEQ ID NO: 45, and the CDR3 of the Vβ domain may comprise or consist of the amino acid sequence of SEQ ID NO:48; or
(v) the CDR3 of the Vα domain may comprise or consist of the amino acid sequence of SEQ ID NO: 59, and the CDR3 of the Vβ domain may comprise or consist of the amino acid sequence of SEQ ID NO:62; or
(vi) the CDR3 of the Vα domain may comprise or consist of the amino acid sequence of SEQ ID NO: 73, and the CDR3 of the Vβ domain may comprise or consist of the amino acid sequence of SEQ ID NO:76; or
(vii) the CDR3 of the Vα domain may comprise or consist of the amino acid sequence of SEQ ID NO: 87, and the CDR3 of the Vβ domain may comprise or consist of the amino acid sequence of SEQ ID NO:90; or
(viii) the CDR3 of the Vα domain may comprise or consist of the amino acid sequence of SEQ ID NO: 101, and the CDR3 of the Vβ domain may comprise or consist of the amino acid sequence of SEQ ID NO: 104; or
(ix) the CDR3 of the Vα domain may comprise or consist of the amino acid sequence of SEQ ID NO: 115, and the CDR3 of the Vβ domain may comprise or consist of the amino acid sequence of SEQ ID NO: 118.

### Suitably:

(i) the Vα domain may comprise an amino acid sequence having at least 80% sequence identity to, comprising, or consisting of, SEQ ID NO: 7; and the Vβ domain may comprise an amino acid sequence having at least 80% sequence identity to, comprising, or consisting of, SEQ ID NO: 9; or
(ii) the Vα domain may comprise an amino acid sequence having at least 80% sequence identity to, comprising, or consisting of, SEQ ID NO: 21; and the Vβ domain may comprise an amino acid sequence having at least 80% sequence identity to, comprising, or consisting of, SEQ ID NO: 23; or
(iii) the Vα domain may comprise an amino acid sequence having at least 80% sequence identity to, comprising, or consisting of, SEQ ID NO: 35; and the Vβ domain may comprise an amino acid sequence having at least 80% sequence identity to, comprising, or consisting of, SEQ ID NO: 37; or
(iv) the Vα domain may comprise an amino acid sequence having at least 80% sequence identity to, comprising, or consisting of, SEQ ID NO: 49; and the Vβ domain may comprise an amino acid sequence having at least 80% sequence identity to, comprising, or consisting of, SEQ ID NO: 51; or
(v) the Vα domain may comprise an amino acid sequence having at least 80% sequence identity to, comprising, or consisting of, SEQ ID NO: 63; and the Vβ domain may comprise an amino acid sequence having at least 80% sequence identity to, comprising, or consisting of, SEQ ID NO: 65; or
(vi) the Vα domain may comprise an amino acid sequence having at least 80% sequence identity to, comprising, or consisting of, SEQ ID NO: 77; and the Vβ domain may comprise an amino acid sequence having at least 80% sequence identity to, comprising, or consisting of, SEQ ID NO: 79; or
(vii) the Vα domain may comprise an amino acid sequence having at least 80% sequence identity to, comprising, or consisting of, SEQ ID NO: 91; and the Vβ domain may comprise an amino acid sequence having at least 80% sequence identity to, comprising, or consisting of, SEQ ID NO: 93; or
(viii) the Vα domain may comprise an amino acid sequence having at least 80% sequence identity to, comprising, or consisting of, SEQ ID NO: 105; and the Vβ domain may comprise an amino acid sequence having at least 80% sequence identity to, comprising, or consisting of, SEQ ID NO: 107; or
(ix) the Vα domain may comprise an amino acid sequence having at least 80% sequence identity to, comprising, or consisting of, SEQ ID NO: 119; and the Vβ domain may comprise an amino acid sequence having at least 80% sequence identity to, comprising, or consisting of, SEQ ID NO: 121.

Suitably, the MAGE antigen may be a MAGE-A1 antigen, a MAGE-A9 antigen, a MAGE-A3 antigen or a MAGE-A6 antigen. In other words, the antigen may be derived from MAGE-A1, MAGE-A9, MAGE-A3 and/or MAGE-A6.

Suitably, the MAGE antigen may comprise an amino acid sequence selected from the group consisting of: KVLEYVIKV (SEQ ID NO: 127), VRFFFPSL (SEQ ID NO: 128), YVGKEHMFY (SEQ ID NO: 129), LTQDLVQEKYLEY (SEQ ID NO: 130), SLFRAVITK (SEQ ID NO: 131), RVRFFFPSL (SEQ ID NO: 132), EVDPIGHLY (SEQ ID NO: 133), EVDPIGHVY (SEQ ID NO: 134) and EVDPIGHXY (SEQ ID NO: 183).

Suitably, the encoded binding protein may be capable of specifically binding to a peptide:HLA complex selected from the group consisting of: a KVLEYVIKV:HLA-A*02:01 complex (SEQ ID NO: 127), a VRFFFPSL:HLA-C*07:02 complex (SEQ ID NO: 128), a YVGKEHMFY:HLA-A*01:01 complex (SEQ ID NO: 129), a LTQDLVQEKYLEY:HLA-A*01:01 complex (SEQ ID NO: 130), a SLFRAVITK:HLA-A*03:01 (SEQ ID NO: 131), a RVRFFFPSL:HLA-B*07:02 complex (SEQ ID NO: 132), a EVDPIGHLY:HLA-B*35:01 complex (SEQ ID NO: 133), a EVDPIGHVY:HLA-B*35:01 complex (SEQ ID NO: 134) and EVDPIGHXY:HLA-B*35:01 complex (SEQ ID NO: 183).

Suitably, the nucleic acid sequence may be codon optimised for expression in a host cell, optionally wherein the host cell is a human cell.

Suitably the nucleic acid composition may further comprise a TCR α chain constant domain and/or a TCR β chain constant domain.

Suitably, the encoded binding protein may comprise a TCR, an antigen binding fragment of a TCR, a chimeric antigen receptor (CAR), or an ImmTAC.

Suitably, the antigen binding fragment of a TCR may be a single chain TCR (scTCR) or a chimeric TCR dimer in which the antigen binding fragment of the TCR is linked to an alternative transmembrane and intracellular signalling domain.

The invention also provides a vector system comprising a nucleic acid composition according to the invention.

Suitably, the vector may be a plasmid, a viral vector, or a cosmid, optionally wherein the vector is selected from the group consisting of a retrovirus, lentivirus, adeno-associated virus, adenovirus, vaccinia virus, canary poxvirus, herpes virus, minicircle vector and synthetic DNA or RNA.

The invention also provides a modified cell comprising a nucleic acid composition according to the invention, or a vector system according to the invention.

Suitably, the modified cell may be selected from the group consisting of a CD8 T cell, a CD4 T cell, an NK cell, an NK-T cell, a gamma-delta T cell, a hematopoietic stem cell, an inducible pluripotent stem cell, a progenitor cell, a T cell line and a NK-92 cell line.

Suitably, the modified cell may be a human cell.

The invention also provides a pharmaceutical composition comprising a nucleic acid composition according to the invention, a vector system according to the invention, or a modified cell according to the invention, and a pharmaceutically acceptable excipient, adjuvant, diluent and/or carrier.

The invention also provides a pharmaceutical composition according to the invention for use in inducing or enhancing an immune response in human subject diagnosed with a MAGE associated disease or condition.

The invention also provides a pharmaceutical composition according to the invention for use in stimulating a cell mediated immune response to a target cell population or tissue in a human subject.

The invention also provides a pharmaceutical composition according to the invention for use in providing anti-tumor immunity to a human subject.

The invention also provides a pharmaceutical composition according to the invention for use in treating an human subject having a disease or condition associated with an elevated level of HLA-restricted MAGE antigen.

Suitably, the human subject may have at least one tumor.

Suitably, the subject may have been diagnosed with a MAGE associated disease or condition.

Suitably, the MAGE associated disease or condition may be a hematological malignancy or a solid tumor.

Suitably, the hematological malignancy may be selected from the group consisting of: Multiple myeloma, plasma cell leukemia, Amyloidosis (AL), Acute lymphoblastoid leukemia (ALL), Chronic lymphocytic leukemia (CLL), Waldenstrom macroglobulinemia, Acute myeloid leukemia (AML), Myeloid dysplastic syndrome (MDS) and α cell lymphoma, optionally wherein the B cell lymphoma is selected from the group consisting of: Diffuse large B cell lymphoma (DLBCL), High grade B cell lymphoma, Mantel cell lymphoma (MCL), Follicular lymphoma (FL) and Burkitt Lymphoma.

Suitably, the hematological malignancy may be multiple myeloma.

Suitably, the solid tumor may be selected from the group consisting of: Melanoma, Lung carcinoma, Bladder carcinoma, Ovarian carcinoma, Head and neck carcinoma, Breast carcinoma, Sarcoma, Uveal melanoma and Uterine carcinoma, optionally wherein the solid tumor is selected from the group consisting of: non-small cell lung carcinoma, head and neck squamous cell carcinoma, invasive breast carcinoma and synovial sarcoma.

The invention also provides a method of generating a binding protein that is capable of specifically binding to a peptide containing a MAGE antigen and does not bind to a peptide that does not contain the MAGE antigen, comprising contacting a nucleic acid composition according to the invention with a cell under conditions in which the nucleic acid composition is incorporated and expressed by the cell.

Suitably, the method may be ex vivo.

The invention also provides an isolated nucleic acid sequence comprising or consisting of the nucleotide sequence of any one of SEQ ID NOs: 8, 10, 12, 14, 22, 24, 26, 28, 36, 38, 40, 42, 50, 52, 54, 56, 64, 66, 68, 70, 78, 80, 82, 84, 92, 94, 96, 98, 106, 108, 110, 112, 120, 122, 124 or 126.

The invention also provides an isolated nucleic acid sequence comprising or consisting of the nucleotide sequence of any one of SEQ ID NOs: 8, 10, 12, 14, 22, 24, 26, 28, 36, 38, 40, 42, 50, 52, 54, 56, 64, 66, 68, 70, 78, 80, 82, 84, 92, 94, 96, 98, 106, 108, 110, 112, 120, 122, 124 or 126 for use in therapy.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows reactivity against CT-positive tumor cell lines. T cell clones were stimulated with tumor cell lines of different origin expressing the target gene including; multiple myeloma (RPMI8226, U266, L363, OPM-2), osteosarcoma (U2-OS, ZK-58, Saos-2), melanoma (88.23, MEL01.14, SK2.3, 518A2), chronic myeloid leukemia k562, and prostate carcinoma cell line PC-3M-PRO4. The Burkitt lymphoma Raji, negative for all CT antigens, was included as negative control. CT-gene expression levels, measured by qPCR, are depicted between brackets as percentage relative to HKGs. Targeted HLA was naturally expressed (+) or transduced (Td) into the target cell. T-cell reactivity was demonstrated by IFN-γ production, as measured by ELISA, after an overnight co-culture with 1:4 E:T ratio. To confirm proper HLA expression and recognition capacity of the targets an allo-HLA reactive T cell clone was included for each HLA specificity. Values and error bars represent mean and standard deviations of technical duplicates. T cell clones with different specificities were tested in separate experiments and are depicted in different graphs.

FIG. 2 shows TCR expression in transduced primary CD8⁺ T cells. TCR-T cells were stained with murine TCR-β APC (right) and pHLA-tetramer PE (left). In the graph the delta mean fluorescent intensity (MFI)(sample MFI−control MFI) of the tetramer and mTCRβ stain are depicted. Untransduced or CMV (pp65 NLV/A2) TCR-T cells were included as negative control (depicted in grey). Tetramer staining confirmed TCR expression on surface of all TCR-T cells, with lower gMFI of the MAGE-A1 VRF/C7 TCR and MAGE-A3 EVD/B35 TCR compared to other selected TCRs.

FIG. 3 shows cytotoxicity of TCR-T cells against malignant cell lines. Cytotoxicity of the TCR-T cells against multiple tumor cell lines was determined by 6 hour ⁵¹Cr-release assay with E:T ratio of 9:1 or 1:1. T cell clones were stimulated with tumor cell lines of different origin including; multiple myeloma (L363, U266, RPMI8226, UM9), melanoma (518A2, SK2.3), osteosarcoma cell line Saos-2, mammary carcinoma cell line CAMA-1, and prostate carcinoma cell line PC-3M-PRO4. Target cells naturally express target HLA or were transduced with target HLA alleles (+HLA). The CT-gene expression levels, measured by qPCR, are depicted between brackets as percentage relative to HKGs. CMV (pp65 NLV/A2) TCR-T cells were included as negative control and allo-HLA reactive T cell clones as positive control for HLA expression and killing capacity. Values and error bars represent mean and standard deviations of technical triplicates and experiments are representatives of at least two independent experiments.

FIG. 4 shows reactivity of the CT-specific TCRs against early-passage melanoma cell lines. (A) To determine recognition of early-passage melanoma cell lines (passage≤10), TCR-T cells were stimulated in an E:T ratio of 1:2. All melanomas (MEL) naturally expressed the HLA of interest except for MEL18.07 that was transduced with HLA-A1. IFN-γ production, as measured by ELISA, was determined after an overnight stimulation in at least two independent experiments. An allo-HLA reactive T cell clone was included for each target HLA as a positive control for HLA expression and recognition capacity of target cells. The CT-gene expression levels, measured by qPCR, are depicted between brackets as percentage relative to HKGs. Values and error bars represent mean and standard deviations of technical duplicates. (B) Cytotoxicity of the TCR-T cells was analysed by a 6-hour ⁵¹Cr-release assay with E:T ratios of 9:1 and 1:1 in at least two independent experiments. Values and error bars represent mean and standard deviations of technical triplicates.

FIG. 5 shows in vivo antitumor efficacy of MAGE-A1 HLA-A2, -A3 and -C7 restricted TCRs. NSG mice engrafted with 2×10⁶ U266 cells were i.v. injected with 5×10⁶ TCR-T cells 14 days after tumor injection. T cells were transduced with the 4F7 (MAGE-A1 KVL/A2) TCR, 3H4 (MAGE-A1 SLF/A3) TCR, 10C1 (MAGE-A1 VRF/C7) TCR or CMV (pp65 NLV/A2) TCR and enriched for mTCRβ expression by MACS. Tumor growth was visualized by bioluminescence imaging 1-2 times per week. (A) Mean of average tumor outgrowth of the dorsal and ventral side of mice treated with 4F7 (MAGE-A1 KVL/A2) TCR-T cells (black, n=6)), 10C1 (MAGE-A1 VRF/C7) TCR-T cells (red, n=6) and CMV TCR-T cells (white, n=4) treated mice. (B) Tumor outgrowth of representative mice treated with MAGE-A1 KVL/A2 TCR-T cell, MAGE-A1 VRF/C7 TCR-T cell and CMV TCR-T cell treated mice measured on the ventral side. (C) Mean of average tumor outgrowth of the dorsal and ventral side of mice treated with 3H4 (MAGE-A1 SLF/A3) TCR-T cells (red, n=5) and CMV TCR-T cells (white, n=4). (D) Kaplan-Meier plot of MAGE-A1 SLF/A3 TCR-T cell and CMV TCR-T cell treated mice.

FIG. 6 shows peptide sequence validation of matching tandem mass spectra of eluted (top) and synthetically (bottom) generated peptides. Two examples of matching tandem mass spectra of (A) the YVGKEHMF peptide (SEQ ID NO: 177) presented in HLA-A*24:02 and (B) the SMLGDGHSMPK peptide (SEQ ID NO: 169) presented in HLA-A*03:01. Both depicted peptides are derived from MAGE-A9.

FIG. 7 shows that T cell clones were selected based on peptide titration experiment and specific tumor cell line recognition. 6/18 T cell clones recognizing MAGE-A1 KVL/A2 are depicted in this graph that were (A) overnight co-cultured with KVL peptide loaded Raji cells at an E:T ratio of 1:4. Target cells were pre-incubated for at least 30 minutes at 37° C. with titrated peptide concentrations starting at 1 μM. These 6 T cell clones are a representative for T cell clone selection process of all 192 CT-specific T cell clones. (B) In a second screening, the T cell clones were stimulated with different tumor cell lines including osteosarcoma (OST), multiple myeloma (MM), melanoma (MEL) and cervical carcinoma (CER) in a 1:4 E:T ratio. The MAGE-A1 expression (depicted in parenthesis) was determined by qPCR and relative to HKGs. T cell clones 4A2 and 4F7 were reactive against low peptide concentrations and able to strictly recognize the MAGE expressing tumor cell lines and selected for further analysis. 4/18 MAGE-A1 KVL/A2 recognizing T cell clones are depicted as representatives. Values and error bars represent mean and standard deviations of technical duplicates.

FIG. 8 shows target gene expression levels of MAGE-A1, MAGE-A3/A6 and MAGE-A9 mRNA levels measured by qPCR for multiple cell lines with different origin including; multiple myeloma (MM), melanoma (MEL), prostate carcinoma (PC), fibroblast (FC), Burkitt lymphoma (BL), renal cell carcinoma (RCC), ovarium carcinoma (OVCA), colon carcinoma (CC), cervical carcinoma (CER, pulmonary carcinoma (PUL), leukemic T-cell lymphoblast (LT), bile duct carcinoma (BILE), osteosarcoma (OST), acute myeloid leukaemia (AML), chronic myeloid leukaemia (CML) cell lines and mammary carcinoma (MAM). Gene expression>0.01 is depicted in the graph relative to housekeeping genes (HKG). Values<0.01 relative gene expression are not depicted. MAGE-A3 and MAGE-A6 gene expression could not be discriminated from each other by qPCR.

FIG. 9 shows EBV-LCL panels used to determine off-target reactivity against other peptide-HLA complexes. T cell clones were overnight stimulated with multiple EBV-LCLs at an E:T ratio of 1:6. These EBV-LCL panels comprised of different EBV-LCLs expressing all HLA alleles with a prevalence of >1% in the Caucasian population. Peptide loaded HLA Td Raji cells were included as positive control (Ct) for T-cell function. The 4A2 clone (MAGE-A1 KVL/A2) demonstrated reactivity against 2 EBV-LCLs, the HLA restriction of this reactivity could not be identified, and therefore this T cell clone was excluded from further analyses. The 3G2 clone (MAGE-A1 RVR/B7) demonstrated reactivity against the HLA-A*68:02 positive EBV-LCLs. Values and error bars represent mean and standard deviations of technical duplicates.

FIG. 10 shows recognition of specific MAGE-A family members by the selected T cell clones. T cell clones were stimulated with Raji transduced with the different MAGE-A family members including; MAGE-A1 (A1), MAGE-A2 (A2), MAGE-A3 (A3), MAGE-A4 (A4), MAGE-A6 (A6), MAGE-A8 (A8), MAGE-A9 (A9), MAGE-A10 (A10), MAGE-A11 (A11) and MAGE-A12 (A12). Cytokine production was measured by IFN-γ ELISA after an overnight co-culture at E:T ratio of 1:4. In all experiments an allo-HLA reactive T-cell clone (white bars) was included to confirm proper HLA expression and stimulatory capacity of the targets. Values and error bars represent mean and standard deviations of technical duplicates.

FIG. 11 shows cross-reactivity against a look-a-like peptide in the context of the targeted HLA determined by an overnight co-culture of T cells with tumor cell lines from different tissue origin at an ET ratio of 1:4. The cell lines included originated from acute myeloid leukemia (AML), bile duct carcinoma (BILE), colon carcinoma (CC), cervical carcinoma (CER), T-cell leukemia (LT), melanoma (MEL), multiple myeloma (MM), ovarian carcinoma (OC), prostate carcinoma (PC) and pulmonary carcinoma (PUL). In addition, stimulated T cells (T), keratinocytes (K), fibroblasts (F) and fibroblasts cell lines (FC) were included. All included targets expressed the MAGE-A genes of interest<1% relative to HKGs and naturally expressed the HLA of interest or were HLA Td (+HLA). IFN-γ production was measured by ELISA after an overnight co-culture assay. Proper HLA presentation and recognition capacity of the target cells was confirmed by allo-HLA reactive T cell clones (not depicted in this figure). Values and error bars represent mean and standard deviations of technical duplicates.

FIG. 12 shows positive and negative controls included for each target in the cytotoxicity assays. CMV (pp65 NLV/A2) Td CD8⁺ T cell was included as a negative control and an allo-HLA reactive T cell clone against the HLA of interest as a positive control. The tumor cell lines included were; Multiple myeloma (MM), melanoma (MEL), osteosarcoma (OST), prostate carcinoma (PC), and mammary carcinoma (MAM). Transduced (Td) HLA alleles are depicted in the graphs and naturally expressed HLA alleles are depicted between brackets. Cytotoxicity was assessed by an 6-hour chromium-51 release assay with E:T ratio 9:1 and 1:1. Technical triplicates are depicted in the graph and the values are a representatives of at least two independent experiments.

FIG. 13 shows MAGE-A1, MAGE-A3/A6 and MAGE-A9 mRNA expression levels of early passage melanomas (passage≤10) as measured by qPCR. MAGE-A3 and MAGE-A6 gene expression could not be discriminated from each other by qPCR. Gene expression>0.001% is depicted in the graph.

FIG. 14 shows reactivity of the 6G4 MAGE-A1 LTQ/A1 TCR against tumor cell lines. (A) The 6G4 MAGE-A1 LTQ/A1 Td CD8⁺ T cell were overnight stimulated with different MAGE-A1 expressing tumor cell lines including; melanoma (MEL), multiple myeloma (MM), cervical carcinoma (CER) and osteosarcoma (OST) cell lines at an E:T ratio of 1:4. In addition, 1 early passage melanoma samples was included (MEL13.05). An allo-HLA reactive T cell clone was included to confirm proper HLA expression and recognition capacity of the targets. MAGE-A1 gene expression levels, measured by qPCR, are depicted between brackets as percentage relative to housekeeping genes. Recognition was determined by IFN-γ production as measured by ELISA. Values and error bars represent mean and standard deviations of technical duplicates. (B) Cytotoxicity was assessed by a 6-hour chromium-51 release assay at E:T ratio 9:1 and 1:1. Technical triplicates are depicted in the graph and the values are a representatives of at least two independent experiments.

FIG. 15 shows pHLA-tetramer binding of MAGE-A1 TCR transgenic CD8 T cells. CD8 T cells isolated from healthy donor PBMCs were retrovirally transduced with MAGE-A1 HLA-A2 restricted TCR 4F7, or TCR D1 or a negative control CMV pp65 HLA-A2 restricted TCR. TCR D1 is TCR 1388.2b from WO 2018/170338. All TCRs contained murine TCR constant domains (mTCR). (A) Bulk TCR transduced T cells from one donor were stained with mTCR-APC antibody and MAGE-A1 pHLA Tetramer-PE on day 7 after activation and analyzed by flow cytometry. PE and APC Mean fluorescent intensities (MFI) of mTCR positive cells are depicted. (B,C) TCR transduced CD8 T cells from 3 donors were MACS enriched for transgenic TCR expression using anti-mTCR MACS on day 7 after cell activation, cells were counterstained with MAGE-A1 pHLA Tetramer-PE and analyzed by flow cytometry. (B) Overlays of MAGE-A1 pHLA-Tetramer-PE binding by MAGE A1 and CMV TCR transgenic T cells from 3 donors. Cells were gated on mTCR expression. (C) Summary of mTCR expression and pHLA-Tetramer binding of TCR D1 versus TCR 4F7 transgenic T cells (n=3). Data obtained in the same experiment as shown in (B). mTCR expression and tetramer binding was statistically compared using a paired t-test.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

Nucleic Acid Compositions that Encode Binding Protein Components

An isolated nucleic acid composition that encodes a melanoma-associated antigen (MAGE) antigen-specific binding protein having a TCR α chain variable (Vα) domain and a TCR β chain variable (Vβ) domain is provided herein, the composition comprising:
(a) a nucleic acid sequence that encodes a TCR Vα domain comprising a CDR3 amino acid sequence; and
(b) a nucleic acid sequence that encodes a TCR Vβ domain comprising a CDR3 amino acid sequence,
wherein the CDR3 sequences together specifically bind to MAGE (e.g. MAGE-A1, MAGE-A9, MAGE-A3 and/or MAGE-A6).

As would be clear to a person of skill in the art, the CDR3 amino acid sequences described herein specifically bind to their target (in this case a MAGE peptide, for example a MAGE-A1 peptide, a MAGE-A9 peptide, a MAGE-A3 peptide and/or a MAGE-A6 peptide), when the target (i.e. the appropriate MAGE peptide) is presented in the context of HLA. The binding proteins (and CDR3 sequences specifically described herein) are therefore capable of specifically binding to an appropriate MAGE peptide:HLA complex. These complexes are described in more detail elsewhere herein.

The invention provides an isolated nucleic acid composition that encodes a binding protein comprising T cell receptor (TCR) components that specifically bind a MAGE antigen (e.g. to a MAGE-A1, a MAGE-A9, a MAGE-A3 and/or a MAGE-A6 antigen). The encoded binding protein is therefore capable of specifically binding to a peptide containing a MAGE antigen (e.g. a MAGE antigen comprising an amino acid sequence selected from the group consisting of: SEQ ID NO: 127 to 134, and SEQ ID NO: 183) and does not bind to a peptide that does not contain a MAGE antigen (e.g. it does not bind to a peptide that does not contain a MAGE antigen comprising an amino acid sequence selected from the group consisting of: SEQ ID NO: 127 to 134 and SEQ ID NO: 183).

The nucleic acid composition comprises (a) a nucleic acid sequence that encodes a TCR Vα domain with the specified features described herein and (b) a nucleic acid sequence that encodes a TCR Vβ domain with the specified features described herein. The encoded TCR components form a MAGE antigen-specific binding protein.

The nucleic acid sequences of (a) and (b) above may be distinct nucleic acid sequences within the nucleic acid composition. The TCR components of the binding protein may therefore be encoded by two (or more) nucleic acid sequences (with distinct nucleotide sequences) which, together, encode all of the TCR components of the binding protein. In other words, some of the TCR components may be encoded by one nucleic acid sequence in the nucleic acid composition, and others may be encoded by another (distinct) nucleic acid sequence within the nucleic acid composition.

Alternatively, the nucleic acid sequences of (a) and (b) may be part of a single nucleic acid sequence. The TCR components of the binding protein may therefore all be encoded by a single nucleic acid sequence (for example with a single open reading frame, or with multiple (e.g. 2 or more, three or more etc.) open reading frames).

Nucleic acid sequences described herein may form part of a larger nucleic acid sequence that encodes a larger component part of a functioning binding protein. For example, a nucleic acid sequence that encodes a TCR Vα domain with the specified features described herein may be part of a larger nucleic acid sequence that encodes a functional TCR α chain (including the constant domain). As another example, a nucleic acid sequence that encodes a TCR Vβ domain with the specified features described herein may be part of a larger nucleic acid sequence that encodes a functional TCR β chain (including the constant domain). As a further example, both nucleic acid sequences (a) and (b) above may be part of a larger nucleic acid sequence that encodes a combination of a functional TCR α chain (including the constant domain) and a functional TCR β chain (including the constant domain), optionally wherein the sequence encoding the functional TCR α chain is separated from the sequence encoding the functional TCR β chain by a linker sequence that enables coordinate expression of two proteins or polypeptides in the same nucleic acid sequence. More details on this are provided below.

The nucleic acid sequences described herein may alternatively encode a small component of a T cell receptor e.g. a TCR Vα domain, or a TCR Vβ domain, only. The nucleic acid sequences may be considered as “building blocks” that provide essential components for peptide binding specificity. The nucleic acid sequences described herein may be incorporated into a distinct nucleic acid sequence (e.g. a vector) that encodes the other elements of a functional binding protein such as a TCR, such that when the nucleic acid sequence described herein is incorporated, a new nucleic acid sequence is generated that encodes e.g. a TCR α chain and/or a TCR β chain that specifically binds to a MAGE antigen (e.g, wherein the MAGE antigen comprises an amino acid sequence selected from the group consisting of: SEQ ID NO: 127 to 134 SEQ ID NO: 183). The nucleic acid sequences described herein therefore have utility as essential components that confer binding specificity for a MAGE antigen, and thus can be used to generate a larger nucleic acid sequence encoding a binding protein with the required antigen binding activity and specificity.

The nucleic acid sequences described herein may be codon optimised for expression in a host cell, for example they may be codon optimised for expression in a human cell, such as a cell of the immune system, a inducible pluripotent stem cell (iPSC), a hematopoietic stem cell, a T cell, a primary T cell, a T cell line, a NK cell, or a natural killer T cell (Scholten et al, Clin. Immunol. 119:135, 2006). The T cell can be a CD4+ or a CD8+ T cell. Codon optimisation is a well-known method in the art for maximizing expression of a nucleic acid sequence in a particular host cell. For instance, one or more cysteine residues may also be introduced into the encoded TCR alpha and beta chain components (e.g. to reduce the risk of mispairing with endogenous TCR chains).

In one example, the nucleic acid sequences described herein are codon optimised for expression in a suitable host cell, and/or are modified to introduce codons encoding one or more cysteine amino acids (e.g. into the constant domain of the encoded TCR alpha chain and/or the encoded TCR beta chain) to reduce the risk of mispairing with endogenous TCR chains. In one example, the nucleic acid sequences described herein are codon optimised for expression in a suitable host cell, optionally wherein the host cell is a human cell.

In certain examples, a TCR constant domain is modified to enhance pairing of desired TCR chains. For example, enhanced pairing between a heterologous TCR α chain and a heterologous TCR β chain due to a modification may result in the preferential assembly of a TCR comprising two heterologous chains over an undesired mispairing of a heterologous TCR chain with an endogenous TCR chain (see, e.g., Govers et al, Trends Mol. Med. 16(2):11 (2010)). Exemplary modifications to enhance pairing of heterologous TCR chains include the introduction of complementary cysteine residues in each of the heterologous TCR α chain and β chain.

A binding protein that is encoded by the nucleic acid compositions described herein is specific for a MAGE antigen and comprises MAGE antigen specific-TCR components. However, the encoded binding protein is not limited to being a TCR. Other appropriate binding proteins that comprise the specified MAGE antigen specific-TCR components are also encompassed. For example, the encoded binding protein may comprise a TCR, an antigen binding fragment of a TCR, a chimeric antigen receptor (CAR), or an immTAC. TCRs, antigen binding fragments thereof, CARs and ImmTACs are well defined in the art. A non-limiting example of an antigen binding fragment of a TCR is a single chain TCR (scTCR) or a chimeric dimer composed of the antigen binding fragments of the TCR α and TCR β chain linked to transmembrane and intracellular domains of a dimeric complex so that the complex is a chimeric dimer TCR (cdTCR). An ImmTAC comprises a TCR connected to an anti-CD3 antibody. ImmTACs are therefore bispecific, combining MAGE-recognizing TCR components with immune activating complexes.

In certain examples, an antigen-binding fragment of a TCR comprises a single chain TCR (scTCR), which comprises both the TCR Vα and TCR Vβ domains, but only a single TCR constant domain. In other examples, an antigen-binding fragment of a TCR comprises a chimeric TCR dimer in which the antigen binding fragment of the TCR is linked to an alternative transmembrane and intracellular signalling domain (where the alternative transmembrane and intracellular signalling domain are not naturally found in TCRs). In further examples, an antigen-binding fragment of a TCR or a chimeric antigen receptor is chimeric (e.g., comprises amino acid residues or motifs from more than one donor or species), humanized (e.g., comprises residues from a non-human organism that are altered or substituted so as to reduce the risk of immunogenicity in a human), or human.

“Chimeric antigen receptor” (CAR) refers to a fusion protein that is engineered to contain two or more naturally-occurring amino acid sequences linked together in a way that does not occur naturally or does not occur naturally in a host cell, which fusion protein can function as a receptor when present on a surface of a cell. CARs described herein include an extracellular portion comprising an antigen binding domain (i.e., obtained or derived from an immunoglobulin or immunoglobulin-like molecule, such as an scFv derived from an antibody or TCR specific for an antigen (e.g. a cancer antigen etc), or an antigen binding domain derived or obtained from a killer immunoreceptor from an NK cell) linked to a transmembrane domain and one or more intracellular signalling domains (optionally containing co-stimulatory domain(s)) (see, e.g., Sadelain et al, Cancer Discov., 3(4):388 (2013); see also Harris and Kranz, Trends Pharmacol. Sci., 37(3):220 (2016), and Stone et al, Cancer Immunol. Immunother., 63(11): 1163 (2014)).

Methods for producing engineered TCRs are described in, for example, Bowerman et al, Mol. Immunol, 5(15):3000 (2009). Methods for making CARs are well known in the art and are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; and Brentjens et al, 2007, Clin. Cancer Res. 73:5426.

The binding proteins described herein may also be expressed as part of a transgene construct that encodes additional accessory proteins, such as a safety switch protein, a tag, a selection marker, a CD8 co-receptor β-chain, α-chain or both, or any combination thereof.

A T cell receptor (TCR) is a molecule found on the surface of T cells (T lymphocytes) that is responsible for recognising a peptide that is bound to (presented by) a major histocompatibility complex (MHC) molecule on a target cell. The invention is directed to nucleic acid compositions that encode binding proteins comprising TCR components that interact with a particular peptide in the context of the appropriate serotype of MHC, i.e. a MAGE antigen in the context of HLA-A*02:01, HLA-C*07:02, HLA-A*01:01, HLA-A*03:01, HLA-B*07:02 and/or HLA-B*35:01 (in other words, the encoded binding protein is capable of specifically binding to a MAGE antigen: specific HLA complex). In an example, the invention is directed to nucleic acid compositions that encode binding proteins comprising TCR components that interact with a particular peptide in the context of the appropriate serotype of MHC, i.e. KVLEYVIKV (SEQ ID NO: 127) in the context of HLA-A*02:01; VRFFFPSL (SEQ ID NO: 128) in the context of HLA-C*07:02; YVGKEHMFY (SEQ ID NO: 129) in the context of HLA-A*01:01; LTQDLVQEKYLEY (SEQ ID NO: 130) in the context of HLA-A*01:01; SLFRAVITK (SEQ ID NO: 131) in the context of HLA-A*03:01; RVRFFFPSL (SEQ ID NO: 132) in the context of HLA-B*07:02; EVDPIGHLY (SEQ ID NO: 133) in the context of HLA-B*35:01; and/or EVDPIGHVY (SEQ ID NO: 134) in the context of HLA-B*35:01.

HLA-A*02:01 is a globally common human leukocyte antigen serotype within the HLA-A serotype group. Peptides that are presented by HLA-A*02:01 to TCRs are described as being “HLA-A*02:01 restricted”.

HLA-A*01:01, and HLA-B*35:01 are also common human leukocyte antigen serotypes within the HLA-A and HLA-B serotype groups. Peptides that are presented by HLA-A*01:01 to TCRs are described as being “HLA-A*01:01 restricted”. Similarly, peptides that are presented by HLA-B*35:01 to TCRs are described as being “HLA-B*35:01 restricted”.

HLA-C*07:02, HLA-A*03:01 and HLA-B*07:02 are also common human leukocyte antigen serotypes within the HLA-A, HLA-C and HLA-B serotype groups. Peptides that are presented by HLA-C*07:02 to TCRs are described as being “HLA-C*07:02 restricted”. Similarly, peptides that are presented by HLA-A*03:01 to TCRs are described as being “HLA-A*03:01 restricted”. Similarly, peptides that are presented by HLA-B*07:02 to TCRs are described as being “HLA-B*07:02 restricted”.

HLA-A*01:01 is also referred to herein as HLA-A1. Similarly, HLA-A*02:01 is also referred to herein as HLA-A2; HLA-C*07:02 is also referred to herein as HLA-C7; HLA-A*03:01 is also referred to herein as HLA-A3; HLA-B*07:02 is also referred to herein as HLA-B7; and HLA-B*35:01 is also referred to herein as HLA-B35.

As described herein, the inventors have identified several MAGE derived peptides presented on malignant cells in HLA-A*02:01, HLA-C*07:02, HLA-A*01:01, HLA-A*03:01, HLA-B*07:02 and/or HLA-B*35:01. Specifically, the inventors identified the MAGE derived peptides SEQ ID NO: 127 to 134.

Accordingly, the MAGE antigen specifically bound by a binding protein described herein may comprise an amino acid sequence selected from the group consisting of: SEQ ID NO: 127 to 134. The antigen may be an antigenic fragment (i.e. a portion) of an amino acid sequence selected from the group consisting of: SEQ ID NO: 127 to 134, it may consist of an amino acid sequence selected from the group consisting of: SEQ ID NO: 127 to 134, or it may comprise (i.e. include within a longer sequence) an amino acid sequence selected from the group consisting of: SEQ ID NO: 127 to 134.

The inventors identified that the MAGE derived peptide KVLEYVIKV (SEQ ID NO: 127) is capable of being presented by HLA-A*02:01; that the MAGE derived peptide VRFFFPSL (SEQ ID NO: 128) is capable of being presented by HLA-C*07:02; that the MAGE derived peptide YVGKEHMFY (SEQ ID NO: 129) is capable of being presented by HLA-A*01:01; that the MAGE derived peptide LTQDLVQEKYLEY (SEQ ID NO: 130) is capable of being presented by HLA-A*01:01; that the MAGE derived peptide SLFRAVITK (SEQ ID NO: 131) is capable of being presented by HLA-A*03:01; that the MAGE derived peptide RVRFFFPSL (SEQ ID NO: 132) is capable of being presented by HLA-B*07:02; that the MAGE derived peptide EVDPIGHLY (SEQ ID NO: 133) is capable of being presented by HLA-B*35:01; and that the MAGE derived peptide EVDPIGHVY (SEQ ID NO: 134) is capable of being presented by HLA-B*35:01.

Accordingly, in one example, the encoded binding protein is capable of specifically binding to a peptide:HLA complex selected from the group consisting of: a KVLEYVIKV:HLA-A*02:01 complex (SEQ ID NO: 127), a VRFFFPSL:HLA-C*07:02 complex (SEQ ID NO: 128), a YVGKEHMFY:HLA-A*01:01 complex (SEQ ID NO: 129), a LTQDLVQEKYLEY:HLA-A*01:01 complex (SEQ ID NO: 130), a SLFRAVITK:HLA-A*03:01 (SEQ ID NO: 131), a RVRFFFPSL:HLA-B*07:02 complex (SEQ ID NO: 132), a EVDPIGHLY:HLA-B*35:01 complex (SEQ ID NO: 133) and a EVDPIGHVY:HLA-B*35:01 complex (SEQ ID NO: 134).

In one example, the MAGE derived peptide of the peptide:HLA complex comprises an antigenic fragment of an amino acid sequence selected from the group consisting of: SEQ ID NO: 127 to 134. In a further example, the MAGE derived peptide of the peptide:HLA complex comprises or consists of an amino acid sequence selected from the group consisting of: SEQ ID NO: 127 to 134.

The TCR is composed of two different polypeptide chains. In humans, 95% of TCRs consist of an alpha (α) chain and a beta (β) chain (encoded by TRA and TRB respectively). When the TCR engages with a peptide in the context of HLA (e.g. in the context of HLA-A*02:01, HLA-C*07:02, HLA-A*01:01, HLA-A*03:01, HLA-B*07:02 and/or HLA-B*35:01, as appropriate), the T cell is activated through signal transduction.

The alpha and beta chains of the TCR are highly variable in sequence. Each chain is composed of two extracellular domains, a variable domain (V) and a constant domain (C). The constant domain is proximal to the T cell membrane followed by a transmembrane region and a short cytoplasmic tail while the variable domain binds to the peptide/HLA complex.

An isolated nucleic acid composition that encodes a MAGE antigen-specific binding protein is provided herein having a TCR α chain variable (Vα) domain and a TCR β chain variable (Vβ) domain. In one example the nucleic acid composition described herein may comprise a TCR α chain constant domain and/or a TCR β chain constant domain.

The variable domain of each chain has three hypervariable regions (also called complementarity determining regions (CDRs)). Accordingly, the TCR alpha variable domain (referred to herein as a TCR Vα domain, TCR V alpha domain, Vα domain or V alpha domain, alpha variable domain etc) comprises a CDR1, a CDR2 and CDR3 region. Similarly, the TCR beta variable domain (referred to herein as a TCR Vβ domain, TCR V beta domain, Vβ domain or V beta domain, beta variable domain etc) also comprises a (different) CDR1, CDR2, and CDR3 region. In each of the alpha and beta variable domains it is CDR3 that is mainly responsible for recognizing the peptide being presented by the HLA molecules.

As will be clear to a person of skill in the art, the phrase “TCR α chain variable domain” refers to the variable (V) domain (extracellular domain) of a TCR alpha chain, and thus includes three hypervariable regions (CDR1, CDR2 and the specified CDR3), as well as the intervening sequences, but does not include the constant (C) domain of the alpha chain, which does not form part of the variable domain.

As will be clear to a person of skill in the art, the phrase “TCR β chain variable domain” refers to the variable (V) domain (extracellular domain) of a TCR beta chain, and thus includes three hypervariable regions (CDR1, CDR2 and the specified CDR3), as well as the intervening sequences, but does not include the constant (C) domain of the beta chain, which does not form part of the variable domain.

### TCR Components

The isolated nucleic acid composition described herein encodes a MAGE antigen-specific binding protein. As discussed herein, the inventors have identified several TCRs that specifically bind to a MAGE antigen selected from KVLEYVIKV (SEQ ID NO: 127), VRFFFPSL (SEQ ID NO: 128), YVGKEHMFY (SEQ ID NO: 129), LTQDLVQEKYLEY (SEQ ID NO: 130), SLFRAVITK (SEQ ID NO: 131), RVRFFFPSL (SEQ ID NO: 132), EVDPIGHLY (SEQ ID NO: 133) and EVDPIGHVY (SEQ ID NO: 134).

(i) TCR Components that Interact with KVLEYVIKV (SEQ ID NO: 127)

As provided elsewhere herein, the inventors identified TCR clone CT27.4F7 (4F7) which interacts with KVLEYVIKV (SEQ ID NO: 127) in the context of HLA-A*02:01. The sequences provided herein that correspond to TCR clone CT27.4F7 are SEQ ID NO:s 1 to 14.

In one embodiment, an isolated nucleic acid composition that encodes a MAGE antigen-specific binding protein having a TCR α chain variable (Vα) domain and a TCR β chain variable (Vβ) domain is provided, the composition comprising:
a nucleic acid sequence that encodes a TCR Vα domain comprising a CDR3 amino acid sequence having at least 80% sequence identity to SEQ ID NO:3, or a functional fragment thereof; and a nucleic acid sequence that encodes a TCR Vβ domain comprising a CDR3 amino acid sequence having at least 80% sequence identity to SEQ ID NO:6, or a functional fragment thereof, wherein the CDR3 sequences together specifically bind to MAGE-A1.

An example of an appropriate TCR Vα domain CDR3 amino acid sequence that confers specific binding to a MAGE antigen, in particular to a MAGE-A1 antigen (e.g. to KVLEYVIKV (SEQ ID NO: 127)), is shown in SEQ ID NO:3. As would be clear to a person of skill in the art, variants of the amino acid sequence shown in SEQ ID NO:3 may also be functional (i.e. retain their ability to confer specific binding to a MAGE-A1 antigen (e.g. to the peptide KVLEYVIKV (SEQ ID NO: 127)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 3, i.e. they may have at least 80%, at least 81%, at least 90%, or 100% sequence identity to SEQ ID NO: 3. Suitably, percent identity is calculated as the percentage of identity to the entire length of the reference sequence (e.g. SEQ ID NO:3). In other words, appropriate (functional) Vα domain CDR3 amino acid sequences may vary from the sequence shown in SEQ ID NO:3 by one or several (e.g. two etc) amino acids.

As stated above, functional variants of SEQ ID NO:3 retain their ability to confer specific binding to a MAGE-A1 antigen (e.g. the peptide shown in SEQ ID NO: 127) when the CDR3 is part of TCR Vα domain.

Functional variants may be naturally occurring, synthetic, or synthetically improved functional variants of SEQ ID NO:3. The term “variant” also encompasses homologues and fragments. Functional variants will typically contain only conservative substitutions of one, two or more amino acids of SEQ ID NO:3, or substitution, deletion or insertion of non-critical amino acids in non-critical regions of the CDR3.

Non-functional variants are amino acid sequence variants of SEQ ID NO: 3 that do not specifically bind to a MAGE-A1 antigen (e.g. the peptide shown in SEQ ID NO: 127). Non-functional variants will typically contain a non-conservative substitution, a deletion, or insertion or premature truncation of the amino acid sequence of SEQ ID NO:3 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 3. In examples where the TCR Vα domain CDR3 has the amino acid sequence of SEQ ID NO:3, the CDR3 may be encoded by any appropriate nucleic acid sequence.

The encoded TCR Vα domain may comprise, in addition to the specified CDR3, a CDR1 comprising an amino acid sequence of SEQ ID NO: 1, or a functional variant thereof (i.e, wherein the variant retains the ability to specifically bind to the MAGE-A1 antigen (e.g. the peptide shown in SEQ ID NO:127)). Such functional variants may be naturally occurring, synthetic, or synthetically improved functional variants of SEQ ID NO:1. The term “variant” also encompasses homologues and fragments. Functional variants will typically contain only conservative substitutions of one or more amino acids of SEQ ID NO:1, or substitution, deletion or insertion of non-critical amino acids in non-critical regions of the protein.

Non-functional variants are amino acid sequence variants of SEQ ID NO: 1 that do not specifically bind to a MAGE-A1 antigen (e.g. the peptide shown in SEQ ID NO: 127). Non-functional variants will typically contain a non-conservative substitution, a deletion, or insertion or premature truncation of the amino acid sequence of SEQ ID NO: 1 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 1, i.e. it may have at least 80%, at least 83%, or 100% sequence identity to SEQ ID NO: 1. Suitably, percent identity is calculated as the percentage of identity to the entire length of the reference sequence (e.g. SEQ ID NO:1). In other words, appropriate functional Vα domain CDR1 amino acid sequences may vary from the sequence shown in SEQ ID NO: 1 by one or several amino acids. As stated previously, the variant may comprise an amino acid substitution such as a conservative amino acid substitution compared to the sequence shown in SEQ ID NO:1. As stated above, functional variants of SEQ ID NO: 1 retain the ability to specifically bind to a MAGE-A1 antigen (e.g. the peptide shown in SEQ ID NO: 127) when the CDR1 is part of TCR Vα domain).

In one example, the CDR1 of the Vα domain comprises or consists of the amino acid sequence of SEQ ID NO: 1. In examples where the TCR Vα domain CDR1 has the amino acid sequence of SEQ ID NO:1, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 2, or a functional variant thereof (i.e, wherein the variant retains the ability to specifically bind to HLA-A*02:01). Such functional variants may be naturally occurring, synthetic, or synthetically improved functional variants of SEQ ID NO:2. The term “variant” also encompasses homologues and fragments. Functional variants will typically contain only conservative substitutions of one or more amino acids of SEQ ID NO:2, or substitution, deletion or insertion of non-critical amino acids in non-critical regions of the protein.

Non-functional variants are amino acid sequence variants of SEQ ID NO: 2 that do not specifically bind to HLA-A*02:01. Non-functional variants will typically contain a non-conservative substitution, a deletion, or insertion or premature truncation of the amino acid sequence of SEQ ID NO: 2 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 2, i.e. it may have at least 80%, at least 85%, or 100% sequence identity to SEQ ID NO: 2. Suitably, percent identity is calculated as the percentage of identity to the entire length of the reference sequence (e.g. SEQ ID NO:2). In other words, appropriate (functional) Vα domain CDR2 amino acid sequences may vary from the sequence shown in SEQ ID NO:2 by one or several amino acids. As stated previously, the variant may comprise an amino acid substitution such as a conservative amino acid substitution compared to the sequence shown in SEQ ID NO:2). As stated above, a functional variant of SEQ ID NO: 2 retains the ability to specifically bind to HLA-A*02:01.

In one example, the CDR2 of the Vα domain comprises or consists of the amino acid sequence of SEQ ID NO: 2. In examples where the TCR Vα domain CDR2 has the amino acid sequence of SEQ ID NO:2, the CDR2 may be encoded by any appropriate nucleic acid sequence.

The encoded TCR Vα domain may therefore comprise the CDRs mentioned in detail above (by SEQ ID specifically i.e. SEQ ID NO:3, SEQ ID NO: 1 and SEQ ID NO: 2, or functional variants thereof), with appropriate intervening sequences between the CDRs.

The encoded TCR Vα domain may comprise an amino acid sequence of SEQ ID NO:7, or a functional variant thereof (i.e, wherein the variant TCR Vα domain retains the ability to specifically bind to a MAGE-A1 antigen (e.g. the peptide shown in SEQ ID NO: 127) when part of a binding protein described herein). Such functional variants may be naturally occurring, synthetic, or synthetically improved functional variants of SEQ ID NO:7. The term “variant” also encompasses homologues and fragments. Functional variants will typically contain only conservative substitutions of one or more amino acids of SEQ ID NO:7, or substitution, deletion or insertion of non-critical amino acids in non-critical regions of the protein.

Non-functional variants are amino acid sequence variants of SEQ ID NO: 7 that do not specifically bind to a MAGE-A1 antigen (e.g. the peptide shown in SEQ ID NO: 127). Non-functional variants will typically contain a non-conservative substitution, a deletion, or insertion or premature truncation of the amino acid sequence of SEQ ID NO:7 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 7, whilst retaining the ability to specifically bind to a MAGE-A1 antigen (e.g. the peptide shown in SEQ ID NO: 127). In other words, a functional TCR Vα domain with one or several amino acid substitutions compared to the sequence of SEQ ID NO:7 is also encompassed. As stated previously, the amino acid substitution may be a conservative amino acid substitution. The variability in sequence compared to SEQ ID NO:7 may all be in regions of the TCR Vα domain that do not form CDRs (i.e. the variant may have the CDRs of SEQ ID NO: 3, SEQ ID NO: 1 and/or SEQ ID NO: 2, and still have 25% (or less) sequence variability compared to SEQ ID NO:7). In other words, the sequence of the CDRs of SEQ ID NO: 7 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 7).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 7, wherein the TCR Vα domain comprises a CDR3 having an amino acid sequence of SEQ ID NO: 3. In this example, the TCR Vα domain CDR1 may have an amino acid sequence of SEQ ID NO: 1 and the TCR Vα domain CDR2 may have an amino acid sequence of SEQ ID NO: 2.

As another example, the encoded TCR Vα domain may comprise an amino acid sequence having the amino acid sequence of SEQ ID NO: 7, with 0 to 10 (or 0 to 5) amino acid substitutions, insertions or deletions), wherein the TCR Vα domain comprises a CDR3 having an amino acid sequence of SEQ ID NO: 3. In this example, the TCR Vα domain CDR1 may have an amino acid sequence of SEQ ID NO: 1 and the TCR Vα domain CDR2 may have an amino acid sequence of SEQ ID NO: 2.

In examples where the TCR Vα domain has the amino acid sequence of SEQ ID NO:7, the TCR Vα domain may be encoded by the nucleic acid sequence of SEQ ID NO:8, or a genetically degenerate sequence thereof (i.e. other nucleic acid sequences that encode the same protein as a result of the degeneracy of the genetic code).

The phrase “genetically degenerate sequence thereof” is used interchangeably with “derivative thereof” herein.

For the avoidance of doubt, the nucleic acid sequence encoding the TCR Vα domain may also encode a TCR α chain constant domain. An example of a suitable constant domain (for either a TCR α chain or a TCR β chain) is encoded in the MP71-TCR-flex retroviral vector. However, the invention is not limited to this specific constant domain, and encompasses any appropriate TCR α chain constant domain. The constant domain may be murine derived, human derived or humanised. Methods for identifying or generating appropriate constant domains are well known to a person of skill in the art and are well within their routine capabilities.

By way of example only, the constant domain may be encoded by or derived from a vector, such as a lentiviral, retroviral or plasmid vector but also adenovirus, adeno-associated virus, vaccinia virus, canary poxvirus or herpes virus vectors in which murine or human constant domains are pre-cloned. Recently, minicircles have also been described for TCR gene transfer (non-viral Sleeping Beauty transposition from minicircle vectors as published by R Monjezi, et al., 2017). Moreover, naked (synthetic) DNA/RNA can also be used to introduce the TCR. As an example, a pMSGV retroviral vector with pre-cloned TCR-Ca and Cb genes as described in L V Coren et al., BioTechniques 2015 may be used to provide an appropriate constant domain. Alternatively, single stranded or double stranded DNA or RNA can be inserted by homologous directed repair into the TCR locus (see Roth et al 2018 Nature vol 559; page 405). As a further option, non-homologous end joining is possible.

An example of a specific TCR α chain amino acid sequence that includes a TCR Vα domain described herein with an appropriate constant domain is shown in SEQ ID NO: 11. Appropriate functional variants of SEQ ID NO: 11 are also encompassed (e.g. variants having at least 75% (e.g. at least 75%, at least 80%, at least 85%, at least 90%, at least 95% etc) sequence identity to the amino acid sequence of SEQ ID NO: 11, wherein the variant TCR α chain amino acid sequence retains its ability to specifically bind to a MAGE-A1 antigen (e.g. the peptide shown in SEQ ID NO:127) when part of a binding protein described herein). In other words, a functional TCR α chain with one or several amino acid substitutions compared to the sequence of SEQ ID NO: 11 is also encompassed. As stated previously, the amino acid substitution may be a conservative amino acid substitution. The variability in sequence compared to SEQ ID NO: 11 may all be in regions of the TCR α chain that do not form CDRs (i.e. the variant may have the CDRs of SEQ ID NO: 3, SEQ ID NO: 1 and/or SEQ ID NO: 2, and still have 25% (or less) sequence variability compared to SEQ ID NO:11). In other words, the sequence of the CDRs of SEQ ID NO: 11 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 11).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 11, wherein the TCR α chain comprises a CDR3 having an amino acid sequence of SEQ ID NO: 3. In this example, the TCR α chain CDR1 may have an amino acid sequence of SEQ ID NO: 1 and the TCR α chain CDR2 may have an amino acid sequence of SEQ ID NO: 2.

In examples where the TCR α chain has the amino acid sequence of SEQ ID NO: 11, the TCR α chain may be encoded by the nucleic acid sequence of SEQ ID NO: 12, or a genetically degenerate sequence thereof (i.e. other nucleic acid sequences that encode the same protein as a result of the degeneracy of the genetic code). It is noted that SEQ ID NO: 12 is the nucleic acid sequence for TCR α chain of clone CT27.4F7 (4F7).

In one example, the nucleic acid composition provided herein comprises a nucleic acid sequence that encodes a TCR Vα domain comprising a CDR3 amino acid sequence having at least 80% sequence identity to SEQ ID NO:3, or a functional fragment thereof.

In another example, the CDR3 of the Vα domain of a nucleic acid composition provided herein comprises or consists of the amino acid sequence of SEQ ID NO: 3.

In another example, the Vα domain of a nucleic acid composition provided herein comprises an amino acid sequence having at least 80% sequence identity to, comprising, or consisting of, SEQ ID NO: 7.

As provided above, the inventors identified TCR clone CT27.4F7 (4F7) which interacts with KVLEYVIKV (SEQ ID NO: 127) in the context of HLA-A*02:01. The sequences provided herein that correspond to TCR clone CT27.4F7 are SEQ ID NO:s 1 to 14.

Accordingly, an example of an appropriate TCR Vβ domain CDR3 amino acid sequence that confers specific binding to a MAGE antigen, in particular to a MAGE-A1 antigen (e.g. to KVLEYVIKV (SEQ ID NO: 127)), is shown in SEQ ID NO:6. As would be clear to a person of skill in the art, variants of the amino acid sequence shown in SEQ ID NO:6 may also be functional (i.e. retain their ability to confer specific binding to a MAGE-A1 antigen (e.g. the peptide shown in SEQ ID NO: 127) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 6, i.e. they may have at least 80%, at least 83%, at least 91%, or 100% sequence identity to SEQ ID NO: 6. Suitably, percent identity is calculated as the percentage of identity to the entire length of the reference sequence (e.g. SEQ ID NO: 6). In other words, appropriate (functional) Vβ domain CDR3 amino acid sequences may vary from the sequence shown in SEQ ID NO: 6 by one or several (e.g. two) amino acids. As stated above, functional variants of SEQ ID NO: 6 retain their ability to confer specific binding to a MAGE-A1 antigen (e.g. the peptide shown in SEQ ID NO: 127) when the CDR3 is part of TCR Vβ domain.

Functional variants may be naturally occurring, synthetic, or synthetically improved functional variants of SEQ ID NO: 6. The term “variant” also encompasses homologues and fragments. Functional variants will typically contain only conservative substitutions of one or more amino acids of SEQ ID NO: 6, or substitution, deletion or insertion of non-critical amino acids in non-critical regions of the CDR3.

Non-functional variants are amino acid sequence variants of SEQ ID NO: 6 that do not specifically bind to a MAGE-A1 antigen (e.g. the peptide shown in SEQ ID NO: 127). Non-functional variants will typically contain a non-conservative substitution, a deletion, or insertion or premature truncation of the amino acid sequence of SEQ ID NO: 6 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 6. In examples where the TCR Vβ domain CDR3 has the amino acid sequence of SEQ ID NO:6, the CDR3 may be encoded by any appropriate nucleic acid sequence.

The encoded TCR Vβ domain may comprise, in addition to the specified CDR3, a CDR1 comprising an amino acid sequence of SEQ ID NO: 4, or a functional variant thereof (i.e, wherein the variant retains the ability to specifically bind to a MAGE-A1 antigen (e.g. the peptide shown in SEQ ID NO: 127)). Such functional variants may be naturally occurring, synthetic, or synthetically improved functional variants of SEQ ID NO: 4. The term “variant” also encompasses homologues and fragments. Functional variants will typically contain only conservative substitutions of one or more amino acids of SEQ ID NO: 4, or substitution, deletion or insertion of non-critical amino acids in non-critical regions of the protein.

Non-functional variants are amino acid sequence variants of SEQ ID NO: 4 that do not specifically bind to a MAGE-A1 antigen (e.g. the peptide shown in SEQ ID NO: 127). Non-functional variants will typically contain a non-conservative substitution, a deletion, or insertion or premature truncation of the amino acid sequence of SEQ ID NO: 4 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 4, i.e. it may have at least 80%, or 100% sequence identity to SEQ ID NO: 4. Suitably, percent identity is calculated as the percentage of identity to the entire length of the reference sequence (e.g. SEQ ID NO: 4). In other words, appropriate (functional) Vβ domain CDR1 amino acid sequences may vary from the sequence shown in SEQ ID NO:4 by one or several amino acids. As stated previously, the variant may comprise an amino acid substitution such as a conservative amino acid substitution compared to the sequence shown in SEQ ID NO:4). As stated above, functional variants of SEQ ID NO: 4 retain the ability to specifically bind to a MAGE-A1 antigen (e.g. the peptide shown in SEQ ID NO: 127) when the CDR1 is part of TCR Vβ domain).

In one example, the CDR1 of the Vβ domain comprises or consists of the amino acid sequence of SEQ ID NO: 4. In examples where the TCR Vα domain CDR1 has the amino acid sequence of SEQ ID NO:4, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 5, or a functional variant thereof (i.e, wherein the variant retains the ability to specifically bind to HLA-A*02:01). Such functional variants may be naturally occurring, synthetic, or synthetically improved functional variants of SEQ ID NO: 5. The term “variant” also encompasses homologues and fragments. Functional variants will typically contain only conservative substitutions of one or more amino acids of SEQ ID NO: 5, or substitution, deletion or insertion of non-critical amino acids in non-critical regions of the protein.

Non-functional variants are amino acid sequence variants of SEQ ID NO: 5 that do not specifically bind to HLA-A*02:01. Non-functional variants will typically contain a non-conservative substitution, a deletion, or insertion or premature truncation of the amino acid sequence of SEQ ID NO: 5 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 5, i.e. it may have at least 80%, at least 83%, or 100% sequence identity to SEQ ID NO: 5. Suitably, percent identity is calculated as the percentage of identity to the entire length of the reference sequence (e.g. SEQ ID NO: 5). In other words, appropriate (functional) Vβ domain CDR2 amino acid sequences may vary from the sequence shown in SEQ ID NO: 5 by one or several amino acids. As stated previously, the variant may comprise an amino acid substitution such as a conservative amino acid substitution compared to the sequence shown in SEQ ID NO: 5. As stated above, a functional variant of SEQ ID NO: 5 retains the ability to specifically bind to HLA-A*02:01.

In one example, the CDR2 of the Vβ domain comprises or consists of the amino acid sequence of SEQ ID NO: 5. In examples where the TCR Vβ domain CDR2 has the amino acid sequence of SEQ ID NO:5, the CDR2 may be encoded by any appropriate nucleic acid sequence.

The encoded TCR Vβ domain may therefore comprise the CDRs mentioned in detail above (by SEQ ID specifically i.e. SEQ ID NO:6, SEQ ID NO: 4 and SEQ ID NO: 5, or functional variants thereof), with appropriate intervening sequences between the CDRs.

The encoded TCR Vβ domain may have an amino acid sequence of SEQ ID NO: 9, or a functional variant thereof (i.e, wherein the variant TCR Vβ domain retains the ability to specifically bind to a MAGE-A1 antigen (e.g. the peptide shown in SEQ ID NO: 127) when part of a binding protein described herein). Such functional variants may be naturally occurring, synthetic, or synthetically improved functional variants of SEQ ID NO: 9. The term “variant” also encompasses homologues and fragments. Functional variants will typically contain only conservative substitutions of one or more amino acids of SEQ ID NO: 9, or substitution, deletion or insertion of non-critical amino acids in non-critical regions of the protein.

Non-functional variants are amino acid sequence variants of SEQ ID NO: 9 that do not specifically bind to a MAGE-A1 antigen (e.g. the peptide shown in SEQ ID NO: 127). Non-functional variants will typically contain a non-conservative substitution, a deletion, or insertion or premature truncation of the amino acid sequence of SEQ ID NO:9 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 9, whilst retaining the ability to specifically bind to a MAGE-A1 antigen (e.g. the peptide shown in SEQ ID NO: 127). In other words, a functional TCR Vβ domain with one or several amino acid substitutions compared to the sequence of SEQ ID NO: 9 is also encompassed. As stated previously, the amino acid substitution may be a conservative amino acid substitution. The variability in sequence compared to SEQ ID NO:9 may all be in regions of the TCR Vβ domain that do not form CDRs (i.e. the variant may have the CDRs of SEQ ID NO: 6, SEQ ID NO: 4 and/or SEQ ID NO: 5, and still have 25% (or less) sequence variability compared to SEQ ID NO: 9). In other words, the sequence of the CDRs of SEQ ID NO: 9 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 9).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 9, wherein the TCR Vβ domain comprises a CDR3 having an amino acid sequence of SEQ ID NO: 6. In this example, the TCR Vβ domain CDR1 may have an amino acid sequence of SEQ ID NO:4 and the TCR Vβ domain CDR2 may have an amino acid sequence of SEQ ID NO: 5.

In examples where the TCR Vβ domain has the amino acid sequence of SEQ ID NO:9, the TCR Vβ domain may be encoded by the nucleic acid sequence of SEQ ID NO: 10, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 13. Appropriate functional variants of SEQ ID NO: 13 are also encompassed (e.g. variants having at least 75% (e.g. at least 75%, at least 80%, at least 85%, at least 90%, at least 95% etc) sequence identity to the amino acid sequence of SEQ ID NO: 13, wherein the variant TCR β chain amino acid sequence retains its ability to specifically bind to a MAGE-A1 antigen (e.g. the peptide shown in SEQ ID NO: 127) when part of a binding protein described herein). In other words, a functional TCR β chain with one or several amino acid substitutions compared to the sequence of SEQ ID NO: 13 is also encompassed. As stated previously, the amino acid substitution may be a conservative amino acid substitution. The variability in sequence compared to SEQ ID NO: 13 may all be in regions of the TCR β chain that do not form CDRs (i.e. the variant may have the CDRs of SEQ ID NO: 6, SEQ ID NO: 4 and/or SEQ ID NO: 5, and still have 25% (or less) sequence variability compared to SEQ ID NO:13). In other words, the sequence of the CDRs of SEQ ID NO: 13 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 13).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 13, wherein the TCR β chain comprises a CDR3 having an amino acid sequence of SEQ ID NO: 6. In this example, the TCR β chain CDR1 may have an amino acid sequence of SEQ ID NO: 4 and the TCR β chain CDR2 may have an amino acid sequence of SEQ ID NO: 5.

In examples where the TCR β chain has the amino acid sequence of SEQ ID NO: 13, the TCR β chain may be encoded by the nucleic acid sequence of SEQ ID NO: 14, or a genetically degenerate sequence thereof (i.e. other nucleic acid sequences that encode the same protein as a result of the degeneracy of the genetic code). It is noted that SEQ ID NO: 14 is the nucleic acid sequence for TCR β chain of clone CT27.4F7 (4F7).

In an example, the nucleic acid composition provided herein comprises a nucleic acid sequence that encodes a TCR Vβ domain comprising a CDR3 amino acid sequence having at least 80% sequence identity to SEQ ID NO:6, or a functional fragment thereof.

In another example, the CDR3 of the Vβ domain of a nucleic acid composition provided herein comprises or consists of the amino acid sequence of SEQ ID NO:6.

In a further example, the Vβ domain of a nucleic acid composition provided herein comprises an amino acid sequence having at least 80% sequence identity to, comprising, or consisting of, SEQ ID NO: 9.

The TCR Vβ domain sequences derived from TCR clone CT27.4F7 discussed above are particularly compatible with the TCR Vα domain sequences derived from TCR clone CT27.4F7 discussed elsewhere herein.

Accordingly, in one example, a nucleic acid composition described herein encodes a MAGE-A1 antigen-specific binding protein having TCR Vα domain comprising a CDR3 amino acid sequence having at least 80% sequence identity to SEQ ID NO:3, or a functional fragment thereof; and a nucleic acid sequence that encodes a TCR Vβ domain comprising a CDR3 amino acid sequence having at least 80% sequence identity to SEQ ID NO:6, or a functional fragment thereof.

In a particular example, a nucleic acid composition described herein encodes a MAGE-A1 antigen-specific binding protein having a TCR Vα domain with a CDR3 amino acid sequence comprising or consisting of the amino acid sequence of SEQ ID NO: 3; and a TCR Vβ domain with a CDR3 comprising or consisting of the amino acid sequence of SEQ ID NO:6. In addition, the MAGE-A1 antigen may comprise or consist of the sequence shown in SEQ ID NO: 127.

Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 7; and the Vβ domain may comprise an amino acid sequence having at least 80% sequence identity to, comprising, or consisting of, SEQ ID NO: 9. In one example, the Vα domain comprises the amino acid sequence of SEQ ID NO: 7 and the Vβ domain comprises the amino acid sequence of SEQ ID NO: 9. In such cases, the Vα domain may be encoded by a nucleic acid sequence comprising the sequence of SEQ ID NO: 8; and the Vβ domain may be encoded by a nucleic acid sequence comprising the sequence of SEQ ID NO: 10.

In this particular example, the TCR Vα domain may include a CDR1 amino acid sequence comprising or consisting of the amino acid sequence of SEQ ID NO: 1 and a CDR2 amino acid sequence comprising or consisting of the amino acid sequence of SEQ ID NO:2. Furthermore, the TCR Vβ domain may include a CDR1 amino acid sequence comprising or consisting of the amino acid sequence of SEQ ID NO:4 and a CDR2 amino acid sequence comprising or consisting of the amino acid sequence of SEQ ID NO: 5.

For the avoidance of doubt, this particular example encompasses components of TCR clone CT27.4F7 exemplified herein. The different components of TCR clone CT27.4F7 and their respective SEQ ID Nos are summarised in Table 6 below.

As stated in more detail elsewhere herein, the nucleic acid composition described herein encodes both a TCR Vα domain and a TCR Vβ domain, which form the binding protein that is capable of specifically binding to a MAGE antigen. In examples where the TCR Vα domain and the TCR Vβ domain are encoded by the same nucleic acid sequence, the TCR Vα domain and TCR Vβ domain may be joined together via a linker, e.g. a linker that enables expression of two proteins or polypeptides from the same vector. By way of example, a linker comprising a porcine teschovirus-1 2A (P2A) sequence may be used, such as 2A sequences from foot-and-mouth disease virus (F2A), equine rhinitis A virus (E2A) or Thosea asigna virus (T2A) as published by A. L. Szymczak et al., Nature Biotechnology 22, 589-594 (2004) or 2A-like sequences. 2A and 2A-like sequences are linkers that are cleavable once the nucleic acid molecule has been transcribed and translated. Another example of a linker is an internal ribosomal entry sites (IRES) which enables translation of two proteins or polypeptides from the same transcript. Any other appropriate linker may also be used. As a further example, the nucleic acid sequence encoding the TCR Vα domain and nucleic acid sequence encoding the TCR Vβ domain may be cloned into a vector with dual internal promoters (see e.g. S Jones et al., Human Gene Ther 2009). The identification of appropriate linkers and vectors that enable expression of both the TCR Vα domain and the TCR Vβ domain is well within the routine capabilities of a person of skill in the art.

Additional appropriate polypeptide domains may also be encoded by the nucleic acid sequences that encode the TCR Vα domain and/or the TCR Vβ domain. By way of example only, the nucleic acid sequence may comprise a membrane targeting sequence that provides for transport of the encoded polypeptide to the cell surface membrane of the modified cell. Other appropriate additional domains are well known and are described, for example, in WO2016/071758.

In one example, the nucleic acid composition described herein may encode a soluble TCR. For example, the nucleic acid composition may encode the variable domain of the TCR alpha and beta chains respectively together with an immune-modulator molecule such as a CD3 agonist (e.g. an anti-CD3 scFv). The CD3 antigen is present on mature human T cells, thymocytes and a subset of natural killer cells. It is associated with the TCR and is involved in signal transduction of the TCR. Antibodies specific for the human CD3 antigen are well known. One such antibody is the murine monoclonal antibody OKT3, which is the first monoclonal antibody approved by the FDA. Other antibodies specific for CD3 have also been reported (see e.g. WO2004/106380; U.S. Patent Application Publication No. 2004/0202657; U.S. Pat. No. 6,750,325). Immune mobilising mTCR Against Cancer (ImmTAC; Immunocore Limited, Milton Partk, Abington, Oxon, United Kingdom) are bifunctional proteins that combine affinity monoclonal T cell receptor (mTCR) targeting with a therapeutic mechanism of action (i.e., an anti-CD3 scFv). In another example, a soluble TCR of the invention may be combined with a radioisotope or a toxic drug. Appropriate radioisotopes and/or toxic drugs are well known in the art and are readily identifiable by a person of ordinary skill in the art.

In one example, the nucleic acid composition may encode a chimeric single chain TCR wherein the TCR alpha chain variable domain is linked to the TCR beta chain variable domain and a constant domain which is e.g. fused to the CD3 zeta signalling domain. In this example, the linker is non-cleavable. In an alternative embodiment, the nucleic acid composition may encode a chimeric two chain TCR in which the TCR alpha chain variable domain and the TCR beta chain variable domain are each linked to a CD3 zeta signalling domain or other transmembrane and intracellular domains. Methods for preparing such single chain TCRs and two chain TCRs are well known in the art; see for example RA Willemsen et al, Gene Therapy 2000.

(ii) TCR Components that Interact with VRFFFPSL (SEQ ID NO: 128)

As provided elsewhere herein, the inventors have also identified TCR clone CT31.10C1 (10C1) which interacts with VRFFFPSL (SEQ ID NO: 128) in the context of HLA-C*07:02. The sequences provided herein that correspond to TCR clone CT31.10C1 are SEQ ID NO:s 15 to 28.

In one embodiment, an isolated nucleic acid composition that encodes a MAGE antigen-specific binding protein having a TCR α chain variable (Vα) domain and a TCR β chain variable (Vβ) domain is provided, the composition comprising:
a nucleic acid sequence that encodes a TCR Vα domain comprising a CDR3 amino acid sequence having at least 80% sequence identity to SEQ ID NO:17, or a functional fragment thereof; and a nucleic acid sequence that encodes a TCR Vβ domain comprising a CDR3 amino acid sequence having at least 80% sequence identity to SEQ ID NO: 20, or a functional fragment thereof, wherein the CDR3 sequences together specifically bind to MAGE-A1.

Accordingly, another example of an appropriate TCR Vα domain CDR3 amino acid sequence that confers specific binding to a MAGE antigen, in particular a MAGE-A1 antigen (e.g. to VRFFFPSL (SEQ ID NO: 128)), is shown in SEQ ID NO: 17. As would be clear to a person of skill in the art, variants of the amino acid sequence shown in SEQ ID NO:17 may also be functional (i.e. retain their ability to confer specific binding to a MAGE-A1 antigen (e.g. to the peptide VRFFFPSL (SEQ ID NO: 128)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 17, i.e. they may have at least 80%, at least 83%, at least 91%, or 100% sequence identity to SEQ ID NO:17. Suitably, percent identity is calculated as the percentage of identity to the entire length of the reference sequence (e.g. SEQ ID NO:17). In other words, appropriate (functional) Vα domain CDR3 amino acid sequences may vary from the sequence shown in SEQ ID NO:17 by one or several (e.g. two etc) amino acids.

As stated above, functional variants of SEQ ID NO:17 retain their ability to confer specific binding to a MAGE-A1 antigen (e.g. the peptide shown in SEQ ID NO: 128) when the CDR3 is part of TCR Vα domain.

Functional variants may be naturally occurring, synthetic, or synthetically improved functional variants of SEQ ID NO: 17. The term “variant” also encompasses homologues and fragments.

Functional variants will typically contain only conservative substitutions of one, two or more amino acids of SEQ ID NO: 17, or substitution, deletion or insertion of non-critical amino acids in non-critical regions of the CDR3.

Non-functional variants are amino acid sequence variants of SEQ ID NO:17 that do not specifically bind to a MAGE-A1 antigen (e.g. to the peptide shown in SEQ ID NO: 128). Non-functional variants will typically contain a non-conservative substitution, a deletion, or insertion or premature truncation of the amino acid sequence of SEQ ID NO: 17 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 17. In examples where the TCR Vα domain CDR3 has the amino acid sequence of SEQ ID NO: 17, the CDR3 may be encoded by any appropriate nucleic acid sequence.

The encoded TCR Vα domain may comprise, in addition to the specified CDR3, a CDR1 comprising an amino acid sequence of SEQ ID NO: 15, or a functional variant thereof (i.e, wherein the variant retains the ability to specifically bind to a MAGE-A1 antigen (e.g. the peptide shown in SEQ ID NO: 128)). Such functional variants may be naturally occurring, synthetic, or synthetically improved functional variants of SEQ ID NO: 15. The term “variant” also encompasses homologues and fragments. Functional variants will typically contain only conservative substitutions of one or more amino acids of SEQ ID NO:15, or substitution, deletion or insertion of non-critical amino acids in non-critical regions of the protein.

Non-functional variants are amino acid sequence variants of SEQ ID NO: 15 that do not specifically bind to a MAGE-A1 antigen (e.g. the peptide shown in SEQ ID NO:128). Non-functional variants will typically contain a non-conservative substitution, a deletion, or insertion or premature truncation of the amino acid sequence of SEQ ID NO: 15 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 15, i.e. it may have at least 80%, at least 83%, or 100% sequence identity to SEQ ID NO: 15. Suitably, percent identity is calculated as the percentage of identity to the entire length of the reference sequence (e.g. SEQ ID NO: 15). In other words, appropriate functional Vα domain CDR1 amino acid sequences may vary from the sequence shown in SEQ ID NO: 15 by one or several amino acids. As stated previously, the variant may comprise an amino acid substitution such as a conservative amino acid substitution compared to the sequence shown in SEQ ID NO: 15. As stated above, functional variants of SEQ ID NO: 15 retain the ability to specifically bind to a MAGE-A1 antigen (e.g. the peptide shown in SEQ ID NO: 128) when the CDR1 is part of TCR Vα domain).

In one example, the CDR1 of the Vα domain comprises or consists of the amino acid sequence of SEQ ID NO: 15. In examples where the TCR Vα domain CDR1 has the amino acid sequence of SEQ ID NO: 15, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 16, or a functional variant thereof (i.e, wherein the variant retains the ability to specifically bind to HLA-C*07:02). Such functional variants may be naturally occurring, synthetic, or synthetically improved functional variants of SEQ ID NO: 16. The term “variant” also encompasses homologues and fragments. Functional variants will typically contain only conservative substitutions of one or more amino acids of SEQ ID NO: 16, or substitution, deletion or insertion of non-critical amino acids in non-critical regions of the protein.

Non-functional variants are amino acid sequence variants of SEQ ID NO:16 that do not specifically bind to HLA-C*07:02. Non-functional variants will typically contain a non-conservative substitution, a deletion, or insertion or premature truncation of the amino acid sequence of SEQ ID NO: 16 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 16, i.e. it may have at least 80%, at least 88%, or 100% sequence identity to SEQ ID NO:16. Suitably, percent identity is calculated as the percentage of identity to the entire length of the reference sequence (e.g. SEQ ID NO: 16). In other words, appropriate (functional) Vα domain CDR2 amino acid sequences may vary from the sequence shown in SEQ ID NO: 16 by one or several amino acids. As stated previously, the variant may comprise an amino acid substitution such as a conservative amino acid substitution compared to the sequence shown in SEQ ID NO: 16). As stated above, a functional variant of SEQ ID NO: 16 retains the ability to specifically bind to HLA-C*07:02.

In one example, the CDR2 of the Vα domain comprises or consists of the amino acid sequence of SEQ ID NO: 16. In examples where the TCR Vα domain CDR2 has the amino acid sequence of SEQ ID NO: 16, the CDR2 may be encoded by any appropriate nucleic acid sequence.

The encoded TCR Vα domain may therefore comprise the CDRs mentioned in detail above (by SEQ ID specifically i.e. SEQ ID NO: 17, SEQ ID NO: 15 and SEQ ID NO: 16, or functional variants thereof), with appropriate intervening sequences between the CDRs.

The encoded TCR Vα domain may comprise an amino acid sequence of SEQ ID NO:21, or a functional variant thereof (i.e, wherein the variant TCR Vα domain retains the ability to specifically bind to a MAGE-A1 antigen (e.g. the peptide shown in SEQ ID NO: 128) when part of a binding protein described herein). Such functional variants may be naturally occurring, synthetic, or synthetically improved functional variants of SEQ ID NO:21. The term “variant” also encompasses homologues and fragments. Functional variants will typically contain only conservative substitutions of one or more amino acids of SEQ ID NO:21, or substitution, deletion or insertion of non-critical amino acids in non-critical regions of the protein.

Non-functional variants are amino acid sequence variants of SEQ ID NO:21 that do not specifically bind to a MAGE-A1 antigen (e.g. the peptide shown in SEQ ID NO: 128). Non-functional variants will typically contain a non-conservative substitution, a deletion, or insertion or premature truncation of the amino acid sequence of SEQ ID NO:21 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO:21, whilst retaining the ability to specifically bind to a MAGE-A1 antigen (e.g. the peptide shown in SEQ ID NO: 128). In other words, a functional TCR Vα domain with one or several amino acid substitutions compared to the sequence of SEQ ID NO:21 is also encompassed. As stated previously, the amino acid substitution may be a conservative amino acid substitution. The variability in sequence compared to SEQ ID NO:21 may all be in regions of the TCR Vα domain that do not form CDRs (i.e. the variant may have the CDRs of SEQ ID NO: 17, SEQ ID NO: 15 and/or SEQ ID NO: 16, and still have 25% (or less) sequence variability compared to SEQ ID NO:21). In other words, the sequence of the CDRs of SEQ ID NO: 21 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 21).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 21, wherein the TCR Vα domain comprises a CDR3 having an amino acid sequence of SEQ ID NO: 17. In this example, the TCR Vα domain CDR1 may have an amino acid sequence of SEQ ID NO: 15 and the TCR Vα domain CDR2 may have an amino acid sequence of SEQ ID NO: 16.

As another example, the encoded TCR Vα domain may comprise an amino acid sequence having the amino acid sequence of SEQ ID NO: 21, with 0 to 10 (or 0 to 5) amino acid substitutions, insertions or deletions, wherein the TCR Vα domain comprises a CDR3 having an amino acid sequence of SEQ ID NO: 17. In this example, the TCR Vα domain CDR1 may have an amino acid sequence of SEQ ID NO: 15 and the TCR Vα domain CDR2 may have an amino acid sequence of SEQ ID NO: 16.

In examples where the TCR Vα domain has the amino acid sequence of SEQ ID NO:21, the TCR Vα domain may be encoded by the nucleic acid sequence of SEQ ID NO: 22, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

An example of a specific TCR α chain amino acid sequence that includes a TCR Vα domain described herein with an appropriate constant domain is shown in SEQ ID NO: 25. Appropriate functional variants of SEQ ID NO: 25 are also encompassed (e.g. variants having at least 75% (e.g. at least 75%, at least 80%, at least 85%, at least 90%, at least 95% etc) sequence identity to the amino acid sequence of SEQ ID NO: 25, wherein the variant TCR α chain amino acid sequence retains its ability to specifically bind to a MAGE-A1 antigen (e.g. the peptide shown in SEQ ID NO:128) when part of a binding protein described herein). In other words, a functional TCR α chain with one or several amino acid substitutions compared to the sequence of SEQ ID NO:25 is also encompassed. As stated previously, the amino acid substitution may be a conservative amino acid substitution. The variability in sequence compared to SEQ ID NO: 25 may all be in regions of the TCR α chain that do not form CDRs (i.e. the variant may have the CDRs of SEQ ID NO: 17, SEQ ID NO: 15 and/or SEQ ID NO: 16, and still have 25% (or less) sequence variability compared to SEQ ID NO:25). In other words, the sequence of the CDRs of SEQ ID NO: 25 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 25).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 25, wherein the TCR α chain comprises a CDR3 having an amino acid sequence of SEQ ID NO: 17. In this example, the TCR α chain CDR1 may have an amino acid sequence of SEQ ID NO: 15 and the TCR α chain CDR2 may have an amino acid sequence of SEQ ID NO: 16.

In examples where the TCR α chain has the amino acid sequence of SEQ ID NO:25, the TCR α chain may be encoded by the nucleic acid sequence of SEQ ID NO:26, or a genetically degenerate sequence thereof (i.e. other nucleic acid sequences that encode the same protein as a result of the degeneracy of the genetic code). It is noted that SEQ ID NO:26 is the nucleic acid sequence for TCR α chain of clone CT31.10C1.

In one example, the nucleic acid composition provided herein comprises a nucleic acid sequence that encodes a TCR Vα domain comprising a CDR3 amino acid sequence having at least 80% sequence identity to SEQ ID NO: 17, or a functional fragment thereof.

In another example, the CDR3 of the Vα domain of a nucleic acid composition provided herein comprises or consists of the amino acid sequence of SEQ ID NO: 17.

In another example, the Vα domain of a nucleic acid composition provided herein comprises an amino acid sequence having at least 80% sequence identity to, comprising, or consisting of, SEQ ID NO: 21.

As provided elsewhere herein, the inventors have identified TCR clone CT31.10C1 which interacts with VRFFFPSL (SEQ ID NO: 128) in the context of HLA-C*07:02. The sequences provided herein that correspond to TCR clone CT31.10C1 are SEQ ID NO:s 15 to 28.

An example of an appropriate TCR Vβ domain CDR3 amino acid sequence that confers specific binding to a MAGE antigen, in particular a MAGE-A1 antigen (e.g. to VRFFFPSL (SEQ ID NO: 128)), is shown in SEQ ID NO:20. As would be clear to a person of skill in the art, variants of the amino acid sequence shown in SEQ ID NO:20 may also be functional (i.e. retain their ability to confer specific binding to a MAGE-A1 antigen (i.e., the peptide shown in SEQ ID NO: 128) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 20, i.e. they may have at least 80%, at least 84%, at least 92%, or 100% sequence identity to SEQ ID NO: 20. Suitably, percent identity is calculated as the percentage of identity to the entire length of the reference sequence (e.g. SEQ ID NO: 20). In other words, appropriate (functional) Vβ domain CDR3 amino acid sequences may vary from the sequence shown in SEQ ID NO: 20 by one or several (e.g. two) amino acids. As stated above, functional variants of SEQ ID NO: 20 retain their ability to confer specific binding to a MAGE-A1 antigen (e.g. the peptide shown in SEQ ID NO: 128) when the CDR3 is part of TCR Vβ domain.

Functional variants may be naturally occurring, synthetic, or synthetically improved functional variants of SEQ ID NO: 20. The term “variant” also encompasses homologues and fragments. Functional variants will typically contain only conservative substitutions of one or more amino acids of SEQ ID NO: 20, or substitution, deletion or insertion of non-critical amino acids in non-critical regions of the CDR3.

Non-functional variants are amino acid sequence variants of SEQ ID NO: 20 that do not specifically bind to a MAGE-A1 antigen (e.g. the peptide shown in SEQ ID NO: 128). Non-functional variants will typically contain a non-conservative substitution, a deletion, or insertion or premature truncation of the amino acid sequence of SEQ ID NO: 20 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 20. In examples where the TCR Vβ domain CDR3 has the amino acid sequence of SEQ ID NO:20, the CDR3 may be encoded by any appropriate nucleic acid sequence.

The encoded TCR Vβ domain may comprise, in addition to the specified CDR3, a CDR1 comprising an amino acid sequence of SEQ ID NO: 18, or a functional variant thereof (i.e, wherein the variant retains the ability to specifically bind to a MAGE-A1 antigen (e.g. the peptide shown in SEQ ID NO: 128)). Such functional variants may be naturally occurring, synthetic, or synthetically improved functional variants of SEQ ID NO: 18. The term “variant” also encompasses homologues and fragments. Functional variants will typically contain only conservative substitutions of one or more amino acids of SEQ ID NO: 18, or substitution, deletion or insertion of non-critical amino acids in non-critical regions of the protein.

Non-functional variants are amino acid sequence variants of SEQ ID NO: 18 that do not specifically bind to a MAGE-A1 antigen (e.g. the peptide shown in SEQ ID NO: 128). Non-functional variants will typically contain a non-conservative substitution, a deletion, or insertion or premature truncation of the amino acid sequence of SEQ ID NO: 18 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 18, i.e. it may have at least 80%, or 100% sequence identity to SEQ ID NO: 18. Suitably, percent identity is calculated as the percentage of identity to the entire length of the reference sequence (e.g. SEQ ID NO: 18). In other words, appropriate (functional) Vβ domain CDR1 amino acid sequences may vary from the sequence shown in SEQ ID NO: 18 by one or several amino acids. As stated previously, the variant may comprise an amino acid substitution such as a conservative amino acid substitution compared to the sequence shown in SEQ ID NO: 18. As stated above, functional variants of SEQ ID NO:

18 retain the ability to specifically bind to a MAGE-A1 antigen (e.g. the peptide shown in SEQ ID NO: 128) when the CDR1 is part of TCR Vβ domain).

In one example, the CDR1 of the Vβ domain comprises or consists of the amino acid sequence of SEQ ID NO: 18. In examples where the TCR Vα domain CDR1 has the amino acid sequence of SEQ ID NO: 18, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 19, or a functional variant thereof (i.e, wherein the variant retains the ability to specifically bind to HLA-C*07:02). Such functional variants may be naturally occurring, synthetic, or synthetically improved functional variants of SEQ ID NO: 19. The term “variant” also encompasses homologues and fragments. Functional variants will typically contain only conservative substitutions of one or more amino acids of SEQ ID NO: 19, or substitution, deletion or insertion of non-critical amino acids in non-critical regions of the protein.

Non-functional variants are amino acid sequence variants of SEQ ID NO: 19 that do not specifically bind to HLA-C*07:02. Non-functional variants will typically contain a non-conservative substitution, a deletion, or insertion or premature truncation of the amino acid sequence of SEQ ID NO: 19 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 19, i.e. it may have at least 80%, at least 83%, or 100% sequence identity to SEQ ID NO: 19. Suitably, percent identity is calculated as the percentage of identity to the entire length of the reference sequence (e.g. SEQ ID NO: 19). In other words, appropriate (functional) Vβ domain CDR2 amino acid sequences may vary from the sequence shown in SEQ ID NO: 19 by one or several amino acids. As stated previously, the variant may comprise an amino acid substitution such as a conservative amino acid substitution compared to the sequence shown in SEQ ID NO: 19. As stated above, a functional variant of SEQ ID NO: 19 retains the ability to specifically bind to HLA-C*07:02.

In one example, the CDR2 of the Vβ domain comprises or consists of the amino acid sequence of SEQ ID NO: 19. In examples where the TCR Vβ domain CDR2 has the amino acid sequence of SEQ ID NO: 19, the CDR2 may be encoded by any appropriate nucleic acid sequence.

The encoded TCR Vβ domain may therefore comprise the CDRs mentioned in detail above (by SEQ ID specifically i.e. SEQ ID NO:20, SEQ ID NO: 18 and SEQ ID NO: 19, or functional variants thereof), with appropriate intervening sequences between the CDRs.

The encoded TCR Vβ domain may have an amino acid sequence of SEQ ID NO: 23, or a functional variant thereof (i.e, wherein the variant TCR Vβ domain retains the ability to specifically bind to a MAGE-A1 antigen (e.g. the peptide shown in SEQ ID NO: 128) when part of a binding protein described herein). Such functional variants may be naturally occurring, synthetic, or synthetically improved functional variants of SEQ ID NO: 23. The term “variant” also encompasses homologues and fragments. Functional variants will typically contain only conservative substitutions of one or more amino acids of SEQ ID NO: 23, or substitution, deletion or insertion of non-critical amino acids in non-critical regions of the protein.

Non-functional variants are amino acid sequence variants of SEQ ID NO: 23 that do not specifically bind to a MAGE-A1 antigen (e.g. the peptide shown in SEQ ID NO: 128). Non-functional variants will typically contain a non-conservative substitution, a deletion, or insertion or premature truncation of the amino acid sequence of SEQ ID NO:23 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 23, whilst retaining the ability to specifically bind to a MAGE-A1 antigen (e.g. the peptide shown in SEQ ID NO: 128). In other words, a functional TCR Vβ domain with one or several amino acid substitutions compared to the sequence of SEQ ID NO: 23 is also encompassed. As stated previously, the amino acid substitution may be a conservative amino acid substitution. The variability in sequence compared to SEQ ID NO:23 may all be in regions of the TCR Vβ domain that do not form CDRs (i.e. the variant may have the CDRs of SEQ ID NO: 20, SEQ ID NO: 18 and/or SEQ ID NO: 19, and still have 25% (or less) sequence variability compared to SEQ ID NO: 23). In other words, the sequence of the CDRs of SEQ ID NO: 23 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 23).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 23, wherein the TCR Vβ domain comprises a CDR3 having an amino acid sequence of SEQ ID NO: 20. In this example, the TCR Vβ domain CDR1 may have an amino acid sequence of SEQ ID NO: 18 and the TCR Vβ domain CDR2 may have an amino acid sequence of SEQ ID NO: 19.

In examples where the TCR Vβ domain has the amino acid sequence of SEQ ID NO:23, the TCR Vβ domain may be encoded by the nucleic acid sequence of SEQ ID NO:24, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 27. Appropriate functional variants of SEQ ID NO: 27 are also encompassed (e.g. variants having at least 75% (e.g. at least 75%, at least 80%, at least 85%, at least 90%, at least 95% etc) sequence identity to the amino acid sequence of SEQ ID NO: 27, wherein the variant TCR β chain amino acid sequence retains its ability to specifically bind to a MAGE-A1 antigen (e.g. the peptide shown in SEQ ID NO: 128) when part of a binding protein described herein). In other words, a functional TCR β chain with one or several amino acid substitutions compared to the sequence of SEQ ID NO: 27 is also encompassed. As stated previously, the amino acid substitution may be a conservative amino acid substitution. The variability in sequence compared to SEQ ID NO: 27 may all be in regions of the TCR β chain that do not form CDRs (i.e. the variant may have the CDRs of SEQ ID NO: 20, SEQ ID NO: 18 and/or SEQ ID NO: 19, and still have 25% (or less) sequence variability compared to SEQ ID NO:27). In other words, the sequence of the CDRs of SEQ ID NO: 27 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 27).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 27, wherein the TCR β chain comprises a CDR3 having an amino acid sequence of SEQ ID NO: 20. In this example, the TCR β chain CDR1 may have an amino acid sequence of SEQ ID NO: 18 and the TCR β chain CDR2 may have an amino acid sequence of SEQ ID NO: 19.

In examples where the TCR β chain has the amino acid sequence of SEQ ID NO:27, the TCR β chain may be encoded by the nucleic acid sequence of SEQ ID NO:28, or a genetically degenerate sequence thereof (i.e. other nucleic acid sequences that encode the same protein as a result of the degeneracy of the genetic code). It is noted that SEQ ID NO:28 is the nucleic acid sequence for TCR β chain of clone CT31.10C1.

In an example, the nucleic acid composition provided herein comprises a nucleic acid sequence that encodes a TCR Vβ domain comprising a CDR3 amino acid sequence having at least 80% sequence identity to SEQ ID NO:20, or a functional fragment thereof.

In another example, the CDR3 of the Vβ domain of a nucleic acid composition provided herein comprises or consists of the amino acid sequence of SEQ ID NO:20.

In a further example, the Vβ domain of a nucleic acid composition provided herein comprises an amino acid sequence having at least 80% sequence identity to, comprising, or consisting of, SEQ ID NO: 23.

The TCR Vβ domain sequences derived from TCR clone CT31.10C1 discussed above are particularly compatible with the TCR Vα domain sequences derived from TCR clone CT31.10C1 discussed elsewhere herein.

Accordingly, in one example, a nucleic acid composition described herein encodes a MAGE-A1 antigen-specific binding protein having TCR Vα domain comprising a CDR3 amino acid sequence having at least 80% sequence identity to SEQ ID NO: 17, or a functional fragment thereof; and a nucleic acid sequence that encodes a TCR Vβ domain comprising a CDR3 amino acid sequence having at least 80% sequence identity to SEQ ID NO:20, or a functional fragment thereof.

In a particular example, a nucleic acid composition described herein encodes a MAGE-A1 antigen-specific binding protein having a TCR Vα domain with a CDR3 amino acid sequence comprising or consisting of the amino acid sequence of SEQ ID NO: 17; and a TCR Vβ domain with a CDR3 comprising or consisting of the amino acid sequence of SEQ ID NO:20. In addition, the MAGE-A1 antigen may comprise or consist of the sequence shown in SEQ ID NO: 128. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 21; and the Vβ domain may comprise an amino acid sequence having at least 80% sequence identity to, comprising, or consisting of, SEQ ID NO: 23. In one example, the Vα domain comprises the amino acid sequence of SEQ ID NO: 21 and the Vβ domain comprises the amino acid sequence of SEQ ID NO: 23. In such cases, the Vα domain may be encoded by a nucleic acid sequence comprising the sequence of SEQ ID NO: 22; and the Vβ domain may be encoded by a nucleic acid sequence comprising the sequence of SEQ ID NO: 24.

In this particular example, the TCR Vα domain may include a CDR1 amino acid sequence comprising or consisting of the amino acid sequence of SEQ ID NO: 15 and a CDR2 amino acid sequence comprising or consisting of the amino acid sequence of SEQ ID NO: 16. Furthermore, the TCR Vβ domain may include a CDR1 amino acid sequence comprising or consisting of the amino acid sequence of SEQ ID NO: 18 and a CDR2 amino acid sequence comprising or consisting of the amino acid sequence of SEQ ID NO: 19.

For the avoidance of doubt, this particular example encompasses components of TCR clone CT31.10C1 exemplified herein. The different components of TCR clone CT31.10C1 and their respective SEQ ID Nos are summarised in Table 7 below.

As stated in more detail elsewhere herein, the nucleic acid composition described herein encodes both a TCR Vα domain and a TCR Vβ domain, which form the binding protein that is capable of specifically binding to a MAGE antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

(iii) TCR Components that Interact with YVGKEHMFY (SEQ ID NO: 129)

As provided elsewhere herein, the inventors have also identified TCR clone CT43.2D8 (2D8) which interacts with YVGKEHMFY (SEQ ID NO: 129) in the context of HLA-A*01:01. The sequences provided herein that correspond to TCR clone CT43.2D8 are SEQ ID NO:s 29 to 42.

In one embodiment, an isolated nucleic acid composition that encodes a MAGE antigen-specific binding protein having a TCR α chain variable (Vα) domain and a TCR β chain variable (Vβ) domain is provided, the composition comprising:
a nucleic acid sequence that encodes a TCR Vα domain comprising a CDR3 amino acid sequence having at least 80% sequence identity to SEQ ID NO:31, or a functional fragment thereof; and a nucleic acid sequence that encodes a TCR Vβ domain comprising a CDR3 amino acid sequence having at least 80% sequence identity to SEQ ID NO:34, or a functional fragment thereof, wherein the CDR3 sequences together specifically bind to MAGE-A9.

An example of an appropriate TCR Vα domain CDR3 amino acid sequence that confers specific binding to a MAGE antigen, particularly a MAGE-A9 antigen (e.g. to YVGKEHMFY (SEQ ID NO: 129)), is shown in SEQ ID NO:31. As would be clear to a person of skill in the art, variants of the amino acid sequence shown in SEQ ID NO:31 may also be functional (i.e. retain their ability to confer specific binding to a MAGE-A9 antigen (e.g. to the peptide YVGKEHMFY (SEQ ID NO: 129)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 31, i.e. they may have at least 80%, at least 83%, at least 91%, or 100% sequence identity to SEQ ID NO: 31. Suitably, percent identity is calculated as the percentage of identity to the entire length of the reference sequence (e.g. SEQ ID NO:31). In other words, appropriate (functional) Vα domain CDR3 amino acid sequences may vary from the sequence shown in SEQ ID NO:31 by one or several (e.g. two etc) amino acids.

As stated above, functional variants of SEQ ID NO: 31 retain their ability to confer specific binding to a MAGE-A9 antigen (i.e., the peptide shown in SEQ ID NO: 129) when the CDR3 is part of TCR Vα domain.

Functional variants may be naturally occurring, synthetic, or synthetically improved functional variants of SEQ ID NO: 31. The term “variant” also encompasses homologues and fragments. Functional variants will typically contain only conservative substitutions of one, two or more amino acids of SEQ ID NO: 31, or substitution, deletion or insertion of non-critical amino acids in non-critical regions of the CDR3.

Non-functional variants are amino acid sequence variants of SEQ ID NO: 31 that do not specifically bind to a MAGE-A9 antigen (i.e. the peptide shown in SEQ ID NO: 129). Non-functional variants will typically contain a non-conservative substitution, a deletion, or insertion or premature truncation of the amino acid sequence of SEQ ID NO: 31 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 31. In examples where the TCR Vα domain CDR3 has the amino acid sequence of SEQ ID NO: 31, the CDR3 may be encoded by any appropriate nucleic acid sequence.

The encoded TCR Vα domain may comprise, in addition to the specified CDR3, a CDR1 comprising an amino acid sequence of SEQ ID NO: 29, or a functional variant thereof (i.e, wherein the variant retains the ability to specifically bind to a MAGE-A9 antigen (e.g. the peptide shown in SEQ ID NO: 129)). Such functional variants may be naturally occurring, synthetic, or synthetically improved functional variants of SEQ ID NO: 29. The term “variant” also encompasses homologues and fragments. Functional variants will typically contain only conservative substitutions of one or more amino acids of SEQ ID NO: 29, or substitution, deletion or insertion of non-critical amino acids in non-critical regions of the protein.

Non-functional variants are amino acid sequence variants of SEQ ID NO: 29 that do not specifically bind to a MAGE-A9 antigen (e.g. the peptide shown in SEQ ID NO: 129). Non-functional variants will typically contain a non-conservative substitution, a deletion, or insertion or premature truncation of the amino acid sequence of SEQ ID NO: 29 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 29, i.e. it may have at least 80%, at least 83%, or 100% sequence identity to SEQ ID NO: 29. Suitably, percent identity is calculated as the percentage of identity to the entire length of the reference sequence (e.g. SEQ ID NO: 29). In other words, appropriate functional Vα domain CDR1 amino acid sequences may vary from the sequence shown in SEQ ID NO: 29 by one or several amino acids. As stated previously, the variant may comprise an amino acid substitution such as a conservative amino acid substitution compared to the sequence shown in SEQ ID NO: 29. As stated above, functional variants of SEQ ID NO: 29 retain the ability to specifically bind to a MAGE-A9 antigen (e.g. the peptide shown in SEQ ID NO: 129) when the CDR1 is part of TCR Vα domain).

In one example, the CDR1 of the Vα domain comprises or consists of the amino acid sequence of SEQ ID NO: 29. In examples where the TCR Vα domain CDR1 has the amino acid sequence of SEQ ID NO: 29, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 30, or a functional variant thereof (i.e, wherein the variant retains the ability to specifically bind to HLA-A*01:01). Such functional variants may be naturally occurring, synthetic, or synthetically improved functional variants of SEQ ID NO: 30. The term “variant” also encompasses homologues and fragments. Functional variants will typically contain only conservative substitutions of one or more amino acids of SEQ ID NO: 30, or substitution, deletion or insertion of non-critical amino acids in non-critical regions of the protein.

Non-functional variants are amino acid sequence variants of SEQ ID NO: 30 that do not specifically bind to HLA-A*01:01. Non-functional variants will typically contain a non-conservative substitution, a deletion, or insertion or premature truncation of the amino acid sequence of SEQ ID NO: 30 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 30, i.e. it may have at least 80%, at least 85%, or 100% sequence identity to SEQ ID NO: 30. Suitably, percent identity is calculated as the percentage of identity to the entire length of the reference sequence (e.g. SEQ ID NO:30). In other words, appropriate (functional) Vα domain CDR2 amino acid sequences may vary from the sequence shown in SEQ ID NO:30 by one or several amino acids. As stated previously, the variant may comprise an amino acid substitution such as a conservative amino acid substitution compared to the sequence shown in SEQ ID NO: 30. As stated above, a functional variant of SEQ ID NO: 30 retains the ability to specifically bind to HLA-A*01:01.

In one example, the CDR2 of the Vα domain comprises or consists of the amino acid sequence of SEQ ID NO: 30. In examples where the TCR Vα domain CDR2 has the amino acid sequence of SEQ ID NO:30, the CDR2 may be encoded by any appropriate nucleic acid sequence.

The encoded TCR Vα domain may therefore comprise the CDRs mentioned in detail above (by SEQ ID specifically i.e. SEQ ID NO: 31, SEQ ID NO: 29 and SEQ ID NO: 30, or functional variants thereof), with appropriate intervening sequences between the CDRs.

The encoded TCR Vα domain may comprise an amino acid sequence of SEQ ID NO: 35, or a functional variant thereof (i.e, wherein the variant TCR Vα domain retains the ability to specifically bind to a MAGE-A9 antigen (e.g. the peptide shown in SEQ ID NO: 129) when part of a binding protein described herein). Such functional variants may be naturally occurring, synthetic, or synthetically improved functional variants of SEQ ID NO: 35. The term “variant” also encompasses homologues and fragments. Functional variants will typically contain only conservative substitutions of one or more amino acids of SEQ ID NO: 35, or substitution, deletion or insertion of non-critical amino acids in non-critical regions of the protein.

Non-functional variants are amino acid sequence variants of SEQ ID NO: 35 that do not specifically bind to a MAGE-A9 antigen (e.g. the peptide shown in SEQ ID NO: 129). Non-functional variants will typically contain a non-conservative substitution, a deletion, or insertion or premature truncation of the amino acid sequence of SEQ ID NO: 35 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 35, whilst retaining the ability to specifically bind to a MAGE-A9 antigen (e.g. the peptide shown in SEQ ID NO: 129). In other words, a functional TCR Vα domain with one or several amino acid substitutions compared to the sequence of SEQ ID NO: 35 is also encompassed. As stated previously, the amino acid substitution may be a conservative amino acid substitution. The variability in sequence compared to SEQ ID NO: 35 may all be in regions of the TCR Vα domain that do not form CDRs (i.e. the variant may have the CDRs of SEQ ID NO: 31, SEQ ID NO: 29 and/or SEQ ID NO: 30, and still have 25% (or less) sequence variability compared to SEQ ID NO: 35). In other words, the sequence of the CDRs of SEQ ID NO: 35 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 35).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 35, wherein the TCR Vα domain comprises a CDR3 having an amino acid sequence of SEQ ID NO: 31. In this example, the TCR Vα domain CDR1 may have an amino acid sequence of SEQ ID NO: 29 and the TCR Vα domain CDR2 may have an amino acid sequence of SEQ ID NO: 30.

As another example, the encoded TCR Vα domain may comprise an amino acid sequence having the amino acid sequence of SEQ ID NO: 35, with 0 to 10 (or 0 to 5) amino acid substitutions, insertions or deletions, wherein the TCR Vα domain comprises a CDR3 having an amino acid sequence of SEQ ID NO: 31. In this example, the TCR Vα domain CDR1 may have an amino acid sequence of SEQ ID NO: 29 and the TCR Vα domain CDR2 may have an amino acid sequence of SEQ ID NO: 30.

In examples where the TCR Vα domain has the amino acid sequence of SEQ ID NO: 35, the TCR Vα domain may be encoded by the nucleic acid sequence of SEQ ID NO: 36, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

An example of a specific TCR α chain amino acid sequence that includes a TCR Vα domain described herein with an appropriate constant domain is shown in SEQ ID NO: 39. Appropriate functional variants of SEQ ID NO: 39 are also encompassed (e.g. variants having at least 75% (e.g. at least 75%, at least 80%, at least 85%, at least 90%, at least 95% etc) sequence identity to the amino acid sequence of SEQ ID NO: 39, wherein the variant TCR α chain amino acid sequence retains its ability to specifically bind to a MAGE-A9 antigen (e.g. the peptide shown in SEQ ID NO: 129) when part of a binding protein described herein). In other words, a functional TCR α chain with one or several amino acid substitutions compared to the sequence of SEQ ID NO: 39 is also encompassed. As stated previously, the amino acid substitution may be a conservative amino acid substitution. The variability in sequence compared to SEQ ID NO: 39 may all be in regions of the TCR α chain that do not form CDRs (i.e. the variant may have the CDRs of SEQ ID NO: 31, SEQ ID NO: 29 and/or SEQ ID NO: 30, and still have 25% (or less) sequence variability compared to SEQ ID NO: 39). In other words, the sequence of the CDRs of SEQ ID NO: 39 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 39).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 39, wherein the TCR α chain comprises a CDR3 having an amino acid sequence of SEQ ID NO: 31. In this example, the TCR α chain CDR1 may have an amino acid sequence of SEQ ID NO: 29 and the TCR α chain CDR2 may have an amino acid sequence of SEQ ID NO: 30.

In examples where the TCR α chain has the amino acid sequence of SEQ ID NO: 39, the TCR a chain may be encoded by the nucleic acid sequence of SEQ ID NO: 40, or a genetically degenerate sequence thereof (i.e. other nucleic acid sequences that encode the same protein as a result of the degeneracy of the genetic code). It is noted that SEQ ID NO:40 is the nucleic acid sequence for TCR α chain of clone CT43.2D8.

In one example, the nucleic acid composition provided herein comprises a nucleic acid sequence that encodes a TCR Vα domain comprising a CDR3 amino acid sequence having at least 80% sequence identity to SEQ ID NO:31, or a functional fragment thereof.

In another example, the CDR3 of the Vα domain of a nucleic acid composition provided herein comprises or consists of the amino acid sequence of SEQ ID NO: 31.

In another example, the Vα domain of a nucleic acid composition provided herein comprises an amino acid sequence having at least 80% sequence identity to, comprising, or consisting of, SEQ ID NO: 35.

As provided above, the inventors identified TCR clone CT43.2D8 which interacts with YVGKEHMFY (SEQ ID NO: 129) in the context of HLA-A*01:01. The sequences provided herein that correspond to TCR clone CT43.2D8 are SEQ ID NO:s 29 to 42.

An example of an appropriate TCR Vβ domain CDR3 amino acid sequence that confers specific binding to a MAGE antigen, in particular a MAGE-A9 antigen (e.g. to YVGKEHMFY (SEQ ID NO: 129)), is shown in SEQ ID NO:34. As would be clear to a person of skill in the art, variants of the amino acid sequence shown in SEQ ID NO:34 may also be functional (i.e. retain their ability to confer specific binding to a MAGE-A9 antigen (i.e. the peptide shown in SEQ ID NO: 129) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 34, i.e. they may have at least 80%, at least 83%, at least 91%, or 100% sequence identity to SEQ ID NO: 34. Suitably, percent identity is calculated as the percentage of identity to the entire length of the reference sequence (e.g. SEQ ID NO: 34). In other words, appropriate (functional) Vβ domain CDR3 amino acid sequences may vary from the sequence shown in SEQ ID NO: 34 by one or several (e.g. two) amino acids. As stated above, functional variants of SEQ ID NO: 34 retain their ability to confer specific binding to a MAGE-A9 antigen (e.g. the peptide shown in SEQ ID NO: 129) when the CDR3 is part of TCR Vβ domain.

Functional variants may be naturally occurring, synthetic, or synthetically improved functional variants of SEQ ID NO: 34. The term “variant” also encompasses homologues and fragments. Functional variants will typically contain only conservative substitutions of one or more amino acids of SEQ ID NO: 34, or substitution, deletion or insertion of non-critical amino acids in non-critical regions of the CDR3.

Non-functional variants are amino acid sequence variants of SEQ ID NO: 34 that do not specifically bind to a MAGE-A9 antigen (e.g. the peptide shown in SEQ ID NO: 129). Non-functional variants will typically contain a non-conservative substitution, a deletion, or insertion or premature truncation of the amino acid sequence of SEQ ID NO: 34 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 34. In examples where the TCR Vβ domain CDR3 has the amino acid sequence of SEQ ID NO:34, the CDR3 may be encoded by any appropriate nucleic acid sequence.

The encoded TCR Vβ domain may comprise, in addition to the specified CDR3, a CDR1 comprising an amino acid sequence of SEQ ID NO: 32, or a functional variant thereof (i.e, wherein the variant retains the ability to specifically bind to a MAGE-A9 antigen (e.g. the peptide shown in SEQ ID NO: 129)). Such functional variants may be naturally occurring, synthetic, or synthetically improved functional variants of SEQ ID NO: 32. The term “variant” also encompasses homologues and fragments. Functional variants will typically contain only conservative substitutions of one or more amino acids of SEQ ID NO: 32, or substitution, deletion or insertion of non-critical amino acids in non-critical regions of the protein.

Non-functional variants are amino acid sequence variants of SEQ ID NO: 32 that do not specifically bind to a MAGE-A9 antigen (e.g. the peptide shown in SEQ ID NO: 129). Non-functional variants will typically contain a non-conservative substitution, a deletion, or insertion or premature truncation of the amino acid sequence of SEQ ID NO: 32 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 32, i.e. it may have at least 80%, or 100% sequence identity to SEQ ID NO: 32. Suitably, percent identity is calculated as the percentage of identity to the entire length of the reference sequence (e.g. SEQ ID NO: 32). In other words, appropriate (functional) Vβ domain CDR1 amino acid sequences may vary from the sequence shown in SEQ ID NO:32 by one or several amino acids. As stated previously, the variant may comprise an amino acid substitution such as a conservative amino acid substitution compared to the sequence shown in SEQ ID NO:32. As stated above, functional variants of SEQ ID NO: 32 retain the ability to specifically bind to a MAGE-A9 antigen (e.g. the peptide shown in SEQ ID NO: 129) when the CDR1 is part of TCR Vβ domain).

In one example, the CDR1 of the Vβ domain comprises or consists of the amino acid sequence of SEQ ID NO: 32. In examples where the TCR Vα domain CDR1 has the amino acid sequence of SEQ ID NO:32, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 33, or a functional variant thereof (i.e, wherein the variant retains the ability to specifically bind to HLA-A*01:01). Such functional variants may be naturally occurring, synthetic, or synthetically improved functional variants of SEQ ID NO: 33. The term “variant” also encompasses homologues and fragments. Functional variants will typically contain only conservative substitutions of one or more amino acids of SEQ ID NO: 33, or substitution, deletion or insertion of non-critical amino acids in non-critical regions of the protein.

Non-functional variants are amino acid sequence variants of SEQ ID NO: 33 that do not specifically bind to HLA-A*01:01. Non-functional variants will typically contain a non-conservative substitution, a deletion, or insertion or premature truncation of the amino acid sequence of SEQ ID NO: 33 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 33, i.e. it may have at least 80%, at least 83%, or 100% sequence identity to SEQ ID NO: 33. Suitably, percent identity is calculated as the percentage of identity to the entire length of the reference sequence (e.g. SEQ ID NO: 33). In other words, appropriate (functional) Vβ domain CDR2 amino acid sequences may vary from the sequence shown in SEQ ID NO: 33 by one or several amino acids. As stated previously, the variant may comprise an amino acid substitution such as a conservative amino acid substitution compared to the sequence shown in SEQ ID NO: 33. As stated above, a functional variant of SEQ ID NO: 33 retains the ability to specifically bind to HLA-A*01:01.

In one example, the CDR2 of the Vβ domain comprises or consists of the amino acid sequence of SEQ ID NO: 33. In examples where the TCR Vβ domain CDR2 has the amino acid sequence of SEQ ID NO:33, the CDR2 may be encoded by any appropriate nucleic acid sequence.

The encoded TCR Vβ domain may therefore comprise the CDRs mentioned in detail above (by SEQ ID specifically i.e. SEQ ID NO:34, SEQ ID NO: 32 and SEQ ID NO: 33, or functional variants thereof), with appropriate intervening sequences between the CDRs.

The encoded TCR Vβ domain may have an amino acid sequence of SEQ ID NO: 37, or a functional variant thereof (i.e, wherein the variant TCR Vβ domain retains the ability to specifically bind to a MAGE-A9 antigen (e.g. the peptide shown in SEQ ID NO: 129) when part of a binding protein described herein). Such functional variants may be naturally occurring, synthetic, or synthetically improved functional variants of SEQ ID NO: 37. The term “variant” also encompasses homologues and fragments. Functional variants will typically contain only conservative substitutions of one or more amino acids of SEQ ID NO: 37, or substitution, deletion or insertion of non-critical amino acids in non-critical regions of the protein.

Non-functional variants are amino acid sequence variants of SEQ ID NO: 37 that do not specifically bind to a MAGE-A9 antigen (e.g. the peptide shown in SEQ ID NO: 129). Non-functional variants will typically contain a non-conservative substitution, a deletion, or insertion or premature truncation of the amino acid sequence of SEQ ID NO:37 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 37, whilst retaining the ability to specifically bind to a MAGE-A9 antigen (e.g. the peptide shown in SEQ ID NO: 129). In other words, a functional TCR Vβ domain with one or several amino acid substitutions compared to the sequence of SEQ ID NO: 37 is also encompassed. As stated previously, the amino acid substitution may be a conservative amino acid substitution. The variability in sequence compared to SEQ ID NO:37 may all be in regions of the TCR Vβ domain that do not form CDRs (i.e. the variant may have the CDRs of SEQ ID NO: 34, SEQ ID NO: 32 and/or SEQ ID NO: 33, and still have 25% (or less) sequence variability compared to SEQ ID NO: 37). In other words, the sequence of the CDRs of SEQ ID NO: 37 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 37).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 37, wherein the TCR Vβ domain comprises a CDR3 having an amino acid sequence of SEQ ID NO: 34. In this example, the TCR Vβ domain CDR1 may have an amino acid sequence of SEQ ID NO:32 and the TCR Vβ domain CDR2 may have an amino acid sequence of SEQ ID NO: 33.

In examples where the TCR Vβ domain has the amino acid sequence of SEQ ID NO:37, the TCR Vβ domain may be encoded by the nucleic acid sequence of SEQ ID NO:38, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 41. Appropriate functional variants of SEQ ID NO: 41 are also encompassed (e.g. variants having at least 75% (e.g. at least 75%, at least 80%, at least 85%, at least 90%, at least 95% etc) sequence identity to the amino acid sequence of SEQ ID NO: 41, wherein the variant TCR β chain amino acid sequence retains its ability to specifically bind to a MAGE-A9 antigen (e.g. the peptide shown in SEQ ID NO: 129) when part of a binding protein described herein). In other words, a functional TCR β chain with one or several amino acid substitutions compared to the sequence of SEQ ID NO: 41 is also encompassed. As stated previously, the amino acid substitution may be a conservative amino acid substitution. The variability in sequence compared to SEQ ID NO: 41 may all be in regions of the TCR β chain that do not form CDRs (i.e. the variant may have the CDRs of SEQ ID NO: 34, SEQ ID NO: 32 and/or SEQ ID NO: 33, and still have 25% (or less) sequence variability compared to SEQ ID NO:41). In other words, the sequence of the CDRs of SEQ ID NO: 41 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 41).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 41, wherein the TCR β chain comprises a CDR3 having an amino acid sequence of SEQ ID NO: 34. In this example, the TCR β chain CDR1 may have an amino acid sequence of SEQ ID NO: 32 and the TCR β chain CDR2 may have an amino acid sequence of SEQ ID NO: 33.

In examples where the TCR β chain has the amino acid sequence of SEQ ID NO:41, the TCR β chain may be encoded by the nucleic acid sequence of SEQ ID NO: 42, or a genetically degenerate sequence thereof (i.e. other nucleic acid sequences that encode the same protein as a result of the degeneracy of the genetic code). It is noted that SEQ ID NO:42 is the nucleic acid sequence for TCR β chain of clone CT43.2D8.

In an example, the nucleic acid composition provided herein comprises a nucleic acid sequence that encodes a TCR Vβ domain comprising a CDR3 amino acid sequence having at least 80% sequence identity to SEQ ID NO:34, or a functional fragment thereof.

In another example, the CDR3 of the Vβ domain of a nucleic acid composition provided herein comprises or consists of the amino acid sequence of SEQ ID NO:34.

In a further example, the Vβ domain of a nucleic acid composition provided herein comprises an amino acid sequence having at least 80% sequence identity to, comprising, or consisting of, SEQ ID NO: 37.

The TCR Vβ domain sequences derived from TCR clone CT43.2D8 discussed above are particularly compatible with the TCR Vα domain sequences derived from TCR clone CT43.2D8 discussed elsewhere herein.

Accordingly, in one example, a nucleic acid composition described herein encodes a MAGE-A9 antigen-specific binding protein having TCR Vα domain comprising a CDR3 amino acid sequence having at least 80% sequence identity to SEQ ID NO:31, or a functional fragment thereof; and a nucleic acid sequence that encodes a TCR Vβ domain comprising a CDR3 amino acid sequence having at least 80% sequence identity to SEQ ID NO:34, or a functional fragment thereof.

In a particular example, a nucleic acid composition described herein encodes a MAGE-A9 antigen-specific binding protein having a TCR Vα domain with a CDR3 amino acid sequence comprising or consisting of the amino acid sequence of SEQ ID NO: 31; and a TCR Vβ domain with a CDR3 comprising or consisting of the amino acid sequence of SEQ ID NO:34. In addition, the MAGE-A9 antigen may comprise or consist of the sequence shown in SEQ ID NO: 129. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 35; and the Vβ domain may comprise an amino acid sequence having at least 80% sequence identity to, comprising, or consisting of, SEQ ID NO: 37. In one example, the Vα domain comprises the amino acid sequence of SEQ ID NO: 35 and the Vβ domain comprises the amino acid sequence of SEQ ID NO: 37. In such cases, the Vα domain may be encoded by a nucleic acid sequence comprising the sequence of SEQ ID NO: 36; and the Vβ domain may be encoded by a nucleic acid sequence comprising the sequence of SEQ ID NO: 38.

In this particular example, the TCR Vα domain may include a CDR1 amino acid sequence comprising or consisting of the amino acid sequence of SEQ ID NO: 29 and a CDR2 amino acid sequence comprising or consisting of the amino acid sequence of SEQ ID NO:30. Furthermore, the TCR Vβ domain may include a CDR1 amino acid sequence comprising or consisting of the amino acid sequence of SEQ ID NO:32 and a CDR2 amino acid sequence comprising or consisting of the amino acid sequence of SEQ ID NO: 33.

For the avoidance of doubt, this particular example encompasses components of TCR clone CT43.2D8 exemplified herein. The different components of TCR clone CT43.2D8 and their respective SEQ ID Nos are summarised in Table 8 below.

As stated in more detail elsewhere herein, the nucleic acid composition described herein encodes both a TCR Vα domain and a TCR Vβ domain, which form the binding protein that is capable of specifically binding to a MAGE antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

(iv) TCR Components that Interact with LTQDLVQEKYLEY (SEQ ID NO: 130)

As provided elsewhere herein, the inventors have also identified TCR clone CT44.6G4 (6G4) which interacts with LTQDLVQEKYLEY (SEQ ID NO: 130) in the context of HLA-A*01:01. The sequences provided herein that correspond to TCR clone CT44.6G4 are SEQ ID NO:s 43 to 56.

In one embodiment, an isolated nucleic acid composition that encodes a MAGE antigen-specific binding protein having a TCR α chain variable (Vα) domain and a TCR β chain variable (Vβ) domain is provided, the composition comprising:
a nucleic acid sequence that encodes a TCR Vα domain comprising a CDR3 amino acid sequence having at least 80% sequence identity to SEQ ID NO:45, or a functional fragment thereof; and a nucleic acid sequence that encodes a TCR Vβ domain comprising a CDR3 amino acid sequence having at least 80% sequence identity to SEQ ID NO:48, or a functional fragment thereof, wherein the CDR3 sequences together specifically bind to MAGE-A1.

An example of an appropriate TCR Vα domain CDR3 amino acid sequence that confers specific binding to a MAGE antigen, in particular a MAGE-A1 antigen (e.g. to LTQDLVQEKYLEY (SEQ ID NO: 130)), is shown in SEQ ID NO: 45. As would be clear to a person of skill in the art, variants of the amino acid sequence shown in SEQ ID NO: 45 may also be functional (i.e. retain their ability to confer specific binding to a MAGE-A1 antigen (e.g. to the peptide LTQDLVQEKYLEY (SEQ ID NO: 130)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 45, i.e. they may have at least 80%, at least 84%, at least 92%, or 100% sequence identity to SEQ ID NO: 45. Suitably, percent identity is calculated as the percentage of identity to the entire length of the reference sequence (e.g. SEQ ID NO: 45). In other words, appropriate (functional) Vα domain CDR3 amino acid sequences may vary from the sequence shown in SEQ ID NO: 45 by one or several (e.g. two etc) amino acids.

As stated above, functional variants of SEQ ID NO: 45 retain their ability to confer specific binding to a MAGE-A1 antigen (i.e. the peptide shown in SEQ ID NO: 130) when the CDR3 is part of TCR Vα domain.

Functional variants may be naturally occurring, synthetic, or synthetically improved functional variants of SEQ ID NO: 45. The term “variant” also encompasses homologues and fragments.

Functional variants will typically contain only conservative substitutions of one, two or more amino acids of SEQ ID NO: 45, or substitution, deletion or insertion of non-critical amino acids in non-critical regions of the CDR3.

Non-functional variants are amino acid sequence variants of SEQ ID NO: 45 that do not specifically bind to a MAGE-A1 antigen (i.e. the peptide shown in SEQ ID NO: 130). Non-functional variants will typically contain a non-conservative substitution, a deletion, or insertion or premature truncation of the amino acid sequence of SEQ ID NO: 45 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 45. In examples where the TCR Vα domain CDR3 has the amino acid sequence of SEQ ID NO: 45, the CDR3 may be encoded by any appropriate nucleic acid sequence.

The encoded TCR Vα domain may comprise, in addition to the specified CDR3, a CDR1 comprising an amino acid sequence of SEQ ID NO: 43, or a functional variant thereof (i.e. wherein the variant retains the ability to specifically bind to a MAGE-A1 antigen (e.g. the peptide shown in SEQ ID NO: 130)). Such functional variants may be naturally occurring, synthetic, or synthetically improved functional variants of SEQ ID NO: 43. The term “variant” also encompasses homologues and fragments. Functional variants will typically contain only conservative substitutions of one or more amino acids of SEQ ID NO: 43, or substitution, deletion or insertion of non-critical amino acids in non-critical regions of the protein.

Non-functional variants are amino acid sequence variants of SEQ ID NO: 43 that do not specifically bind to a MAGE-A1 antigen (e.g. the peptide shown in SEQ ID NO: 130). Non-functional variants will typically contain a non-conservative substitution, a deletion, or insertion or premature truncation of the amino acid sequence of SEQ ID NO: 43 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 43, i.e. it may have at least 80%, at least 83%, or 100% sequence identity to SEQ ID NO: 43. Suitably, percent identity is calculated as the percentage of identity to the entire length of the reference sequence (e.g. SEQ ID NO: 43). In other words, appropriate functional Vα domain CDR1 amino acid sequences may vary from the sequence shown in SEQ ID NO: 43 by one or several amino acids. As stated previously, the variant may comprise an amino acid substitution such as a conservative amino acid substitution compared to the sequence shown in SEQ ID NO: 43. As stated above, functional variants of SEQ ID NO: 43 retain the ability to specifically bind to a MAGE-A1 antigen (e.g. the peptide shown in SEQ ID NO: 130) when the CDR1 is part of TCR Vα domain).

In one example, the CDR1 of the Vα domain comprises or consists of the amino acid sequence of SEQ ID NO: 43. In examples where the TCR Vα domain CDR1 has the amino acid sequence of SEQ ID NO: 43, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 44, or a functional variant thereof (i.e, wherein the variant retains the ability to specifically bind to HLA-A*01:01). Such functional variants may be naturally occurring, synthetic, or synthetically improved functional variants of SEQ ID NO: 44. The term “variant” also encompasses homologues and fragments. Functional variants will typically contain only conservative substitutions of one or more amino acids of SEQ ID NO: 44, or substitution, deletion or insertion of non-critical amino acids in non-critical regions of the protein.

Non-functional variants are amino acid sequence variants of SEQ ID NO: 44 that do not specifically bind to HLA-A*01:01. Non-functional variants will typically contain a non-conservative substitution, a deletion, or insertion or premature truncation of the amino acid sequence of SEQ ID NO: 44 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 44, i.e. it may have at least 80%, at least 85%, or 100% sequence identity to SEQ ID NO: 44. Suitably, percent identity is calculated as the percentage of identity to the entire length of the reference sequence (e.g. SEQ ID NO: 44). In other words, appropriate (functional) Vα domain CDR2 amino acid sequences may vary from the sequence shown in SEQ ID NO: 44 by one or several amino acids. As stated previously, the variant may comprise an amino acid substitution such as a conservative amino acid substitution compared to the sequence shown in SEQ ID NO: 44. As stated above, a functional variant of SEQ ID NO: 44 retains the ability to specifically bind to HLA-A*01:01.

In one example, the CDR2 of the Vα domain comprises or consists of the amino acid sequence of SEQ ID NO: 44. In examples where the TCR Vα domain CDR2 has the amino acid sequence of SEQ ID NO: 44, the CDR2 may be encoded by any appropriate nucleic acid sequence.

The encoded TCR Vα domain may therefore comprise the CDRs mentioned in detail above (by SEQ ID specifically i.e. SEQ ID NO:45, SEQ ID NO: 43 and SEQ ID NO: 44, or functional variants thereof), with appropriate intervening sequences between the CDRs.

The encoded TCR Vα domain may comprise an amino acid sequence of SEQ ID NO: 49, or a functional variant thereof (i.e, wherein the variant TCR Vα domain retains the ability to specifically bind to a MAGE-A1 antigen (e.g. the peptide shown in SEQ ID NO: 130) when part of a binding protein described herein). Such functional variants may be naturally occurring, synthetic, or synthetically improved functional variants of SEQ ID NO: 49. The term “variant” also encompasses homologues and fragments. Functional variants will typically contain only conservative substitutions of one or more amino acids of SEQ ID NO: 49, or substitution, deletion or insertion of non-critical amino acids in non-critical regions of the protein.

Non-functional variants are amino acid sequence variants of SEQ ID NO: 49 that do not specifically bind to a MAGE-A1 antigen (e.g. the peptide shown in SEQ ID NO: 130). Non-functional variants will typically contain a non-conservative substitution, a deletion, or insertion or premature truncation of the amino acid sequence of SEQ ID NO: 49 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 49, whilst retaining the ability to specifically bind to a MAGE-A1 antigen (e.g. the peptide shown in SEQ ID NO: 130). In other words, a functional TCR Vα domain with one or several amino acid substitutions compared to the sequence of SEQ ID NO: 49 is also encompassed. As stated previously, the amino acid substitution may be a conservative amino acid substitution. The variability in sequence compared to SEQ ID NO: 49 may all be in regions of the TCR Vα domain that do not form CDRs (i.e. the variant may have the CDRs of SEQ ID NO: 45, SEQ ID NO: 43 and/or SEQ ID NO: 44, and still have 25% (or less) sequence variability compared to SEQ ID NO:49). In other words, the sequence of the CDRs of SEQ ID NO: 49 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 49).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 49, wherein the TCR Vα domain comprises a CDR3 having an amino acid sequence of SEQ ID NO: 45. In this example, the TCR Vα domain CDR1 may have an amino acid sequence of SEQ ID NO: 43 and the TCR Vα domain CDR2 may have an amino acid sequence of SEQ ID NO: 44.

As another example, the encoded TCR Vα domain may comprise an amino acid sequence having the amino acid sequence of SEQ ID NO: 49, with 0 to 10 (or 0 to 5) amino acid substitutions, insertions or deletions, wherein the TCR Vα domain comprises a CDR3 having an amino acid sequence of SEQ ID NO: 45. In this example, the TCR Vα domain CDR1 may have an amino acid sequence of SEQ ID NO: 43 and the TCR Vα domain CDR2 may have an amino acid sequence of SEQ ID NO: 44.

In examples where the TCR Vα domain has the amino acid sequence of SEQ ID NO: 49, the TCR Vα domain may be encoded by the nucleic acid sequence of SEQ ID NO: 50, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

An example of a specific TCR α chain amino acid sequence that includes a TCR Vα domain described herein with an appropriate constant domain is shown in SEQ ID NO: 53. Appropriate functional variants of SEQ ID NO:53 are also encompassed (e.g. variants having at least 75% (e.g. at least 75%, at least 80%, at least 85%, at least 90%, at least 95% etc) sequence identity to the amino acid sequence of SEQ ID NO: 53, wherein the variant TCR α chain amino acid sequence retains its ability to specifically bind to a MAGE-A1 antigen (e.g. the peptide shown in SEQ ID NO: 130) when part of a binding protein described herein). In other words, a functional TCR α chain with one or several amino acid substitutions compared to the sequence of SEQ ID NO:53 is also encompassed. As stated previously, the amino acid substitution may be a conservative amino acid substitution. The variability in sequence compared to SEQ ID NO: 53 may all be in regions of the TCR α chain that do not form CDRs (i.e. the variant may have the CDRs of SEQ ID NO: 45, SEQ ID NO: 43 and/or SEQ ID NO: 44, and still have 25% (or less) sequence variability compared to SEQ ID NO: 53). In other words, the sequence of the CDRs of SEQ ID NO: 53 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 53).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 53, wherein the TCR α chain comprises a CDR3 having an amino acid sequence of SEQ ID NO: 45. In this example, the TCR α chain CDR1 may have an amino acid sequence of SEQ ID NO:43 and the TCR α chain CDR2 may have an amino acid sequence of SEQ ID NO: 44.

In examples where the TCR α chain has the amino acid sequence of SEQ ID NO:53, the TCR α chain may be encoded by the nucleic acid sequence of SEQ ID NO:54, or a genetically degenerate sequence thereof (i.e. other nucleic acid sequences that encode the same protein as a result of the degeneracy of the genetic code). It is noted that SEQ ID NO:54 is the nucleic acid sequence for TCR α chain of clone CT44.6G4.

In one example, the nucleic acid composition provided herein comprises a nucleic acid sequence that encodes a TCR Vα domain comprising a CDR3 amino acid sequence having at least 80% sequence identity to SEQ ID NO:45, or a functional fragment thereof.

In another example, the CDR3 of the Vα domain of a nucleic acid composition provided herein comprises or consists of the amino acid sequence of SEQ ID NO: 45.

In another example, the Vα domain of a nucleic acid composition provided herein comprises an amino acid sequence having at least 80% sequence identity to, comprising, or consisting of, SEQ ID NO: 49.

As provided above, the inventors identified TCR clone CT44.6G4 which interacts with LTQDLVQEKYLEY (SEQ ID NO: 130) in the context of HLA-A*01:01. The sequences provided herein that correspond to TCR clone CT44.6G4 are SEQ ID NO:s 43 to 56.

An example of an appropriate TCR Vβ domain CDR3 amino acid sequence that confers specific binding to a MAGE antigen, in particular a MAGE-A1 antigen (e.g. to LTQDLVQEKYLEY (SEQ ID NO: 130)), is shown in SEQ ID NO:48. As would be clear to a person of skill in the art, variants of the amino acid sequence shown in SEQ ID NO:48 may also be functional (i.e. retain their ability to confer specific binding to a MAGE-A1 antigen (i.e. the peptide shown in SEQ ID NO: 130) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 48, i.e. they may have at least 80%, at least 86%, at least 93%, or 100% sequence identity to SEQ ID NO: 48. Suitably, percent identity is calculated as the percentage of identity to the entire length of the reference sequence (e.g. SEQ ID NO: 48). In other words, appropriate (functional) Vβ domain CDR3 amino acid sequences may vary from the sequence shown in SEQ ID NO: 48 by one or several (e.g. two) amino acids. As stated above, functional variants of SEQ ID NO: 48 retain their ability to confer specific binding to a MAGE-A1 antigen (e.g. the peptide shown in SEQ ID NO: 130) when the CDR3 is part of TCR Vβ domain.

Functional variants may be naturally occurring, synthetic, or synthetically improved functional variants of SEQ ID NO: 48. The term “variant” also encompasses homologues and fragments. Functional variants will typically contain only conservative substitutions of one or more amino acids of SEQ ID NO: 48, or substitution, deletion or insertion of non-critical amino acids in non-critical regions of the CDR3.

Non-functional variants are amino acid sequence variants of SEQ ID NO: 48 that do not specifically bind to a MAGE-A1 antigen (e.g. the peptide shown in SEQ ID NO: 130). Non-functional variants will typically contain a non-conservative substitution, a deletion, or insertion or premature truncation of the amino acid sequence of SEQ ID NO: 48 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 48. In examples where the TCR Vβ domain CDR3 has the amino acid sequence of SEQ ID NO:48, the CDR3 may be encoded by any appropriate nucleic acid sequence.

The encoded TCR Vβ domain may comprise, in addition to the specified CDR3, a CDR1 comprising an amino acid sequence of SEQ ID NO: 46, or a functional variant thereof (i.e, wherein the variant retains the ability to specifically bind to a MAGE-A1 antigen (e.g. the peptide shown in SEQ ID NO: 130)). Such functional variants may be naturally occurring, synthetic, or synthetically improved functional variants of SEQ ID NO: 46. The term “variant” also encompasses homologues and fragments. Functional variants will typically contain only conservative substitutions of one or more amino acids of SEQ ID NO: 46, or substitution, deletion or insertion of non-critical amino acids in non-critical regions of the protein.

Non-functional variants are amino acid sequence variants of SEQ ID NO: 46 that do not specifically bind to a MAGE-A1 antigen (e.g. the peptide shown in SEQ ID NO: 130). Non-functional variants will typically contain a non-conservative substitution, a deletion, or insertion or premature truncation of the amino acid sequence of SEQ ID NO: 46 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 46, i.e. it may have at least 80%, or 100% sequence identity to SEQ ID NO: 46. Suitably, percent identity is calculated as the percentage of identity to the entire length of the reference sequence (e.g. SEQ ID NO: 46). In other words, appropriate (functional) Vβ domain CDR1 amino acid sequences may vary from the sequence shown in SEQ ID NO:46 by one or several amino acids. As stated previously, the variant may comprise an amino acid substitution such as a conservative amino acid substitution compared to the sequence shown in SEQ ID NO:46. As stated above, functional variants of SEQ ID NO: 46 retain the ability to specifically bind to a MAGE-A1 antigen (e.g. the peptide shown in SEQ ID NO: 130) when the CDR1 is part of TCR Vβ domain).

In one example, the CDR1 of the Vβ domain comprises or consists of the amino acid sequence of SEQ ID NO: 46. In examples where the TCR Vα domain CDR1 has the amino acid sequence of SEQ ID NO:46, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 47, or a functional variant thereof (i.e, wherein the variant retains the ability to specifically bind to HLA-A*01:01). Such functional variants may be naturally occurring, synthetic, or synthetically improved functional variants of SEQ ID NO: 47. The term “variant” also encompasses homologues and fragments. Functional variants will typically contain only conservative substitutions of one or more amino acids of SEQ ID NO: 47, or substitution, deletion or insertion of non-critical amino acids in non-critical regions of the protein.

Non-functional variants are amino acid sequence variants of SEQ ID NO: 47 that do not specifically bind to HLA-A*01:01. Non-functional variants will typically contain a non-conservative substitution, a deletion, or insertion or premature truncation of the amino acid sequence of SEQ ID NO: 47 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 47, i.e. it may have at least 80%, at least 83%, or 100% sequence identity to SEQ ID NO: 47. Suitably, percent identity is calculated as the percentage of identity to the entire length of the reference sequence (e.g. SEQ ID NO: 47). In other words, appropriate (functional) Vβ domain CDR2 amino acid sequences may vary from the sequence shown in SEQ ID NO: 47 by one or several amino acids. As stated previously, the variant may comprise an amino acid substitution such as a conservative amino acid substitution compared to the sequence shown in SEQ ID NO: 47. As stated above, a functional variant of SEQ ID NO: 47 retains the ability to specifically bind to HLA-A*01:01.

In one example, the CDR2 of the Vβ domain comprises or consists of the amino acid sequence of SEQ ID NO: 47. In examples where the TCR Vβ domain CDR2 has the amino acid sequence of SEQ ID NO:47, the CDR2 may be encoded by any appropriate nucleic acid sequence.

The encoded TCR Vβ domain may therefore comprise the CDRs mentioned in detail above (by SEQ ID specifically i.e. SEQ ID NO:48, SEQ ID NO: 46 and SEQ ID NO: 47, or functional variants thereof), with appropriate intervening sequences between the CDRs.

The encoded TCR Vβ domain may have an amino acid sequence of SEQ ID NO: 51, or a functional variant thereof (i.e, wherein the variant TCR Vβ domain retains the ability to specifically bind to a MAGE-A1 antigen (e.g. the peptide shown in SEQ ID NO: 130) when part of a binding protein described herein). Such functional variants may be naturally occurring, synthetic, or synthetically improved functional variants of SEQ ID NO: 51. The term “variant” also encompasses homologues and fragments. Functional variants will typically contain only conservative substitutions of one or more amino acids of SEQ ID NO: 51, or substitution, deletion or insertion of non-critical amino acids in non-critical regions of the protein.

Non-functional variants are amino acid sequence variants of SEQ ID NO: 51 that do not specifically bind to a MAGE-A1 antigen (e.g. the peptide shown in SEQ ID NO: 130). Non-functional variants will typically contain a non-conservative substitution, a deletion, or insertion or premature truncation of the amino acid sequence of SEQ ID NO:51 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 51, whilst retaining the ability to specifically bind to a MAGE-A1 antigen (e.g. the peptide shown in SEQ ID NO: 130). In other words, a functional TCR Vβ domain with one or several amino acid substitutions compared to the sequence of SEQ ID NO: 51 is also encompassed. As stated previously, the amino acid substitution may be a conservative amino acid substitution. The variability in sequence compared to SEQ ID NO:51 may all be in regions of the TCR Vβ domain that do not form CDRs (i.e. the variant may have the CDRs of SEQ ID NO: 48, SEQ ID NO: 46 and/or SEQ ID NO: 47, and still have 25% (or less) sequence variability compared to SEQ ID NO: 51). In other words, the sequence of the CDRs of SEQ ID NO: 51 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 51).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 51, wherein the TCR Vβ domain comprises a CDR3 having an amino acid sequence of SEQ ID NO: 48. In this example, the TCR Vβ domain CDR1 may have an amino acid sequence of SEQ ID NO:46 and the TCR Vβ domain CDR2 may have an amino acid sequence of SEQ ID NO: 47.

In examples where the TCR Vβ domain has the amino acid sequence of SEQ ID NO:51, the TCR Vβ domain may be encoded by the nucleic acid sequence of SEQ ID NO:52, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 55. Appropriate functional variants of SEQ ID NO: 55 are also encompassed (e.g. variants having at least 75% (e.g. at least 75%, at least 80%, at least 85%, at least 90%, at least 95% etc) sequence identity to the amino acid sequence of SEQ ID NO: 55, wherein the variant TCR β chain amino acid sequence retains its ability to specifically bind to a MAGE-A1 antigen (e.g. the peptide shown in SEQ ID NO: 130) when part of a binding protein described herein). In other words, a functional TCR β chain with one or several amino acid substitutions compared to the sequence of SEQ ID NO: 55 is also encompassed. As stated previously, the amino acid substitution may be a conservative amino acid substitution. The variability in sequence compared to SEQ ID NO: 55 may all be in regions of the TCR β chain that do not form CDRs (i.e. the variant may have the CDRs of SEQ ID NO: 48, SEQ ID NO: 46 and/or SEQ ID NO: 47, and still have 25% (or less) sequence variability compared to SEQ ID NO:55). In other words, the sequence of the CDRs of SEQ ID NO: 55 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 55).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 55, wherein the TCR β chain comprises a CDR3 having an amino acid sequence of SEQ ID NO: 48. In this example, the TCR β chain CDR1 may have an amino acid sequence of SEQ ID NO: 46 and the TCR β chain CDR2 may have an amino acid sequence of SEQ ID NO: 47.

In examples where the TCR β chain has the amino acid sequence of SEQ ID NO:55, the TCR β chain may be encoded by the nucleic acid sequence of SEQ ID NO:56, or a genetically degenerate sequence thereof (i.e. other nucleic acid sequences that encode the same protein as a result of the degeneracy of the genetic code). It is noted that SEQ ID NO:56 is the nucleic acid sequence for TCR β chain of clone CT44.6G4.

In an example, the nucleic acid composition provided herein comprises a nucleic acid sequence that encodes a TCR Vβ domain comprising a CDR3 amino acid sequence having at least 80% sequence identity to SEQ ID NO:48, or a functional fragment thereof.

In another example, the CDR3 of the Vβ domain of a nucleic acid composition provided herein comprises or consists of the amino acid sequence of SEQ ID NO:48.

In a further example, the Vβ domain of a nucleic acid composition provided herein comprises an amino acid sequence having at least 80% sequence identity to, comprising, or consisting of, SEQ ID NO: 51.

The TCR Vβ domain sequences derived from TCR clone CT44.6G4 discussed above are particularly compatible with the TCR Vα domain sequences derived from TCR clone CT44.6G4 discussed elsewhere herein.

Accordingly, in one example, a nucleic acid composition described herein encodes a MAGE-A1 antigen-specific binding protein having TCR Vα domain comprising a CDR3 amino acid sequence having at least 80% sequence identity to SEQ ID NO:45, or a functional fragment thereof; and a nucleic acid sequence that encodes a TCR Vβ domain comprising a CDR3 amino acid sequence having at least 80% sequence identity to SEQ ID NO:48, or a functional fragment thereof.

In a particular example, a nucleic acid composition described herein encodes a MAGE-A1 antigen-specific binding protein having a TCR Vα domain with a CDR3 amino acid sequence comprising or consisting of the amino acid sequence of SEQ ID NO: 45; and a TCR Vβ domain with a CDR3 comprising or consisting of the amino acid sequence of SEQ ID NO:48. In addition, the MAGE-A1 antigen may comprise or consist of the sequence shown in SEQ ID NO: 130. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 49; and the Vβ domain may comprise an amino acid sequence having at least 80% sequence identity to, comprising, or consisting of, SEQ ID NO: 51. In one example, the Vα domain comprises the amino acid sequence of SEQ ID NO: 49 and the Vβ domain comprises the amino acid sequence of SEQ ID NO: 51. In such cases, the Vα domain may be encoded by a nucleic acid sequence comprising the sequence of SEQ ID NO: 50; and the Vβ domain may be encoded by a nucleic acid sequence comprising the sequence of SEQ ID NO: 52.

In this particular example, the TCR Vα domain may include a CDR1 amino acid sequence comprising or consisting of the amino acid sequence of SEQ ID NO: 43 and a CDR2 amino acid sequence comprising or consisting of the amino acid sequence of SEQ ID NO:44. Furthermore, the TCR Vβ domain may include a CDR1 amino acid sequence comprising or consisting of the amino acid sequence of SEQ ID NO:46 and a CDR2 amino acid sequence comprising or consisting of the amino acid sequence of SEQ ID NO: 47.

For the avoidance of doubt, this particular example encompasses components of TCR clone CT44.6G4 exemplified herein. The different components of TCR clone CT44.6G4 and their respective SEQ ID Nos are summarised in Table 9 below.

As stated in more detail elsewhere herein, the nucleic acid composition described herein encodes both a TCR Vα domain and a TCR Vβ domain, which form the binding protein that is capable of specifically binding to a MAGE antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

(v) TCR Components that Interact with SLFRAVITK (SEQ ID NO: 131)

As provided elsewhere herein, the inventors have also identified TCR clone CT23.3H4 (3H4) which interacts with SLFRAVITK (SEQ ID NO: 131) in the context of HLA-A*03:01. The sequences provided herein that correspond to TCR clone CT23.3H4 are SEQ ID NO:s 57 to 70.

In one embodiment, an isolated nucleic acid composition that encodes a MAGE antigen-specific binding protein having a TCR α chain variable (Vα) domain and a TCR β chain variable (Vβ) domain is provided, the composition comprising:
a nucleic acid sequence that encodes a TCR Vα domain comprising a CDR3 amino acid sequence having at least 80% sequence identity to SEQ ID NO:59, or a functional fragment thereof; and a nucleic acid sequence that encodes a TCR Vβ domain comprising a CDR3 amino acid sequence having at least 80% sequence identity to SEQ ID NO: 62, or a functional fragment thereof, wherein the CDR3 sequences together specifically bind to MAGE-A1.

An example of an appropriate TCR Vα domain CDR3 amino acid sequence that confers specific binding to a MAGE antigen, in particular a MAGE-A1 antigen (e.g. to SLFRAVITK (SEQ ID NO: 131)), is shown in SEQ ID NO: 59. As would be clear to a person of skill in the art, variants of the amino acid sequence shown in SEQ ID NO: 59 may also be functional (i.e. retain their ability to confer specific binding to a MAGE-A1 antigen (e.g. to the peptide SLFRAVITK (SEQ ID NO: 131)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 59, i.e. they may have at least 80%, at least 84%, at least 92%, or 100% sequence identity to SEQ ID NO: 59. Suitably, percent identity is calculated as the percentage of identity to the entire length of the reference sequence (e.g. SEQ ID NO: 59). In other words, appropriate (functional) Vα domain CDR3 amino acid sequences may vary from the sequence shown in SEQ ID NO: 59 by one or several (e.g. two etc) amino acids.

As stated above, functional variants of SEQ ID NO: 59 retain their ability to confer specific binding to a MAGE-A1 antigen (i.e. the peptide shown in SEQ ID NO: 131) when the CDR3 is part of TCR Vα domain.

Functional variants may be naturally occurring, synthetic, or synthetically improved functional variants of SEQ ID NO: 59. The term “variant” also encompasses homologues and fragments. Functional variants will typically contain only conservative substitutions of one, two or more amino acids of SEQ ID NO: 59, or substitution, deletion or insertion of non-critical amino acids in non-critical regions of the CDR3.

Non-functional variants are amino acid sequence variants of SEQ ID NO: 59 that do not specifically bind to a MAGE-A1 antigen (i.e. the peptide shown in SEQ ID NO: 131). Non-functional variants will typically contain a non-conservative substitution, a deletion, or insertion or premature truncation of the amino acid sequence of SEQ ID NO: 59 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 59. In examples where the TCR Vα domain CDR3 has the amino acid sequence of SEQ ID NO: 59, the CDR3 may be encoded by any appropriate nucleic acid sequence.

The encoded TCR Vα domain may comprise, in addition to the specified CDR3, a CDR1 comprising an amino acid sequence of SEQ ID NO: 57, or a functional variant thereof (i.e, wherein the variant retains the ability to specifically bind to a MAGE-A1 antigen (e.g. the peptide shown in SEQ ID NO: 131)). Such functional variants may be naturally occurring, synthetic, or synthetically improved functional variants of SEQ ID NO: 57. The term “variant” also encompasses homologues and fragments. Functional variants will typically contain only conservative substitutions of one or more amino acids of SEQ ID NO: 57, or substitution, deletion or insertion of non-critical amino acids in non-critical regions of the protein.

Non-functional variants are amino acid sequence variants of SEQ ID NO: 57 that do not specifically bind to a MAGE-A1 antigen (e.g. the peptide shown in SEQ ID NO: 131). Non-functional variants will typically contain a non-conservative substitution, a deletion, or insertion or premature truncation of the amino acid sequence of SEQ ID NO: 57 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 57, i.e. it may have at least 80%, at least 83%, or 100% sequence identity to SEQ ID NO: 57. Suitably, percent identity is calculated as the percentage of identity to the entire length of the reference sequence (e.g. SEQ ID NO: 57). In other words, appropriate functional Vα domain CDR1 amino acid sequences may vary from the sequence shown in SEQ ID NO: 57 by one or several amino acids. As stated previously, the variant may comprise an amino acid substitution such as a conservative amino acid substitution compared to the sequence shown in SEQ ID NO: 57. As stated above, functional variants of SEQ ID NO: 57 retain the ability to specifically bind to a MAGE-A1 antigen (e.g. the peptide shown in SEQ ID NO: 131) when the CDR1 is part of TCR Vα domain.

In one example, the CDR1 of the Vα domain comprises or consists of the amino acid sequence of SEQ ID NO: 57. In examples where the TCR Vα domain CDR1 has the amino acid sequence of SEQ ID NO: 57, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 58, or a functional variant thereof (i.e, wherein the variant retains the ability to specifically bind to HLA-A*03:01). Such functional variants may be naturally occurring, synthetic, or synthetically improved functional variants of SEQ ID NO: 58. The term “variant” also encompasses homologues and fragments. Functional variants will typically contain only conservative substitutions of one or more amino acids of SEQ ID NO: 58, or substitution, deletion or insertion of non-critical amino acids in non-critical regions of the protein.

Non-functional variants are amino acid sequence variants of SEQ ID NO: 58 that do not specifically bind to HLA-A*03:01. Non-functional variants will typically contain a non-conservative substitution, a deletion, or insertion or premature truncation of the amino acid sequence of SEQ ID NO: 58 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 58, i.e. it may have at least 80%, at least 83%, or 100% sequence identity to SEQ ID NO: 58. Suitably, percent identity is calculated as the percentage of identity to the entire length of the reference sequence (e.g. SEQ ID NO: 58). In other words, appropriate (functional) Vα domain CDR2 amino acid sequences may vary from the sequence shown in SEQ ID NO: 58 by one or several amino acids. As stated previously, the variant may comprise an amino acid substitution such as a conservative amino acid substitution compared to the sequence shown in SEQ ID NO: 58. As stated above, a functional variant of SEQ ID NO: 58 retains the ability to specifically bind to HLA-A*03:01.

In one example, the CDR2 of the Vα domain comprises or consists of the amino acid sequence of SEQ ID NO: 58. In examples where the TCR Vα domain CDR2 has the amino acid sequence of SEQ ID NO: 58, the CDR2 may be encoded by any appropriate nucleic acid sequence.

The encoded TCR Vα domain may therefore comprise the CDRs mentioned in detail above (by SEQ ID specifically i.e. SEQ ID NO:59, SEQ ID NO: 57 and SEQ ID NO: 58, or functional variants thereof), with appropriate intervening sequences between the CDRs.

The encoded TCR Vα domain may comprise an amino acid sequence of SEQ ID NO: 63, or a functional variant thereof (i.e, wherein the variant TCR Vα domain retains the ability to specifically bind to a MAGE-A1 antigen (e.g. the peptide shown in SEQ ID NO: 131) when part of a binding protein described herein). Such functional variants may be naturally occurring, synthetic, or synthetically improved functional variants of SEQ ID NO: 63. The term “variant” also encompasses homologues and fragments. Functional variants will typically contain only conservative substitutions of one or more amino acids of SEQ ID NO: 63, or substitution, deletion or insertion of non-critical amino acids in non-critical regions of the protein.

Non-functional variants are amino acid sequence variants of SEQ ID NO: 63 that do not specifically bind to a MAGE-A1 antigen (e.g. the peptide shown in SEQ ID NO: 131). Non-functional variants will typically contain a non-conservative substitution, a deletion, or insertion or premature truncation of the amino acid sequence of SEQ ID NO: 63 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 63, whilst retaining the ability to specifically bind to a MAGE-A1 antigen (e.g. the peptide shown in SEQ ID NO: 131). In other words, a functional TCR Vα domain with one or several amino acid substitutions compared to the sequence of SEQ ID NO: 63 is also encompassed. As stated previously, the amino acid substitution may be a conservative amino acid substitution. The variability in sequence compared to SEQ ID NO: 63 may all be in regions of the TCR Vα domain that do not form CDRs (i.e. the variant may have the CDRs of SEQ ID NO: 59, SEQ ID NO: 57 and/or SEQ ID NO: 58, and still have 25% (or less) sequence variability compared to SEQ ID NO: 63). In other words, the sequence of the CDRs of SEQ ID NO: 63 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 63).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 63, wherein the TCR Vα domain comprises a CDR3 having an amino acid sequence of SEQ ID NO: 59. In this example, the TCR Vα domain CDR1 may have an amino acid sequence of SEQ ID NO: 57 and the TCR Vα domain CDR2 may have an amino acid sequence of SEQ ID NO: 58.

As another example, the encoded TCR Vα domain may comprise an amino acid sequence having the amino acid sequence of SEQ ID NO: 63, with 0 to 10 (or 0 to 5) amino acid substitutions, insertions or deletions, wherein the TCR Vα domain comprises a CDR3 having an amino acid sequence of SEQ ID NO: 59. In this example, the TCR Vα domain CDR1 may have an amino acid sequence of SEQ ID NO: 57 and the TCR Vα domain CDR2 may have an amino acid sequence of SEQ ID NO: 58.

In examples where the TCR Vα domain has the amino acid sequence of SEQ ID NO: 63, the TCR Vα domain may be encoded by the nucleic acid sequence of SEQ ID NO: 64, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

An example of a specific TCR α chain amino acid sequence that includes a TCR Vα domain described herein with an appropriate constant domain is shown in SEQ ID NO: 67. Appropriate functional variants of SEQ ID NO:67 are also encompassed (e.g. variants having at least 75% (e.g. at least 75%, at least 80%, at least 85%, at least 90%, at least 95% etc) sequence identity to the amino acid sequence of SEQ ID NO: 67, wherein the variant TCR α chain amino acid sequence retains its ability to specifically bind to a MAGE-A1 antigen (e.g. the peptide shown in SEQ ID NO: 131) when part of a binding protein described herein). In other words, a functional TCR α chain with one or several amino acid substitutions compared to the sequence of SEQ ID NO:67 is also encompassed. As stated previously, the amino acid substitution may be a conservative amino acid substitution. The variability in sequence compared to SEQ ID NO: 67 may all be in regions of the TCR α chain that do not form CDRs (i.e. the variant may have the CDRs of SEQ ID NO: 59, SEQ ID NO: 57 and/or SEQ ID NO: 58, and still have 25% (or less) sequence variability compared to SEQ ID NO: 67). In other words, the sequence of the CDRs of SEQ ID NO: 67 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 67).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 67, wherein the TCR α chain comprises a CDR3 having an amino acid sequence of SEQ ID NO: 59. In this example, the TCR α chain CDR1 may have an amino acid sequence of SEQ ID NO:57 and the TCR α chain CDR2 may have an amino acid sequence of SEQ ID NO: 58.

In examples where the TCR α chain has the amino acid sequence of SEQ ID NO: 67, the TCR α chain may be encoded by the nucleic acid sequence of SEQ ID NO: 68, or a genetically degenerate sequence thereof (i.e. other nucleic acid sequences that encode the same protein as a result of the degeneracy of the genetic code). It is noted that SEQ ID NO:68 is the nucleic acid sequence for TCR α chain of clone CT23.3H4.

In one example, the nucleic acid composition provided herein comprises a nucleic acid sequence that encodes a TCR Vα domain comprising a CDR3 amino acid sequence having at least 80% sequence identity to SEQ ID NO:59, or a functional fragment thereof.

In another example, the CDR3 of the Vα domain of a nucleic acid composition provided herein comprises or consists of the amino acid sequence of SEQ ID NO: 59.

In another example, the Vα domain of a nucleic acid composition provided herein comprises an amino acid sequence having at least 80% sequence identity to, comprising, or consisting of, SEQ ID NO: 63.

As provided above, the inventors identified TCR clone CT23.3H4 which interacts with SLFRAVITK (SEQ ID NO: 131) in the context of HLA-A*03:01. The sequences provided herein that correspond to TCR clone CT23.3H4 are SEQ ID NO:s 57 to 70.

An example of an appropriate TCR Vβ domain CDR3 amino acid sequence that confers specific binding to a MAGE-A1 antigen (e.g. to SLFRAVITK (SEQ ID NO: 131)) is shown in SEQ ID NO:62. As would be clear to a person of skill in the art, variants of the amino acid sequence shown in SEQ ID NO:62 may also be functional (i.e. retain their ability to confer specific binding to a MAGE-A1 antigen (i.e. the peptide shown in SEQ ID NO: 131) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 62, i.e. they may have at least 80%, at least 85%, at least 92%, or 100% sequence identity to SEQ ID NO: 62. Suitably, percent identity is calculated as the percentage of identity to the entire length of the reference sequence (e.g. SEQ ID NO: 62). In other words, appropriate (functional) Vβ domain CDR3 amino acid sequences may vary from the sequence shown in SEQ ID NO: 62 by one or several (e.g. two) amino acids. As stated above, functional variants of SEQ ID NO: 62 retain their ability to confer specific binding to a MAGE-A1 antigen (e.g. the peptide shown in SEQ ID NO: 131) when the CDR3 is part of TCR Vβ domain.

Functional variants may be naturally occurring, synthetic, or synthetically improved functional variants of SEQ ID NO: 62. The term “variant” also encompasses homologues and fragments. Functional variants will typically contain only conservative substitutions of one or more amino acids of SEQ ID NO: 62, or substitution, deletion or insertion of non-critical amino acids in non-critical regions of the CDR3.

Non-functional variants are amino acid sequence variants of SEQ ID NO: 62 that do not specifically bind to a MAGE-A1 antigen (e.g. the peptide shown in SEQ ID NO: 131). Non-functional variants will typically contain a non-conservative substitution, a deletion, or insertion or premature truncation of the amino acid sequence of SEQ ID NO: 62 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 62. In examples where the TCR Vβ domain CDR3 has the amino acid sequence of SEQ ID NO:62, the CDR3 may be encoded by any appropriate nucleic acid sequence.

The encoded TCR Vβ domain may comprise, in addition to the specified CDR3, a CDR1 comprising an amino acid sequence of SEQ ID NO: 60, or a functional variant thereof (i.e, wherein the variant retains the ability to specifically bind to a MAGE-A1 antigen (e.g. the peptide shown in SEQ ID NO: 131)). Such functional variants may be naturally occurring, synthetic, or synthetically improved functional variants of SEQ ID NO: 60. The term “variant” also encompasses homologues and fragments. Functional variants will typically contain only conservative substitutions of one or more amino acids of SEQ ID NO: 60, or substitution, deletion or insertion of non-critical amino acids in non-critical regions of the protein.

Non-functional variants are amino acid sequence variants of SEQ ID NO: 60 that do not specifically bind to a MAGE-A1 antigen (e.g. the peptide shown in SEQ ID NO: 131). Non-functional variants will typically contain a non-conservative substitution, a deletion, or insertion or premature truncation of the amino acid sequence of SEQ ID NO: 60 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 60, i.e. it may have at least 80%, at least 83%, or 100% sequence identity to SEQ ID NO: 60. Suitably, percent identity is calculated as the percentage of identity to the entire length of the reference sequence (e.g. SEQ ID NO: 60). In other words, appropriate (functional) Vβ domain CDR1 amino acid sequences may vary from the sequence shown in SEQ ID NO:60 by one or several amino acids. As stated previously, the variant may comprise an amino acid substitution such as a conservative amino acid substitution compared to the sequence shown in SEQ ID NO:60. As stated above, functional variants of SEQ ID NO: 60 retain the ability to specifically bind to a MAGE-A1 antigen (e.g. the peptide shown in SEQ ID NO: 131) when the CDR1 is part of TCR Vβ domain.

In one example, the CDR1 of the Vβ domain comprises or consists of the amino acid sequence of SEQ ID NO: 60. In examples where the TCR Vα domain CDR1 has the amino acid sequence of SEQ ID NO:60, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 61, or a functional variant thereof (i.e, wherein the variant retains the ability to specifically bind to HLA-A*03:01). Such functional variants may be naturally occurring, synthetic, or synthetically improved functional variants of SEQ ID NO: 61. The term “variant” also encompasses homologues and fragments. Functional variants will typically contain only conservative substitutions of one or more amino acids of SEQ ID NO: 61, or substitution, deletion or insertion of non-critical amino acids in non-critical regions of the protein.

Non-functional variants are amino acid sequence variants of SEQ ID NO: 61 that do not specifically bind to HLA-A*03:01. Non-functional variants will typically contain a non-conservative substitution, a deletion, or insertion or premature truncation of the amino acid sequence of SEQ ID NO: 61 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 61, i.e. it may have at least 80%, at least 83, or 100% sequence identity to SEQ ID NO: 61. Suitably, percent identity is calculated as the percentage of identity to the entire length of the reference sequence (e.g. SEQ ID NO: 61). In other words, appropriate (functional) Vβ domain CDR2 amino acid sequences may vary from the sequence shown in SEQ ID NO: 61 by one or several amino acids. As stated previously, the variant may comprise an amino acid substitution such as a conservative amino acid substitution compared to the sequence shown in SEQ ID NO: 61. As stated above, a functional variant of SEQ ID NO: 61 retains the ability to specifically bind to HLA-A*03:01.

In one example, the CDR2 of the Vβ domain comprises or consists of the amino acid sequence of SEQ ID NO: 61. In examples where the TCR Vβ domain CDR2 has the amino acid sequence of SEQ ID NO:61, the CDR2 may be encoded by any appropriate nucleic acid sequence.

The encoded TCR Vβ domain may therefore comprise the CDRs mentioned in detail above (by SEQ ID specifically i.e. SEQ ID NO:62, SEQ ID NO: 60 and SEQ ID NO: 61, or functional variants thereof), with appropriate intervening sequences between the CDRs.

The encoded TCR Vβ domain may have an amino acid sequence of SEQ ID NO: 65, or a functional variant thereof (i.e, wherein the variant TCR Vβ domain retains the ability to specifically bind to a MAGE-A1 antigen (e.g. the peptide shown in SEQ ID NO: 131) when part of a binding protein described herein). Such functional variants may be naturally occurring, synthetic, or synthetically improved functional variants of SEQ ID NO: 65. The term “variant” also encompasses homologues and fragments. Functional variants will typically contain only conservative substitutions of one or more amino acids of SEQ ID NO: 65, or substitution, deletion or insertion of non-critical amino acids in non-critical regions of the protein.

Non-functional variants are amino acid sequence variants of SEQ ID NO: 65 that do not specifically bind to a MAGE-A1 antigen (e.g. the peptide shown in SEQ ID NO: 131). Non-functional variants will typically contain a non-conservative substitution, a deletion, or insertion or premature truncation of the amino acid sequence of SEQ ID NO:65 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 65, whilst retaining the ability to specifically bind to a MAGE-A1 antigen (e.g. the peptide shown in SEQ ID NO: 131). In other words, a functional TCR Vβ domain with one or several amino acid substitutions compared to the sequence of SEQ ID NO: 65 is also encompassed. As stated previously, the amino acid substitution may be a conservative amino acid substitution. The variability in sequence compared to SEQ ID NO:65 may all be in regions of the TCR Vβ domain that do not form CDRs (i.e. the variant may have the CDRs of SEQ ID NO: 62, SEQ ID NO: 60 and/or SEQ ID NO: 61, and still have 25% (or less) sequence variability compared to SEQ ID NO: 65). In other words, the sequence of the CDRs of SEQ ID NO: 65 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 65).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 65, wherein the TCR Vβ domain comprises a CDR3 having an amino acid sequence of SEQ ID NO: 62. In this example, the TCR Vβ domain CDR1 may have an amino acid sequence of SEQ ID NO:60 and the TCR Vβ domain CDR2 may have an amino acid sequence of SEQ ID NO: 61.

In examples where the TCR Vβ domain has the amino acid sequence of SEQ ID NO:65, the TCR Vβ domain may be encoded by the nucleic acid sequence of SEQ ID NO: 66, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 69. Appropriate functional variants of SEQ ID NO: 69 are also encompassed (e.g. variants having at least 75% (e.g. at least 75%, at least 80%, at least 85%, at least 90%, at least 95% etc) sequence identity to the amino acid sequence of SEQ ID NO: 69, wherein the variant TCR β chain amino acid sequence retains its ability to specifically bind to a MAGE-A1 antigen (e.g. the peptide shown in SEQ ID NO: 131) when part of a binding protein described herein). In other words, a functional TCR β chain with one or several amino acid substitutions compared to the sequence of SEQ ID NO: 69 is also encompassed. As stated previously, the amino acid substitution may be a conservative amino acid substitution. The variability in sequence compared to SEQ ID NO: 69 may all be in regions of the TCR β chain that do not form CDRs (i.e. the variant may have the CDRs of SEQ ID NO: 62, SEQ ID NO: 60 and/or SEQ ID NO: 61, and still have 25% (or less) sequence variability compared to SEQ ID NO:69. In other words, the sequence of the CDRs of SEQ ID NO: 69 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 69).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 69, wherein the TCR β chain comprises a CDR3 having an amino acid sequence of SEQ ID NO: 62. In this example, the TCR β chain CDR1 may have an amino acid sequence of SEQ ID NO: 60 and the TCR β chain CDR2 may have an amino acid sequence of SEQ ID NO: 61.

In examples where the TCR β chain has the amino acid sequence of SEQ ID NO:69, the TCR chain may be encoded by the nucleic acid sequence of SEQ ID NO:70, or a genetically degenerate sequence thereof (i.e. other nucleic acid sequences that encode the same protein as a result of the degeneracy of the genetic code). It is noted that SEQ ID NO:70 is the nucleic acid sequence for TCR β chain of clone CT23.3H4.

In an example, the nucleic acid composition provided herein comprises a nucleic acid sequence that encodes a TCR Vβ domain comprising a CDR3 amino acid sequence having at least 80% sequence identity to SEQ ID NO:62, or a functional fragment thereof.

In another example, the CDR3 of the Vβ domain of a nucleic acid composition provided herein comprises or consists of the amino acid sequence of SEQ ID NO:62.

In a further example, the Vβ domain of a nucleic acid composition provided herein comprises an amino acid sequence having at least 80% sequence identity to, comprising, or consisting of, SEQ ID NO: 65.

The TCR Vβ domain sequences derived from TCR clone CT23.3H4 discussed above are particularly compatible with the TCR Vα domain sequences derived from TCR clone CT23.3H4 discussed elsewhere herein.

Accordingly, in one example, a nucleic acid composition described herein encodes a MAGE-A1 antigen-specific binding protein having TCR Vα domain comprising a CDR3 amino acid sequence having at least 80% sequence identity to SEQ ID NO:59, or a functional fragment thereof; and a nucleic acid sequence that encodes a TCR Vβ domain comprising a CDR3 amino acid sequence having at least 80% sequence identity to SEQ ID NO:62, or a functional fragment thereof.

In a particular example, a nucleic acid composition described herein encodes a MAGE-A1 antigen-specific binding protein having a TCR Vα domain with a CDR3 amino acid sequence comprising or consisting of the amino acid sequence of SEQ ID NO: 59; and a TCR Vβ domain with a CDR3 comprising or consisting of the amino acid sequence of SEQ ID NO:62. In addition, the MAGE-A1 antigen may comprise or consist of the sequence shown in SEQ ID NO: 131. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 63; and the Vβ domain may comprise an amino acid sequence having at least 80% sequence identity to, comprising, or consisting of, SEQ ID NO: 65. In one example, the Vα domain comprises the amino acid sequence of SEQ ID NO: 63 and the Vβ domain comprises the amino acid sequence of SEQ ID NO: 65. In such cases, the Vα domain may be encoded by a nucleic acid sequence comprising the sequence of SEQ ID NO: 64; and the Vβ domain may be encoded by a nucleic acid sequence comprising the sequence of SEQ ID NO: 66.

In this particular example, the TCR Vα domain may include a CDR1 amino acid sequence comprising or consisting of the amino acid sequence of SEQ ID NO:57 and a CDR2 amino acid sequence comprising or consisting of the amino acid sequence of SEQ ID NO:58. Furthermore, the TCR Vβ domain may include a CDR1 amino acid sequence comprising or consisting of the amino acid sequence of SEQ ID NO:60 and a CDR2 amino acid sequence comprising or consisting of the amino acid sequence of SEQ ID NO: 61.

For the avoidance of doubt, this particular example encompasses components of TCR clone CT23.3H4 exemplified herein. The different components of TCR clone CT23.3H4 and their respective SEQ ID Nos are summarised in Table 10 below.

As stated in more detail elsewhere herein, the nucleic acid composition described herein encodes both a TCR Vα domain and a TCR Vβ domain, which form the binding protein that is capable of specifically binding to a MAGE antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

(vi) Alternative TCR Components that Interact with SLFRAVITK (SEQ ID NO: 131)

As provided elsewhere herein, the inventors have also identified TCR clone MRM23.3B2 (3B2) which interacts with SLFRAVITK (SEQ ID NO: 131) in the context of HLA-A*03:01. The sequences provided herein that correspond to TCR clone MRM23.3B2 are SEQ ID NO:s 71 to 84.

In one embodiment, an isolated nucleic acid composition that encodes a MAGE antigen-specific binding protein having a TCR α chain variable (Vα) domain and a TCR β chain variable (Vβ) domain is provided, the composition comprising:
a nucleic acid sequence that encodes a TCR Vα domain comprising a CDR3 amino acid sequence having at least 80% sequence identity to SEQ ID NO:73, or a functional fragment thereof; and a nucleic acid sequence that encodes a TCR Vβ domain comprising a CDR3 amino acid sequence having at least 80% sequence identity to SEQ ID NO: 76, or a functional fragment thereof, wherein the CDR3 sequences together specifically bind to MAGE-A1.

An example of an appropriate TCR Vα domain CDR3 amino acid sequence that confers specific binding to a MAGE antigen in particular a MAGE-A1 antigen (e.g. to SLFRAVITK (SEQ ID NO: 131)), is shown in SEQ ID NO: 73. As would be clear to a person of skill in the art, variants of the amino acid sequence shown in SEQ ID NO: 73 may also be functional (i.e. retain their ability to confer specific binding to a MAGE-A1 antigen (e.g. to the peptide SLFRAVITK (SEQ ID NO: 131)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 73, i.e. they may have at least 80%, at least 84%, at least 92%, or 100% sequence identity to SEQ ID NO: 73. Suitably, percent identity is calculated as the percentage of identity to the entire length of the reference sequence (e.g. SEQ ID NO: 73). In other words, appropriate (functional) Vα domain CDR3 amino acid sequences may vary from the sequence shown in SEQ ID NO: 73 by one or several (e.g. two etc) amino acids.

As stated above, functional variants of SEQ ID NO: 73 retain their ability to confer specific binding to a MAGE-A1 antigen (i.e. the peptide shown in SEQ ID NO: 131) when the CDR3 is part of TCR Vα domain.

Functional variants may be naturally occurring, synthetic, or synthetically improved functional variants of SEQ ID NO: 73. The term “variant” also encompasses homologues and fragments. Functional variants will typically contain only conservative substitutions of one, two or more amino acids of SEQ ID NO: 73, or substitution, deletion or insertion of non-critical amino acids in non-critical regions of the CDR3.

Non-functional variants are amino acid sequence variants of SEQ ID NO: 73 that do not specifically bind to a MAGE-A1 antigen (i.e. the peptide shown in SEQ ID NO: 131). Non-functional variants will typically contain a non-conservative substitution, a deletion, or insertion or premature truncation of the amino acid sequence of SEQ ID NO: 73 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 73. In examples where the TCR Vα domain CDR3 has the amino acid sequence of SEQ ID NO: 73, the CDR3 may be encoded by any appropriate nucleic acid sequence.

The encoded TCR Vα domain may comprise, in addition to the specified CDR3, a CDR1 comprising an amino acid sequence of SEQ ID NO: 71, or a functional variant thereof (i.e, wherein the variant retains the ability to specifically bind to a MAGE-A1 antigen (e.g. the peptide shown in SEQ ID NO: 131)). Such functional variants may be naturally occurring, synthetic, or synthetically improved functional variants of SEQ ID NO: 71. The term “variant” also encompasses homologues and fragments. Functional variants will typically contain only conservative substitutions of one or more amino acids of SEQ ID NO: 71, or substitution, deletion or insertion of non-critical amino acids in non-critical regions of the protein.

Non-functional variants are amino acid sequence variants of SEQ ID NO: 71 that do not specifically bind to a MAGE-A1 antigen (e.g. the peptide shown in SEQ ID NO: 131). Non-functional variants will typically contain a non-conservative substitution, a deletion, or insertion or premature truncation of the amino acid sequence of SEQ ID NO: 71 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 71, i.e. it may have at least 80%, at least 83%, or 100% sequence identity to SEQ ID NO: 71. Suitably, percent identity is calculated as the percentage of identity to the entire length of the reference sequence (e.g. SEQ ID NO: 71). In other words, appropriate functional Vα domain CDR1 amino acid sequences may vary from the sequence shown in SEQ ID NO: 71 by one or several amino acids. As stated previously, the variant may comprise an amino acid substitution such as a conservative amino acid substitution compared to the sequence shown in SEQ ID NO: 71. As stated above, functional variants of SEQ ID NO: 71 retain the ability to specifically bind to a MAGE-A1 antigen (e.g. the peptide shown in SEQ ID NO: 131) when the CDR1 is part of TCR Vα domain.

In one example, the CDR1 of the Vα domain comprises or consists of the amino acid sequence of SEQ ID NO: 71. In examples where the TCR Vα domain CDR1 has the amino acid sequence of SEQ ID NO: 71, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 72, or a functional variant thereof (i.e, wherein the variant retains the ability to specifically bind to HLA-A*03:01). Such functional variants may be naturally occurring, synthetic, or synthetically improved functional variants of SEQ ID NO: 72. The term “variant” also encompasses homologues and fragments. Functional variants will typically contain only conservative substitutions of one or more amino acids of SEQ ID NO: 72, or substitution, deletion or insertion of non-critical amino acids in non-critical regions of the protein.

Non-functional variants are amino acid sequence variants of SEQ ID NO: 72 that do not specifically bind to HLA-A*03:01. Non-functional variants will typically contain a non-conservative substitution, a deletion, or insertion or premature truncation of the amino acid sequence of SEQ ID NO: 72 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 72, i.e. it may have at least 80%, or 100% sequence identity to SEQ ID NO: 72. Suitably, percent identity is calculated as the percentage of identity to the entire length of the reference sequence (e.g. SEQ ID NO: 72). In other words, appropriate (functional) Vα domain CDR2 amino acid sequences may vary from the sequence shown in SEQ ID NO: 72 by one or several amino acids. As stated previously, the variant may comprise an amino acid substitution such as a conservative amino acid substitution compared to the sequence shown in SEQ ID NO: 72. As stated above, a functional variant of SEQ ID NO: 72 retains the ability to specifically bind to HLA-A*03:01.

In one example, the CDR2 of the Vα domain comprises or consists of the amino acid sequence of SEQ ID NO: 72. In examples where the TCR Vα domain CDR2 has the amino acid sequence of SEQ ID NO: 72, the CDR2 may be encoded by any appropriate nucleic acid sequence.

The encoded TCR Vα domain may therefore comprise the CDRs mentioned in detail above (by SEQ ID specifically i.e. SEQ ID NO:73, SEQ ID NO: 71 and SEQ ID NO: 72, or functional variants thereof), with appropriate intervening sequences between the CDRs.

The encoded TCR Vα domain may comprise an amino acid sequence of SEQ ID NO: 77, or a functional variant thereof (i.e, wherein the variant TCR Vα domain retains the ability to specifically bind to a MAGE-A1 antigen (e.g. the peptide shown in SEQ ID NO: 131) when part of a binding protein described herein). Such functional variants may be naturally occurring, synthetic, or synthetically improved functional variants of SEQ ID NO: 77. The term “variant” also encompasses homologues and fragments. Functional variants will typically contain only conservative substitutions of one or more amino acids of SEQ ID NO: 77, or substitution, deletion or insertion of non-critical amino acids in non-critical regions of the protein.

Non-functional variants are amino acid sequence variants of SEQ ID NO: 77 that do not specifically bind to a MAGE-A1 antigen (e.g. the peptide shown in SEQ ID NO: 131). Non-functional variants will typically contain a non-conservative substitution, a deletion, or insertion or premature truncation of the amino acid sequence of SEQ ID NO: 77 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 77, whilst retaining the ability to specifically bind to a MAGE-A1 antigen (e.g. the peptide shown in SEQ ID NO: 131). In other words, a functional TCR Vα domain with one or several amino acid substitutions compared to the sequence of SEQ ID NO: 77 is also encompassed. As stated previously, the amino acid substitution may be a conservative amino acid substitution. The variability in sequence compared to SEQ ID NO: 77 may all be in regions of the TCR Vα domain that do not form CDRs (i.e. the variant may have the CDRs of SEQ ID NO: 73, SEQ ID NO: 71 and/or SEQ ID NO: 72, and still have 25% (or less) sequence variability compared to SEQ ID NO:77). In other words, the sequence of the CDRs of SEQ ID NO: 77 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 77).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 77, wherein the TCR Vα domain comprises a CDR3 having an amino acid sequence of SEQ ID NO: 73. In this example, the TCR Vα domain CDR1 may have an amino acid sequence of SEQ ID NO: 71 and the TCR Vα domain CDR2 may have an amino acid sequence of SEQ ID NO: 72.

As another example, the encoded TCR Vα domain may comprise an amino acid sequence having the amino acid sequence of SEQ ID NO: 77, with 0 to 10 (or 0 to 5) amino acid substitutions, insertions or deletions, wherein the TCR Vα domain comprises a CDR3 having an amino acid sequence of SEQ ID NO: 73. In this example, the TCR Vα domain CDR1 may have an amino acid sequence of SEQ ID NO: 71 and the TCR Vα domain CDR2 may have an amino acid sequence of SEQ ID NO: 72.

In examples where the TCR Vα domain has the amino acid sequence of SEQ ID NO: 77, the TCR Vα domain may be encoded by the nucleic acid sequence of SEQ ID NO: 78, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

An example of a specific TCR α chain amino acid sequence that includes a TCR Vα domain described herein with an appropriate constant domain is shown in SEQ ID NO: 81. Appropriate functional variants of SEQ ID NO:81 are also encompassed (e.g. variants having at least 75% (e.g. at least 75%, at least 80%, at least 85%, at least 90%, at least 95% etc) sequence identity to the amino acid sequence of SEQ ID NO: 81, wherein the variant TCR α chain amino acid sequence retains its ability to specifically bind to a MAGE-A1 antigen (e.g. the peptide shown in SEQ ID NO: 131) when part of a binding protein described herein). In other words, a functional TCR α chain with one or several amino acid substitutions compared to the sequence of SEQ ID NO:81 is also encompassed. As stated previously, the amino acid substitution may be a conservative amino acid substitution. The variability in sequence compared to SEQ ID NO: 81 may all be in regions of the TCR α chain that do not form CDRs (i.e. the variant may have the CDRs of SEQ ID NO: 73, SEQ ID NO: 71 and/or SEQ ID NO: 72, and still have 25% (or less) sequence variability compared to SEQ ID NO: 81). In other words, the sequence of the CDRs of SEQ ID NO: 81 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 81).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 81, wherein the TCR α chain comprises a CDR3 having an amino acid sequence of SEQ ID NO: 73. In this example, the TCR α chain CDR1 may have an amino acid sequence of SEQ ID NO:71 and the TCR α chain CDR2 may have an amino acid sequence of SEQ ID NO: 72.

In examples where the TCR α chain has the amino acid sequence of SEQ ID NO: 81, the TCR α chain may be encoded by the nucleic acid sequence of SEQ ID NO: 82, or a genetically degenerate sequence thereof (i.e. other nucleic acid sequences that encode the same protein as a result of the degeneracy of the genetic code). It is noted that SEQ ID NO: 82 is the nucleic acid sequence for TCR α chain of clone MRM23.3B2.

In one example, the nucleic acid composition provided herein comprises a nucleic acid sequence that encodes a TCR Vα domain comprising a CDR3 amino acid sequence having at least 80% sequence identity to SEQ ID NO:73, or a functional fragment thereof.

In another example, the CDR3 of the Vα domain of a nucleic acid composition provided herein comprises or consists of the amino acid sequence of SEQ ID NO: 73.

In another example, the Vα domain of a nucleic acid composition provided herein comprises an amino acid sequence having at least 80% sequence identity to, comprising, or consisting of, SEQ ID NO: 77.

As provided above, the inventors identified TCR clone MRM23.3B2 which interacts with SLFRAVITK (SEQ ID NO: 131) in the context of HLA-A*03:01. The sequences provided herein that correspond to TCR clone MRM23.3B2 are SEQ ID NO:s 71 to 84.

An example of an appropriate TCR Vβ domain CDR3 amino acid sequence that confers specific binding to a MAGE antigen, in particular a MAGE-A1 antigen (e.g. to SLFRAVITK (SEQ ID NO: 131)), is shown in SEQ ID NO:76. As would be clear to a person of skill in the art, variants of the amino acid sequence shown in SEQ ID NO:76 may also be functional (i.e. retain their ability to confer specific binding to a MAGE-A1 antigen (i.e. the peptide shown in SEQ ID NO: 131) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 76, i.e. they may have at least 80%, at least 86%, at least 93%, or 100% sequence identity to SEQ ID NO: 76. Suitably, percent identity is calculated as the percentage of identity to the entire length of the reference sequence (e.g. SEQ ID NO: 76). In other words, appropriate (functional) Vβ domain CDR3 amino acid sequences may vary from the sequence shown in SEQ ID NO: 76 by one or several (e.g. two) amino acids. As stated above, functional variants of SEQ ID NO: 76 retain their ability to confer specific binding to a MAGE-A1 antigen (e.g. the peptide shown in SEQ ID NO: 131) when the CDR3 is part of TCR Vβ domain.

Functional variants may be naturally occurring, synthetic, or synthetically improved functional variants of SEQ ID NO: 76. The term “variant” also encompasses homologues and fragments. Functional variants will typically contain only conservative substitutions of one or more amino acids of SEQ ID NO: 76, or substitution, deletion or insertion of non-critical amino acids in non-critical regions of the CDR3.

Non-functional variants are amino acid sequence variants of SEQ ID NO: 76 that do not specifically bind to a MAGE-A1 antigen (e.g. the peptide shown in SEQ ID NO: 131). Non-functional variants will typically contain a non-conservative substitution, a deletion, or insertion or premature truncation of the amino acid sequence of SEQ ID NO: 76 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 76. In examples where the TCR Vβ domain CDR3 has the amino acid sequence of SEQ ID NO:76, the CDR3 may be encoded by any appropriate nucleic acid sequence.

The encoded TCR Vβ domain may comprise, in addition to the specified CDR3, a CDR1 comprising an amino acid sequence of SEQ ID NO: 74, or a functional variant thereof (i.e, wherein the variant retains the ability to specifically bind to a MAGE-A1 antigen (e.g. the peptide shown in SEQ ID NO: 131)). Such functional variants may be naturally occurring, synthetic, or synthetically improved functional variants of SEQ ID NO: 74. The term “variant” also encompasses homologues and fragments. Functional variants will typically contain only conservative substitutions of one or more amino acids of SEQ ID NO: 74, or substitution, deletion or insertion of non-critical amino acids in non-critical regions of the protein.

Non-functional variants are amino acid sequence variants of SEQ ID NO: 74 that do not specifically bind to a MAGE-A1 antigen (e.g. the peptide shown in SEQ ID NO: 131). Non-functional variants will typically contain a non-conservative substitution, a deletion, or insertion or premature truncation of the amino acid sequence of SEQ ID NO: 74 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 74, i.e. it may have at least 80%, at least 83%, or 100% sequence identity to SEQ ID NO: 74. Suitably, percent identity is calculated as the percentage of identity to the entire length of the reference sequence (e.g. SEQ ID NO: 74). In other words, appropriate (functional) Vβ domain CDR1 amino acid sequences may vary from the sequence shown in SEQ ID NO:74 by one or several amino acids. As stated previously, the variant may comprise an amino acid substitution such as a conservative amino acid substitution compared to the sequence shown in SEQ ID NO:74. As stated above, functional variants of SEQ ID NO: 74 retain the ability to specifically bind to a MAGE-A1 antigen (e.g. the peptide shown in SEQ ID NO: 131) when the CDR1 is part of TCR Vβ domain.

In one example, the CDR1 of the Vβ domain comprises or consists of the amino acid sequence of SEQ ID NO: 74. In examples where the TCR Vα domain CDR1 has the amino acid sequence of SEQ ID NO:74, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 75, or a functional variant thereof (i.e, wherein the variant retains the ability to specifically bind to HLA-A*03:01). Such functional variants may be naturally occurring, synthetic, or synthetically improved functional variants of SEQ ID NO: 75. The term “variant” also encompasses homologues and fragments. Functional variants will typically contain only conservative substitutions of one or more amino acids of SEQ ID NO: 75, or substitution, deletion or insertion of non-critical amino acids in non-critical regions of the protein.

Non-functional variants are amino acid sequence variants of SEQ ID NO: 75 that do not specifically bind to HLA-A*03:01. Non-functional variants will typically contain a non-conservative substitution, a deletion, or insertion or premature truncation of the amino acid sequence of SEQ ID NO: 75 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 75, i.e. it may have at least 80%, at least 83%, or 100% sequence identity to SEQ ID NO: 75. Suitably, percent identity is calculated as the percentage of identity to the entire length of the reference sequence (e.g. SEQ ID NO: 75). In other words, appropriate (functional) Vβ domain CDR2 amino acid sequences may vary from the sequence shown in SEQ ID NO: 75 by one or several amino acids. As stated previously, the variant may comprise an amino acid substitution such as a conservative amino acid substitution compared to the sequence shown in SEQ ID NO: 75. As stated above, a functional variant of SEQ ID NO: 75 retains the ability to specifically bind to HLA-A*03:01.

In one example, the CDR2 of the Vβ domain comprises or consists of the amino acid sequence of SEQ ID NO: 75. In examples where the TCR Vβ domain CDR2 has the amino acid sequence of SEQ ID NO:75, the CDR2 may be encoded by any appropriate nucleic acid sequence.

The encoded TCR Vβ domain may therefore comprise the CDRs mentioned in detail above (by SEQ ID specifically i.e. SEQ ID NO:76, SEQ ID NO: 74 and SEQ ID NO: 75, or functional variants thereof), with appropriate intervening sequences between the CDRs.

The encoded TCR Vβ domain may have an amino acid sequence of SEQ ID NO: 79, or a functional variant thereof (i.e, wherein the variant TCR Vβ domain retains the ability to specifically bind to a MAGE-A1 antigen (e.g. the peptide shown in SEQ ID NO: 131) when part of a binding protein described herein). Such functional variants may be naturally occurring, synthetic, or synthetically improved functional variants of SEQ ID NO: 79. The term “variant” also encompasses homologues and fragments. Functional variants will typically contain only conservative substitutions of one or more amino acids of SEQ ID NO: 79, or substitution, deletion or insertion of non-critical amino acids in non-critical regions of the protein.

Non-functional variants are amino acid sequence variants of SEQ ID NO: 79 that do not specifically bind to a MAGE-A1 antigen (e.g. the peptide shown in SEQ ID NO: 131). Non-functional variants will typically contain a non-conservative substitution, a deletion, or insertion or premature truncation of the amino acid sequence of SEQ ID NO:79 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 79, whilst retaining the ability to specifically bind to a MAGE-A1 antigen (e.g. the peptide shown in SEQ ID NO: 131). In other words, a functional TCR Vβ domain with one or several amino acid substitutions compared to the sequence of SEQ ID NO: 79 is also encompassed. As stated previously, the amino acid substitution may be a conservative amino acid substitution. The variability in sequence compared to SEQ ID NO:79 may all be in regions of the TCR Vβ domain that do not form CDRs (i.e. the variant may have the CDRs of SEQ ID NO: 76, SEQ ID NO: 74 and/or SEQ ID NO: 75, and still have 25% (or less) sequence variability compared to SEQ ID NO: 79). In other words, the sequence of the CDRs of SEQ ID NO: 79 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 79).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 79, wherein the TCR Vβ domain comprises a CDR3 having an amino acid sequence of SEQ ID NO: 76. In this example, the TCR Vβ domain CDR1 may have an amino acid sequence of SEQ ID NO:74 and the TCR Vβ domain CDR2 may have an amino acid sequence of SEQ ID NO: 75.

In examples where the TCR Vβ domain has the amino acid sequence of SEQ ID NO:79, the TCR Vβ domain may be encoded by the nucleic acid sequence of SEQ ID NO: 80, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 83. Appropriate functional variants of SEQ ID NO: 83 are also encompassed (e.g. variants having at least 75% (e.g. at least 75%, at least 80%, at least 85%, at least 90%, at least 95% etc) sequence identity to the amino acid sequence of SEQ ID NO: 83, wherein the variant TCR β chain amino acid sequence retains its ability to specifically bind to a MAGE-A1 antigen (e.g. the peptide shown in SEQ ID NO: 131) when part of a binding protein described herein). In other words, a functional TCR β chain with one or several amino acid substitutions compared to the sequence of SEQ ID NO: 83 is also encompassed. As stated previously, the amino acid substitution may be a conservative amino acid substitution. The variability in sequence compared to SEQ ID NO: 83 may all be in regions of the TCR β chain that do not form CDRs (i.e. the variant may have the CDRs of SEQ ID NO: 76, SEQ ID NO: 74 and/or SEQ ID NO: 75, and still have 25% (or less) sequence variability compared to SEQ ID NO:83. In other words, the sequence of the CDRs of SEQ ID NO: 83 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 83).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 83, wherein the TCR β chain comprises a CDR3 having an amino acid sequence of SEQ ID NO: 76. In this example, the TCR β chain CDR1 may have an amino acid sequence of SEQ ID NO: 74 and the TCR β chain CDR2 may have an amino acid sequence of SEQ ID NO: 75.

In examples where the TCR β chain has the amino acid sequence of SEQ ID NO:83, the TCR β chain may be encoded by the nucleic acid sequence of SEQ ID NO:84, or a genetically degenerate sequence thereof (i.e. other nucleic acid sequences that encode the same protein as a result of the degeneracy of the genetic code). It is noted that SEQ ID NO:84 is the nucleic acid sequence for TCR β chain of clone MRM23.3B2.

In an example, the nucleic acid composition provided herein comprises a nucleic acid sequence that encodes a TCR Vβ domain comprising a CDR3 amino acid sequence having at least 80% sequence identity to SEQ ID NO:76, or a functional fragment thereof.

In another example, the CDR3 of the Vβ domain of a nucleic acid composition provided herein comprises or consists of the amino acid sequence of SEQ ID NO:76.

In a further example, the Vβ domain of a nucleic acid composition provided herein comprises an amino acid sequence having at least 80% sequence identity to, comprising, or consisting of, SEQ ID NO: 79.

The TCR Vβ domain sequences derived from TCR clone MRM23.3B2 discussed above are particularly compatible with the TCR Vα domain sequences derived from TCR clone MRM23.3B2 discussed elsewhere herein.

Accordingly, in one example, a nucleic acid composition described herein encodes a MAGE-A1 antigen-specific binding protein having TCR Vα domain comprising a CDR3 amino acid sequence having at least 80% sequence identity to SEQ ID NO:73, or a functional fragment thereof; and a nucleic acid sequence that encodes a TCR Vβ domain comprising a CDR3 amino acid sequence having at least 80% sequence identity to SEQ ID NO:76, or a functional fragment thereof.

In a particular example, a nucleic acid composition described herein encodes a MAGE-A1 antigen-specific binding protein having a TCR Vα domain with a CDR3 amino acid sequence comprising or consisting of the amino acid sequence of SEQ ID NO: 73; and a TCR Vβ domain with a CDR3 comprising or consisting of the amino acid sequence of SEQ ID NO:76. In addition, the MAGE-A1 antigen may comprise or consist of the sequence shown in SEQ ID NO: 131. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 77; and the Vβ domain may comprise an amino acid sequence having at least 80% sequence identity to, comprising, or consisting of, SEQ ID NO: 79. In one example, the Vα domain comprises the amino acid sequence of SEQ ID NO: 77 and the Vβ domain comprises the amino acid sequence of SEQ ID NO: 79. In such cases, the Vα domain may be encoded by a nucleic acid sequence comprising the sequence of SEQ ID NO: 78; and the Vβ domain may be encoded by a nucleic acid sequence comprising the sequence of SEQ ID NO: 80.

In this particular example, the TCR Vα domain may include a CDR1 amino acid sequence comprising or consisting of the amino acid sequence of SEQ ID NO:71 and a CDR2 amino acid sequence comprising or consisting of the amino acid sequence of SEQ ID NO:72. Furthermore, the TCR Vβ domain may include a CDR1 amino acid sequence comprising or consisting of the amino acid sequence of SEQ ID NO:74 and a CDR2 amino acid sequence comprising or consisting of the amino acid sequence of SEQ ID NO: 75.

For the avoidance of doubt, this particular example encompasses components of TCR clone MRM23.3B2 exemplified herein. The different components of TCR clone MRM23.3B2 and their respective SEQ ID Nos are summarised in Table 11 below.

As stated in more detail elsewhere herein, the nucleic acid composition described herein encodes both a TCR Vα domain and a TCR Vβ domain, which form the binding protein that is capable of specifically binding to a MAGE antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

(vii) TCR Components that Interact with RVRFFFPSL (SEQ ID NO: 132)

As provided elsewhere herein, the inventors have also identified TCR clone CT12.3G2 (3G2) which interacts with RVRFFFPSL (SEQ ID NO: 132) in the context of HLA-B*07:02. The sequences provided herein that correspond to TCR clone CT12.3G2 are SEQ ID NO:s 85 to 98.

In one embodiment, an isolated nucleic acid composition that encodes a MAGE antigen-specific binding protein having a TCR α chain variable (Vα) domain and a TCR β chain variable (Vβ) domain is provided, the composition comprising:
a nucleic acid sequence that encodes a TCR Vα domain comprising a CDR3 amino acid sequence having at least 80% sequence identity to SEQ ID NO:87, or a functional fragment thereof; and a nucleic acid sequence that encodes a TCR Vβ domain comprising a CDR3 amino acid sequence having at least 80% sequence identity to SEQ ID NO: 90, or a functional fragment thereof, wherein the CDR3 sequences together specifically bind to MAGE-A1.

An example of an appropriate TCR Vα domain CDR3 amino acid sequence that confers specific binding to a MAGE antigen, in particular a MAGE-A1 antigen (e.g. to RVRFFFPSL (SEQ ID NO: 132)), is shown in SEQ ID NO: 87. As would be clear to a person of skill in the art, variants of the amino acid sequence shown in SEQ ID NO: 87 may also be functional (i.e. retain their ability to confer specific binding to a MAGE-A1 antigen (e.g. to the peptide RVRFFFPSL (SEQ ID NO: 132)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 87, i.e. they may have at least 80%, at least 82%, at least 88%, at least 94%, or 100% sequence identity to SEQ ID NO: 87. Suitably, percent identity is calculated as the percentage of identity to the entire length of the reference sequence (e.g. SEQ ID NO: 87). In other words, appropriate (functional) Vα domain CDR3 amino acid sequences may vary from the sequence shown in SEQ ID NO: 87 by one or several (e.g. two etc) amino acids.

As stated above, functional variants of SEQ ID NO: 87 retain their ability to confer specific binding to a MAGE-A1 antigen (i.e. the peptide shown in SEQ ID NO: 132) when the CDR3 is part of TCR Vα domain.

Functional variants may be naturally occurring, synthetic, or synthetically improved functional variants of SEQ ID NO: 87. The term “variant” also encompasses homologues and fragments. Functional variants will typically contain only conservative substitutions of one, two or more amino acids of SEQ ID NO: 87, or substitution, deletion or insertion of non-critical amino acids in non-critical regions of the CDR3.

Non-functional variants are amino acid sequence variants of SEQ ID NO: 87 that do not specifically bind to a MAGE-A1 antigen (i.e. the peptide shown in SEQ ID NO: 132). Non-functional variants will typically contain a non-conservative substitution, a deletion, or insertion or premature truncation of the amino acid sequence of SEQ ID NO: 87 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 87. In examples where the TCR Vα domain CDR3 has the amino acid sequence of SEQ ID NO: 87, the CDR3 may be encoded by any appropriate nucleic acid sequence.

The encoded TCR Vα domain may comprise, in addition to the specified CDR3, a CDR1 comprising an amino acid sequence of SEQ ID NO: 85, or a functional variant thereof (i.e, wherein the variant retains the ability to specifically bind to a MAGE-A1 antigen (e.g. the peptide shown in SEQ ID NO: 132)). Such functional variants may be naturally occurring, synthetic, or synthetically improved functional variants of SEQ ID NO: 85. The term “variant” also encompasses homologues and fragments. Functional variants will typically contain only conservative substitutions of one or more amino acids of SEQ ID NO: 85, or substitution, deletion or insertion of non-critical amino acids in non-critical regions of the protein.

Non-functional variants are amino acid sequence variants of SEQ ID NO: 85 that do not specifically bind to a MAGE-A1 antigen (e.g. the peptide shown in SEQ ID NO: 132). Non-functional variants will typically contain a non-conservative substitution, a deletion, or insertion or premature truncation of the amino acid sequence of SEQ ID NO: 85 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 85, i.e. it may have at least 80%, at least 85%, or 100% sequence identity to SEQ ID NO: 85. Suitably, percent identity is calculated as the percentage of identity to the entire length of the reference sequence (e.g. SEQ ID NO: 85). In other words, appropriate functional Vα domain CDR1 amino acid sequences may vary from the sequence shown in SEQ ID NO: 85 by one or several amino acids. As stated previously, the variant may comprise an amino acid substitution such as a conservative amino acid substitution compared to the sequence shown in SEQ ID NO: 85. As stated above, functional variants of SEQ ID NO: 85 retain the ability to specifically bind to a MAGE-A1 antigen (e.g. the peptide shown in SEQ ID NO: 132) when the CDR1 is part of TCR Vα domain.

In one example, the CDR1 of the Vα domain comprises or consists of the amino acid sequence of SEQ ID NO: 85. In examples where the TCR Vα domain CDR1 has the amino acid sequence of SEQ ID NO: 85, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 86, or a functional variant thereof (i.e, wherein the variant retains the ability to specifically bind to HLA-B*07:02). Such functional variants may be naturally occurring, synthetic, or synthetically improved functional variants of SEQ ID NO: 86. The term “variant” also encompasses homologues and fragments. Functional variants will typically contain only conservative substitutions of one or more amino acids of SEQ ID NO: 86, or substitution, deletion or insertion of non-critical amino acids in non-critical regions of the protein.

Non-functional variants are amino acid sequence variants of SEQ ID NO: 86 that do not specifically bind to HLA-B*07:02. Non-functional variants will typically contain a non-conservative substitution, a deletion, or insertion or premature truncation of the amino acid sequence of SEQ ID NO: 86 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 86, i.e. it may have at least 80%, at least 87%, or 100% sequence identity to SEQ ID NO: 86. Suitably, percent identity is calculated as the percentage of identity to the entire length of the reference sequence (e.g. SEQ ID NO: 86). In other words, appropriate (functional) Vα domain CDR2 amino acid sequences may vary from the sequence shown in SEQ ID NO: 86 by one or several amino acids. As stated previously, the variant may comprise an amino acid substitution such as a conservative amino acid substitution compared to the sequence shown in SEQ ID NO: 86. As stated above, a functional variant of SEQ ID NO: 86 retains the ability to specifically bind to HLA-B*07:02.

In one example, the CDR2 of the Vα domain comprises or consists of the amino acid sequence of SEQ ID NO: 86. In examples where the TCR Vα domain CDR2 has the amino acid sequence of SEQ ID NO: 86, the CDR2 may be encoded by any appropriate nucleic acid sequence.

The encoded TCR Vα domain may therefore comprise the CDRs mentioned in detail above (by SEQ ID specifically i.e. SEQ ID NO:87, SEQ ID NO: 85 and SEQ ID NO: 86, or functional variants thereof), with appropriate intervening sequences between the CDRs.

The encoded TCR Vα domain may comprise an amino acid sequence of SEQ ID NO: 91, or a functional variant thereof (i.e, wherein the variant TCR Vα domain retains the ability to specifically bind to a MAGE-A1 antigen (e.g. the peptide shown in SEQ ID NO: 132) when part of a binding protein described herein). Such functional variants may be naturally occurring, synthetic, or synthetically improved functional variants of SEQ ID NO: 91. The term “variant” also encompasses homologues and fragments. Functional variants will typically contain only conservative substitutions of one or more amino acids of SEQ ID NO: 91, or substitution, deletion or insertion of non-critical amino acids in non-critical regions of the protein.

Non-functional variants are amino acid sequence variants of SEQ ID NO: 91 that do not specifically bind to a MAGE-A1 antigen (e.g. the peptide shown in SEQ ID NO: 132). Non-functional variants will typically contain a non-conservative substitution, a deletion, or insertion or premature truncation of the amino acid sequence of SEQ ID NO: 91 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 91, whilst retaining the ability to specifically bind to a MAGE-A1 antigen (e.g. the peptide shown in SEQ ID NO: 132). In other words, a functional TCR Vα domain with one or several amino acid substitutions compared to the sequence of SEQ ID NO: 91 is also encompassed. As stated previously, the amino acid substitution may be a conservative amino acid substitution. The variability in sequence compared to SEQ ID NO: 91 may all be in regions of the TCR Vα domain that do not form CDRs (i.e. the variant may have the CDRs of SEQ ID NO: 87, SEQ ID NO: 85 and/or SEQ ID NO: 86, and still have 25% (or less) sequence variability compared to SEQ ID NO:91). In other words, the sequence of the CDRs of SEQ ID NO: 91 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 91).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 91, wherein the TCR Vα domain comprises a CDR3 having an amino acid sequence of SEQ ID NO: 87. In this example, the TCR Vα domain CDR1 may have an amino acid sequence of SEQ ID NO: 85 and the TCR Vα domain CDR2 may have an amino acid sequence of SEQ ID NO: 86.

As another example, the encoded TCR Vα domain may comprise an amino acid sequence having the amino acid sequence of SEQ ID NO: 91, with 0 to 10 (or 0 to 5) amino acid substitutions, insertions or deletions, wherein the TCR Vα domain comprises a CDR3 having an amino acid sequence of SEQ ID NO: 87. In this example, the TCR Vα domain CDR1 may have an amino acid sequence of SEQ ID NO: 85 and the TCR Vα domain CDR2 may have an amino acid sequence of SEQ ID NO: 86.

In examples where the TCR Vα domain has the amino acid sequence of SEQ ID NO: 91, the TCR Vα domain may be encoded by the nucleic acid sequence of SEQ ID NO: 92, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

An example of a specific TCR α chain amino acid sequence that includes a TCR Vα domain described herein with an appropriate constant domain is shown in SEQ ID NO: 95. Appropriate functional variants of SEQ ID NO:95 are also encompassed (e.g. variants having at least 75% (e.g. at least 75%, at least 80%, at least 85%, at least 90%, at least 95% etc) sequence identity to the amino acid sequence of SEQ ID NO: 95, wherein the variant TCR α chain amino acid sequence retains its ability to specifically bind to a MAGE-A1 antigen (e.g. the peptide shown in SEQ ID NO: 132) when part of a binding protein described herein). In other words, a functional TCR α chain with one or several amino acid substitutions compared to the sequence of SEQ ID NO:95 is also encompassed. As stated previously, the amino acid substitution may be a conservative amino acid substitution. The variability in sequence compared to SEQ ID NO: 95 may all be in regions of the TCR α chain that do not form CDRs (i.e. the variant may have the CDRs of SEQ ID NO: 87, SEQ ID NO: 85 and/or SEQ ID NO: 86, and still have 25% (or less) sequence variability compared to SEQ ID NO: 95). In other words, the sequence of the CDRs of SEQ ID NO: 95 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 95).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 95, wherein the TCR α chain comprises a CDR3 having an amino acid sequence of SEQ ID NO: 87. In this example, the TCR α chain CDR1 may have an amino acid sequence of SEQ ID NO:85 and the TCR α chain CDR2 may have an amino acid sequence of SEQ ID NO: 86.

In examples where the TCR α chain has the amino acid sequence of SEQ ID NO: 95, the TCR a chain may be encoded by the nucleic acid sequence of SEQ ID NO: 96, or a genetically degenerate sequence thereof (i.e. other nucleic acid sequences that encode the same protein as a result of the degeneracy of the genetic code). It is noted that SEQ ID NO:96 is the nucleic acid sequence for TCR α chain of clone CT12.3G2.

In one example, the nucleic acid composition provided herein comprises a nucleic acid sequence that encodes a TCR Vα domain comprising a CDR3 amino acid sequence having at least 80% sequence identity to SEQ ID NO:87, or a functional fragment thereof.

In another example, the CDR3 of the Vα domain of a nucleic acid composition provided herein comprises or consists of the amino acid sequence of SEQ ID NO: 87.

In another example, the Vα domain of a nucleic acid composition provided herein comprises an amino acid sequence having at least 80% sequence identity to, comprising, or consisting of, SEQ ID NO: 91.

As provided above, the inventors identified TCR clone CT12.3G2 which interacts with RVRFFFPSL (SEQ ID NO: 132) in the context of HLA-B*07:02. The sequences provided herein that correspond to TCR clone CT12.3G2 are SEQ ID NO:s 85 to 98.

An example of an appropriate TCR Vβ domain CDR3 amino acid sequence that confers specific binding to a MAGE antigen, in particular a MAGE-A1 antigen (e.g. to RVRFFFPSL (SEQ ID NO: 132)), is shown in SEQ ID NO:90. As would be clear to a person of skill in the art, variants of the amino acid sequence shown in SEQ ID NO:90 may also be functional (i.e. retain their ability to confer specific binding to a MAGE-A1 antigen (i.e. the peptide shown in SEQ ID NO: 132) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 90, i.e. they may have at least 80%, at least 83%, at least 91%, or 100% sequence identity to SEQ ID NO: 90. Suitably, percent identity is calculated as the percentage of identity to the entire length of the reference sequence (e.g. SEQ ID NO: 90). In other words, appropriate (functional) Vβ domain CDR3 amino acid sequences may vary from the sequence shown in SEQ ID NO: 90 by one or several (e.g. two) amino acids. As stated above, functional variants of SEQ ID NO: 90 retain their ability to confer specific binding to a MAGE-A1 antigen (e.g. the peptide shown in SEQ ID NO: 132) when the CDR3 is part of TCR Vβ domain.

Functional variants may be naturally occurring, synthetic, or synthetically improved functional variants of SEQ ID NO: 90. The term “variant” also encompasses homologues and fragments. Functional variants will typically contain only conservative substitutions of one or more amino acids of SEQ ID NO: 90, or substitution, deletion or insertion of non-critical amino acids in non-critical regions of the CDR3.

Non-functional variants are amino acid sequence variants of SEQ ID NO: 90 that do not specifically bind to a MAGE-A1 antigen (e.g. the peptide shown in SEQ ID NO: 132). Non-functional variants will typically contain a non-conservative substitution, a deletion, or insertion or premature truncation of the amino acid sequence of SEQ ID NO: 90 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 90. In examples where the TCR Vβ domain CDR3 has the amino acid sequence of SEQ ID NO:90, the CDR3 may be encoded by any appropriate nucleic acid sequence.

The encoded TCR Vβ domain may comprise, in addition to the specified CDR3, a CDR1 comprising an amino acid sequence of SEQ ID NO: 88, or a functional variant thereof (i.e, wherein the variant retains the ability to specifically bind to a MAGE-A1 antigen (e.g. the peptide shown in SEQ ID NO: 132)). Such functional variants may be naturally occurring, synthetic, or synthetically improved functional variants of SEQ ID NO: 88. The term “variant” also encompasses homologues and fragments. Functional variants will typically contain only conservative substitutions of one or more amino acids of SEQ ID NO: 88, or substitution, deletion or insertion of non-critical amino acids in non-critical regions of the protein.

Non-functional variants are amino acid sequence variants of SEQ ID NO: 88 that do not specifically bind to a MAGE-A1 antigen (e.g. the peptide shown in SEQ ID NO: 132). Non-functional variants will typically contain a non-conservative substitution, a deletion, or insertion or premature truncation of the amino acid sequence of SEQ ID NO: 88 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 88, i.e. it may have at least 80%, or 100% sequence identity to SEQ ID NO: 88. Suitably, percent identity is calculated as the percentage of identity to the entire length of the reference sequence (e.g. SEQ ID NO: 88). In other words, appropriate (functional) Vβ domain CDR1 amino acid sequences may vary from the sequence shown in SEQ ID NO:88 by one or several amino acids. As stated previously, the variant may comprise an amino acid substitution such as a conservative amino acid substitution compared to the sequence shown in SEQ ID NO:88. As stated above, functional variants of SEQ ID NO: 88 retain the ability to specifically bind to a MAGE-A1 antigen (e.g. the peptide shown in SEQ ID NO: 132) when the CDR1 is part of TCR Vβ domain.

In one example, the CDR1 of the Vβ domain comprises or consists of the amino acid sequence of SEQ ID NO: 88. In examples where the TCR Vα domain CDR1 has the amino acid sequence of SEQ ID NO:88, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 89, or a functional variant thereof (i.e, wherein the variant retains the ability to specifically bind to HLA-B*07:02). Such functional variants may be naturally occurring, synthetic, or synthetically improved functional variants of SEQ ID NO: 89. The term “variant” also encompasses homologues and fragments. Functional variants will typically contain only conservative substitutions of one or more amino acids of SEQ ID NO: 89, or substitution, deletion or insertion of non-critical amino acids in non-critical regions of the protein.

Non-functional variants are amino acid sequence variants of SEQ ID NO: 89 that do not specifically bind to HLA-B*07:02. Non-functional variants will typically contain a non-conservative substitution, a deletion, or insertion or premature truncation of the amino acid sequence of SEQ ID NO: 89 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 89, i.e. it may have at least 80%, at least 83%, or 100% sequence identity to SEQ ID NO: 89. Suitably, percent identity is calculated as the percentage of identity to the entire length of the reference sequence (e.g. SEQ ID NO: 89). In other words, appropriate (functional) Vβ domain CDR2 amino acid sequences may vary from the sequence shown in SEQ ID NO: 89 by one or several amino acids. As stated previously, the variant may comprise an amino acid substitution such as a conservative amino acid substitution compared to the sequence shown in SEQ ID NO: 89. As stated above, a functional variant of SEQ ID NO: 89 retains the ability to specifically bind to HLA-B*07:02.

In one example, the CDR2 of the Vβ domain comprises or consists of the amino acid sequence of SEQ ID NO: 89. In examples where the TCR Vβ domain CDR2 has the amino acid sequence of SEQ ID NO:89, the CDR2 may be encoded by any appropriate nucleic acid sequence.

The encoded TCR Vβ domain may therefore comprise the CDRs mentioned in detail above (by SEQ ID specifically i.e. SEQ ID NO:90, SEQ ID NO: 88 and SEQ ID NO: 89, or functional variants thereof), with appropriate intervening sequences between the CDRs.

The encoded TCR Vβ domain may have an amino acid sequence of SEQ ID NO: 93, or a functional variant thereof (i.e, wherein the variant TCR Vβ domain retains the ability to specifically bind to a MAGE-A1 antigen (e.g. the peptide shown in SEQ ID NO: 132) when part of a binding protein described herein). Such functional variants may be naturally occurring, synthetic, or synthetically improved functional variants of SEQ ID NO: 93. The term “variant” also encompasses homologues and fragments. Functional variants will typically contain only conservative substitutions of one or more amino acids of SEQ ID NO: 93, or substitution, deletion or insertion of non-critical amino acids in non-critical regions of the protein.

Non-functional variants are amino acid sequence variants of SEQ ID NO: 93 that do not specifically bind to a MAGE-A1 antigen (e.g. the peptide shown in SEQ ID NO: 132). Non-functional variants will typically contain a non-conservative substitution, a deletion, or insertion or premature truncation of the amino acid sequence of SEQ ID NO:93 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 93, whilst retaining the ability to specifically bind to a MAGE-A1 antigen (e.g. the peptide shown in SEQ ID NO: 132). In other words, a functional TCR Vβ domain with one or several amino acid substitutions compared to the sequence of SEQ ID NO: 93 is also encompassed. As stated previously, the amino acid substitution may be a conservative amino acid substitution. The variability in sequence compared to SEQ ID NO:93 may all be in regions of the TCR Vβ domain that do not form CDRs (i.e. the variant may have the CDRs of SEQ ID NO: 90, SEQ ID NO: 88 and/or SEQ ID NO: 89, and still have 25% (or less) sequence variability compared to SEQ ID NO: 93). In other words, the sequence of the CDRs of SEQ ID NO: 93 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 93).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 93, wherein the TCR Vβ domain comprises a CDR3 having an amino acid sequence of SEQ ID NO: 90. In this example, the TCR Vβ domain CDR1 may have an amino acid sequence of SEQ ID NO:88 and the TCR Vβ domain CDR2 may have an amino acid sequence of SEQ ID NO: 89.

In examples where the TCR Vβ domain has the amino acid sequence of SEQ ID NO:93, the TCR Vβ domain may be encoded by the nucleic acid sequence of SEQ ID NO: 94, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 97. Appropriate functional variants of SEQ ID NO: 97 are also encompassed (e.g. variants having at least 75% (e.g. at least 75%, at least 80%, at least 85%, at least 90%, at least 95% etc) sequence identity to the amino acid sequence of SEQ ID NO: 97, wherein the variant TCR β chain amino acid sequence retains its ability to specifically bind to a MAGE-A1 antigen (e.g. the peptide shown in SEQ ID NO: 132) when part of a binding protein described herein). In other words, a functional TCR β chain with one or several amino acid substitutions compared to the sequence of SEQ ID NO: 97 is also encompassed. As stated previously, the amino acid substitution may be a conservative amino acid substitution. The variability in sequence compared to SEQ ID NO: 97 may all be in regions of the TCR β chain that do not form CDRs (i.e. the variant may have the CDRs of SEQ ID NO: 90, SEQ ID NO: 88 and/or SEQ ID NO: 89, and still have 25% (or less) sequence variability compared to SEQ ID NO:97. In other words, the sequence of the CDRs of SEQ ID NO: 97 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 97).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 97, wherein the TCR β chain comprises a CDR3 having an amino acid sequence of SEQ ID NO: 90. In this example, the TCR β chain CDR1 may have an amino acid sequence of SEQ ID NO: 88 and the TCR β chain CDR2 may have an amino acid sequence of SEQ ID NO: 89.

In examples where the TCR β chain has the amino acid sequence of SEQ ID NO:97, the TCR β chain may be encoded by the nucleic acid sequence of SEQ ID NO:98, or a genetically degenerate sequence thereof (i.e. other nucleic acid sequences that encode the same protein as a result of the degeneracy of the genetic code). It is noted that SEQ ID NO:98 is the nucleic acid sequence for TCR β chain of clone CT12.3G2.

In an example, the nucleic acid composition provided herein comprises a nucleic acid sequence that encodes a TCR Vβ domain comprising a CDR3 amino acid sequence having at least 80% sequence identity to SEQ ID NO:90, or a functional fragment thereof.

In another example, the CDR3 of the Vβ domain of a nucleic acid composition provided herein comprises or consists of the amino acid sequence of SEQ ID NO:90.

In a further example, the Vβ domain of a nucleic acid composition provided herein comprises an amino acid sequence having at least 80% sequence identity to, comprising, or consisting of, SEQ ID NO: 93.

The TCR Vβ domain sequences derived from TCR clone CT12.3G2 discussed above are particularly compatible with the TCR Vα domain sequences derived from TCR clone CT12.3G2 discussed elsewhere herein.

Accordingly, in one example, a nucleic acid composition described herein encodes a MAGE-A1 antigen-specific binding protein having TCR Vα domain comprising a CDR3 amino acid sequence having at least 80% sequence identity to SEQ ID NO:87, or a functional fragment thereof; and a nucleic acid sequence that encodes a TCR Vβ domain comprising a CDR3 amino acid sequence having at least 80% sequence identity to SEQ ID NO:90, or a functional fragment thereof.

In a particular example, a nucleic acid composition described herein encodes a MAGE-A1 antigen-specific binding protein having a TCR Vα domain with a CDR3 amino acid sequence comprising or consisting of the amino acid sequence of SEQ ID NO: 87; and a TCR Vβ domain with a CDR3 comprising or consisting of the amino acid sequence of SEQ ID NO:90. In addition, the MAGE-A1 antigen may comprise or consist of the sequence shown in SEQ ID NO: 132. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 91; and the Vβ domain may comprise an amino acid sequence having at least 80% sequence identity to, comprising, or consisting of, SEQ ID NO: 93. In one example, the Vα domain comprises the amino acid sequence of SEQ ID NO: 91 and the Vβ domain comprises the amino acid sequence of SEQ ID NO: 93. In such cases, the Vα domain may be encoded by a nucleic acid sequence comprising the sequence of SEQ ID NO: 92; and the Vβ domain may be encoded by a nucleic acid sequence comprising the sequence of SEQ ID NO: 94.

In this particular example, the TCR Vα domain may include a CDR1 amino acid sequence comprising or consisting of the amino acid sequence of SEQ ID NO:85 and a CDR2 amino acid sequence comprising or consisting of the amino acid sequence of SEQ ID NO:86. Furthermore, the TCR Vβ domain may include a CDR1 amino acid sequence comprising or consisting of the amino acid sequence of SEQ ID NO:88 and a CDR2 amino acid sequence comprising or consisting of the amino acid sequence of SEQ ID NO: 89.

For the avoidance of doubt, this particular example encompasses components of TCR clone CT12.3G2 exemplified herein. The different components of TCR clone CT12.3G2 and their respective SEQ ID Nos are summarised in Table 12 below.

As stated in more detail elsewhere herein, the nucleic acid composition described herein encodes both a TCR Vα domain and a TCR Vβ domain, which form the binding protein that is capable of specifically binding to a MAGE antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

(viii) Alternative TCR Components that Interact with RVRFFFPSL (SEQ ID NO: 132)

As provided elsewhere herein, the inventors have also identified TCR clone CT4.20.2C2 (2C2) which interacts with RVRFFFPSL (SEQ ID NO: 132) in the context of HLA-B*07:02. The sequences provided herein that correspond to TCR clone CT4.20.2C2 are SEQ ID NO:s 99 to 112.

In one embodiment, an isolated nucleic acid composition that encodes a MAGE antigen-specific binding protein having a TCR α chain variable (Vα) domain and a TCR β chain variable (Vβ) domain is provided, the composition comprising:
a nucleic acid sequence that encodes a TCR Vα domain comprising a CDR3 amino acid sequence having at least 80% sequence identity to SEQ ID NO:101, or a functional fragment thereof; and a nucleic acid sequence that encodes a TCR Vβ domain comprising a CDR3 amino acid sequence having at least 80% sequence identity to SEQ ID NO: 104, or a functional fragment thereof, wherein the CDR3 sequences together specifically bind to MAGE-A1.

An example of an appropriate TCR Vα domain CDR3 amino acid sequence that confers specific binding to a MAGE antigen, in particular a MAGE-A1 antigen (e.g. to RVRFFFPSL (SEQ ID NO: 132)), is shown in SEQ ID NO: 101. As would be clear to a person of skill in the art, variants of the amino acid sequence shown in SEQ ID NO: 101 may also be functional (i.e. retain their ability to confer specific binding to a MAGE-A1 antigen (e.g. to the peptide RVRFFFPSL (SEQ ID NO: 132)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 101, i.e. they may have at least 80%, at least 84%, at least 92%, or 100% sequence identity to SEQ ID NO: 101. Suitably, percent identity is calculated as the percentage of identity to the entire length of the reference sequence (e.g. SEQ ID NO: 101). In other words, appropriate (functional) Vα domain CDR3 amino acid sequences may vary from the sequence shown in SEQ ID NO: 101 by one or several (e.g. two etc) amino acids.

As stated above, functional variants of SEQ ID NO: 101 retain their ability to confer specific binding to a MAGE-A1 antigen (i.e. the peptide shown in SEQ ID NO: 132) when the CDR3 is part of TCR Vα domain.

Functional variants may be naturally occurring, synthetic, or synthetically improved functional variants of SEQ ID NO: 101. The term “variant” also encompasses homologues and fragments. Functional variants will typically contain only conservative substitutions of one, two or more amino acids of SEQ ID NO: 101, or substitution, deletion or insertion of non-critical amino acids in non-critical regions of the CDR3.

Non-functional variants are amino acid sequence variants of SEQ ID NO: 101 that do not specifically bind to a MAGE-A1 antigen (i.e. the peptide shown in SEQ ID NO: 132). Non-functional variants will typically contain a non-conservative substitution, a deletion, or insertion or premature truncation of the amino acid sequence of SEQ ID NO: 101 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 101. In examples where the TCR Vα domain CDR3 has the amino acid sequence of SEQ ID NO: 101, the CDR3 may be encoded by any appropriate nucleic acid sequence.

The encoded TCR Vα domain may comprise, in addition to the specified CDR3, a CDR1 comprising an amino acid sequence of SEQ ID NO: 99, or a functional variant thereof (i.e, wherein the variant retains the ability to specifically bind to a MAGE-A1 antigen (e.g. the peptide shown in SEQ ID NO: 132)). Such functional variants may be naturally occurring, synthetic, or synthetically improved functional variants of SEQ ID NO: 99. The term “variant” also encompasses homologues and fragments. Functional variants will typically contain only conservative substitutions of one or more amino acids of SEQ ID NO: 99, or substitution, deletion or insertion of non-critical amino acids in non-critical regions of the protein.

Non-functional variants are amino acid sequence variants of SEQ ID NO: 99 that do not specifically bind to a MAGE-A1 antigen (e.g. the peptide shown in SEQ ID NO: 132). Non-functional variants will typically contain a non-conservative substitution, a deletion, or insertion or premature truncation of the amino acid sequence of SEQ ID NO: 99 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 99, i.e. it may have at least 80%, at least 83%, or 100% sequence identity to SEQ ID NO: 99. Suitably, percent identity is calculated as the percentage of identity to the entire length of the reference sequence (e.g. SEQ ID NO: 99). In other words, appropriate functional Vα domain CDR1 amino acid sequences may vary from the sequence shown in SEQ ID NO: 99 by one or several amino acids. As stated previously, the variant may comprise an amino acid substitution such as a conservative amino acid substitution compared to the sequence shown in SEQ ID NO: 99. As stated above, functional variants of SEQ ID NO: 99 retain the ability to specifically bind to a MAGE-A1 antigen (e.g. the peptide shown in SEQ ID NO: 132) when the CDR1 is part of TCR Vα domain.

In one example, the CDR1 of the Vα domain comprises or consists of the amino acid sequence of SEQ ID NO: 99. In examples where the TCR Vα domain CDR1 has the amino acid sequence of SEQ ID NO: 99, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 100, or a functional variant thereof (i.e, wherein the variant retains the ability to specifically bind to HLA-B*07:02). Such functional variants may be naturally occurring, synthetic, or synthetically improved functional variants of SEQ ID NO: 100. The term “variant” also encompasses homologues and fragments. Functional variants will typically contain only conservative substitutions of one or more amino acids of SEQ ID NO: 100, or substitution, deletion or insertion of non-critical amino acids in non-critical regions of the protein.

Non-functional variants are amino acid sequence variants of SEQ ID NO: 100 that do not specifically bind to HLA-B*07:02. Non-functional variants will typically contain a non-conservative substitution, a deletion, or insertion or premature truncation of the amino acid sequence of SEQ ID NO: 100 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 100, i.e. it may have at least 80%, at least 83%, or 100% sequence identity to SEQ ID NO: 100. Suitably, percent identity is calculated as the percentage of identity to the entire length of the reference sequence (e.g. SEQ ID NO: 100). In other words, appropriate (functional) Vα domain CDR2 amino acid sequences may vary from the sequence shown in SEQ ID NO: 100 by one or several amino acids. As stated previously, the variant may comprise an amino acid substitution such as a conservative amino acid substitution compared to the sequence shown in SEQ ID NO: 100. As stated above, a functional variant of SEQ ID NO: 100 retains the ability to specifically bind to HLA-B*07:02.

In one example, the CDR2 of the Vα domain comprises or consists of the amino acid sequence of SEQ ID NO: 100. In examples where the TCR Vα domain CDR2 has the amino acid sequence of SEQ ID NO: 100, the CDR2 may be encoded by any appropriate nucleic acid sequence.

The encoded TCR Vα domain may therefore comprise the CDRs mentioned in detail above (by SEQ ID specifically i.e. SEQ ID NO: 101, SEQ ID NO: 99 and SEQ ID NO: 100, or functional variants thereof), with appropriate intervening sequences between the CDRs.

The encoded TCR Vα domain may comprise an amino acid sequence of SEQ ID NO: 105, or a functional variant thereof (i.e, wherein the variant TCR Vα domain retains the ability to specifically bind to a MAGE-A1 antigen (e.g. the peptide shown in SEQ ID NO: 132) when part of a binding protein described herein). Such functional variants may be naturally occurring, synthetic, or synthetically improved functional variants of SEQ ID NO: 105. The term “variant” also encompasses homologues and fragments. Functional variants will typically contain only conservative substitutions of one or more amino acids of SEQ ID NO: 105, or substitution, deletion or insertion of non-critical amino acids in non-critical regions of the protein.

Non-functional variants are amino acid sequence variants of SEQ ID NO: 105 that do not specifically bind to a MAGE-A1 antigen (e.g. the peptide shown in SEQ ID NO: 132). Non-functional variants will typically contain a non-conservative substitution, a deletion, or insertion or premature truncation of the amino acid sequence of SEQ ID NO: 105 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 105, whilst retaining the ability to specifically bind to a MAGE-A1 antigen (e.g. the peptide shown in SEQ ID NO: 132). In other words, a functional TCR Vα domain with one or several amino acid substitutions compared to the sequence of SEQ ID NO: 105 is also encompassed. As stated previously, the amino acid substitution may be a conservative amino acid substitution. The variability in sequence compared to SEQ ID NO: 105 may all be in regions of the TCR Vα domain that do not form CDRs (i.e. the variant may have the CDRs of SEQ ID NO: 101, SEQ ID NO: 99 and/or SEQ ID NO: 100, and still have 25% (or less) sequence variability compared to SEQ ID NO: 105). In other words, the sequence of the CDRs of SEQ ID NO: 105 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 105).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 105, wherein the TCR Vα domain comprises a CDR3 having an amino acid sequence of SEQ ID NO: 101. In this example, the TCR Vα domain CDR1 may have an amino acid sequence of SEQ ID NO: 99 and the TCR Vα domain CDR2 may have an amino acid sequence of SEQ ID NO: 100.

As another example, the encoded TCR Vα domain may comprise an amino acid sequence having the amino acid sequence of SEQ ID NO: 105, with 0 to 10 (or 0 to 5) amino acid substitutions, insertions or deletions, wherein the TCR Vα domain comprises a CDR3 having an amino acid sequence of SEQ ID NO: 101. In this example, the TCR Vα domain CDR1 may have an amino acid sequence of SEQ ID NO: 99 and the TCR Vα domain CDR2 may have an amino acid sequence of SEQ ID NO: 100.

In examples where the TCR Vα domain has the amino acid sequence of SEQ ID NO: 105, the TCR Vα domain may be encoded by the nucleic acid sequence of SEQ ID NO: 106, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

An example of a specific TCR α chain amino acid sequence that includes a TCR Vα domain described herein with an appropriate constant domain is shown in SEQ ID NO: 109. Appropriate functional variants of SEQ ID NO: 109 are also encompassed (e.g. variants having at least 75% (e.g. at least 75%, at least 80%, at least 85%, at least 90%, at least 95% etc) sequence identity to the amino acid sequence of SEQ ID NO: 109, wherein the variant TCR α chain amino acid sequence retains its ability to specifically bind to a MAGE-A1 antigen (e.g. the peptide shown in SEQ ID NO: 132) when part of a binding protein described herein). In other words, a functional TCR α chain with one or several amino acid substitutions compared to the sequence of SEQ ID NO: 109 is also encompassed. As stated previously, the amino acid substitution may be a conservative amino acid substitution. The variability in sequence compared to SEQ ID NO: 109 may all be in regions of the TCR α chain that do not form CDRs (i.e. the variant may have the CDRs of SEQ ID NO: 101, SEQ ID NO: 99 and/or SEQ ID NO: 100, and still have 25% (or less) sequence variability compared to SEQ ID NO: 109). In other words, the sequence of the CDRs of SEQ ID NO: 109 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 109).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 109, wherein the TCR α chain comprises a CDR3 having an amino acid sequence of SEQ ID NO: 101. In this example, the TCR α chain CDR1 may have an amino acid sequence of SEQ ID NO:99 and the TCR α chain CDR2 may have an amino acid sequence of SEQ ID NO: 100.

In examples where the TCR α chain has the amino acid sequence of SEQ ID NO: 109, the TCR α chain may be encoded by the nucleic acid sequence of SEQ ID NO: 110, or a genetically degenerate sequence thereof (i.e. other nucleic acid sequences that encode the same protein as a result of the degeneracy of the genetic code). It is noted that SEQ ID NO: 110 is the nucleic acid sequence for TCR α chain of clone CT4.20.2C2.

In one example, the nucleic acid composition provided herein comprises a nucleic acid sequence that encodes a TCR Vα domain comprising a CDR3 amino acid sequence having at least 80% sequence identity to SEQ ID NO: 101, or a functional fragment thereof.

In another example, the CDR3 of the Vα domain of a nucleic acid composition provided herein comprises or consists of the amino acid sequence of SEQ ID NO: 101.

In another example, the Vα domain of a nucleic acid composition provided herein comprises an amino acid sequence having at least 80% sequence identity to, comprising, or consisting of, SEQ ID NO: 105.

As provided above, the inventors identified TCR clone CT4.20.2C2 which interacts with RVRFFFPSL (SEQ ID NO: 132) in the context of HLA-B*07:02. The sequences provided herein that correspond to TCR clone CT4.20.2C2 are SEQ ID NO:s 99 to 112.

An example of an appropriate TCR Vβ domain CDR3 amino acid sequence that confers specific binding to a MAGE antigen, in particular a MAGE-A1 antigen (e.g. to RVRFFFPSL (SEQ ID NO: 132)), is shown in SEQ ID NO: 104. As would be clear to a person of skill in the art, variants of the amino acid sequence shown in SEQ ID NO: 104 may also be functional (i.e. retain their ability to confer specific binding to a MAGE-A1 antigen (i.e. the peptide shown in SEQ ID NO: 132) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 104, i.e. they may have at least 80%, at least 86%, at least 93%, or 100% sequence identity to SEQ ID NO: 104. Suitably, percent identity is calculated as the percentage of identity to the entire length of the reference sequence (e.g. SEQ ID NO: 104). In other words, appropriate (functional) Vβ domain CDR3 amino acid sequences may vary from the sequence shown in SEQ ID NO: 104 by one or several (e.g. two) amino acids. As stated above, functional variants of SEQ ID NO: 104 retain their ability to confer specific binding to a MAGE-A1 antigen (e.g. the peptide shown in SEQ ID NO: 132) when the CDR3 is part of TCR Vβ domain.

Functional variants may be naturally occurring, synthetic, or synthetically improved functional variants of SEQ ID NO: 104. The term “variant” also encompasses homologues and fragments. Functional variants will typically contain only conservative substitutions of one or more amino acids of SEQ ID NO: 104, or substitution, deletion or insertion of non-critical amino acids in non-critical regions of the CDR3.

Non-functional variants are amino acid sequence variants of SEQ ID NO: 104 that do not specifically bind to a MAGE-A1 antigen (e.g. the peptide shown in SEQ ID NO: 132). Non-functional variants will typically contain a non-conservative substitution, a deletion, or insertion or premature truncation of the amino acid sequence of SEQ ID NO: 104 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 104. In examples where the TCR Vβ domain CDR3 has the amino acid sequence of SEQ ID NO: 104, the CDR3 may be encoded by any appropriate nucleic acid sequence.

The encoded TCR Vβ domain may comprise, in addition to the specified CDR3, a CDR1 comprising an amino acid sequence of SEQ ID NO: 102, or a functional variant thereof (i.e, wherein the variant retains the ability to specifically bind to a MAGE-A1 antigen (e.g. the peptide shown in SEQ ID NO: 132)). Such functional variants may be naturally occurring, synthetic, or synthetically improved functional variants of SEQ ID NO: 102. The term “variant” also encompasses homologues and fragments. Functional variants will typically contain only conservative substitutions of one or more amino acids of SEQ ID NO: 102, or substitution, deletion or insertion of non-critical amino acids in non-critical regions of the protein.

Non-functional variants are amino acid sequence variants of SEQ ID NO: 102 that do not specifically bind to a MAGE-A1 antigen (e.g. the peptide shown in SEQ ID NO: 132). Non-functional variants will typically contain a non-conservative substitution, a deletion, or insertion or premature truncation of the amino acid sequence of SEQ ID NO: 102 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 102, i.e. it may have at least 80%, or 100% sequence identity to SEQ ID NO: 102. Suitably, percent identity is calculated as the percentage of identity to the entire length of the reference sequence (e.g. SEQ ID NO: 102). In other words, appropriate (functional) Vβ domain CDR1 amino acid sequences may vary from the sequence shown in SEQ ID NO: 102 by one or several amino acids. As stated previously, the variant may comprise an amino acid substitution such as a conservative amino acid substitution compared to the sequence shown in SEQ ID NO:102. As stated above, functional variants of SEQ ID NO: 102 retain the ability to specifically bind to a MAGE-A1 antigen (e.g. the peptide shown in SEQ ID NO: 132) when the CDR1 is part of TCR Vβ domain.

In one example, the CDR1 of the Vβ domain comprises or consists of the amino acid sequence of SEQ ID NO: 102. In examples where the TCR Vα domain CDR1 has the amino acid sequence of SEQ ID NO:102, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 103, or a functional variant thereof (i.e, wherein the variant retains the ability to specifically bind to HLA-B*07:02). Such functional variants may be naturally occurring, synthetic, or synthetically improved functional variants of SEQ ID NO: 103. The term “variant” also encompasses homologues and fragments. Functional variants will typically contain only conservative substitutions of one or more amino acids of SEQ ID NO: 103, or substitution, deletion or insertion of non-critical amino acids in non-critical regions of the protein.

Non-functional variants are amino acid sequence variants of SEQ ID NO: 103 that do not specifically bind to HLA-B*07:02. Non-functional variants will typically contain a non-conservative substitution, a deletion, or insertion or premature truncation of the amino acid sequence of SEQ ID NO: 103 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 103, i.e. it may have at least 80%, at least 83%, or 100% sequence identity to SEQ ID NO: 103. Suitably, percent identity is calculated as the percentage of identity to the entire length of the reference sequence (e.g. SEQ ID NO: 103). In other words, appropriate (functional) Vβ domain CDR2 amino acid sequences may vary from the sequence shown in SEQ ID NO: 103 by one or several amino acids. As stated previously, the variant may comprise an amino acid substitution such as a conservative amino acid substitution compared to the sequence shown in SEQ ID NO: 103. As stated above, a functional variant of SEQ ID NO: 103 retains the ability to specifically bind to HLA-B*07:02.

In one example, the CDR2 of the Vβ domain comprises or consists of the amino acid sequence of SEQ ID NO: 103. In examples where the TCR Vβ domain CDR2 has the amino acid sequence of SEQ ID NO: 103, the CDR2 may be encoded by any appropriate nucleic acid sequence.

The encoded TCR Vβ domain may therefore comprise the CDRs mentioned in detail above (by SEQ ID specifically i.e. SEQ ID NO:104, SEQ ID NO: 102 and SEQ ID NO: 103, or functional variants thereof), with appropriate intervening sequences between the CDRs.

The encoded TCR Vβ domain may have an amino acid sequence of SEQ ID NO: 107, or a functional variant thereof (i.e, wherein the variant TCR Vβ domain retains the ability to specifically bind to a MAGE-A1 antigen (e.g. the peptide shown in SEQ ID NO: 132) when part of a binding protein described herein). Such functional variants may be naturally occurring, synthetic, or synthetically improved functional variants of SEQ ID NO: 107. The term “variant” also encompasses homologues and fragments. Functional variants will typically contain only conservative substitutions of one or more amino acids of SEQ ID NO: 107, or substitution, deletion or insertion of non-critical amino acids in non-critical regions of the protein.

Non-functional variants are amino acid sequence variants of SEQ ID NO: 107 that do not specifically bind to a MAGE-A1 antigen (e.g. the peptide shown in SEQ ID NO: 132). Non-functional variants will typically contain a non-conservative substitution, a deletion, or insertion or premature truncation of the amino acid sequence of SEQ ID NO: 107 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 107, whilst retaining the ability to specifically bind to a MAGE-A1 antigen (e.g. the peptide shown in SEQ ID NO: 132). In other words, a functional TCR Vβ domain with one or several amino acid substitutions compared to the sequence of SEQ ID NO: 107 is also encompassed. As stated previously, the amino acid substitution may be a conservative amino acid substitution. The variability in sequence compared to SEQ ID NO: 107 may all be in regions of the TCR Vβ domain that do not form CDRs (i.e. the variant may have the CDRs of SEQ ID NO: 104, SEQ ID NO: 102 and/or SEQ ID NO: 103, and still have 25% (or less) sequence variability compared to SEQ ID NO: 107). In other words, the sequence of the CDRs of SEQ ID NO: 107 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 107).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 107, wherein the TCR Vβ domain comprises a CDR3 having an amino acid sequence of SEQ ID NO: 104. In this example, the TCR Vβ domain CDR1 may have an amino acid sequence of SEQ ID NO:102 and the TCR Vβ domain CDR2 may have an amino acid sequence of SEQ ID NO: 103.

In examples where the TCR Vβ domain has the amino acid sequence of SEQ ID NO: 107, the TCR Vβ domain may be encoded by the nucleic acid sequence of SEQ ID NO: 108, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 111. Appropriate functional variants of SEQ ID NO: 111 are also encompassed (e.g. variants having at least 75% (e.g. at least 75%, at least 80%, at least 85%, at least 90%, at least 95% etc) sequence identity to the amino acid sequence of SEQ ID NO: 111, wherein the variant TCR β chain amino acid sequence retains its ability to specifically bind to a MAGE-A1 antigen (e.g. the peptide shown in SEQ ID NO: 132) when part of a binding protein described herein). In other words, a functional TCR β chain with one or several amino acid substitutions compared to the sequence of SEQ ID NO: 111 is also encompassed. As stated previously, the amino acid substitution may be a conservative amino acid substitution. The variability in sequence compared to SEQ ID NO: 111 may all be in regions of the TCR β chain that do not form CDRs (i.e. the variant may have the CDRs of SEQ ID NO: 104, SEQ ID NO: 102 and/or SEQ ID NO: 103, and still have 25% (or less) sequence variability compared to SEQ ID NO: 111. In other words, the sequence of the CDRs of SEQ ID NO: 111 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 111).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 111, wherein the TCR β chain comprises a CDR3 having an amino acid sequence of SEQ ID NO: 104. In this example, the TCR β chain CDR1 may have an amino acid sequence of SEQ ID NO: 102 and the TCR β chain CDR2 may have an amino acid sequence of SEQ ID NO: 103.

In examples where the TCR β chain has the amino acid sequence of SEQ ID NO: 111, the TCR β chain may be encoded by the nucleic acid sequence of SEQ ID NO: 112, or a genetically degenerate sequence thereof (i.e. other nucleic acid sequences that encode the same protein as a result of the degeneracy of the genetic code). It is noted that SEQ ID NO:112 is the nucleic acid sequence for TCR β chain of clone CT4.20.2C2.

In an example, the nucleic acid composition provided herein comprises a nucleic acid sequence that encodes a TCR Vβ domain comprising a CDR3 amino acid sequence having at least 80% sequence identity to SEQ ID NO: 104, or a functional fragment thereof.

In another example, the CDR3 of the Vβ domain of a nucleic acid composition provided herein comprises or consists of the amino acid sequence of SEQ ID NO: 104.

In a further example, the Vβ domain of a nucleic acid composition provided herein comprises an amino acid sequence having at least 80% sequence identity to, comprising, or consisting of, SEQ ID NO: 107.

The TCR Vβ domain sequences derived from TCR clone CT4.20.2C2 discussed above are particularly compatible with the TCR Vα domain sequences derived from TCR clone CT4.20.2C2 discussed elsewhere herein.

Accordingly, in one example, a nucleic acid composition described herein encodes a MAGE-A1 antigen-specific binding protein having TCR Vα domain comprising a CDR3 amino acid sequence having at least 80% sequence identity to SEQ ID NO: 101, or a functional fragment thereof; and a nucleic acid sequence that encodes a TCR Vβ domain comprising a CDR3 amino acid sequence having at least 80% sequence identity to SEQ ID NO: 104, or a functional fragment thereof.

In a particular example, a nucleic acid composition described herein encodes a MAGE-A1 antigen-specific binding protein having a TCR Vα domain with a CDR3 amino acid sequence comprising or consisting of the amino acid sequence of SEQ ID NO: 101; and a TCR Vβ domain with a CDR3 comprising or consisting of the amino acid sequence of SEQ ID NO: 104. In addition, the MAGE-A1 antigen may comprise or consist of the sequence shown in SEQ ID NO: 132. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 105; and the Vβ domain may comprise an amino acid sequence having at least 80% sequence identity to, comprising, or consisting of, SEQ ID NO: 107. In one example, the Vα domain comprises the amino acid sequence of SEQ ID NO: 105 and the Vβ domain comprises the amino acid sequence of SEQ ID NO: 107. In such cases, the Vα domain may be encoded by a nucleic acid sequence comprising the sequence of SEQ ID NO: 106; and the Vβ domain may be encoded by a nucleic acid sequence comprising the sequence of SEQ ID NO: 108.

In this particular example, the TCR Vα domain may include a CDR1 amino acid sequence comprising or consisting of the amino acid sequence of SEQ ID NO:99 and a CDR2 amino acid sequence comprising or consisting of the amino acid sequence of SEQ ID NO:100. Furthermore, the TCR Vβ domain may include a CDR1 amino acid sequence comprising or consisting of the amino acid sequence of SEQ ID NO: 102 and a CDR2 amino acid sequence comprising or consisting of the amino acid sequence of SEQ ID NO: 103.

For the avoidance of doubt, this particular example encompasses components of TCR clone CT4.20.2C2 exemplified herein. The different components of TCR clone CT4.20.2C2 and their respective SEQ ID Nos are summarised in Table 13 below.

As stated in more detail elsewhere herein, the nucleic acid composition described herein encodes both a TCR Vα domain and a TCR Vβ domain, which form the binding protein that is capable of specifically binding to a MAGE antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

(ix) TCR Components that Interact with EVDPIGHXY (SEQ ID NO: 183)

As provided elsewhere herein, the inventors have also identified TCR clone CT23.2H9 which interacts with EVDPIGHXY (SEQ ID NO: 183) in the context of HLA-B*35:01. The sequences provided herein that correspond to TCR clone CT23.2H9 are SEQ ID NO:s 113 to 126. SEQ ID NO: 183 encompasses EVDPIGHLY (SEQ ID NO: 133) and EVDPIGHVY (SEQ ID NO: 134).

In one embodiment, an isolated nucleic acid composition that encodes a MAGE antigen-specific binding protein having a TCR α chain variable (Vα) domain and a TCR β chain variable (Vβ) domain is provided, the composition comprising:
a nucleic acid sequence that encodes a TCR Vα domain comprising a CDR3 amino acid sequence having at least 80% sequence identity to SEQ ID NO:115, or a functional fragment thereof; and a nucleic acid sequence that encodes a TCR Vβ domain comprising a CDR3 amino acid sequence having at least 80% sequence identity to SEQ ID NO: 118, or a functional fragment thereof, wherein the CDR3 sequences together specifically bind to MAGE A3 and/or MAGE A6.

An example of an appropriate TCR Vα domain CDR3 amino acid sequence that confers specific binding to a MAGE A3 and/or a MAGE A6 antigen (e.g. to EVDPIGHXY (SEQ ID NO: 183)) is shown in SEQ ID NO: 115. As would be clear to a person of skill in the art, variants of the amino acid sequence shown in SEQ ID NO: 115 may also be functional (i.e. retain their ability to confer specific binding to a MAGE A3 and/or a MAGE A6 antigen (e.g. to the peptide EVDPIGHXY (SEQ ID NO: 183)) when the CDR3 is part of TCR Vα domain). Such functional variants are therefore encompassed herein.

For example, appropriate (functional) Vα domain CDR3 amino acid sequences may have at least 80% sequence identity to SEQ ID NO: 115, i.e. they may have at least 80%, at least 84%, at least 92%, or 100% sequence identity to SEQ ID NO: 115. Suitably, percent identity is calculated as the percentage of identity to the entire length of the reference sequence (e.g. SEQ ID NO: 115). In other words, appropriate (functional) Vα domain CDR3 amino acid sequences may vary from the sequence shown in SEQ ID NO: 115 by one or several (e.g. two etc) amino acids.

As stated above, functional variants of SEQ ID NO: 115 retain their ability to confer specific binding to a MAGE A3 and/or a MAGE A6 antigen (i.e. the peptide shown in SEQ ID NO: 183) when the CDR3 is part of TCR Vα domain.

Functional variants may be naturally occurring, synthetic, or synthetically improved functional variants of SEQ ID NO: 115. The term “variant” also encompasses homologues and fragments. Functional variants will typically contain only conservative substitutions of one, two or more amino acids of SEQ ID NO: 115, or substitution, deletion or insertion of non-critical amino acids in non-critical regions of the CDR3.

Non-functional variants are amino acid sequence variants of SEQ ID NO: 115 that do not specifically bind to a MAGE A3 or a MAGE A6 antigen (i.e. the peptide shown in SEQ ID NO: 183). Non-functional variants will typically contain a non-conservative substitution, a deletion, or insertion or premature truncation of the amino acid sequence of SEQ ID NO: 115 or a substitution, insertion or deletion in critical amino acids or critical regions. Methods for identifying functional and non-functional variants are well known to a person of ordinary skill in the art.

In one example, the CDR3 of the Vα domain comprises or consists of the amino acid sequence of SEQ ID NO: 115. In examples where the TCR Vα domain CDR3 has the amino acid sequence of SEQ ID NO: 115, the CDR3 may be encoded by any appropriate nucleic acid sequence.

The encoded TCR Vα domain may comprise, in addition to the specified CDR3, a CDR1 comprising an amino acid sequence of SEQ ID NO: 113, or a functional variant thereof (i.e, wherein the variant retains the ability to specifically bind to a MAGE A3 and/or a MAGE A6 antigen (e.g. the peptide shown in SEQ ID NO: 183)). Such functional variants may be naturally occurring, synthetic, or synthetically improved functional variants of SEQ ID NO: 113. The term “variant” also encompasses homologues and fragments. Functional variants will typically contain only conservative substitutions of one or more amino acids of SEQ ID NO: 113, or substitution, deletion or insertion of non-critical amino acids in non-critical regions of the protein.

Non-functional variants are amino acid sequence variants of SEQ ID NO: 113 that do not specifically bind to a MAGE A3 or a MAGE A6 antigen (e.g. the peptide shown in SEQ ID NO: 183). Non-functional variants will typically contain a non-conservative substitution, a deletion, or insertion or premature truncation of the amino acid sequence of SEQ ID NO: 113 or a substitution, insertion or deletion in critical amino acids or critical regions. Methods for identifying functional and non-functional variants are well known to a person of ordinary skill in the art.

For example, appropriate functional Vα domain CDR1 amino acid sequences may have at least 80% sequence identity to SEQ ID NO: 113, i.e. it may have at least 80%, at least 83%, or 100% sequence identity to SEQ ID NO: 113. Suitably, percent identity is calculated as the percentage of identity to the entire length of the reference sequence (e.g. SEQ ID NO: 113). In other words, appropriate functional Vα domain CDR1 amino acid sequences may vary from the sequence shown in SEQ ID NO: 113 by one or several amino acids. As stated previously, the variant may comprise an amino acid substitution such as a conservative amino acid substitution compared to the sequence shown in SEQ ID NO: 113. As stated above, functional variants of SEQ ID NO: 113 retain the ability to specifically bind to a MAGE A3 and/or a MAGE A6 antigen (e.g. the peptide shown in SEQ ID NO: 183) when the CDR1 is part of TCR Vα domain.

In one example, the CDR1 of the Vα domain comprises or consists of the amino acid sequence of SEQ ID NO: 113. In examples where the TCR Vα domain CDR1 has the amino acid sequence of SEQ ID NO: 113, the CDR1 may be encoded by any appropriate nucleic acid sequence.

The encoded TCR Vα domain may also comprise, in addition to the specified CDR3 (and optionally the specified CDR1 above), a CDR2 comprising an amino acid sequence of SEQ ID NO: 114, or a functional variant thereof (i.e, wherein the variant retains the ability to specifically bind to HLA-B*35:01). Such functional variants may be naturally occurring, synthetic, or synthetically improved functional variants of SEQ ID NO: 114. The term “variant” also encompasses homologues and fragments. Functional variants will typically contain only conservative substitutions of one or more amino acids of SEQ ID NO: 114, or substitution, deletion or insertion of non-critical amino acids in non-critical regions of the protein.

Non-functional variants are amino acid sequence variants of SEQ ID NO: 114 that do not specifically bind to HLA-B*35:01. Non-functional variants will typically contain a non-conservative substitution, a deletion, or insertion or premature truncation of the amino acid sequence of SEQ ID NO: 114 or a substitution, insertion or deletion in critical amino acids or critical regions. Methods for identifying functional and non-functional variants are well known to a person of ordinary skill in the art.

For example, appropriate functional Vα domain CDR2 amino acid sequences may have at least 80% sequence identity to SEQ ID NO: 114, i.e. it may have at least 80%, or 100% sequence identity to SEQ ID NO: 114. Suitably, percent identity is calculated as the percentage of identity to the entire length of the reference sequence (e.g. SEQ ID NO: 114). In other words, appropriate (functional) Vα domain CDR2 amino acid sequences may vary from the sequence shown in SEQ ID NO: 114 by one or several amino acids. As stated previously, the variant may comprise an amino acid substitution such as a conservative amino acid substitution compared to the sequence shown in SEQ ID NO: 114. As stated above, a functional variant of SEQ ID NO: 114 retains the ability to specifically bind to HLA-B*35:01.

In one example, the CDR2 of the Vα domain comprises or consists of the amino acid sequence of SEQ ID NO: 114. In examples where the TCR Vα domain CDR2 has the amino acid sequence of SEQ ID NO: 114, the CDR2 may be encoded by any appropriate nucleic acid sequence.

The encoded TCR Vα domain may therefore comprise the CDRs mentioned in detail above (by SEQ ID specifically i.e. SEQ ID NO: 115, SEQ ID NO: 113 and SEQ ID NO: 114, or functional variants thereof), with appropriate intervening sequences between the CDRs.

The encoded TCR Vα domain may comprise an amino acid sequence of SEQ ID NO: 119, or a functional variant thereof (i.e, wherein the variant TCR Vα domain retains the ability to specifically bind to a MAGE A3 and/or a MAGE A6 antigen (e.g. the peptide shown in SEQ ID NO: 183) when part of a binding protein described herein). Such functional variants may be naturally occurring, synthetic, or synthetically improved functional variants of SEQ ID NO: 119. The term “variant” also encompasses homologues and fragments. Functional variants will typically contain only conservative substitutions of one or more amino acids of SEQ ID NO: 119, or substitution, deletion or insertion of non-critical amino acids in non-critical regions of the protein.

Non-functional variants are amino acid sequence variants of SEQ ID NO: 119 that do not specifically bind to a MAGE A3 or a MAGE A6 antigen (e.g. the peptide shown in SEQ ID NO: 183). Non-functional variants will typically contain a non-conservative substitution, a deletion, or insertion or premature truncation of the amino acid sequence of SEQ ID NO: 119 or a substitution, insertion or deletion in critical amino acids or critical regions. Methods for identifying functional and non-functional variants are well known to a person of ordinary skill in the art.

In one example, the encoded TCR Vα domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 119, whilst retaining the ability to specifically bind to a MAGE A3 and/or a MAGE A6 antigen (e.g. the peptide shown in SEQ ID NO: 183). In other words, a functional TCR Vα domain with one or several amino acid substitutions compared to the sequence of SEQ ID NO: 119 is also encompassed. As stated previously, the amino acid substitution may be a conservative amino acid substitution. The variability in sequence compared to SEQ ID NO: 119 may all be in regions of the TCR Vα domain that do not form CDRs (i.e. the variant may have the CDRs of SEQ ID NO: 115, SEQ ID NO: 113 and/or SEQ ID NO: 114, and still have 25% (or less) sequence variability compared to SEQ ID NO: 119). In other words, the sequence of the CDRs of SEQ ID NO: 119 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 119).

As an example, the encoded TCR Vα domain may comprise an amino acid sequence having at least 75% (e.g. at least 75%, at least 80%, at least 85%, at least 90%, at least 95% etc) sequence identity to the amino acid sequence of SEQ ID NO: 119, wherein the TCR Vα domain comprises a CDR3 having an amino acid sequence of SEQ ID NO: 115. In this example, the TCR Vα domain CDR1 may have an amino acid sequence of SEQ ID NO: 113 and the TCR Vα domain CDR2 may have an amino acid sequence of SEQ ID NO: 114.

As another example, the encoded TCR Vα domain may comprise an amino acid sequence having the amino acid sequence of SEQ ID NO: 119, with 0 to 10 (or 0 to 5) amino acid substitutions, insertions or deletions, wherein the TCR Vα domain comprises a CDR3 having an amino acid sequence of SEQ ID NO: 115. In this example, the TCR Vα domain CDR1 may have an amino acid sequence of SEQ ID NO: 113 and the TCR Vα domain CDR2 may have an amino acid sequence of SEQ ID NO: 114.

In examples where the TCR Vα domain has the amino acid sequence of SEQ ID NO: 119, the TCR Vα domain may be encoded by the nucleic acid sequence of SEQ ID NO: 120, or a genetically degenerate sequence thereof (i.e. other nucleic acid sequences that encode the same protein as a result of the degeneracy of the genetic code).

For the avoidance of doubt, the nucleic acid sequence encoding the TCR Vα domain may also encode a TCR α chain constant domain. Examples of suitable constant domains are generally discussed above.

An example of a specific TCR α chain amino acid sequence that includes a TCR Vα domain described herein with an appropriate constant domain is shown in SEQ ID NO: 123.

Appropriate functional variants of SEQ ID NO: 123 are also encompassed (e.g. variants having at least 75% (e.g. at least 75%, at least 80%, at least 85%, at least 90%, at least 95% etc) sequence identity to the amino acid sequence of SEQ ID NO: 123, wherein the variant TCR α chain amino acid sequence retains its ability to specifically bind to a MAGE A3 and/or a MAGE A6 antigen (e.g. the peptide shown in SEQ ID NO: 183) when part of a binding protein described herein). In other words, a functional TCR α chain with one or several amino acid substitutions compared to the sequence of SEQ ID NO: 123 is also encompassed. As stated previously, the amino acid substitution may be a conservative amino acid substitution. The variability in sequence compared to SEQ ID NO: 123 may all be in regions of the TCR α chain that do not form CDRs (i.e. the variant may have the CDRs of SEQ ID NO: 115, SEQ ID NO: 113 and/or SEQ ID NO: 114, and still have 25% (or less) sequence variability compared to SEQ ID NO: 123). In other words, the sequence of the CDRs of SEQ ID NO: 123 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 123).

As an example, the encoded TCR α chain may comprise an amino acid sequence having at least 75% (e.g. at least 75%, at least 80%, at least 85%, at least 90%, at least 95% etc) sequence identity to the amino acid sequence of SEQ ID NO: 123, wherein the TCR α chain comprises a CDR3 having an amino acid sequence of SEQ ID NO: 115. In this example, the TCR α chain CDR1 may have an amino acid sequence of SEQ ID NO: 113 and the TCR α chain CDR2 may have an amino acid sequence of SEQ ID NO: 114.

In examples where the TCR α chain has the amino acid sequence of SEQ ID NO: 123, the TCR α chain may be encoded by the nucleic acid sequence of SEQ ID NO: 124, or a genetically degenerate sequence thereof (i.e. other nucleic acid sequences that encode the same protein as a result of the degeneracy of the genetic code). It is noted that SEQ ID NO: 124 is the nucleic acid sequence for TCR α chain of clone CT23.2H9.

In one example, the nucleic acid composition provided herein comprises a nucleic acid sequence that encodes a TCR Vα domain comprising a CDR3 amino acid sequence having at least 80% sequence identity to SEQ ID NO: 115, or a functional fragment thereof.

In another example, the CDR3 of the Vα domain of a nucleic acid composition provided herein comprises or consists of the amino acid sequence of SEQ ID NO: 115.

In another example, the Vα domain of a nucleic acid composition provided herein comprises an amino acid sequence having at least 80% sequence identity to, comprising, or consisting of, SEQ ID NO: 119.

As provided above, the inventors identified TCR clone CT23.2H9 which interacts with EVDPIGHXY (SEQ ID NO: 183)) in the context of HLA-B*35:01. The sequences provided herein that correspond to TCR clone CT23.2H9 are SEQ ID NO:s 113 to 126.

An example of an appropriate TCR Vβ domain CDR3 amino acid sequence that confers specific binding to a MAGE A3 and/or a MAGE A6 antigen (e.g. to EVDPIGHXY (SEQ ID NO: 183)) is shown in SEQ ID NO: 118. As would be clear to a person of skill in the art, variants of the amino acid sequence shown in SEQ ID NO: 118 may also be functional (i.e. retain their ability to confer specific binding to a MAGE A3 and/or a MAGE A6 antigen (i.e. the peptide shown in SEQ ID NO: 183) when the CDR3 is part of TCR Vβ domain). Such functional variants are therefore encompassed herein.

For example, appropriate (functional) Vβ domain CDR3 amino acid sequences may have at least 80% sequence identity to SEQ ID NO: 118, i.e. they may have at least 80%, at least 86%, at least 93%, or 100% sequence identity to SEQ ID NO: 118. Suitably, percent identity is calculated as the percentage of identity to the entire length of the reference sequence (e.g. SEQ ID NO: 118). In other words, appropriate (functional) Vβ domain CDR3 amino acid sequences may vary from the sequence shown in SEQ ID NO: 118 by one or several (e.g. two) amino acids. As stated above, functional variants of SEQ ID NO: 118 retain their ability to confer specific binding to a MAGE A3 and/or a MAGE A6 antigen (e.g. the peptide shown in SEQ ID NO: 183) when the CDR3 is part of TCR Vβ domain.

Functional variants may be naturally occurring, synthetic, or synthetically improved functional variants of SEQ ID NO: 118. The term “variant” also encompasses homologues and fragments. Functional variants will typically contain only conservative substitutions of one or more amino acids of SEQ ID NO: 118, or substitution, deletion or insertion of non-critical amino acids in non-critical regions of the CDR3.

Non-functional variants are amino acid sequence variants of SEQ ID NO: 118 that do not specifically bind to a MAGE A3 or a MAGE A6 antigen (e.g. the peptide shown in SEQ ID NO: 183). Non-functional variants will typically contain a non-conservative substitution, a deletion, or insertion or premature truncation of the amino acid sequence of SEQ ID NO: 118 or a substitution, insertion or deletion in critical amino acids or critical regions. Methods for identifying functional and non-functional variants are well known to a person of ordinary skill in the art.

In one example, the CDR3 of the Vβ domain comprises or consists of the amino acid sequence of SEQ ID NO: 118. In examples where the TCR Vβ domain CDR3 has the amino acid sequence of SEQ ID NO: 118, the CDR3 may be encoded by any appropriate nucleic acid sequence.

The encoded TCR Vβ domain may comprise, in addition to the specified CDR3, a CDR1 comprising an amino acid sequence of SEQ ID NO: 116, or a functional variant thereof (i.e, wherein the variant retains the ability to specifically bind to a MAGE A3 and/or a MAGE A6 antigen (e.g. the peptide shown in SEQ ID NO: 183)). Such functional variants may be naturally occurring, synthetic, or synthetically improved functional variants of SEQ ID NO: 116. The term “variant” also encompasses homologues and fragments. Functional variants will typically contain only conservative substitutions of one or more amino acids of SEQ ID NO: 116, or substitution, deletion or insertion of non-critical amino acids in non-critical regions of the protein.

Non-functional variants are amino acid sequence variants of SEQ ID NO: 116 that do not specifically bind to a MAGE A3 or a MAGE A6 antigen (e.g. the peptide shown in SEQ ID NO: 183). Non-functional variants will typically contain a non-conservative substitution, a deletion, or insertion or premature truncation of the amino acid sequence of SEQ ID NO: 116 or a substitution, insertion or deletion in critical amino acids or critical regions. Methods for identifying functional and non-functional variants are well known to a person of ordinary skill in the art.

For example, appropriate functional Vβ domain CDR1 amino acid sequences may have at least 80% sequence identity to SEQ ID NO: 116, i.e. it may have at least 80%, or 100% sequence identity to SEQ ID NO: 116. Suitably, percent identity is calculated as the percentage of identity to the entire length of the reference sequence (e.g. SEQ ID NO: 116). In other words, appropriate (functional) Vβ domain CDR1 amino acid sequences may vary from the sequence shown in SEQ ID NO: 116 by one or several amino acids. As stated previously, the variant may comprise an amino acid substitution such as a conservative amino acid substitution compared to the sequence shown in SEQ ID NO:116. As stated above, functional variants of SEQ ID NO: 116 retain the ability to specifically bind to a MAGE A3 and/or a MAGE A6 antigen (e.g. the peptide shown in SEQ ID NO: 183) when the CDR1 is part of TCR Vβ domain).

In one example, the CDR1 of the Vβ domain comprises or consists of the amino acid sequence of SEQ ID NO: 116. In examples where the TCR Vα domain CDR1 has the amino acid sequence of SEQ ID NO:116, the CDR1 may be encoded by any appropriate nucleic acid sequence.

The encoded TCR Vβ domain may also comprise, in addition to the specified CDR3 (and optionally the specified CDR1 above), a CDR2 having an amino acid sequence of SEQ ID NO: 117, or a functional variant thereof (i.e, wherein the variant retains the ability to specifically bind to HLA-B*35:01). Such functional variants may be naturally occurring, synthetic, or synthetically improved functional variants of SEQ ID NO: 117. The term “variant” also encompasses homologues and fragments. Functional variants will typically contain only conservative substitutions of one or more amino acids of SEQ ID NO: 117, or substitution, deletion or insertion of non-critical amino acids in non-critical regions of the protein.

Non-functional variants are amino acid sequence variants of SEQ ID NO: 117 that do not specifically bind to HLA-B*35:01. Non-functional variants will typically contain a non-conservative substitution, a deletion, or insertion or premature truncation of the amino acid sequence of SEQ ID NO: 117 or a substitution, insertion or deletion in critical amino acids or critical regions. Methods for identifying functional and non-functional variants are well known to a person of ordinary skill in the art.

For example, appropriate functional Vβ domain CDR2 amino acid sequences may have at least 80% sequence identity to SEQ ID NO: 117, i.e. it may have at least 80%, at least 83, or 100% sequence identity to SEQ ID NO: 117. Suitably, percent identity is calculated as the percentage of identity to the entire length of the reference sequence (e.g. SEQ ID NO: 117). In other words, appropriate (functional) Vβ domain CDR2 amino acid sequences may vary from the sequence shown in SEQ ID NO: 117 by one or several amino acids. As stated previously, the variant may comprise an amino acid substitution such as a conservative amino acid substitution compared to the sequence shown in SEQ ID NO: 117. As stated above, a functional variant of SEQ ID NO: 117 retains the ability to specifically bind to HLA-B*35:01.

In one example, the CDR2 of the Vβ domain comprises or consists of the amino acid sequence of SEQ ID NO: 117. In examples where the TCR Vβ domain CDR2 has the amino acid sequence of SEQ ID NO:117, the CDR2 may be encoded by any appropriate nucleic acid sequence.

The encoded TCR Vβ domain may therefore comprise the CDRs mentioned in detail above (by SEQ ID specifically i.e. SEQ ID NO:118, SEQ ID NO: 116 and SEQ ID NO: 117, or functional variants thereof), with appropriate intervening sequences between the CDRs.

The encoded TCR Vβ domain may have an amino acid sequence of SEQ ID NO: 121, or a functional variant thereof (i.e, wherein the variant TCR Vβ domain retains the ability to specifically bind to a MAGE A3 and/or a MAGE A6 antigen (e.g. the peptide shown in SEQ ID NO: 183) when part of a binding protein described herein). Such functional variants may be naturally occurring, synthetic, or synthetically improved functional variants of SEQ ID NO: 121. The term “variant” also encompasses homologues and fragments. Functional variants will typically contain only conservative substitutions of one or more amino acids of SEQ ID NO: 121, or substitution, deletion or insertion of non-critical amino acids in non-critical regions of the protein.

Non-functional variants are amino acid sequence variants of SEQ ID NO: 121 that do not specifically bind to a MAGE A3 or a MAGE A6 antigen (e.g. the peptide shown in SEQ ID NO: 183). Non-functional variants will typically contain a non-conservative substitution, a deletion, or insertion or premature truncation of the amino acid sequence of SEQ ID NO: 121 or a substitution, insertion or deletion in critical amino acids or critical regions. Methods for identifying functional and non-functional variants are well known to a person of ordinary skill in the art.

In one example, the encoded TCR Vβ domain may have an amino acid sequence having at least 75%, at least 80%, at least 85% or at least 90% (or at least 91%, at least 92%, at least 93%, at least 94%, at least 95%, at least 96%, at least 97%, at least 98%, at least 99%, or 100%) sequence identity to the amino acid sequence of SEQ ID NO: 121, whilst retaining the ability to specifically bind to a MAGE A3 and/or a MAGE A6 antigen (e.g. the peptide shown in SEQ ID NO: 183). In other words, a functional TCR Vβ domain with one or several amino acid substitutions compared to the sequence of SEQ ID NO: 121 is also encompassed. As stated previously, the amino acid substitution may be a conservative amino acid substitution. The variability in sequence compared to SEQ ID NO: 121 may all be in regions of the TCR Vβ domain that do not form CDRs (i.e. the variant may have the CDRs of SEQ ID NO: 118, SEQ ID NO: 116 and/or SEQ ID NO: 117, and still have 25% (or less) sequence variability compared to SEQ ID NO: 121). In other words, the sequence of the CDRs of SEQ ID NO: 121 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 121).

As an example, the encoded TCR Vβ domain may comprise an amino acid sequence having at least 75% (e.g. at least 75%, at least 80%, at least 85%, at least 90%, at least 95% etc) sequence identity to the amino acid sequence of SEQ ID NO: 121, wherein the TCR Vβ domain comprises a CDR3 having an amino acid sequence of SEQ ID NO: 118. In this example, the TCR Vβ domain CDR1 may have an amino acid sequence of SEQ ID NO: 116 and the TCR Vβ domain CDR2 may have an amino acid sequence of SEQ ID NO: 117.

In examples where the TCR Vβ domain has the amino acid sequence of SEQ ID NO: 121, the TCR Vβ domain may be encoded by the nucleic acid sequence of SEQ ID NO: 122, or a genetically degenerate sequence thereof (i.e. other nucleic acid sequences that encode the same protein as a result of the degeneracy of the genetic code).

For the avoidance of doubt, the nucleic acid sequence encoding the TCR Vβ domain may also encode a TCR β chain constant domain. Examples of suitable constant domains are generally discussed above.

An example of a specific TCR β chain amino acid sequence that includes a TCR Vβ domain described herein and an appropriate constant domain is shown in SEQ ID NO: 125. Appropriate functional variants of SEQ ID NO: 125 are also encompassed (e.g. variants having at least 75% (e.g. at least 75%, at least 80%, at least 85%, at least 90%, at least 95% etc) sequence identity to the amino acid sequence of SEQ ID NO: 125, wherein the variant TCR β chain amino acid sequence retains its ability to specifically bind to a MAGE A3 and/or a MAGE A6 antigen (e.g. the peptide shown in SEQ ID NO: 183) when part of a binding protein described herein). In other words, a functional TCR β chain with one or several amino acid substitutions compared to the sequence of SEQ ID NO: 125 is also encompassed. As stated previously, the amino acid substitution may be a conservative amino acid substitution. The variability in sequence compared to SEQ ID NO: 125 may all be in regions of the TCR β chain that do not form CDRs (i.e. the variant may have the CDRs of SEQ ID NO: 118, SEQ ID NO: 116 and/or SEQ ID NO: 117, and still have 25% (or less) sequence variability compared to SEQ ID NO: 125. In other words, the sequence of the CDRs of SEQ ID NO: 125 may be retained whilst the rest of the sequence is varied, as appropriate within the “at least 75% identity” parameters specified above. Suitably, percent identity can be calculated as the percentage of identity to the entire length of the reference sequence (e.g. SEQ ID NO: 125).

As an example, the encoded TCR β chain may comprise an amino acid sequence having at least 75% (e.g. at least 75%, at least 80%, at least 85%, at least 90%, at least 95% etc) sequence identity to the amino acid sequence of SEQ ID NO: 125, wherein the TCR β chain comprises a CDR3 having an amino acid sequence of SEQ ID NO: 118. In this example, the TCR β chain CDR1 may have an amino acid sequence of SEQ ID NO: 116 and the TCR β chain CDR2 may have an amino acid sequence of SEQ ID NO: 117.

In examples where the TCR β chain has the amino acid sequence of SEQ ID NO:125, the TCR β chain may be encoded by the nucleic acid sequence of SEQ ID NO: 126, or a genetically degenerate sequence thereof (i.e. other nucleic acid sequences that encode the same protein as a result of the degeneracy of the genetic code). It is noted that SEQ ID NO: 126 is the nucleic acid sequence for TCR β chain of clone CT23.2H9.

In an example, the nucleic acid composition provided herein comprises a nucleic acid sequence that encodes a TCR Vβ domain comprising a CDR3 amino acid sequence having at least 80% sequence identity to SEQ ID NO: 118, or a functional fragment thereof.

In another example, the CDR3 of the Vβ domain of a nucleic acid composition provided herein comprises or consists of the amino acid sequence of SEQ ID NO:118.

In a further example, the Vβ domain of a nucleic acid composition provided herein comprises an amino acid sequence having at least 80% sequence identity to, comprising, or consisting of, SEQ ID NO: 121.

The TCR Vβ domain sequences derived from TCR clone CT23.2H9 discussed above are particularly compatible with the TCR Vα domain sequences derived from TCR clone CT23.2H9 discussed elsewhere herein.

Accordingly, in one example, a nucleic acid composition described herein encodes a MAGE A3 and/or a MAGE A6 antigen-specific binding protein having TCR Vα domain comprising a CDR3 amino acid sequence having at least 80% sequence identity to SEQ ID NO:115, or a functional fragment thereof; and a nucleic acid sequence that encodes a TCR Vβ domain comprising a CDR3 amino acid sequence having at least 80% sequence identity to SEQ ID NO: 118, or a functional fragment thereof.

In a particular example, a nucleic acid composition described herein encodes a MAGE A3 and/or a MAGE A6 antigen-specific binding protein having a TCR Vα domain with a CDR3 amino acid sequence comprising or consisting of the amino acid sequence of SEQ ID NO: 115; and a TCR Vβ domain with a CDR3 comprising or consisting of the amino acid sequence of SEQ ID NO:118. In addition, the MAGE A3 and/or a MAGE A6 antigen may comprise or consist of the sequence shown in SEQ ID NO: 183. Furthermore, the TCR Vα domain may be part of a TCR α chain having a constant domain and the TCR Vβ domain may be part of a TCR β chain having a constant domain.

In this particular example, the Vα domain may comprise an amino acid sequence having at least 80% sequence identity to, comprising, or consisting of, SEQ ID NO: 119; and the Vβ domain may comprise an amino acid sequence having at least 80% sequence identity to, comprising, or consisting of, SEQ ID NO: 121. In one example, the Vα domain comprises the amino acid sequence of SEQ ID NO: 119 and the Vβ domain comprises the amino acid sequence of SEQ ID NO: 121. In such cases, the Vα domain may be encoded by a nucleic acid sequence comprising the sequence of SEQ ID NO: 120; and the Vβ domain may be encoded by a nucleic acid sequence comprising the sequence of SEQ ID NO: 122.

In this particular example, the TCR Vα domain may include a CDR1 amino acid sequence comprising or consisting of the amino acid sequence of SEQ ID NO:113 and a CDR2 amino acid sequence comprising or consisting of the amino acid sequence of SEQ ID NO:114. Furthermore, the TCR Vβ domain may include a CDR1 amino acid sequence comprising or consisting of the amino acid sequence of SEQ ID NO:116 and a CDR2 amino acid sequence comprising or consisting of the amino acid sequence of SEQ ID NO: 117.

For the avoidance of doubt, this particular example encompasses components of TCR clone CT23.2H9 exemplified herein. The different components of TCR clone CT23.2H9 and their respective SEQ ID Nos are summarised in Table 14 below.

As stated in more detail elsewhere herein, the nucleic acid composition described herein encodes both a TCR Vα domain and a TCR Vβ domain, which form the binding protein that is capable of specifically binding to a MAGE A3 and/or a MAGE A6 antigen. In examples where the TCR Vα domain and the TCR Vβ domain are encoded by the same nucleic acid sequence, the TCR Vα domain and TCR Vβ domain may be joined together via a linker. Suitable linkers are discussed generally elsewhere herein. Additional appropriate polypeptide domains that may also be encoded by the nucleic acid sequences that encode the TCR Vα domain and/or the TCR Vβ domain are also discussed generally elsewhere herein.

In one example, the nucleic acid composition described herein may encode a soluble TCR or a chimeric single chain TCR wherein the TCR alpha chain variable domain is linked to the TCR beta chain variable domain and a constant domain which is e.g. fused to the CD3 zeta signalling domain. These are discussed generally in more detail elsewhere herein.

### Vector Systems

A vector system is also provided which includes a nucleic acid composition described herein. The vector system may have one or more vectors. As discussed previously, the binding protein components that are encoded by the nucleic acid composition may be encoded by one or more nucleic acid sequences in the nucleic acid composition. In examples where all of the binding protein components are encoded by a single nucleic acid sequence, the nucleic acid sequence may be present within a single vector (and thus the vector system described herein may comprise of one vector only). In examples where the binding protein components are encoded by two or more nucleic acid sequences (wherein the plurality of nucleic acid sequences, together, encode all of the components of the binding protein) these two or more nucleic acid sequences may be present within one vector (e.g. in different open reading frames of the vector), or may be distributed over two or more vectors. In this example, the vector system will comprise a plurality of distinct vectors (i.e. vectors with different nucleotide sequences).

Accordingly, in one example, a vector system is provided, comprising a nucleic acid composition described herein.

Any appropriate vector can be used. By way of example only, the vector may be a plasmid, a cosmid, or a viral vector, such as a retroviral vector or a lentiviral vector. Adenovirus, adeno-associated virus, vaccinia virus, canary poxvirus, herpes virus, minicircle vectors and naked (synthetic) DNA/RNA may also be used (for details on minicircle vectors, see for example non-viral Sleeping Beauty transposition from minicircle vectors as published by R Monjezi et al., Leukemia 2017). Alternatively, single stranded or double stranded DNA or RNA can be used to transfect lymphocytes with a TCR of interest (see Roth et al 2018 Nature vol 559; page 405).

In one example, the vector is a plasmid, a viral vector, or a cosmid, optionally wherein the vector is selected from the group consisting of a retrovirus, lentivirus, adeno-associated virus, adenovirus, vaccinia virus, canary poxvirus, herpes virus, minicircle vector and synthetic DNA or RNA.

As used herein, the term “vector” refers to a nucleic acid sequence capable of transporting another nucleic acid sequence to which it has been operably linked. The vector can be capable of autonomous replication or it can integrate into a host DNA. The vector may include restriction enzyme sites for insertion of recombinant DNA and may include one or more selectable markers or suicide genes. The vector can be a nucleic acid sequence in the form of a plasmid, a bacteriophage or a cosmid. Preferably the vector is suitable for expression in a cell (i.e. the vector is an “expression vector”). Preferably, the vector is suitable for expression in a human T cell such as a CD8⁺ T cell or CD4⁺ T cell, or stem cell, iPS cell, or NK cell. In certain aspects, the vector is a viral vector, such as a retroviral vector, a lentiviral vector or an adeno-associated vector. Optionally, the vector is selected from the group consisting of an adenovirus, vaccinia virus, canary poxvirus, herpes virus, minicircle vector and synthetic DNA or synthetic RNA.

Preferably the (expression) vector is capable of propagation in a host cell and is stably transmitted to future generations.

The vector may comprise regulatory sequences. “Regulatory sequences” as used herein, refers to, DNA or RNA elements that are capable of controlling gene expression. Examples of expression control sequences include promoters, enhancers, silencers, TATA-boxes, internal ribosomal entry sites (IRES), attachment sites for transcription factors, transcriptional terminators, polyadenylation sites etc. Optionally, the vector includes one or more regulatory sequences operatively linked to the nucleic acid sequence to be expressed. Regulatory sequences include those which direct constitutive expression, as well as tissue-specific regulatory and/or inducible sequences.

Optionally, the vector comprises the nucleic acid sequence of interest operably linked to a promoter. “Promoter”, as used herein, refers to the nucleotide sequences in DNA to which RNA polymerase binds to start transcription. The promoter may be inducible or constitutively expressed. Alternatively, the promoter is under the control of a repressor or stimulatory protein. The promoter may be one that is not naturally found in the host cell (e.g. it may be an exogenous promoter). The skilled person in the art is well aware of appropriate promoters for use in the expression of target proteins, wherein the selected promoter will depend on the host cell.

“Operably linked” refers to a single or a combination of the below-described control elements together with a coding sequence in a functional relationship with one another, for example, in a linked relationship so as to direct expression of the coding sequence.

The vector may comprise a transcriptional terminator. “Transcriptional terminator” as used herein, refers to a DNA element, which terminates the function of RNA polymerases responsible for transcribing DNA into RNA. Preferred transcriptional terminators are characterized by a run of T residues preceded by a GC rich dyad symmetrical region.

The vector may comprise a translational control element. “Translational control element”, as used herein, refers to DNA or RNA elements that control the translation of mRNA. Preferred translational control elements are ribosome binding sites. Preferably, the translational control element is from a homologous system as the promoter, for example a promoter and its associated ribozyme binding site. Preferred ribosome binding sites are known, and will depend on the chosen host cell.

The vector may comprise restriction enzyme recognition sites. “Restriction enzyme recognition site” as used herein, refers to a motif on the DNA recognized by a restriction enzyme.

The vector may comprise a selectable marker. “Selectable marker” as used herein, refers to proteins that, when expressed in a host cell, confer a phenotype onto the cell which allows selection of the cell expressing said selectable marker gene. Generally this may be a protein that confers a new beneficial property onto the host cell (e.g. antibiotic resistance) or a protein that is expressed on the cell surface and thus accessible for antibody binding. Appropriate selectable markers are well known in the art.

Optionally, the vector may also comprise a suicide gene. “Suicide gene” as used herein, encodes a protein that induce death of the modified cell upon treatment with specific drugs. By way of example, suicide can be induced in cells modified by the herpes simplex virus thymidine kinase gene upon treatment with specific nucleoside analogs including ganciclovir, cells modified by human CD20 upon treatment with anti-CD20 monoclonal antibody and cells modified with inducible Caspase9 (iCasp9) upon treatment with AP1903 (reviewed by B S Jones, L S Lamb, F Goldman, A Di Stasi; Improving the safety of cell therapy products by suicide gene transfer. Front Pharmacol. (2014) 5:254). Appropriate suicide genes are well known in the art.

Preferably the vector comprises those genetic elements which are necessary for expression of the binding proteins described herein by a host cell. The elements required for transcription and translation in the host cell include a promoter, a coding region for the protein(s) of interest, and a transcriptional terminator.

A person of skill in the art will be well aware of the molecular techniques available for the preparation of (expression) vectors and how the (expression) vectors may be transduced or transfected into an appropriate host cell (thereby generating a modified cell described further below). The (expression) vector system described herein can be introduced into cells by conventional techniques such as transformation, transfection or transduction.

“Transformation”, “transfection” and “transduction” refer generally to techniques for introducing foreign (exogenous) nucleic acid sequences into a host cell, and therefore encompass methods such as electroporation, microinjection, gene gun delivery, transduction with retroviral, lentiviral or adeno-associated vectors, lipofection, superfection etc. The specific method used typically depends on both the type of vector and the cell. Appropriate methods for introducing nucleic acid sequences and vectors into host cells such as human cells are well known in the art; see for example Sambrook et al (1989) Molecular Cloning, A Laboratory Manual, Cold Spring Harbor Laboratory, Cold Spring Harbor, N.Y; Ausubel et al (1987) Current Protocols in Molecular Biology, John Wiley and Sons, Inc., NY; Cohen et al (1972) Proc. Natl. Acad. Sci. USA 69, 2110; Luchansky et al (1988) Mol. Microbiol. 2, 637-646. Further conventional methods that are suitable for preparing expression vectors and introducing them into appropriate host cells are described in detail in WO2016/071758 for example.

It is understood that it some examples, the host cell is contacted with the vector system (e.g. viral vector) in vitro, ex vivo, and in some examples, the host cell is contacted with the vector system (e.g. viral vector) in vivo.

The term “host cell” includes any cell into which the nucleic acid composition or vector system described herein may be introduced. Once a nucleic acid molecule or vector system has been introduced into the cell, it may be referred to as a “modified cell” herein. Once the nucleic acid molecule or vector is introduced into the host cell, the resultant modified cell should be capable of expressing the encoded binding protein (and e.g. correctly localising the encoded binding protein for its intended function e.g. transporting the encoded binding protein to the cell surface).

The nucleic acid composition or vector system may be introduced into the cell using any conventional method known in the art. For example, the nucleic acid composition or vector system may be introduced using CRISPR technology. Insertion of the nucleic acid sequences at the endogenous TCR locus by engineering with CRISPR/Cas9 and homologous directed repair (HDR) or non-homologous end joining (NHEJ) is therefore encompassed. Other conventional methods such as transfection, transduction or transformation of the cell may also be used.

The term “modified cell” refers to a genetically altered (e.g. recombinant) cell. The modified cell includes at least one exogenous nucleic acid sequence (i.e. a nucleic acid sequence that is not naturally found in the host cell). In the context of the invention, the exogenous sequence comprises at least one of the T cell receptor component parts described herein for any of clones CT27.4F7, CT31.10C1, CT43.2D8, CT44.6G4, CT23.3H4, MRM23.3B2, CT12.3G2, CT4.20.2C2 or CT23.2H9 (e.g. the sequences etc that encode the CDR3 sequences that are specific for a MAGE antigen (e.g. the peptide of SEQ ID NO: 127, 128, 129, 130, 131, 132, or 183 (e.g. 133 or 134))). The term “modified cell” refers to the particular subject cell and also to the progeny or potential progeny of such a cell. Because certain modifications may occur in succeeding generations due to either mutation or environmental influences, such progeny may not, in fact, be identical to the parent cell, but are still included within the scope of the term as used herein.

In one example, a modified cell comprises a nucleic acid composition or a vector system provided herein.

The host cell (and thus the modified cell) is typically a eukaryotic cell, and particularly a human cell (e.g. a T cell such as a CD8⁺ T cell or a CD4⁺ T cell, or a mixture thereof, or a hematopoietic stem cell, an iPSC, or gamma-delta T cell, or a pluripotent stem cell, or a NK-T cell or NK cell). The host cell (and thus the modified cell) may be an autologous or allogeneic cell (e.g. such as a CD8⁺ T cell or a CD4⁺ T cell, or a mixture thereof, or a hematopoietic stem cell, an iPSC, or gamma-delta T cell, or a pluripotent stem cell, or a NK-T cell or NK cell). “Allogeneic cell” refers to a cell derived from a different individual to the individual to which it is later administered. In other words, the host cell (and thus the modified cell) may be an isolated cell from a distinct individual compared to the subject to be treated. “Autologous cell” refers to a cell derived from the individual to which it is also later administered. In other words, the host cell (and thus the modified cell) may be an isolated cell from the subject that is to be treated.

Accordingly, in an example, the modified cell is a human cell.

The host cell (and thus the modified cell) may be any cell that is able to confer anti-tumour immunity after TCR gene transfer. Non limiting examples of appropriate cells include autologous or allogeneic CD8 T cells, CD4 T cells, Natural Killer (NK) cells, NKT cells, gamma-delta T cells, inducible pluripotent stem cells (iPSCs), hematopoietic stem cells or other progenitor cells and any other autologous or allogeneic cell or cell line (NK-92 for example or T cell lines) that is able to confer anti-tumor immunity after TCR gene transfer.

Accordingly, in one example the modified cell is selected from the group consisting of a CD8 T cell, a CD4 T cell, an NK cell, an NK-T cell, a gamma-delta T cell, a hematopoietic stem cell, an inducible pluripotent stem cell, a progenitor cell, a T cell line and a NK-92 cell line.

In the context of the methods of treatment described herein, the host cell (and thus the modified cell) is typically for administration to a HLA-A*02:01, HLA-C*07:02, HLA-A*01:01, HLA-A*03:01, HLA-B*07:02 and/or HLA-B*35:01 positive human subject. In view of this, the host cell (and thus the modified cell) is typically HLA-A*02:01, HLA-C*07:02, HLA-A*01:01, HLA-A*03:01, HLA-B*07:02 and/or HLA-B*35:01 positive but needs to be MAGE antigen negative (i.e. modified cells can either be HLA-A*02:01, HLA-C*07:02, HLA-A*01:01, HLA-A*03:01, HLA-B*07:02 and/or HLA-B*35:01 positive or negative).

In the context of the methods of treatment described herein, the host cell (and thus the modified cell) that is to be administered to the subject can either be autologous or allogeneic.

Advantageously, the modified cell is capable of expressing the binding protein encoded by the nucleic acid composition or vector system described herein (i.e. the TCR component parts) such that the modified cell provides an immunotherapy that specifically targets cells that express a MAGE antigen (e.g. a MAGE-A1, a MAGE-A9, a MAGE-A3 and/or a MAGE-A6 antigen), and thus can be used to treat or prevent MAGE associated diseases or conditions in a corresponding HLA-A*02:01, HLA-C*07:02, HLA-A*01:01, HLA-A*03:01, HLA-B*07:02 and/or HLA-B*35:01 positive human subject. More details on this use are given below.

### Pharmaceutical Compositions

A nucleic acid composition, vector system, modified cell, or isolated nucleic acid sequence described herein may be provided as part of a pharmaceutical composition. Advantageously, such compositions may be administered to a human subject in need thereof (as described elsewhere herein). A particularly suitable composition may be selected based on the HLA serotype of the human subject, as described in detail elsewhere herein.

A pharmaceutical composition may comprise a nucleic acid composition, vector system, modified cell, or isolated nucleic acid sequence described herein along with a pharmaceutically acceptable excipient, adjuvant, diluent and/or carrier.

Compositions may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected nucleic acid composition, vector system, modified cell, or isolated nucleic acid sequence without causing any undesirable biological effects or interacting in a deleterious manner with any of the other components of the pharmaceutical composition in which it is contained.

Excipients are natural or synthetic substances formulated alongside an active ingredient (e.g. a nucleic acid sequence, a nucleic acid composition, vector or vector system, modified cell, or isolated nucleic acid as provided herein), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art.

Diluents are diluting agents. Pharmaceutically acceptable diluents are well known in the art. A suitable diluent is therefore easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

### Treatment of a Subject

Pharmaceutical compositions described herein may advantageously be administered to a HLA-A*02:01, HLA-C*07:02, HLA-A*01:01, HLA-A*03:01, HLA-B*07:02 and/or HLA-B*35:01 positive human subject in need thereof (where certain compositions are more suitable for certain human subjects, based on their HLA status, as described in more detail elsewhere herein).

Typically, the subject in need of treatment has a disease or condition that is associated with an elevated level of HLA-restricted MAGE antigens (i.e. MAGE antigens that are presented at the cell surface in the context of an HLA). For example, MAGE-A1, MAGE-A9, MAGE-A3 and/or MAGE-A6 antigens that are presented at the cell surface in the context of an HLA.

The disease or condition is typically a MAGE associated disease or condition. In one example, the MAGE associated disease or condition may be a hyperproliferative disease or condition. The MAGE associated disease or condition (e.g. the hyperproliferative disease or condition) is typically one in which a HLA-restricted MAGE antigen described herein is presented at the cell surface in the context of a HLA, e.g. a MAGE-A1, MAGE-A9, MAGE-A3 and/or MAGE-A6 antigen presented at the cell surface in the context of an HLA.

In one example, the MAGE associated disease or condition may be a hematological malignancy. In other words, it may be a hematological malignancy with an elevated level of HLA-restricted MAGE antigens (i.e. MAGE antigens that are presented at the cell surface in the context of a HLA, e.g. MAGE-A1, MAGE-A9, MAGE-A3 and/or MAGE-A6 antigens that are presented at the cell surface in the context of an HLA). Examples of appropriate hematological malignancies are well known in the art, and include, for example Multiple myeloma, plasma cell leukemia, Amyloidosis (AL), Acute lymphoblastoid leukemia (ALL), Chronic lymphocytic leukemia (CLL), Waldenstrom macroglobulinemia, Acute myeloid leukemia (AML), Myeloid dysplastic syndrome (MDS) and B cell lymphoma. Examples of B cell lymphomas include Diffuse large B cell lymphoma (DLBCL), High grade B cell lymphoma, Mantel cell lymphoma (MCL), Follicular lymphoma (FL) and Burkitt Lymphoma.

In one example, the MAGE associated disease or condition is multiple myeloma.

In an alternative example, the MAGE associated disease or condition may be a solid tumor. In other words, it may be a solid tumor with an elevated level of HLA-restricted MAGE antigens (i.e. MAGE antigens that are presented at the cell surface in the context of an HLA, e.g. MAGE-A1, MAGE-A9, MAGE-A3 and/or MAGE-A6 antigens that are presented at the cell surface in the context of an HLA). Examples of appropriate solid tumours are well known in the art, and include, for example Melanoma, Lung carcinoma, Bladder carcinoma, Ovarian carcinoma, Head and neck carcinoma, Breast carcinoma, Sarcoma, Uveal melanoma and Uterine carcinoma. For example, non-small cell lung carcinoma, head and neck squamous cell carcinoma, invasive breast carcinoma and synovial sarcoma.

The MAGE associated disease or condition may be a hyperproliferative disease or condition. For example, the MAGE associated disease or condition may be a HLA-restricted MAGE antigen expressing tumor or cancer. In other words, the MAGE associated disease or condition may be a MAGE positive tumor or cancer. For example, the MAGE associated disease or condition may be a MAGE-A1, a MAGE-A9, a MAGE-A3 and/or a MAGE-A6 positive tumor or cancer.

In an example, the pharmaceutical composition provided herein is for use in inducing or enhancing an immune response in human subject diagnosed with a MAGE associated disease or condition.

As would be clear to a person skilled in the art, an appropriate therapy for subject in need thereof (e.g. an appropriate pharmaceutical composition described herein) may be selected based on the HLA serotype of the subject.

In one example, if the subject in need thereof is HLA-A*02:01 positive, an appropriate therapy (e.g. an appropriate pharmaceutical composition described herein) may comprise components of TCR clone CT27.4F7 exemplified herein. Accordingly, TCRs comprising components of TCR clone CT27.4F7 exemplified herein are particularly suitable for administration, or treating, stimulating, providing appropriate immunity (e.g. anti-tumor immunity etc) in HLA-A*02:01 positive human subjects.

In one example, if the subject in need thereof is HLA-C*07:02 positive, an appropriate therapy (e.g. an appropriate pharmaceutical composition described herein) may comprise components of TCR clone CT31.10C1 exemplified herein. Accordingly, TCRs comprising components of TCR clone CT31.10C1 exemplified herein are particularly suitable for administration, or treating, stimulating, providing appropriate immunity (e.g. anti-tumor immunity etc) in HLA-C*07:02 positive human subjects.

In one example, if the subject in need thereof is HLA-A*01:01 positive, an appropriate therapy (e.g. an appropriate pharmaceutical composition described herein) may comprise components of (i) TCR clone CT43.2D8; or (ii) TCR clone CT44.6G4 exemplified herein. Accordingly, TCRs comprising components of (i) TCR clone CT43.2D8; or (ii) TCR clone CT44.6G4 exemplified herein are particularly suitable for administration, or treating, stimulating, providing appropriate immunity (e.g. anti-tumor immunity etc) in HLA-A*01:01 positive human subjects.

In one example, if the subject in need thereof is HLA-A*03:01 positive, an appropriate therapy (e.g. an appropriate pharmaceutical composition described herein) may comprise components of (i) TCR clone CT23.3H4; or (ii) TCR clone MRM23.3B2 exemplified herein. Accordingly, TCRs comprising components of (i) TCR clone CT23.3H4; or (ii) TCR clone MRM23.3B2 exemplified herein are particularly suitable for administration, or treating, stimulating, providing appropriate immunity (e.g. anti-tumor immunity etc) in HLA-A*03:01 positive human subjects.

In one example, if the subject in need thereof is HLA-B*07:02 positive, an appropriate therapy (e.g. an appropriate pharmaceutical composition described herein) may comprise components of (i) TCR clone CT12.3G2; or (ii) TCR clone CT4.20.2C2 exemplified herein. Accordingly, TCRs comprising components of (i) TCR clone CT12.3G2; or (ii) TCR clone CT4.20.2C2 exemplified herein are particularly suitable for administration, or treating, stimulating, providing appropriate immunity (e.g. anti-tumor immunity etc) in HLA-B*07:02 positive human subjects.

In one example, if the subject in need thereof is HLA-B*35:01 positive, an appropriate therapy (e.g. an appropriate pharmaceutical composition described herein) may comprise components of TCR clone CT23.2H9 exemplified herein. Accordingly, TCRs comprising components of TCR clone CT23.3H9 exemplified herein are particularly suitable for administration, or treating, stimulating, providing appropriate immunity (e.g. anti-tumor immunity etc) in HLA-B*35:01 positive human subjects.

The phrase “induced or enhanced immune response” refers to an increase in the immune response (e.g. a cell mediated immune response such as a T cell mediated immune response) of the subject during or after treatment compared to their immune response prior to treatment. An “induced or enhanced” immune response therefore encompasses any measurable increase in the immune response that is directly or indirectly targeted to the disease or condition being treated (or prevented).

In another example, the pharmaceutical composition may be for use in stimulating a cell mediated immune response to a target cell population or tissue in a human subject.

In another example, the pharmaceutical composition may be for use in stimulating a cell mediated immune response to a target cell population or tissue in a human subject. In such an example, the target cell population or tissue may be a HLA-restricted MAGE antigen expressing target cell population or tissue (e.g. a MAGE-A1, a MAGE-A9, a MAGE-A3 and/or a MAGE-A6 antigen expressing target cell population or tissue). Typically, it is a HLA-restricted MAGE antigen expressing target cell population or tissue (e.g. a HLA-restricted MAGE-A1, a HLA-restricted MAGE-A9, a HLA-restricted MAGE-A3 and/or a HLA-restricted MAGE-A6 antigen expressing target cell population or tissue). For example, it may be a target cell population or tissue comprising a HLA-restricted MAGE antigen expressing tumor or cancer. For example, it may be a target cell population or tissue comprising a HLA-restricted MAGE-A1, HLA-restricted MAGE-A9, HLA-restricted MAGE-A3 and/or HLA-restricted MAGE-A6 antigen expressing tumor or cancer.

The pharmaceutical composition may also be for use in providing anti-tumor immunity to a human subject. The pharmaceutical composition is typically for use in providing anti-tumor immunity against a tumor in which a HLA-restricted MAGE antigen described herein is presented at the cell surface in the context of a HLA, e.g. a MAGE-A1, MAGE-A9, MAGE-A3 and/or MAGE-A6 antigen presented at the cell surface in the context of an HLA.

In another example, the pharmaceutical composition may be for use in treating a human subject having a disease or condition associated with an elevated level of HLA-restricted MAGE antigen. For example, the pharmaceutical composition may be for use in treating a human subject having a disease or condition associated with an elevated level of HLA-restricted MAGE-A1, MAGE-A9, MAGE-A3 and/or MAGE-A6 antigen.

A person of skill in the art will be fully aware of MAGE (e.g. MAGE-A1, MAGE-A9, MAGE-A3 and/or MAGE-A6) associated diseases or conditions that may be treated in accordance with the invention. Appropriate examples of such diseases or conditions are discussed elsewhere herein.

As would be clear to a person skilled in the art, the MAGE associated diseases or conditions may comprise at least one tumor (particularly, at least one HLA-restricted MAGE antigen expressing tumor). For example, a HLA-restricted MAGE-A1 antigen, a HLA-restricted MAGE-A9 antigen, a HLA-restricted MAGE-A3 antigen and/or a HLA-restricted MAGE-A6 antigen expressing tumor.

As used herein, the terms “treat”, “treating” and “treatment” are taken to include an intervention performed with the intention of preventing the development or altering the pathology of a condition, disorder or symptom (e.g. a MAGE associated disease or condition). Accordingly, “treatment” refers to both therapeutic treatment and prophylactic or preventative measures, wherein the object is to prevent or slow down (lessen) the targeted condition, disorder or symptom. “Treatment” therefore encompasses a reduction, slowing or inhibition of the amount or concentration of target cells, for example as measured in a sample obtained from the subject, of at least 5%, 10%, 20%, 30%, 40%, 50%, 60%, 70%, 80%, 90% or 100% when compared to the amount or concentration of target cells before treatment. Methods of measuring the amount or concentration of target cells include, for example, qRT-PCR, and quantification of disease specific biomarkers in a sample obtained from the subject.

As used herein the term “subject” refers to an individual, e.g., a human, having or at risk of having a specified condition, disorder or symptom. The subject may be a patient i.e. a subject in need of treatment in accordance with the invention. The subject may have received treatment for the condition, disorder or symptom. Alternatively, the subject has not been treated prior to treatment in accordance with the present invention.

The compositions described herein can be administered to the subject by any conventional route, including injection or by gradual infusion over time. The administration may, for example, be by infusion or by intramuscular, intravascular, intracavity, intracerebral, intralesional, rectal, subcutaneous, intradermal, epidural, intrathecal, percutaneous administration.

The compositions described herein may be in any form suitable for the above modes of administration. For example, compositions comprising modified cells may in any form suitable for infusion. As further examples, suitable forms for parenteral injection (including, subcutaneous, intramuscular, intravascular or infusion) include a sterile solution, suspension or emulsion. Alternatively, the route of administration may be by direct injection into the target area, or by regional delivery or by local delivery. The identification of suitable dosages of the compositions of the invention is well within the routine capabilities of a person of skill in the art.

Advantageously, the compositions described herein may be formulated for use in T cell receptor (TCR) gene transfer, an approach that is rapid, reliable and capable of generating large quantities of T cells with specificity for MAGE antigenic peptides (e.g. the peptides shown in any one of SEQ ID NOs: 127 to 134, or 183), regardless of the patient's pre-existing immune repertoire. Using TCR gene transfer, modified cells suitable for infusion may be generated within a few days.

The compositions described herein are for administration in an effective amount. An “effective amount” is an amount that alone, or together with further doses, produces the desired (therapeutic or non-therapeutic) response. The effective amount to be used will depend, for example, upon the therapeutic (or non-therapeutic) objectives, the route of administration, and the condition of the patient/subject. For example, the suitable dosage of the composition of the invention for a given patient/subject will be determined by the attending physician (or person administering the composition), taking into consideration various factors known to modify the action of the composition of the invention for example severity and type of haematological malignancy, body weight, sex, diet, time and route of administration, other medications and other relevant clinical factors. The dosages and schedules may be varied according to the particular condition, disorder or symptom the overall condition of the patient/subject. Effective dosages may be determined by either in vitro or in vivo methods.

The pharmaceutical compositions described herein are advantageously presented in unit dosage form.

Methods of Generating Binding Proteins (e.g. TCRs)

A method of generating a binding protein that is capable of specifically binding to a peptide containing a MAGE antigen and does not bind to a peptide that does not contain the MAGE antigen is also provided, comprising contacting a nucleic acid composition (or vector system) described herein with a cell under conditions in which the nucleic acid composition is incorporated and expressed by the cell.

In the context of the binding proteins described herein, the MAGE antigen comprises or consists of a sequence comprising an amino acid sequence selected from the group consisting of: SEQ ID NO: 127 to 134, and 183, or a functional fragment or variant thereof.

The method may be carried out on the (host) cell ex vivo or in vitro. Alternatively, the method may be performed in vivo, wherein the nucleic acid composition (or vector system) is administered to the subject and is contacted with the cell in vivo, under conditions in which the nucleic acid sequence is incorporated and expressed by the cell to generate the binding protein. In one example, the method is not a method of treatment of the human or animal body. Appropriate in vivo, in vitro and ex vivo methods for contacting a nucleic acid sequence (or vector systems) with a cell under conditions in which the nucleic acid sequence (or vector) is incorporated and expressed by the cell are well known, as described elsewhere herein.

As stated elsewhere herein, the binding protein comprise a TCR, an antigen binding fragment of a TCR, a ImmTAC or a chimeric antigen receptor (CAR). Further details are provided elsewhere herein.

The binding proteins described herein may be used therapeutically, as described elsewhere herein. Furthermore, the binding proteins may be used in a diagnostic setting, e.g. to detect the presence of MAGE (e.g. MAGE-A1, MAGE-A9, MAGE-A3 and/or MAGE-A6) presented in the context of an appropriate HLA at the cell surface of diseased/malignant tissues.

### General Definitions

As used herein “nucleic acid sequence”, “polynucleotide”, “nucleic acid” and “nucleic acid molecule” are used interchangeably to refer to an oligonucleotide sequence or polynucleotide sequence. The nucleotide sequence may be of genomic, synthetic or recombinant origin, and may be double-stranded or single-stranded (representing the sense or antisense strand). The term “nucleotide sequence” includes genomic DNA, cDNA, synthetic DNA, and RNA (e.g. mRNA) and analogs of the DNA or RNA generated, e.g., by the use of nucleotide analogs. In one example, the nucleotide sequence lacks introns. In other words, it is an intronless nucleic acid sequence. For example, the nucleotide sequence may be a DNA sequence that does not comprise intron sequences.

As used herein, “isolated nucleic acid sequence” or “isolated nucleic acid composition” refers to a nucleic acid sequence that is not in its natural environment when it is linked to its naturally associated sequence(s) that is/are also in its/their natural environment. In other words, an isolated nucleic acid sequence/composition is not a native nucleotide sequence/composition, wherein “native nucleotide sequence/composition” means an entire nucleotide sequence that is in its native environment and when operatively linked to an entire promoter with which it is naturally associated, which promoter is also in its native environment. Such a nucleic acid could be part of a vector and/or such nucleic acid or polypeptide could be part of a composition {e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

The nucleic acid sequences of the invention may be a non-naturally occurring nucleic acid sequence (e.g. it may be that the entire sequence does not occur in its entirety in nature). For example, the nucleic acid sequence of the invention may be operably linked to a promoter, wherein the promoter is not naturally associated with equivalent human nucleic acid sequences in nature (e.g. human TCR sequences or fragments thereof); i.e. it is not the entire promoter that is naturally associated with the nucleic acid in its natural environment. In this context, such promoters may be considered exogenous promoters. Examples of appropriate promoters are described elsewhere.

As used herein “specifically binds” or “specific for” refers to an association or union of a binding protein (e.g., TCR receptor) or a binding domain (or fusion protein thereof) to a target molecule with an affinity or Kₐ (i.e., an equilibrium association constant of a particular binding interaction with units of 1/M) equal to or greater than 10⁵M⁻¹ (which equals the ratio of the on-rate [kₒₙ] to the off-rate [k_{off}] for this association reaction), while not significantly associating or uniting with any other molecules or components in a sample. Binding proteins or binding domains (or fusion proteins thereof) may be classified as “high affinity” binding proteins or binding domains (or fusion proteins thereof) or as “low affinity” binding proteins or binding domains (or fusion proteins thereof). “High affinity” binding proteins or binding domains refer to those binding proteins or binding domains having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. Low affinity” binding proteins or binding domains refer to those binding proteins or binding domains having a Kₐ of up to 10⁷ M⁻¹⋅ up to 10⁶ M⁻¹, up to 10⁵ M⁻¹. Alternatively, affinity can be defined as an equilibrium dissociation constant (K_{d}) of a particular binding interaction with units of M (e.g., 10⁻⁵ M to 10⁻¹³ M).

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof. In certain embodiments, enhanced affinity TCRs can be codon optimized to enhance expression in a particular host cell, such as a cell of the immune system, a inducible pluripotent stem cell (iPSC), a hematopoietic stem cell, a T cell, a primary T cell, a T cell line, a NK cell, or a natural killer T cell (Scholten et al, Clin. Immunol. 119:135, 2006). The T cell can be a CD4+ or a CD8+ T cell, or gamma-delta T cell.

The MAGE protein family is a large, highly conserved group of proteins that share a common MAGE homology domain. As used herein, the terms “MAGE”, “melanoma-associated antigen” and “MAGE protein” refer to a peptide encoded by a MAGE gene. MAGE genes are conserved in all eukaryotes and have rapidly expanded in gene number in mammals. Members of the human MAGE family can be divided into two categories based on tissue expression pattern: Type I MAGEs are considered CTAs and in humans include the MAGE-A, -B, and -C subfamily members which are clustered on the X-chromosome. Type II MAGEs (MAGE-D, -E, -F, -G, -H, -L subfamilies and Necdin) are expressed throughout many tissues in the body and are not restricted to the X chromosome. Both type I and type II MAGEs contain a MAGE homology domain (MHD) that is approximately 170 amino acids. The members of the MAGE-A family are well-known Cancer Testis Antigens (CTA), and are highly expressed on multiple different tumor types as demonstrated by the TCGA Research network (https://www.cancer.gov/tcga).

The upregulation of MAGE in tumors is caused by demethylation of the promoter region of the gene. In normal tissue the expression is limited to the testis where it plays a role in the repression of spermatogonia differentiation. In tumors MAGE expression leads to increased proliferation and migration of tumor cells. Furthermore, MAGE expression is correlated with more aggressive tumors leading to a worse prognosis in patients.

As would be known to a person of skill in the art, MAGE-A1 (MAGA1_HUMAN, UniProt accession number: P43355) may be referred to as melanoma-associated antigen 1, antigen MZ2-E, cancer/testis antigen 1.1, CT1.1 and/or MAGE-1 antigen. A gene encoding MAGE-A1 (and thus a MAGE-A1 antigen) may be referred to as a MAGEA1 gene, a MAGE1 gene, a MAGE1A gene, a CT1.1 gene, a MAGE1 gene, and/or MGC9326.

As would be known to a person of skill in the art, MAGE-A9 (MAGA9_HUMAN, UniProt accession number: P43362) may be referred to as melanoma-associated antigen 9, cancer/testis antigen 1.9, CT1.9 and/or MAGE-9 antigen. A gene encoding MAGE-A9 (and thus a MAGE-A9 antigen) may be referred to as a MAGEA9 gene, a MAGE9 gene, a MAGEA9A gene, a MAGEA9B gene, a CT1.9 gene, a MAGE9 gene and/or MGC8421.

As would be known to a person of skill in the art, MAGE-A6 (MAGA6_HUMAN, UniProt accession number: P43360) may be referred to as melanoma-associated antigen 6, cancer/testis antigen 1.6, CT1.6, MAGE-6 antigen and/or MAGE3B antigen. A gene encoding MAGE-A6 (and thus a MAGE-A6 antigen) may be referred to as a MAGEA6 gene, a MAGE6 gene, a CT1.6 gene, a MAGE-3b gene, a MAGE3B gene and/or a MAGE6 gene.

As would be known to a person of skill in the art, MAGE-A3 (MAGA3_HUMAN, UniProt accession number: P43357) may be referred to as melanoma-associated antigen 3, antigen MZ2-D, cancer/testis antigen 1.3, CT1.3 and/or MAGE-3 antigen. A gene encoding MAGE-A3 (and thus a MAGE-A3 antigen) may be referred to as a MAGEA3 gene, a MAGE3 gene, a CT1.3 gene, a HIP8 gene, a HYPD gene, a MAGE3 gene and/or MGC14613.

In some examples, a protein and a gene encoding said protein may be referred to using the same term (e.g. CT1.1, as mentioned above in the context of MAGE-A1). In examples where a protein and a gene encoding said protein are referred to using the same term (e.g. CT1.1 etc), a person of skill in the art would readily be able to determine whether the protein or the gene was being referred to depending on the context in which the term was mentioned. Typically, gene names are written in italics.

As discussed elsewhere herein, the MAGE antigen may be a MAGE-A1 antigen, a MAGE-A9 antigen, a MAGE-A3 antigen or a MAGE-A6 antigen. In other words, the antigen may be derived from MAGE-A1, MAGE-A9, MAGE-A3 and/or MAGE-A6.

As used herein, the term “MAGE antigen” or “MAGE peptide antigen” or “MAGE-containing peptide antigen” refers to a naturally or synthetically produced peptide portion of a MAGE protein ranging in length from about 7 amino acids, about 8 amino acids, about 9 amino acids, about 10 amino acids, up to about 20 amino acids, which can form a complex with a MHC (e.g., HLA) molecule, and a binding protein of this disclosure specific for a MAGE peptide: MHC (e.g., HLA) complex can specifically bind to such as complex. The MAGE peptide antigen may be a MAGE-A1, MAGE-A9, MAGE-A3 and/or MAGE-A6 peptide antigen. In other words, the MAGE antigen may be derived from a MAGE-A1 protein, a MAGE-A9 protein, MAGE-A3 protein and/or MAGE-A6 protein. Typically, for the purposes of this disclosure, the MAGE peptide antigen comprises or consists of an amino acid sequence selected from the group consisting of: SEQ ID NO: 127 to 134 and 183. Additionally, for the purposes of this disclosure, the MAGE peptide antigen:HLA complex typically comprises a peptide:HLA complex selected from the group consisting of: a KVLEYVIKV:HLA-A*02:01 complex (SEQ ID NO: 127), a VRFFFPSL:HLA-C*07:02 complex (SEQ ID NO: 128), a YVGKEHMFY:HLA-A*01:01 complex (SEQ ID NO: 129), a LTQDLVQEKYLEY:HLA-A*01:01 complex (SEQ ID NO: 130), a SLFRAVITK:HLA-A*03:01 (SEQ ID NO: 131), a RVRFFFPSL:HLA-B*07:02 complex (SEQ ID NO: 132), a EVDPIGHLY:HLA-B*35:01 complex (SEQ ID NO: 133) and a EVDPIGHVY:HLA-B*35:01 complex (SEQ ID NO: 134).

The term “MAGE-specific binding protein,” as used herein, refers to a protein or polypeptide, such as a TCR or CAR, that specifically binds to a MAGE peptide antigen (e.g. a MAGE-A1, MAGE-A9, MAGE-A3 and/or MAGE-A6 peptide antigen) (or to a MAGE peptide antigen:HLA complex, e.g., on a cell surface, e.g. a MAGE-A1:HLA complex, a MAGE-A9:HLA complex, MAGE-A3:HLA complex and/or a MAGE-A6:HLA complex), and does not bind a peptide sequence that does not include the MAGE peptide antigen. Typically, for the purposes of this disclosure, the MAGE peptide antigen comprises or consists of an amino acid sequence selected from the group consisting of: SEQ ID NO: 127 to 134 and 183, and the MAGE peptide antigen:HLA complex comprises a peptide:HLA complex selected from the group consisting of: a KVLEYVIKV:HLA-A*02:01 complex (SEQ ID NO: 127), a VRFFFPSL:HLA-C*07:02 complex (SEQ ID NO: 128), a YVGKEHMFY:HLA-A*01:01 complex (SEQ ID NO: 129), a LTQDLVQEKYLEY:HLA-A*01:01 complex (SEQ ID NO: 130), a SLFRAVITK:HLA-A*03:01 (SEQ ID NO: 131), a RVRFFFPSL:HLA-B*07:02 complex (SEQ ID NO: 132), a EVDPIGHLY:HLA-B*35:01 complex (SEQ ID NO: 133) and a EVDPIGHVY:HLA-B*35:01 complex (SEQ ID NO: 134), as appropriate.

In certain embodiments, a MAGE-specific binding protein specifically binds to a MAGE peptide antigen (e.g. a MAGE-A1, a MAGE-A9, a MAGE-A3 and/or a MAGE-A6 peptide antigen) (or a MAGE peptide antigen:HLA complex e.g. a MAGE-A1:HLA complex, a MAGE-A9:HLA complex, MAGE-A3:HLA complex and/or a MAGE-A6:HLA complex) with a Kd of less than about 10⁻⁸ M, less than about 10⁻⁹ M, less than about 10⁻¹⁰ M, less than about 10⁻¹¹ M, less than about 10⁻¹² M, or less than about 10⁻¹³ M, or with an affinity that is about the same as, at least about the same as, or is greater than at or about the affinity exhibited by an exemplary MAGE-specific binding protein provided herein, such as any of the MAGE-specific TCRs provided herein, for example, as measured by the same assay. In certain embodiments, a MAGE-specific binding protein comprises a MAGE-specific immunoglobulin superfamily binding protein or binding portion thereof. Typically, for the purposes of this disclosure, the MAGE peptide antigen comprises or consists of an amino acid sequence selected from the group consisting of: SEQ ID NO: 127 to 134 and 183, and the MAGE peptide antigen:HLA complex comprises a peptide:HLA complex selected from the group consisting of: a KVLEYVIKV:HLA-A*02:01 complex (SEQ ID NO: 127), a VRFFFPSL:HLA-C*07:02 complex (SEQ ID NO: 128), a YVGKEHMFY:HLA-A*01:01 complex (SEQ ID NO: 129), a LTQDLVQEKYLEY:HLA-A*01:01 complex (SEQ ID NO: 130), a SLFRAVITK:HLA-A*03:01 (SEQ ID NO: 131), a RVRFFFPSL:HLA-B*07:02 complex (SEQ ID NO: 132), a EVDPIGHLY:HLA-B*35:01 complex (SEQ ID NO: 133) and a EVDPIGHVY:HLA-B*35:01 complex (SEQ ID NO: 134), as appropriate.

The selective binding may be in the context of MAGE antigen presentation by HLA-A*02:01, HLA-C*07:02, HLA-A*01:01, HLA-A*03:01, HLA-B*07:02 and/or HLA-B*35:01. In other words, in certain embodiments, a binding protein that “specifically binds to a MAGE antigen” may only do so when it is being presented (i.e. it is bound by) by a specific HLA or is in an equivalent structural formation as when it is being presented by the specific HLA. As discussed elsewhere herein, the inventors identified that the MAGE derived peptide KVLEYVIKV (SEQ ID NO: 127) is capable of being presented by HLA-A*02:01; that the MAGE derived peptide VRFFFPSL (SEQ ID NO: 128) is capable of being presented by HLA-C*07:02; that the MAGE derived peptide YVGKEHMFY (SEQ ID NO: 129) is capable of being presented by HLA-A*01:01; that the MAGE derived peptide LTQDLVQEKYLEY (SEQ ID NO: 130) is capable of being presented by HLA-A*01:01; that the MAGE derived peptide SLFRAVITK (SEQ ID NO: 131) is capable of being presented by HLA-A*03:01; that the MAGE derived peptide RVRFFFPSL (SEQ ID NO: 132) is capable of being presented by HLA-B*07:02; that the MAGE derived peptide EVDPIGHLY (SEQ ID NO: 133) is capable of being presented by HLA-B*35:01; and that the MAGE derived peptide EVDPIGHVY (SEQ ID NO: 134) is capable of being presented by HLA-B*35:01.

Accordingly, in certain examples, a binding protein that “specifically binds to a MAGE antigen”, in particular the peptide of SEQ ID NO: 127, may only do so when it is being presented (i.e. it is bound by) HLA-A*02:01 or is in an equivalent structural formation as when it is being presented by HLA-A*02:01. In another example, a binding protein that “specifically binds to a MAGE antigen”, in particular the peptide of SEQ ID NO: 128 may only do so when it is being presented (i.e. it is bound by) HLA-C*07:02, or is in an equivalent structural formation as when it is being presented by HLA-C*07:02. In another example, a binding protein that “specifically binds to a MAGE antigen”, in particular a peptide of SEQ ID NO: 129 may only do so when it is being presented (i.e. it is bound by) HLA-A*01:01 or is in an equivalent structural formation as when it is being presented by HLA-A*01:01. In another example, a binding protein that “specifically binds to a MAGE antigen”, in particular a peptide of SEQ ID NO: 130 may only do so when it is being presented (i.e. it is bound by) HLA-A*01:01 or is in an equivalent structural formation as when it is being presented by HLA-A*01:01. In another example, a binding protein that “specifically binds to a MAGE antigen”, in particular a peptide of SEQ ID NO: 131 may only do so when it is being presented (i.e. it is bound by) HLA-A*03:01 or is in an equivalent structural formation as when it is being presented by HLA-A*03:01. In another example, a binding protein that “specifically binds to a MAGE antigen”, in particular a peptide of SEQ ID NO: 132 may only do so when it is being presented (i.e. it is bound by) HLA-B*07:02 or is in an equivalent structural formation as when it is being presented by HLA-B*07:02.

In another example, a binding protein that “specifically binds to a MAGE antigen”, in particular a peptide of SEQ ID NO:133 may only do so when it is being presented (i.e. it is bound by) HLA-B*35:01 or is in an equivalent structural formation as when it is being presented by HLA-B*35:01. In another example, a binding protein that “specifically binds to a MAGE antigen”, in particular a peptide of SEQ ID NO: 134 may only do so when it is being presented (i.e. it is bound by) HLA-B*35:01 or is in an equivalent structural formation as when it is being presented by HLA-B*35:01.

By “specifically bind(s) to” as it relates to a T cell receptor, or as it refers to a recombinant T cell receptor, nucleic acid fragment, variant, or analog, or a modified cell, such as, for example, the MAGE T cell receptors, and MAGE-expressing modified cells herein, is meant that the T cell receptor, or fragment thereof, recognizes, or binds selectively to a MAGE antigen (e.g, wherein the MAGE antigen comprises an amino acid sequence selected from the group consisting of: SEQ ID NO: 127 to 134 and 183). Under certain conditions, for example, in an immunoassay, for example an immunoassay discussed herein, the T cell receptor binds to a MAGE antigen (e.g. an amino acid sequence selected from the group consisting of: SEQ ID NO: 127 to 134 and 183) and does not bind in a significant amount to other polypeptides. Thus the T cell receptor may bind to a MAGE antigen (e.g. an amino acid sequence selected from the group consisting of: SEQ ID NO: 127 to 134 and 183) with at least 10, 100, or 1000, fold more affinity than to a control antigenic polypeptide. This binding may also be determined indirectly in the context of a modified T cell that expresses a MAGE TCR. In assays such as, for example, an assay discussed herein, the modified T cell is specifically reactive against a MAGE expressing melanoma cell line (e.g. a SK2.3 cell line) or a MAGE expressing multiple myeloma cell line (e.g. a U266 cell line). Thus, the modified MAGE-TCR expressing T cell may bind to a MAGE expressing melanoma or a MAGE expressing multiple myeloma cell line (e.g. a SK2.3 cell line or a U266 cell line, respectively) with at least 10, 100, or 1000, fold more reactivity when compared to its reactivity against a control cell line that is not a MAGE expressing melanoma or a MAGE expressing multiple myeloma cell line (e.g. a SK2.3 cell line or U266 cell line, respectively)

A “non-essential” (or “non-critical”) amino acid residue is a residue that can be altered from the wild-type sequence of (e.g., the sequence identified by SEQ ID NO herein) without abolishing or, more preferably, without substantially altering a biological activity, whereas an “essential” (or “critical”) amino acid residue results in such a change. For example, amino acid residues that are conserved are predicted to be particularly non-amenable to alteration, except that amino acid residues within the hydrophobic core of domains can generally be replaced by other residues having approximately equivalent hydrophobicity without significantly altering activity.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential (or non-critical) amino acid residue in a protein is preferably replaced with another amino acid residue from the same side chain family. Alternatively, in another embodiment, mutations can be introduced randomly, and the resultant mutants can be screened for activity to identify mutants that retain activity.

Calculations of sequence homology or identity (the terms are used interchangeably herein) between sequences are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970) J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

Alternatively, the percent identity between two amino acid or nucleotide sequences can be determined using the algorithm of Meyers et al. (1989) CABIOS 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-410). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, gapped BLAST can be utilized as described in Altschul et al. (1997, Nucl. Acids Res. 25:3389-3402). When using BLAST and gapped BLAST programs, the default parameters of the respective programs (e.g., XBLAST and NBLAST) can be used. See <http://www.ncbi.nlm.nih.gov>.

The polypeptides and nucleic acid molecules described herein can have amino acid sequences or nucleic acid sequences sufficiently or substantially identical to the sequences identified by SEQ ID NO. The terms “sufficiently identical” or “substantially identical” are used herein to refer to a first amino acid or nucleotide sequence that contains a sufficient or minimum number of identical or equivalent (e.g. with a similar side chain) amino acid residues or nucleotides to a second amino acid or nucleotide sequence such that the first and second amino acid or nucleotide sequences have a common structural domain or common functional activity. In other words, amino acid sequences or nucleic acid sequences having one or several (e.g. two, three, four etc) amino acid or nucleic acid substitutions compared to the corresponding sequences identified by SEQ ID NO may be sufficiently or substantially identical to the sequences identified by SEQ ID NO (provided that they retain the requisite functionality). In such examples, the one or several (e.g. two, three, four etc) amino acid or nucleic acid substitutions may be conservative substitutions. For example, amino acid or nucleotide sequences that contain a common structural domain having at least about 60%, or 65% identity, likely 75% identity, more likely 85%, 90%, 91%, 92%, 93%, 94%, 95%, 96%, 97%, 98% or 99% identity are defined herein as sufficiently or substantially identical.

TCR sequences are defined according to IMGT. See the LeFranc references herein for further details i.e. [1] Lefranc M.-P. “Unique database numbering system for immunogenetic analysis” Immunology Today, 18:509 (1997). [2] Lefranc M.-P. “The IMGT unique numbering for immunoglobulins, T cell Receptors and Ig-like domains” The immunologist, 7, 132-136 (1999). [3] Lefranc M.-P. et al. “IMGT unique numbering for immunoglobulin and Tcell receptor variable domains and Ig superfamily V-like domains” Dev. Comp. Immunol., 27, 55-77 (2003). [4] Lefranc M.-P. et al. “IMGT unique numbering for immunoglobulin and T cell receptor constant domains and Ig superfamily C-like domains” Dev. Comp. Immunol., 2005, 29, 185-203 PMID: 15572068.

As used herein, the term “ex vivo” refers to “outside” the body. The term “in vitro” can be used to encompass “ex vivo” components, compositions and methods.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Example 1—a Library of Novel Cancer Testis Specific T Cell Receptors for T Cell Receptor Gene Therapy

As discussed elsewhere herein, Cancer testis (CT) genes are potential targets for cancer immunotherapy since they are highly expressed in multiple different tumor types as demonstrated by the TCGA Research network (https://www.cancer.gov/tcga). In this study, the inventors sought to expand the library of potent CT-specific TCRs. Advantageously, such a library may eventually be used to select a personalized TCR gene therapy, based on HLA typing of the patient and gene expression of the tumor, for each individual patient. The TCR constructs can be generated as an “off-the-shelf” TCR library that are available when required for transfer into patient's isolated CD8⁺ T cells by either viral transduction (Td) or non-viral editing. This will save production time, money and workload, since not for every individual patient an unique tumor-reactive TCR has to be identified. Moreover, the expanding TCR library makes it possible to treat individual patients with combination therapy targeting multiple different genes. This may enhance the efficacy of the TCR gene therapy by reducing the likelihood of tumor escape.

To identify novel CT-specific TCRs, the inventors selected, by bioinformatics tools, the most promising CT genes to target, and from these genes the inventors identified, by HLA-peptidomics, naturally processed and presented HLA class I peptides. Peptide-HLA (pHLA) tetramers were generated to isolate CT-antigen specific T cells from the allo-HLA repertoire of healthy individuals. Advantageously, the inventors identified several TCRs with an effective and specific recognition profile for MAGE-A1, MAGE-A3, MAGE-A6 and MAGE-A9 in the context of HLA-A*01:01, -A*02:01, -A*03:01, -B*07:02, -B*35:01 and -C*07:02. These novel TCRs allow TCR gene therapy for an increased number of cancer patients, and development of combination TCR gene therapy.

### Methods

### Cell Culture

T cells were cultured in T cell medium (TCM) containing Iscove's modified Dulbecco's medium (IMDM)(Lonza) supplemented with 5% fetal bovine serum (FBS)(Thermo Fisher Scientific), 5% human serum (Sanquin), 100 IU/ml IL-2 (Novartis Pharma), 1.5% L-glutamine (Lonza), and 1% penicillin/streptomycin (pen/strep)(Lonza).

Tumor cell lines and Epstein-Barr Virus transformed B lymphoblastoid cell lines (EBV-LCLs) were cultured in IMDM supplemented with 10% FBS, 1.5% L-glutamine and 1% pen/strep.

Early-passage melanoma cell lines were obtained from the Department of Medical Oncology, LUMC, and cultured in Dulbecco's modified eagle medium (DMEM)(Thermo Fisher Scientific) containing nonessential amino acids (NEAA) supplemented with 7.5% FBS, 1.5% L-glutamine and 1% pen/strep. The Leiden University Medical Center ethical review board approved use of all the human material in this study (approval number B16.039). Materials were obtained after written informed consent in accordance with the Declaration of Helsinki.

Peptide Identification and pHLA Tetramer Generation

Peptides were identified from resected ovarian carcinomas (OvaL1, OvaL10 and OvaL11), multiple myeloma cell lines (U266, UM9 and RPMI8226) and a prostate cell line (C4-2B4). The resected ovarium carcinomas consisted of residual material and were collected anonymously. The carcinoma samples were sliced into small pieces and dead, clotted or non-tumor material was removed. Subsequently, the sliced tumor pieces were added to a C-tube (Miltenyi Biotec) with ice cold buffer and complete Protease Inhibitor (Sigma-Aldrich), but without detergent, to prevent unwanted protease activity. The tumor pieces were dissociated until an almost homogenous cell solution by using a gentleMACS (Miltenyi Biotec) procedure and benzonase (Merck)(125 IU/ml) was added to remove DNA/RNA complexes during lysis. To identify peptides from the resected ovarian carcinomas, multiple myeloma cell lines and prostate cell line, elution experiments were performed followed by high-performance liquid chromatography (HPLC) fractionation and mass spectrometry analysis as previously described [17]. In short, peptide-HLA complexes were purified by immunoaffinity using the anti-HLA-I W6/32 antibody. Peptides were separated from the HLA allele and peptide containing fractions were obtained by size filtration. Subsequently, fractions were separated by strong cation exchange chromatography and freeze dried. Peptide fractions were lyophilized, dissolved in 95/3/0.1 water/acetonitrile/formic acid v/v/v, and subsequently analysed with nanoHPLC-MS/MS. Peptide and protein identification from tandem mass spectra was performed by proteome discoverer version 2.1 (Thermo Fischer Scientific) using the mascot node and the UniProt Homo Sapiens database. Synthetic peptides were ordered for the potential target peptides when meeting the following criteria: 1) peptides are derived from the selected candidate genes; 2) bind in HLA-A1, -A2, -A3, -A11, -A24, -B7, -B35 or -C7 according to NETMHC version 3.4; 3) have minimal Mascot Ion score≥30; 4) are ranked 1 peptides; 5) peptide sequence is unique for the candidate gene. Synthetic peptides were generated in house using standard Fmoc chemistry. Peptide sequences were validated by matching the tandem mass spectra of the eluted peptides with the tandem mass spectra of the synthetic peptides. To generate monomers recombinant HLA-A1, -A2, -A3, -B7, -B35, and -C7 heavy chains (HCs) and human beta-2 microglobulin (B2M) were produced in house in Escherichia coli. PE-labelled pHLA tetramers were generated from the monomers as previously described [18].

### Isolation of CT Specific T Cell Clones

After informed consent was collected, peripheral blood mononuclear cells (PBMCs) from healthy individuals were isolated using ficoll gradient centrifugation. pHLA tetramer positive T cell clones were isolated from 250-750×10⁶ PBMCs from donors negative for the HLA of interest, as previously described [12]. In brief, PBMCs were stained with PE-labeled pHLA tetramers for 10 minutes at 37° C. or 1 hour at 4° C. Subsequently, magnetic-activated cell sorting (MACS) was performed using anti-PE Microbeads (Miltenyi Biotec). The positive fraction was stained with Alexa Fluor700-conjugated anti-CD8 (catalog MHCD0829)(Invitrogen), FITC-conjugated anti-CD4 (catalog 555346)(BD pharmingen), anti-CD14 (catalog 555397)(BD pharmingen), and anti-CD19 (catalog 555412)(BD Biosciences). pHLA tetramer-positive CD8⁺ T cells were single-cell sorted using an Aria III cell sorter (BD Biosciences) in 96 well round-bottom culture plate (Greiner Bio-one) containing irradiated PBMCs (50,000 cells/well)(3500RAD) and EBV-LCLs (5000 cells/well)(5000RAD) in 100 μl TCM supplemented with 0.8 μg/ml phytohemagglutinin (PHA)(Thermo Fisher Scientific). Expanding T cell clones were used in functional assays 7-14 days after stimulation. pHLA tetramer staining was performed using PE-conjugated pHLA tetramers for 15 minutes at 37° C. and fluorescence was measured on an LSRII (BD Biosciences) and analysed using Diva (BD Biosciences) or FlowJo software (TreeStar).

### T-Cell Recognition Assay

Target cell recognition was determined by co-culture of 5,000 T cells with target cells in effector: target (E:T) ratio of 1:2 or 1:4 in 60 μl TCM per well in 384-well flat-bottom culture plates (Greiner Bio-one). To induce HLA expression prior to functional analysis, early passage melanoma cell lines were treated with 100 IU/ml IFN-γ for 48 hours. In peptide titration assays, target cells were pre-incubated for at least 30 minutes at 37° C. with titrated peptide concentrations starting at 1 μM. After overnight co-culture, recognition of target cells was determined by measuring IFN-γ production in the supernatant by enzyme-linked immunosorbent assay (ELISA)(Sanquin/Invitrogen/Diaclone). The supernatant was diluted 5, 25 or 125 times to determine concentrations based on the linear part of the standard curve. The Hamilton Microlab STAR Liquid Handling System (Hamilton company) was used to transfer supernatants from the culture plates to the high-binding 384-well ELISA plates (Greiner Bio-one). ELISA plates were washed extensively between the coating, blocking, detection and readout steps with the Zoom HT LB 920 Microplate Washer (Berthold).

T-cell mediated cytotoxicity was measured using ⁵¹Cr-release experiments. Target cells were labelled for 1 hour at 37° C. with 50 μCi Na₂ ⁵¹CrO₄ (PerkinElmer). Subsequently, target cells were washed and co-cultured in a plate with T cells at various E:T ratios for 6 hours at 37° C. in 96-well round-bottom culture plates (Greiner Bio-one). After incubation, supernatant was harvested, and transferred to Lumaplates (Microbeta 2, PerkinElmer) and dried overnight. Spontaneous and maximum ⁵¹Cr-release was determined using TCM alone or TCM containing 1% Triton-X 100 (Sigma-Aldrich), respectively. ⁵¹Cr-release was measured in counts per minute (CPM) using a 2450 Microbeta2 plate counter (PerkinElmer). The target cell killing was calculated by the formula: (average CPM of the sample−average CPM spontaneous release)/(average CPM maximal release-average CPM spontaneous release)*100.

### TCR Constructs and Retroviral Transduction

TCR usage was determined as previously described with minor modifications [19]. In short, T cells were lysed (ReliaPromega kit), mRNA was isolated, and TCR cDNA was generated using reverse primers in the TCR constant alfa (TCR-α) and beta (TCR-β) regions, SMARTScribe Reverse Transcriptase (Takara, Clontech) and a SA.rt template switching oligo forward primer [20]. Barcoded TCR PCR product was generated in two rounds of PCR. In the first PCR, TCR-α and TCR-β products were generated, in a second PCR the first PCR product was used to include a barcode sequence that allowed discrimination between TCRs of different T cell clones. PCR products of different T cell clones were pooled, after which TCR sequences were identified by HiSeq (GenomeScan). The TCR sequences were analysed using MiXCR software package and the ImMunoGeneTics (IMGT) database [21]. The variable TCR-α and TCR-β fragments of the different CT specific TCRs and CMV TCR specific for the NVLPMVATV peptide binding in HLA-A*02:01 were codon optimized and combined with codon-optimized and cysteine-modified murine TCR-αβ constant domains, and TCR chains were linked by P2A sequence and cloned into MP71 retroviral vector [22]. Transfection of the TCR constructs was performed with plasmid DNA, Fugene HD transfection reagent (Promega) and optimum I medium (Invitrogen Gibco) in Phoenix-A cells. Phoenix-A cells (ATCC) were transfected, and virus-supernatant was harvested 48 hours after transfection, and frozen at −80° C.

### TCR Gene Transfer

Primary CD8⁺ T cells were isolated from peripheral blood by MACS using anti-CD8 MicroBeads (Miltenyi Biotec). T cells (0.3×10⁶ cells/ml) were stimulated with irradiated autologous PBMCs (1×10⁶ cells/well), and 0.8 μg/ml PHA. On day 2, retroviral supernatants were added to 24-well non-tissue cultured treated plates (Greiner Bio-One) precoated with retronectin (Takara) and blocked with 2% human serum albumin (Sanquin). Retroviral supernatant was spun down for 20 minutes, 3000 RPM at 4° C., after which the virus supernatant was removed and 0.3×10⁶ CD8⁺ T cells were added for overnight culture. After overnight culture T cells were transferred to 24-well culture plates (Costar) and 5 days after transduction T cells were enriched by MACS using APC-conjugated anti-mouse TCR-β constant domain (mTCRβ)(BD biosciences) and anti-APC Microbeads (Miltenyi Biotec). pHLA-tetramer staining was performed to confirm TCR-cell surface expression and the Td T cells (TCR-T cells) were used in functional screenings 9-12 days after isolation.

### Quantitative RT-PCR

Total RNA was isolated from 0.1-5×10⁶ target cells using the Reliaprep RNA cell mini prep system according to manufacturer's protocol (Promega). Total RNA was converted to cDNA using Moloney murine leukemia virus reverse transcriptase and Oligo (dT) primer (Invitrogen by Thermo Fisher Scientific). Quantitative reverse transcription polymerase chain reaction (qPCR) was performed using Fast Start TaqDNA Polymerase (Roche) and EvaGreen (Biotium). Gene expression was measured on the Lightcycler 480 (Roche) by forward and reverse primers designed by the inventors (Table 2). Target gene expression was calculated relative to the average expression of housekeeping genes (HKGs): GUSB, PSMB4 and VPS29.

TABLE 2
Primers included in the RT-qPCR experiments.

| Gene | Forward primer | Reverse primer |
|---|---|---|
| MAGE- | GAGTCCTTGTTCCGAGCAGT (SEQ | GGCTCCCTGGCTCGATATTT |
| A1 | ID NO: 135) | (SEQ ID NO: 136) |
| MAGE- | CCTGAGCAACGAGCGACG (SEQ | TCAGAACCTTGCCTCCTCACC |
| A3/A6 | ID NO: 137) | (SEQ ID NO: 138) |
| MAGE- | GATCCTGCGCACTACGAGTT (SEQ | ATGGGTAGCAGATGGGCTCT |
| A9 | ID NO: 139) | (SEQ ID NO: 140) |
| GUSB | ACTGAACAGTCACCGACGAG (SEQ | GGAACGCTGCACTTTTTGGT |
| | ID NO: 141) | (SEQ ID NO: 142) |
| PSMB4 | GTTTCCGCAACATCTCTCGC (SEQ | CATCAATCACCATCTGGCCG |
| | ID NO: 143) | (SEQ ID NO: 144) |
| VPS29 | TGAGAGGAGACTTCGATGAGAATC | TCTGCAACAGGGCTAAGCTG |
| | (SEQ ID NO: 145) | (SEQ ID NO: 146) |

### In Vivo Antitumor Reactivity of MAGE-A1 Specific TCRs

Female NSG mice (NOD.Cg-Prkdc(scid)Il2rg(tm1Wjl)/SzJ, The Jackson Laboratory) were injected intravenous (i.v.) with 2×10⁶ U266 multiple myeloma cells. U266 cells were transduced with Luciferase-TdTomato Red and enriched>98% purity. Tumor growth was measured 1 to 2 times per week after subcutaneous injection of 150 μl 7.5 mM D-luciferine (Cayman Chemical Co.) using a CCD camera (IVIS spectrum, PerkinElmer). On day 14 post engraftment, mice were injected i.v. with 5×10⁶ T cells which were transduced with 4F7-TCR (MAGE-A1 KVL/A2) (n=6), 3H4-TCR (MAGE-A1 SLF/A3) (n=5), 10C1-TCR (MAGE-A1 VRF/C7) (n=6) or irrelevant CMV-TCR (pp65 NLV/A2) (n=4). The TCR-T cells were enriched for mTCRβ expression by MACS before infusion and injected 7 days after an additional restimulation. This in vivo study was performed in accordance with Dutch law for animal experiments and approved by the national Ethical Committee for Animal Research (AVD116002017891).

### Results

### Selection of Promising CT Genes and Candidate CT Peptides

To select the most promising CT genes to target with TCR-T cells, CT-gene expression was evaluated based on online databases in healthy and cancer tissues. First, those CT genes described in the CT database (http://www.cta.lncc.br/) with high expression (RNA seq V2 (log)>6) in TCGA database, and in addition expressed in a substantial percentage (>10%) of certain tumor types were included. Subsequently, only those CT genes with restricted expression in testis and/or placenta demonstrated by the Genotype-Tissue Expression (GTEx), and Human Protein Atlas (HPA) were selected. In total, the inventors selected 12 CT genes for further analysis; MAGE-A1, MAGE-A3, MAGE-A4, MAGE-A6, MAGE-A9, MAGE-A11, MAGE-C1, MAGE-C2, SSX1, SSX2, SSX3 and SSX4.

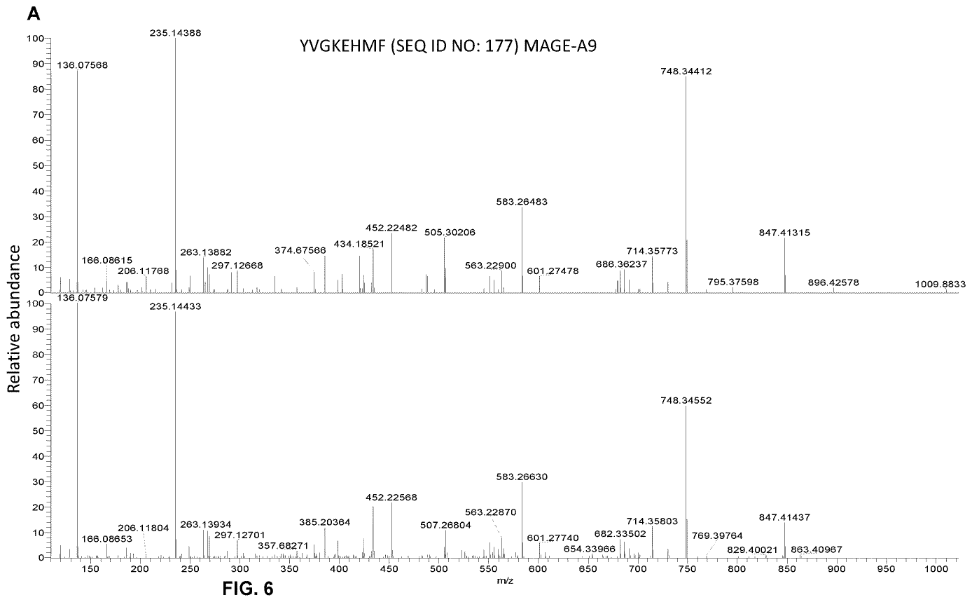

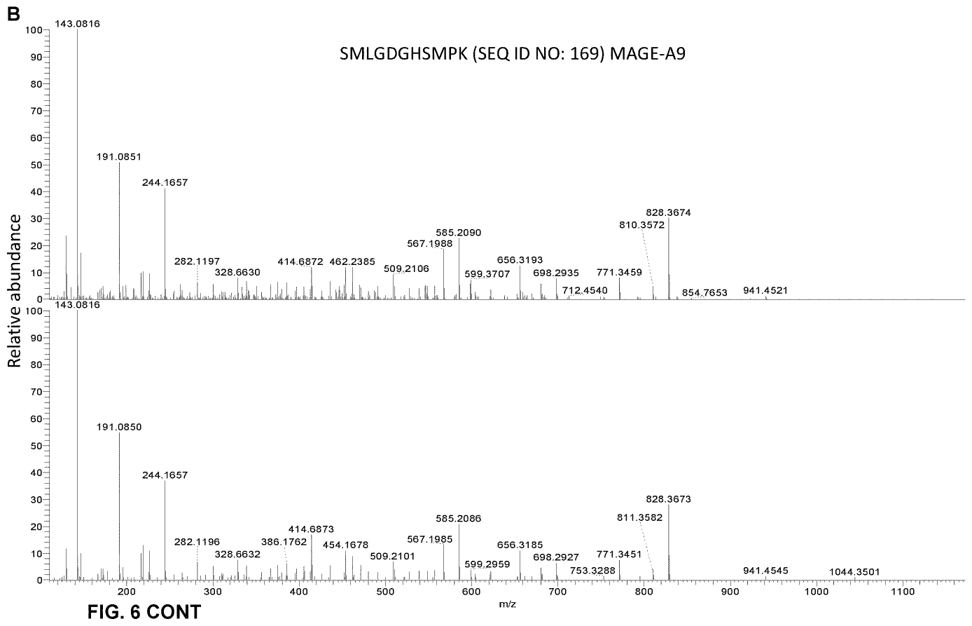

To identify CT peptides that are naturally processed and presented in HLA class I on the cell surface of tumors, HLA peptidomics was performed on 8 resected ovarian cancers, 3 different multiple myeloma cell lines (U266, RPMI8226, and UM9), and the prostate carcinoma cell line C4-2B4 (Table 3). CT peptides were selected with an Ion Score≥30, and validated by comparing mass spectra of eluted peptides and synthetic peptides (example in FIG. 6). Eluted peptides were matched to HLA alleles by combining predicted HLA binding (netMHC3.4) with the HLA typing of the material from which the peptides were originated. Only peptides binding in prevalent HLA alleles (HLA-A*01:01, -A*02:01, -A*03:01, -A*11:01, -A*24:02, -B*07:02, -B*08:01, -B*35:01, -C*07:01, and C*07:02) were selected. To avoid off-target toxicity, peptides with an identical peptide sequence in any other human gene than the selected CT genes as determined by BLAST search (blast.ncbi.nlm.nih.gov) were excluded. By this method 31 different CT peptides were identified, of which most have been described before. Two peptides derived from MAGE-A9, YVGKEHMF (SEQ ID NO: 177) binding in HLA-A*24:02 and SMLGDGHSMPK (SEQ ID NO: 169) binding in HLA-A*03:01 were not described previously according to iedb.org database (FIG. 6). In addition to this set of peptides, 8 peptide-HLA (pHLA) combinations were included based on literature and 3 based on prediction (Table 3). pHLA binding was confirmed for all selected peptides by stable pHLA monomer refolding (data not shown). Most selected and confirmed peptides originated from MAGE-A1 (11 peptides), followed by MAGE-A9 and MAGE-A3 (7 and 6 peptides, respectively). The inventors did not identify peptides from MAGE-C1, SSX1 and SSX3. In total, 42 different pHLA monomers could be generated of which PE-labeled pHLA tetramers were constructed and used for CT specific T-cell isolation.

TABLE 3
Peptide-HLA complexes and selected CT-specific T cell clones

| | | | | Recognition pattern | | |
|---|---|---|---|---|---|---|
| | | | | identified T cell clones | | |
| | | | | | Potent | |
| | HLA- | Target | | Td | & | |
| Target peptide | restriction | gene | Eluted from | gene* | safe** | Final† |
| ALIEVGPDHFC | A*02:01 | MAGE- | OvaL 10 | — | — | |
| (SEQ ID NO: 147) | | C2 | | | | |
| ALKDVEERV | A*02:01 | MAGE- | - | 1 | — | |
| (SEQ ID NO: 148) | | C2 | | | | |
| ALKLKVAEL (SEQ | B*08:01 | MAGE- | RPMI8226 +B8 | — | — | |
| ID NO: 149) | | A9 | | | | |
| ATLPPFMCNK | A*03:01/ | SSX2 | Prediction/ | 1 | — | |
| (SEQ ID NO: 150) | A*11:01 | | UM9 | 1 | — | |
| ALREEEEGV | A*02:01 | MAGE- | U266, C4-2B4, | 1 | — | |
| (SEQ ID NO: 151) | | A1 | OvaL 11 | | | |
| EADPTGHSY | A*01:01 | MAGE- | - | 2 | — | |
| (SEQ ID NO: 152) | | A1 | | | | |
| EVDPIGHLY | A*01:01/ | MAGE- | - | 7 | — | — |
| (SEQ NO: 133) | B*35:01 | A3 | - | 6 | 3 | 1(2H9) |
| FPSLREAAL (SEQ | B*07:02 | MAGE- | U266 and | 2 | — | |
| ID NO: 153) | | A1 | OvaL 11 | | | |
| FVYGEPREL | A*02:01 | MAGE- | U266 | 3 | — | |
| (SEQ ID NO: 154) | | C2 | | | | |
| GLLGDNQIMPK | A*03:01 | MAGE- | U266, | 3 | — | |
| (SEQ ID NO: 155) | | A1/A3/A6 | RPMI8226 +A3 | | | |
| GVYAGREHFV | A*02:01 | MAGE- | U266 | 2 | — | |
| (SEQ ID NO: 156) | | C2 | | | | |
| GVYDGREHTV | A*02:01 | MAGE- | U266, UM9 | 1 | — | |
| (SEQ ID NO: 157) | | A4 | +A2, OvaL10 | | | |
| IMPKAGLLII (SEQ | A*24:02 | MAGE- | OvaL 1 | — | — | |
| ID NO: 158) | | A3 | | | | |
| IVLGVILTK (SEQ | A*03:01 | MAGE- | RPMI8226 +A3 | — | — | |
| ID NO: 159) | | A9 | | | | |
| KASEKIFYV (SEQ | A*02:01 | SSX2 | — | 3 | — | |
| ID NO: 160) | | | | | | |
| KIWEELSVLEV | A*02:01 | MAGE- | U266 | — | — | |
| (SEQ ID NO: 161) | | A3/A6 | | | | |
| KVLEFLAKL (SEQ | A*02:01 | MAGE- | U266 | 3 | — | |
| ID NO: 162) | | C2 | | | | |
| KVLEYVIKV (SEQ | A*02:01 | MAGE- | U266 | 18 | 2 | 1 (4F7) |
| ID NO: 127) | | A1 | | | | |
| LTQDLVQEKYLEY | A*01:01 | MAGE- | RPMI8226 +A1 | 1 | 1 | 1 (6G4) |
| (SEQ ID NO: 130) | | A1 | | | | |
| LVFGIELMEV | A*02:01 | MAGE- | — | 6 | — | |
| (SEQ ID NO: 163) | | A3 | | | | |
| RCFPVIFGK (SEQ | A*03:01 | MAGE- | U266, UM9 +A3 | — | — | |
| ID NO: 164) | | A4 | | | | |
| RIFPKIMPK (SEQ | A*03:01 | SSX4 | U266 | — | — | |
| ID NO: 165) | | | | | | |
| RPADLTRVIM | B*07:02 | MAGE- | Oval10 | — | — | |
| (SEQ ID NO: 166) | | A11 | | | | |
| RVRFFFPSL (SEQ | B*07:02 | MAGE- | U266 | 61 | 3 | 1 (3G2) |
| ID NO: 132) | | A1 | | | | |
| RVRFFFPSLR | A*03:01 | MAGE- | Prediction | — | — | |
| (SEQ ID NO: 167) | | A1 | | | | |
| RVRIAYPSL (SEQ | B*07:02 | MAGE- | U266 | — | — | |
| ID NO: 178) | | A4 | | | | |
| RVRIAYPSLR | A*03:01 | MAGE- | U266 | 7 | — | |
| (SEQ ID NO: 168) | | A4 | | | | |
| SLFRAVITK (SEQ | A*03:01 | MAGE- | U266 | 39 | 2 | 1 (3H4) |
| ID NO: 131) | | A1 | | | | |
| SMLGDGHSMPK | A*03:01 | MAGE- | RPMI8226 +A3 | — | — | |
| (SEQ ID NO: 169) | | A9 | | | | |
| SVMGVYVGK | A*03:01 | MAGE- | RPMI8226 +A3 | — | — | |
| (SEQ ID NO: 170) | | A9 | | | | |
| TLDEKVAEL (SEQ | A*02:01 | MAGE- | U266 | 2 | — | |
| ID NO: 171) | | C2 | | | | |
| TQDLVQEKY | A*01:01 | MAGE- | RPMI8226 +A1 | — | — | |
| (SEQ ID NO: 172) | | A1/B1/B4 | | | | |
| VAELVHFLL (SEQ | A*24:02 | MAGE- | — | 1 | — | |
| ID NO: 173) | | A3 | | | | |
| VIWEVLNAV (SEQ | A*02:01 | MAGE- | — | 5 | — | |
| ID NO: 174) | | C2 | | | | |
| VLGEEQEGV | A*02:01 | MAGE- | RPMI8226 +A2 | — | — | |
| (SEQ ID NO: 175) | | A9 | | | | |
| VRFFFPSL (SEQ | C*07:01/ | MAGE- | Prediction/ | 2 | — | — |
| ID NO: 128) | C*07:02 | A1 | U266 | 13 | 1 | 1(10C1) |
| YPSLREAAL (SEQ | B*07:02 | MAGE- | U266, UM9 | — | — | |
| ID NO: 176) | | A4 | | | | |
| YVGKEHMF (SEQ | A*24:02 | MAGE- | RPMI8226 | — | — | |
| ID NO: 177) | | A9 | +A24 | | | |
| YVGKEHMFY | A*01:01 | MAGE- | RPMI8226 +A1 | 1 | 1 | 1 (2D8) |
| (SEQ ID NO: 129) | | A9 | | | | |

Peptide-HLA complexes and selected CT-specific T cell clones. CT specific peptides were identified from multiple myeloma cell lines U266, UM9 and RPMI8226; prostate cancer cell line C4-2B4, and primary ovarium carcinoma samples OvaL1, OVal10, and Oval11.
*T cell clones produce IFN-γ after stimulation with CT-gene transduced Raji cell;
**T cell clones that show potent and strict recognition against Tumor cell lines panels, EBV-LCL panel and MAGE-A panel;
†The final selection of most promising T cell clones for TCR gene therapy.

Isolation of CT-Specific T Cells from the Allo-HLA Repertoire

To isolate CT-specific T cells, PBMCs (5-10×10⁹ cells) from 54 different HLA-typed healthy donors were incubated with a pool of PE-labeled pHLA tetramers. pHLA-tetramer positive T cells were only searched in those donors negative for the target HLA allele. In total, 22,344 CD8^{pos}tetramer^{pos} cells (ranging between 10 to 1920 per donor) were single-cell sorted and on average 60% could be successfully clonally expanded for 2 weeks. Initially, all isolated T cell clones were screened for peptide specific recognition on peptide-pool loaded Raji Td with HLA alleles of interest (except for HLA-A*03:01 that is naturally expressed on Raji). T cell clones that produced IFN-γ upon co-culture with peptide-pool loaded HLA-Td Raji cells, but not unloaded HLA-Td Raji, were selected. To evaluate the recognition of naturally processed and presented peptides by the selected T cell clones, HLA-Td Raji cells were transduced with CT genes. Those T cell clones (192) that specifically produced IFN-γ upon stimulation with CT-Td Raji were selected for further screening (Table 3).

### Tumor Reactivity of CT-Specific T Cell Clones Against CT-Positive Tumor Cell Lines

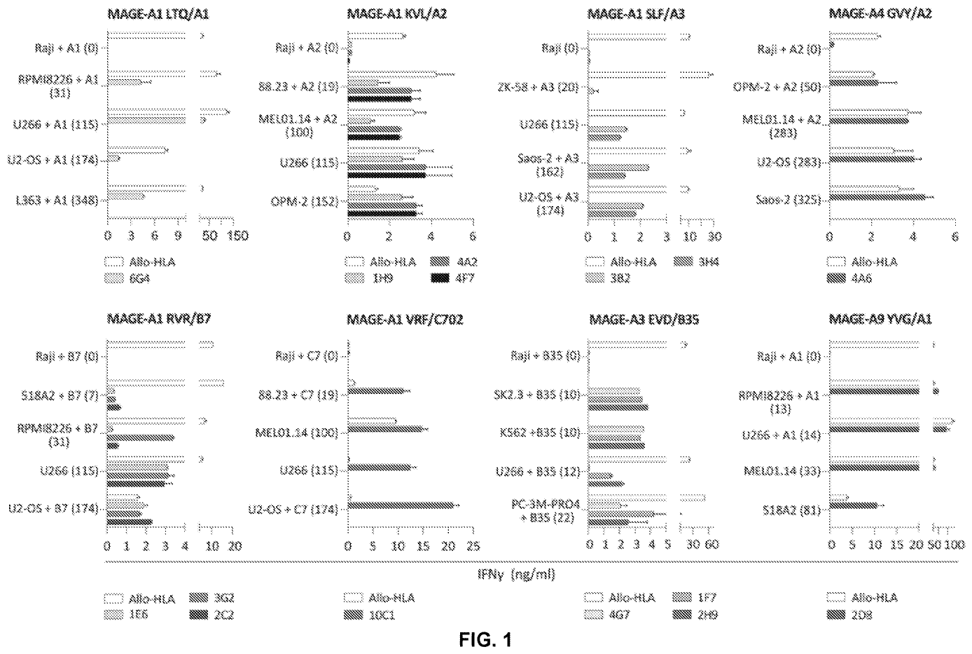

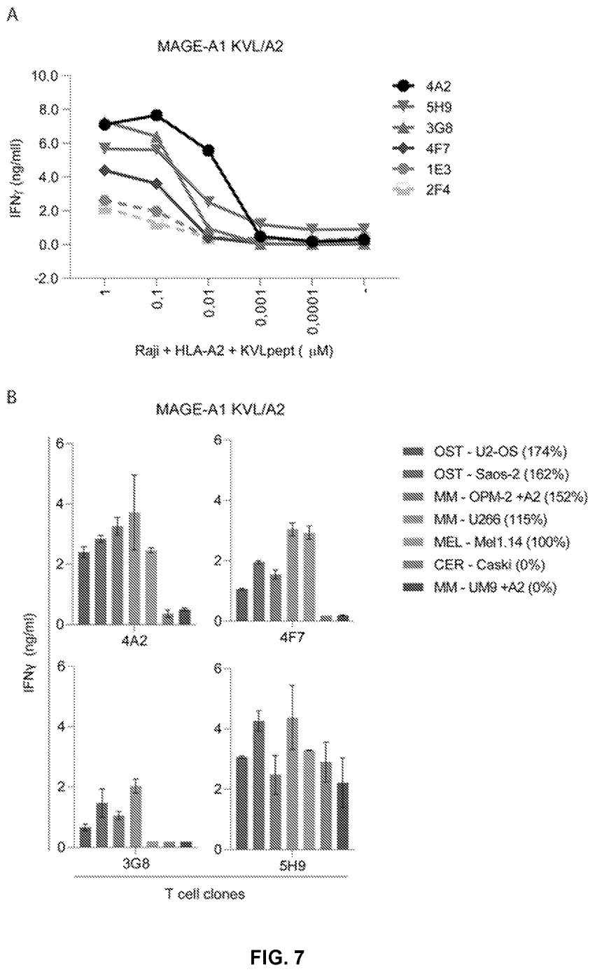

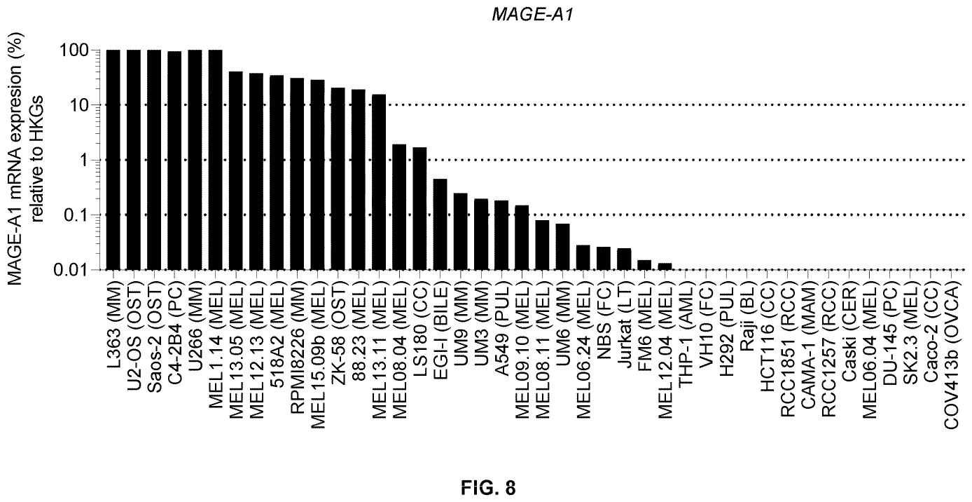

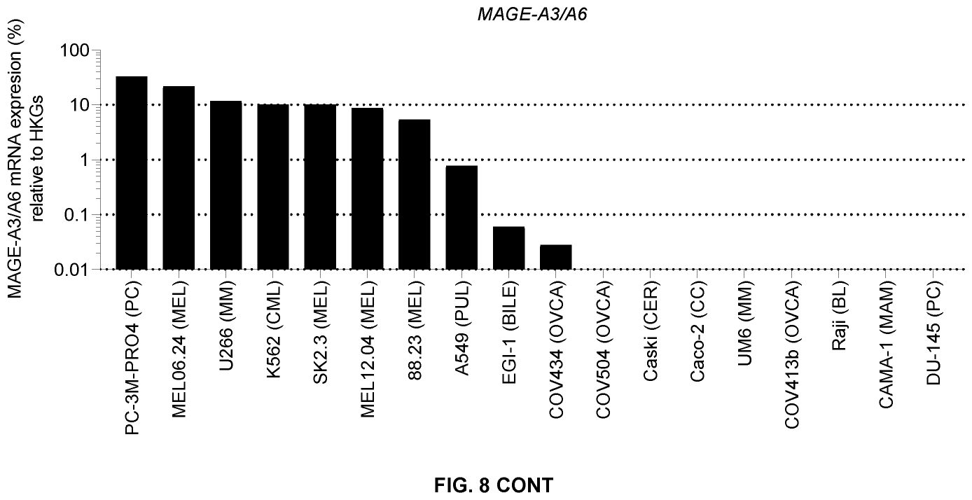

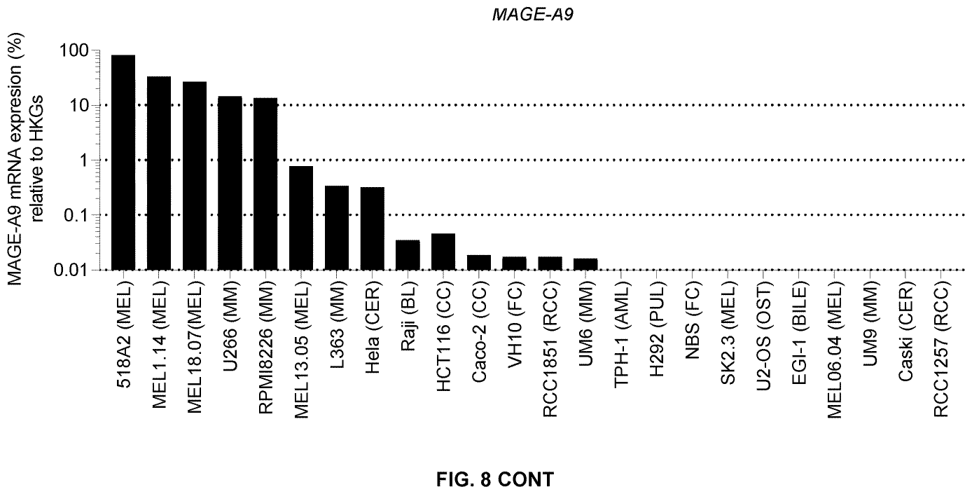

To select those T cell clones with the highest functional avidity peptide titration was performed on all 192 previous selected T cell clones (see, example in FIG. 7A). Subsequently, the T cell clones were screened against CT-positive and -negative tumor cell lines that naturally express or were transduced with HLA alleles of interest. The CT-expression level of each included tumor cell line was determined by qPCR (FIG. 8). T cell clones with low IC50 and strict CT-specific tumor reactivity upon co-culture with the tumor cell lines as measured by IFN-γ production, like 4A2 and 4F7, were selected (FIG. 7B). To confirm stimulatory capacity and HLA expression of the tumor cell lines, allo-HLA reactive T cell clones were included as positive controls. In total, 15 T cell clones specific for MAGE-A1, MAGE-A3, MAGE-A4 or MAGE-A9 in the context of HLA-A*01:01 (A1), HLA-A*02:01 (A2), HLA-A*03:01 (A3), HLA-B*07:02 (B7), HLA-B*35:01 (B35) and/or HLA-C*07:02 (C702) were selected for further investigation (FIG. 1).

Selection of High Affinity TCRs with a Safe Recognition Profile

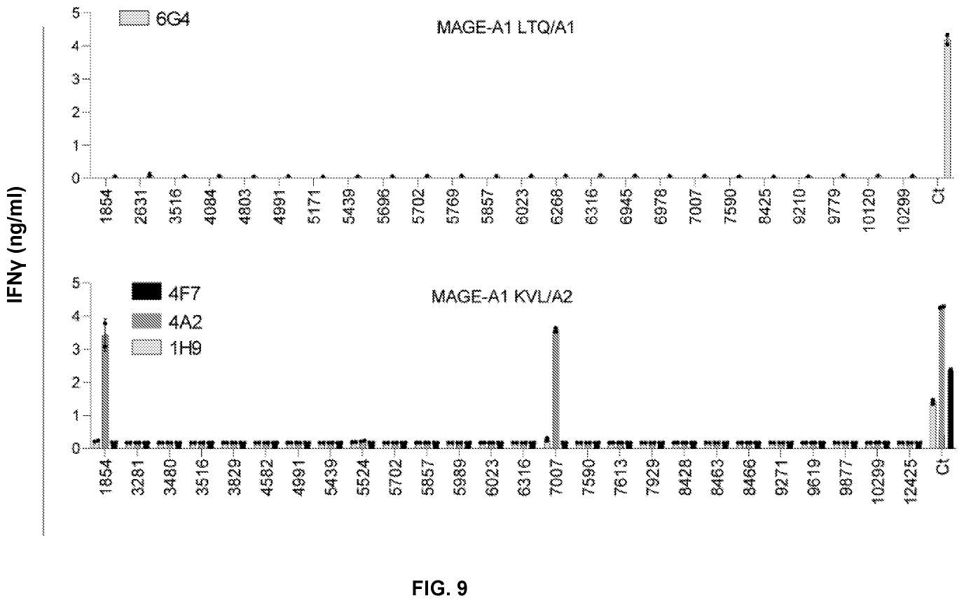

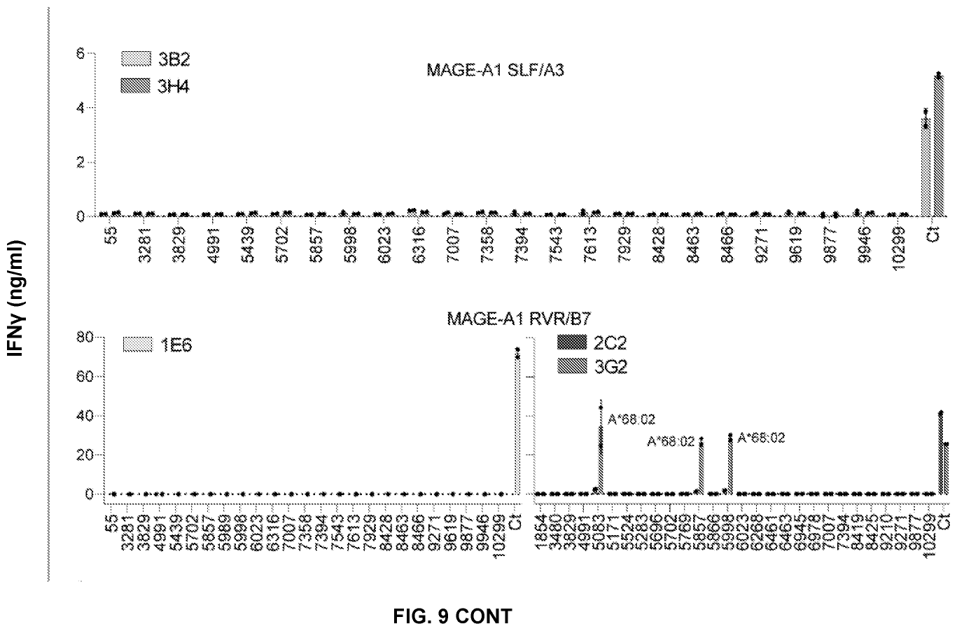

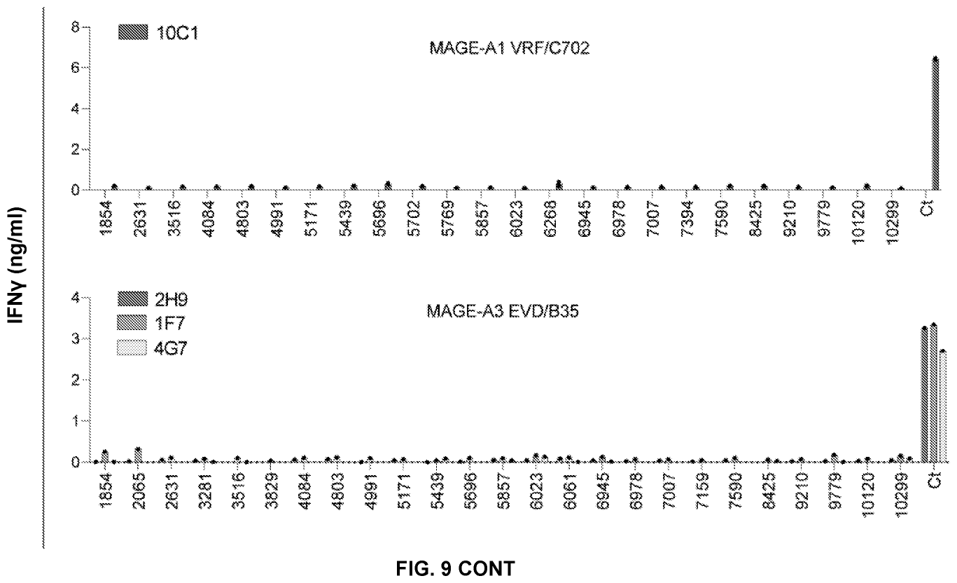

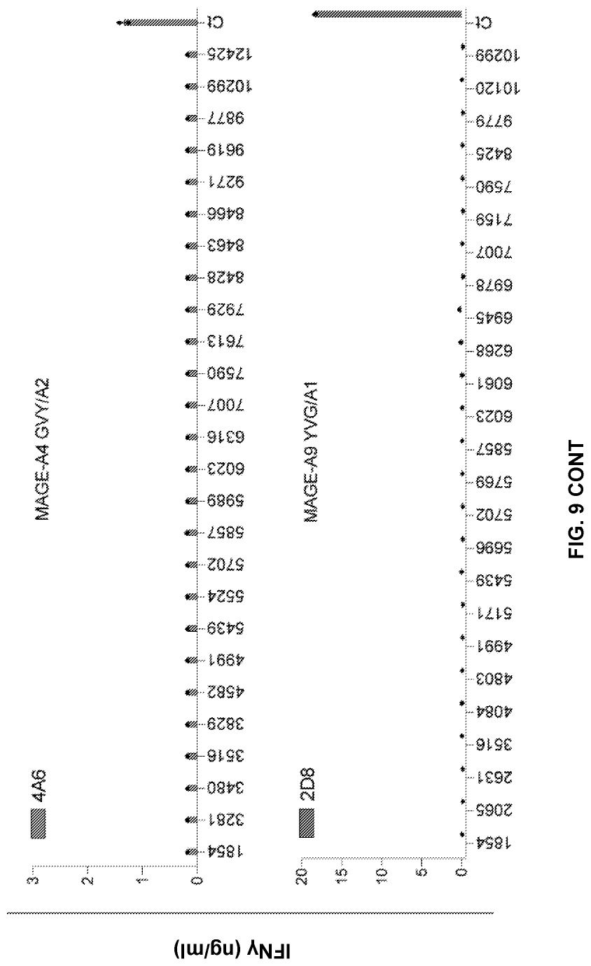

To evaluate the specificity of the 15 selected CT clones, their safety profiles were determined. First, the cross-reactivity of the CT clones for other pHLA complexes was analyzed with EBV-LCL panels. The EBV-LCL panels comprise of different EBV-LCLs expressing all HLA alleles with a prevalence of >1% in the Caucasian population (Table 4). Clone 4A2 (MAGE-A1 KVL/A2) was excluded based on the recognition of 2 EBV-LCLs as determined by IFN-γ production upon overnight co-culture (FIG. 9). The exact peptide-HLA complex that was recognized by the 4A2 clone could not be determined, since the 2 EBV-LCLs recognized did not share an HLA allele. Clone 3G2 (MAGE-A1 RVR/B7) was reactive against HLA-A*68:02 positive EBV-LCL. Since the frequency of this HLA allele is low in the Caucasian population (<1%) this clone was not excluded from further analyses.

TABLE 4
HLA-typing of EBV-LCLs used in EBV-LCL screenings.
HLA-typing of EBV-LCLs used in EBV-LCL screenings

| EBV-LCL | HLA-A | HLA-B | HLA-C |
|---|---|---|---|
| 55 | 01-68 | 08-53 | 04-07 |
| 1854 | 02:01-30:02 | 15:01-39:01 | 03:03-12:03 |
| 2065 | 01:01-02:01 | 37:01-39:01 | 06:02-07:02 |
| 2631 | 02:01-03:01 | 44:02-57:01 | 06:02-07:04 |
| 3281 | 01:01-32:01 | 08:01-45:01 | 06:02-07:01 |
| 3480 | 26:01-01:01 | 38:01-18:01 | 12:03-07:01 |
| 3516 | 03:01-26:01 | 07:02-14:01 | 07:02-08:02 |
| 3829 | 01:01-68:01 | 44:02 | 05:01-07:04 |
| 4084 | 03:01-30:01 | 07:02-38:01 | 07:02-12:03 |
| 4582 | 01-30:01 | 08:01-40:01 | 03:04-07:01 |
| 4803 | 03:01-33:01 | 07:02-14:02 | 07:02-08:02 |
| 4991 | 26:01-31:01 | 14:01-49:01 | 07:01-08:02 |
| 5171 | 02:01-66:01 | 40:01-41:02 | 03:04-17 |
| 5283 | 24:02-66:01 | 14:02-39:06 | 07:02-08:02 |
| 5524 | 02:01-31:01 | 15:01-15:17 | 03:04-07:01 |
| 5439 | 03:01-25:01 | 15:17-18:01 | 07:01-12:03 |
| 5696 | 02:05 | 58:01 | unknown |
| 5702 | 32:01-68:01 | 35:03-52:01 | 12:02-12:03 |
| 5769 | 02:01-68:01 | 35:03-37:01 | 04:01-06:02 |
| 5857 | 30:04-68:02 | 38:01-55:01 | 03:03-12:03 |
| 5866 | 02:01-11:01 | 35:01-51:01 | 04:01-14:02 |
| 5989 | 11:01-68:01 | 38:01-39:01 | 07:02-12:03 |
| 6023 | 03:01-11:01 | 40:02-56:01 | 01:02-02:02 |
| 6268 | 02:01-24:02 | 35:02-44:02 | 04:01-05:01 |
| 6316 | 29:02-30:01 | 13:02-44:03 | 06:02-16:01 |
| 6461 | 02:01 | 40:02 | 02:02 |
| 6463 | 02:01 | 57:01 | 06:02 |
| 6945 | 03:01-25:01 | 18:01-35:01 | 04:01-12:03 |
| 6978 | 02:01-02:05 | 15:01-45:01 | 01:02-06:02 |
| 7007 | 02:05-29:02 | 27:05-44:03 | 01:02-16:01 |
| 7159 | 02:01 | 13:02-44:02 | 05:01-06:02 |
| 7394 | 01:01-32:01 | 35:08 | 04:01 |
| 7590 | 24:02-31:01 | 07:02-35:08 | 04:01-07:02 |
| 7613 | 01:01-24:02 | 15:01-37:01 | 03:03-06:02 |
| 7929 | 01:01 | 35:02-52:01 | 04:01-12:02 |
| 8419 | 02:01-01:01 | 50:01-07:02 | 07:02-06:02 |
| 8425 | 23:01-02:01 | 41:01-40:01 | 17:01-03:04 |
| 8428 | 11:01-01:01 | 57:01-35:01 | 06:02-04:01 |
| 8463 | 11:01-01:01 | 51:01-50:01 | 15:02-06:02 |
| 8466 | 23:01 | 51:01-41:01 | 17:01-15:02 |
| 9210 | 02:01 | 15:01-51:01 | 03:03-15:02 |
| 9271 | 33:01-66:01 | 58:01-58:02 | 03:02-06:02 |
| 9619 | 01:01-33:03 | 44:03-51:01 | 07:06-14:02 |
| 9877 | 01:01-23:01 | 08:01-41:02 | 07:01-17:03 |
| 9779 | 02:01-03:01 | 08:01-50:01 | 06:02-07:01 |
| 10120 | 02:01-24:02 | 07:02-40:02 | 02:02-07:02 |
| 10299 | 02:01-11:01 | 44:05-51:01 | 02:02-14:02 |
| 12425 | 23:01-36:01 | 15:03-53:01 | 02:10-04:01 |

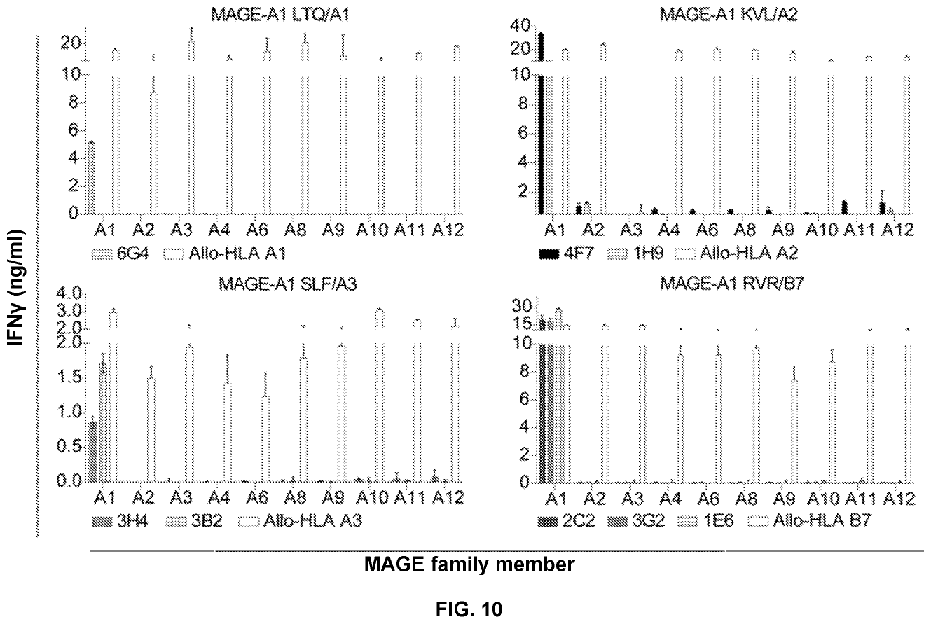

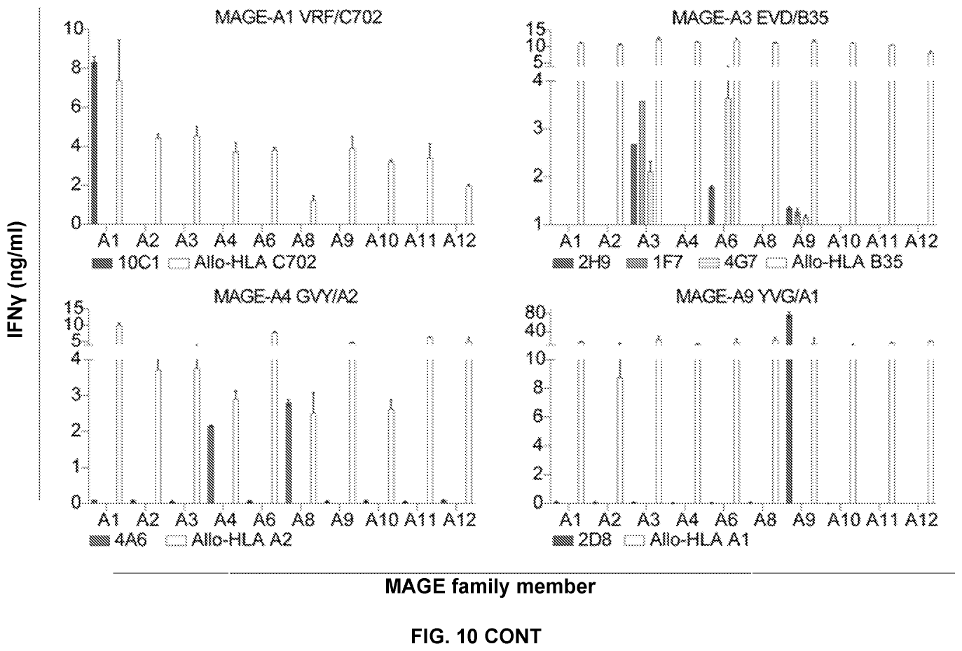

In the subsequent screening, the 14 residual CT clones were tested against Raji cells Td with all MAGE-A family members and relevant HLA alleles to exclude clones recognizing a look-a-like peptide present in one of the non-selected MAGE-A genes. Most CT clones, except for 3, specifically recognized only their CT-target gene as determined by IFN-γ production upon overnight co-culture (FIG. 10). The 2H9 and 4G7 (MAGE-A3 EVD/B35) clones were reactive against MAGE-A3 as well as MAGE-A6. This additional MAGE-A6 reactivity was confirmed by demonstrating that the T cells clones were able to recognize Raji cells loaded with EVDPIGHLY (SEQ ID NO: 133) and EVDPIGHVY (SEQ ID NO: 134) peptide derived from MAGE-A3 and MAGE-A6, respectively (data not shown). Since, MAGE-A6 was previously selected as a safe CT gene, these 2 CT clones were not excluded from further screening. The 4A6 (MAGE-A4 GVY/A2) clone reactive against GVYDGREHTV (SEQ ID NO: 157) peptide also revealed to be reactive against the GLYDGREHSV (SEQ ID NO: 182) peptide derived from MAGE-A8. Since MAGE-A8 is expressed at low levels in cerebellum, thyroid gland, and urinary bladder, according to the online databases GTEx and HPA, the inventors excluded this T cell clone from further screening.

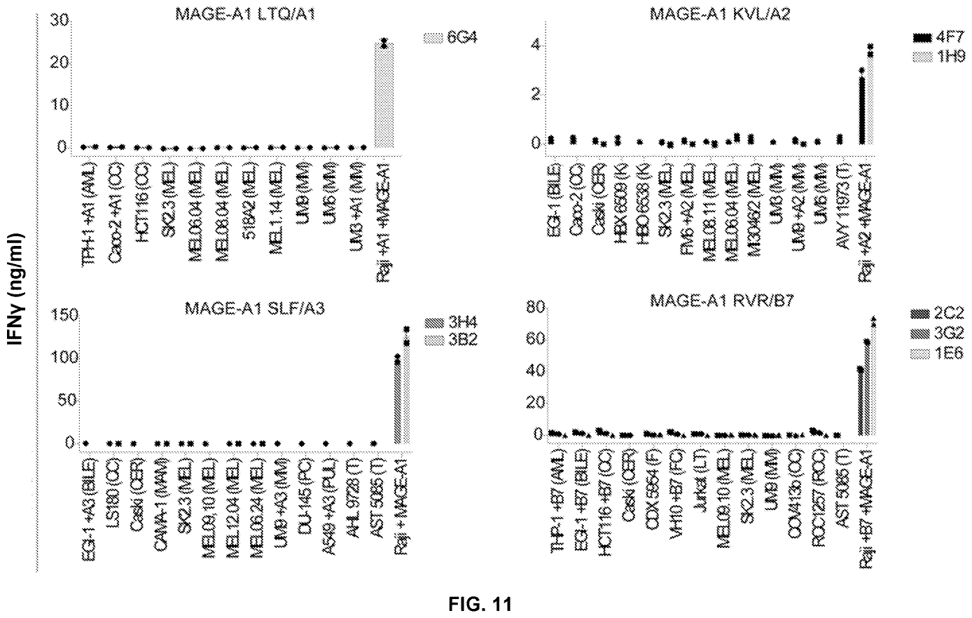

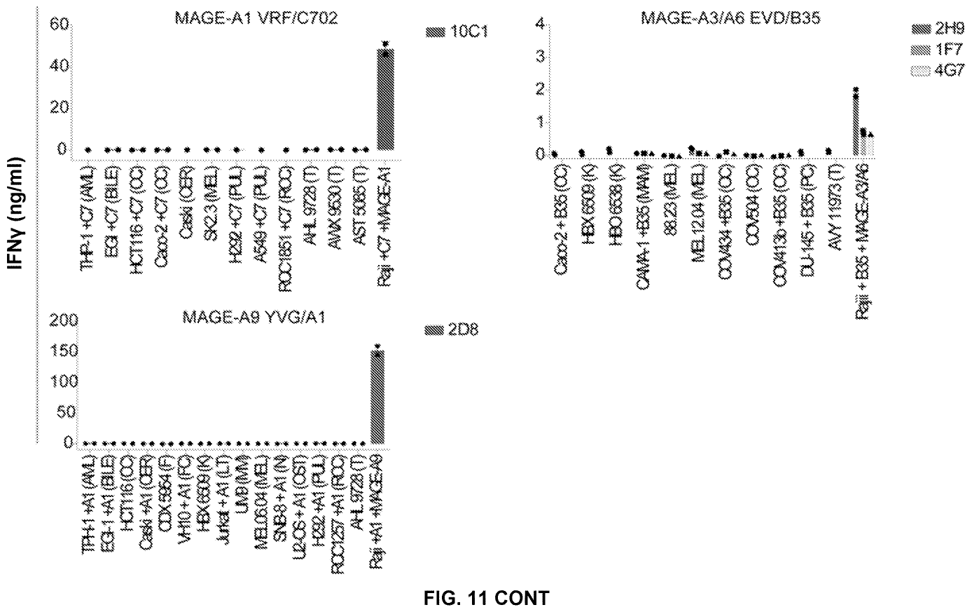

As a last specificity check, the 13 residual CT clones were co-cultured with a variety of CT-negative tumor cell lines from different tissue origin, all expressing the required HLA restriction allele, to exclude cross-reactivity against look-a-like peptides in the context of target HLA. None of the 13 clones produced IFN-γ upon stimulation with one of the CT-negative tumor cell lines, whereas in the same assay Raji Td with HLA and CT-target gene were recognized (FIG. 11). Based on these different screenings the inventors concluded that these 13 residual CT clones recognizing 7 different peptides have a safe reactivity profile. From each different specificity the most potent candidate was selected for TCR gene therapy (Table 5; Table 3).

TABLE 5
TCRs included in TCR library.

| Name | Peptide | Gene | HLA restriction |
|---|---|---|---|
| 6G4 | LTQDLVQEKYLEY (SEQ | MAGE-A1 | HLA-A*01:01 |
| | ID NO: 130) | | |
| 4F7 | KVLEYVIKV (SEQ ID NO: | MAGE-A1 | HLA-A*02:01 |
| | 127) | | |
| 3H4 | SLFRAVITK (SEQ ID NO: | MAGE-A1 | HLA-A*03:01 |
| | 131) | | |
| 3G2 | RVRFFFPSL (SEQ ID | MAGE-A1 | HLA-B*07:02 |
| | NO: 132) | | |
| 10C1 | VRFFFPSL (SEQ ID NO: | MAGE-A1 | HLA-C*07:02 |
| | 128) | | |
| 2H9 | EVDPIGHLY (SEQ ID NO: | MAGE-A3/A6 | HLA-B*35:01 |
| | 133)/EVDPIGHVY (SEQ | | |
| | ID NO: 134) | | |
| 2D8 | YVGKEHMFY (SEQ ID | MAGE-A9 | HLA-A*01:01 |
| | NO: 129) | | |

### Cytotoxicity of TCR Transduced CD8⁺ T Cells

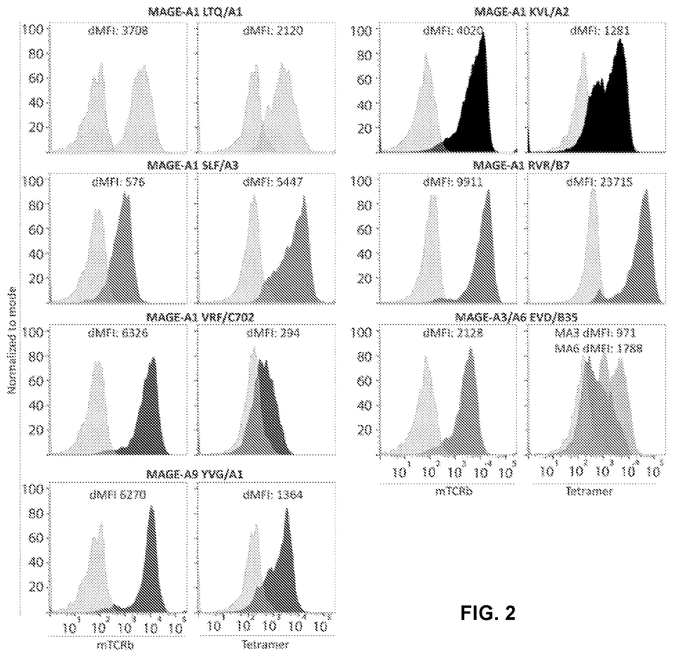

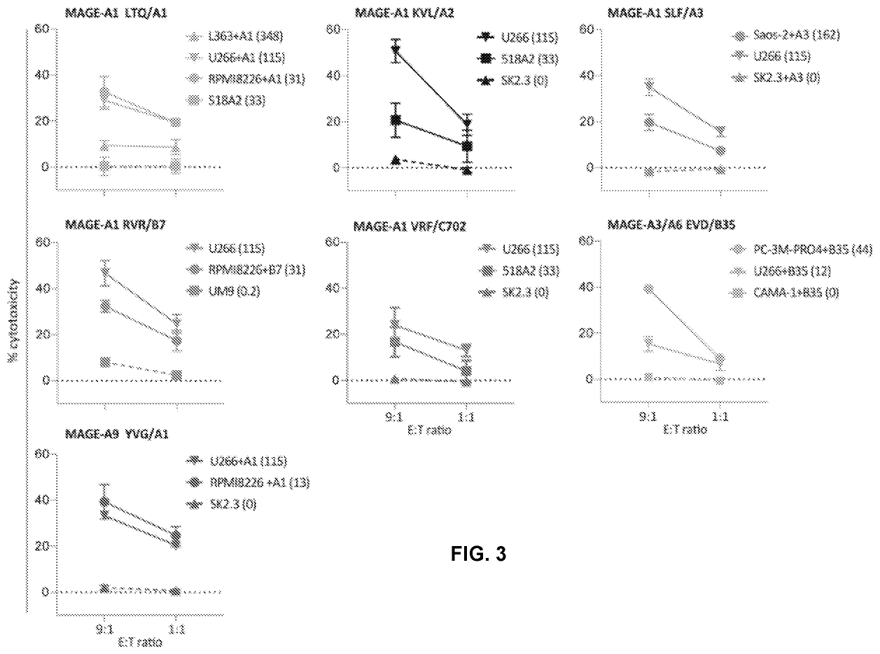

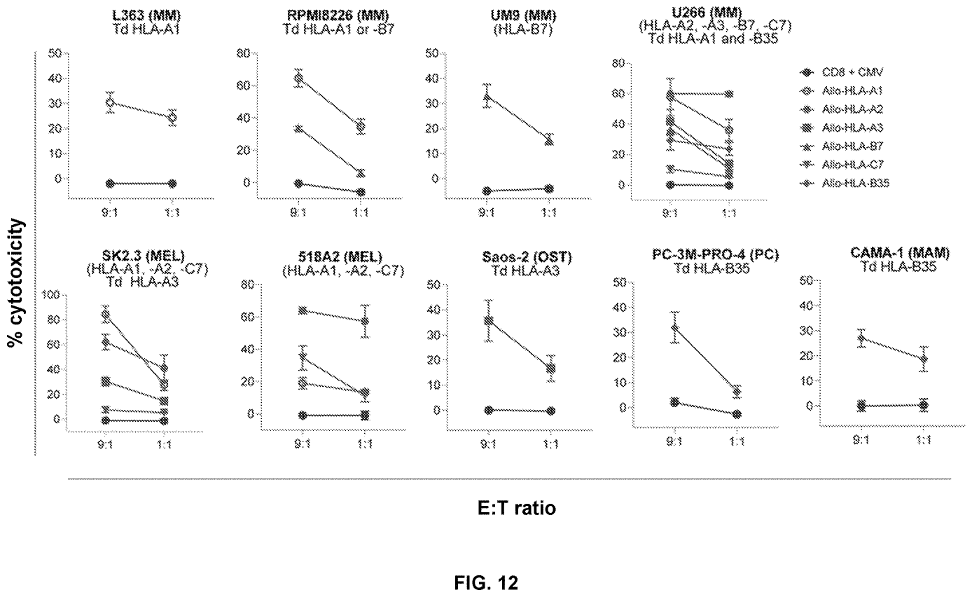

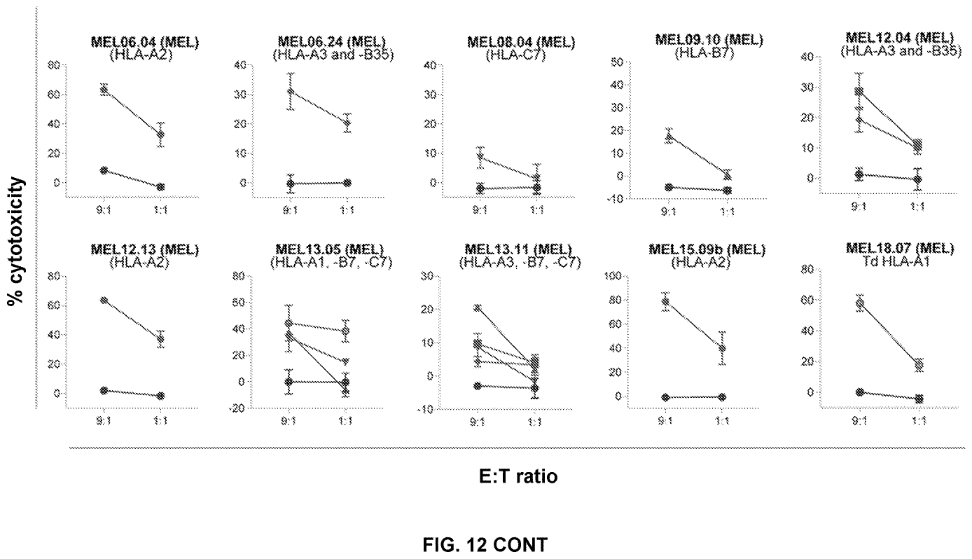

To study the potential for clinical application in TCR gene therapy, the TCRs of these 7 selected T cell clones were sequenced and retrovirally transferred into healthy-donor CD8⁺ T cells. Tetramer and murine TCR-β staining confirmed expression of TCR constructs in Td CD8⁺ T cells (FIG. 2). Tetramer staining of the MAGE-A1 VRF/C702 and MAGE-A3 EVD/B35 TCR had lower gMFI compared to other selected TCRs. Nevertheless, effective and CT-specific cytotoxicity of TCR-T cells was demonstrated by an 6-hour ⁵¹Cr-release assays (FIG. 3). All TCR-T cells demonstrated to be cytotoxic against tumor cell lines expressing the relevant MAGE genes. CMV-TCR T cells and allo-HLA reactive clones were included to demonstrate TCR specific killing, and killing sensitivity of tumor cell lines, respectively (FIG. 12). These results demonstrate that several potent TCRs specific for MAGE-A1, MAGE-A3, MAGE-A6 or MAGE-A9 in the context of HLA-A1, -A2, -A3, -B7, -B35 or -C702 were identified.

### TCR Transfer Reveals Effective Cytokine Production and Killing of Early Passage Melanoma Samples

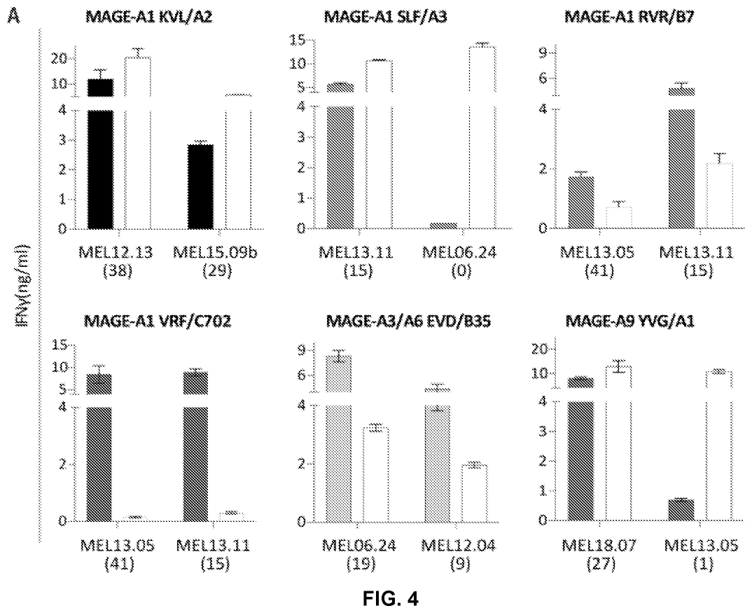

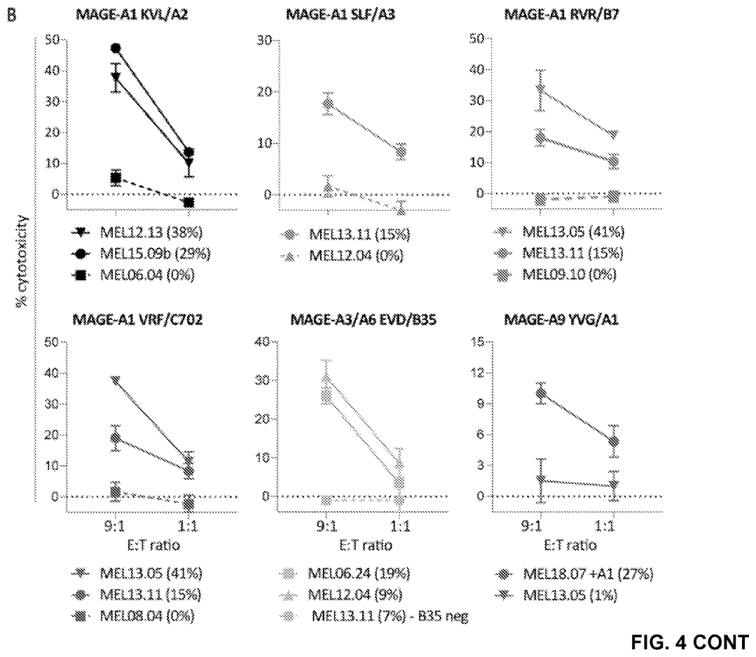

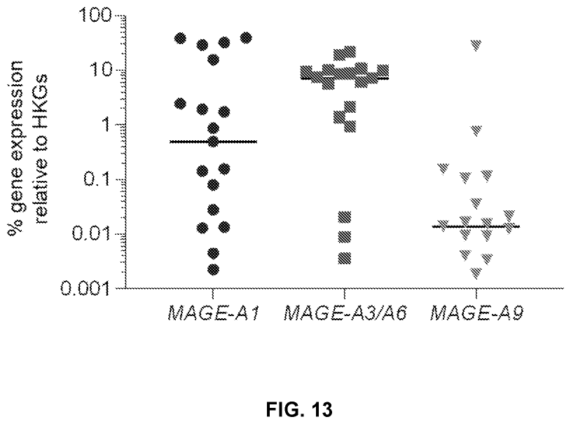

To obtain further insight into the potential clinical effectivity of the TCRs, the anti-tumor reactivity of the TCR-T cells was analyzed against early-passage melanoma cell lines (passage≤10). Expression of the different MAGE genes in these cell lines was measured by qPCR (FIG. 13), and demonstrated to be comparable to what is described in the literature [23]. For each TCR 1-2 early-passage melanoma samples were included expressing both the target gene and HLA of interest. Anti-tumor reactivity of the TCR-T cells was evaluated by measuring the IFN-γ production of the TCR-T cells after an overnight co-culture with the early-passage melanoma cell lines and in an 6-hour chromium release assay. CMV-TCR T cells and allo-HLA reactive clones were included to demonstrate TCR specific killing, and killing sensitivity of the early-passage melanoma cell lines, respectively (FIG. 12). Six TCRs produced high amounts of IFN-γ and effectively lysed all CT-positive early-passage melanoma cell lines upon stimulation (FIG. 4).

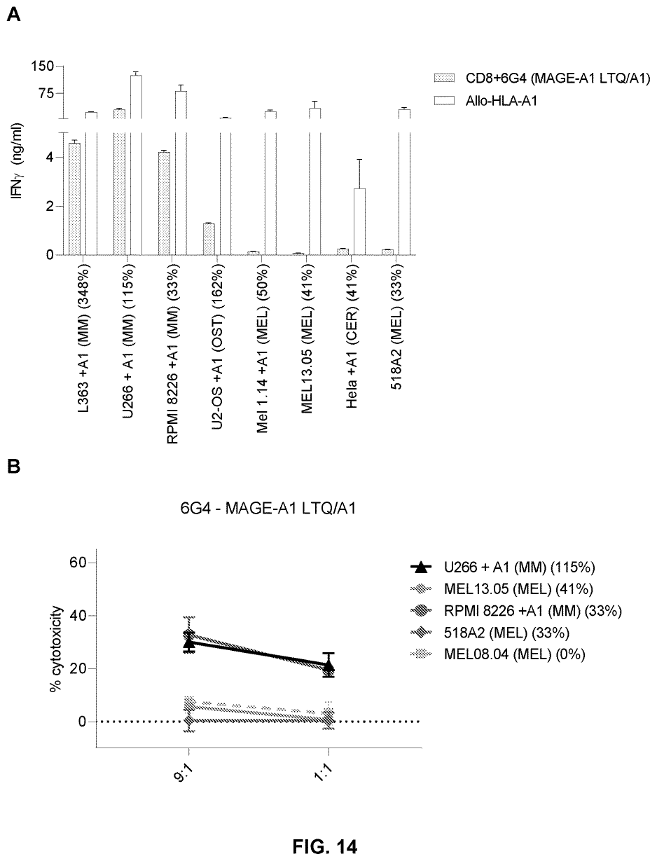

One T cell clone, 6G4 (MAGE-A1 LTQ/A1) strongly recognized all HLA-A1 positive MM cell lines, however limited amounts of IFN-γ were produced upon co-culture with tumor cell lines of non-hematopoietic origin (FIG. 14). Based on the potent anti-MM reactivity this TCR was included as a promising TCR for TCR gene therapy of MM. In conclusion, the inventors identified several promising CT-specific TCRs, reactive against multiple different CT genes and restricted by different HLA alleles for TCR gene therapy.

### Potent Antitumor Reactivity of MAGE-A1 TCRs In Vivo

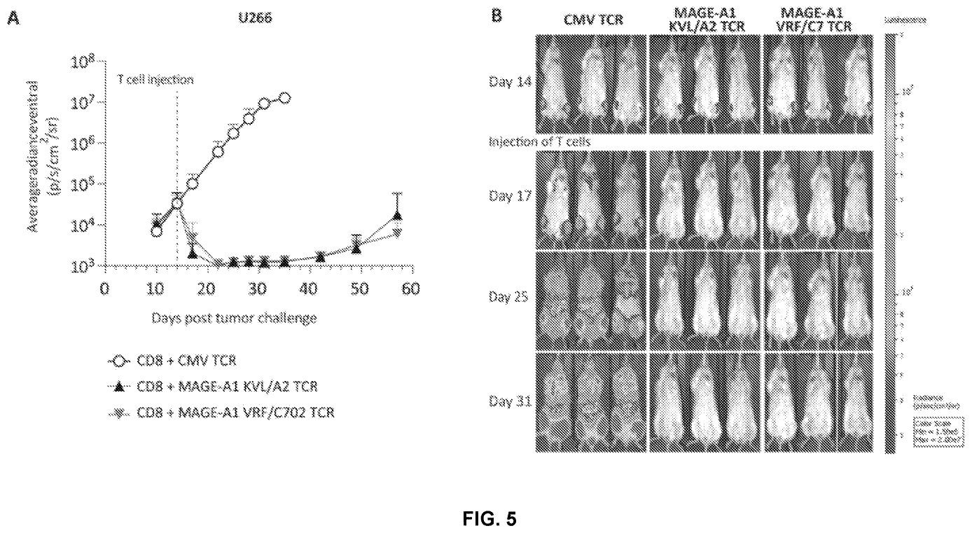

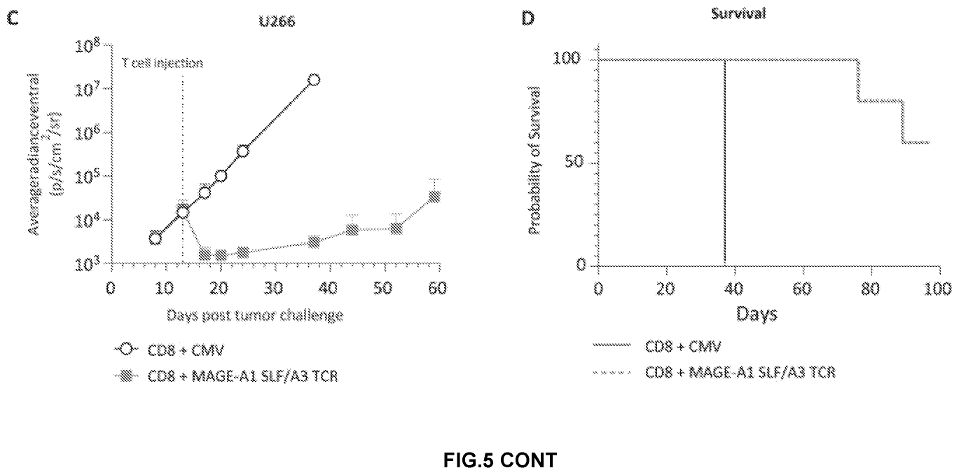

The in vivo efficacy of three of the identified TCRs; 4F7 (MAGE-A1 KVL/A2), 3H4 (MAGE-A1 SLF/A3) and 10C1 (MAGE-A1 VRF/C702), was determined in an orthotopic xenograft model of established multiple myeloma [12]. The multiple myeloma cell line U266 expresses HLA-A*02:01, HLA-A*03:01, HLA-C*07:02 and MAGE-A1. NSG mice were intravenously injected with U266 14 days prior to T cell injection. Treatment with the MAGE-A1 KLV/A2 TCR, MAGE-A1 VRF/C702 TCR, and MAGE-A1 SLF/A3 TCR-T cells demonstrated a major antitumor effect since 3-8 days after T cell infusion the bone marrow located tumor cells were completely eradicated as could be visualized with CCD (FIG. 5). Almost complete tumor eradication was observed up to 43 days after T cell infusion for all mice treated with MAGE-A1 TCR-T cells. For the MAGE-A1 SL/A3 TCR-T cell treated mice the inventors observed that 3 of the 5 mice had no tumor or very low tumor outgrowth and survived 86 days after T cell infusion. These results confirm potent pre-clinical antitumor efficacy of the newly identified MAGE specific TCRs described herein.

Evaluation of TCR 4F7 Transgenic T Cells Compared to TCR D1 (1388.2b) Transgenic T Cells from WO 2018/170338

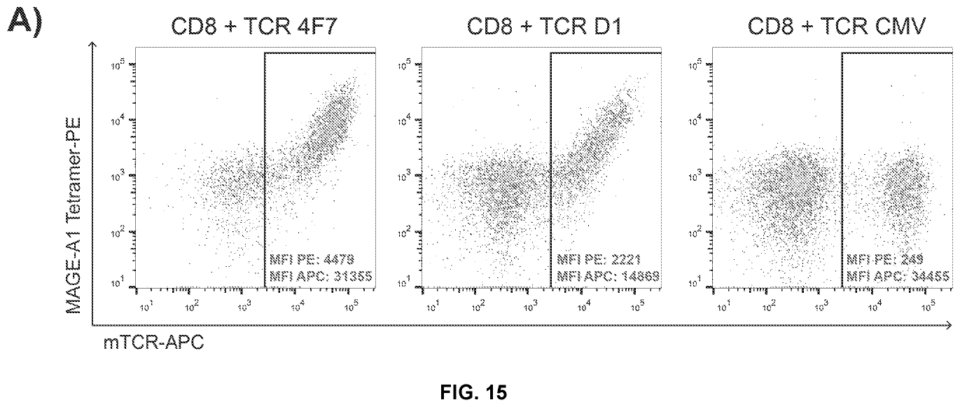

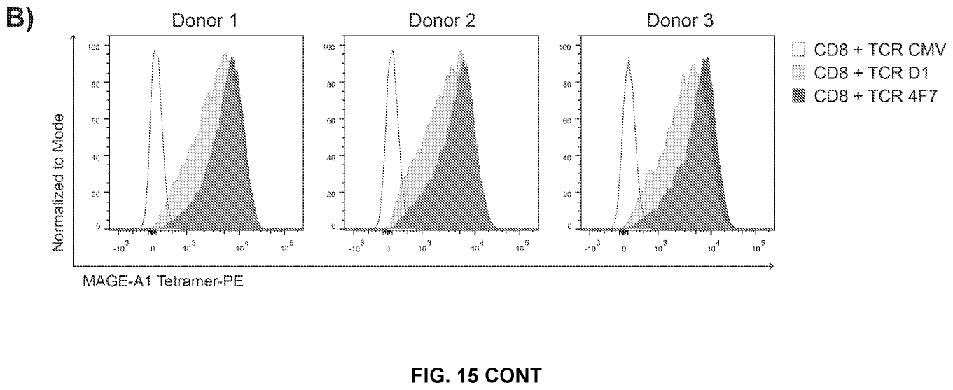

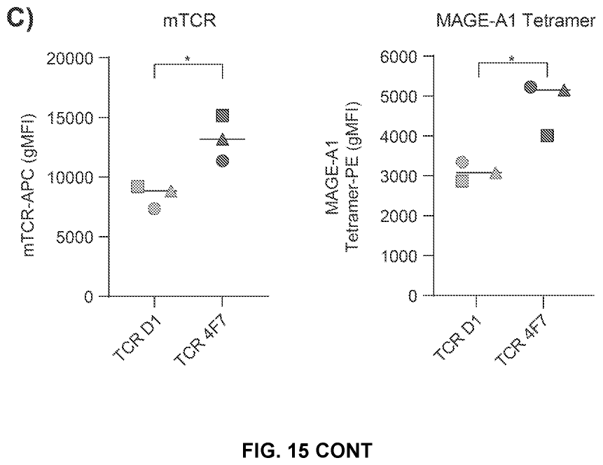

To demonstrate the additional value of the inventor's newly identified TCR 4F7 MAGE-A1 KVL HLA-A*02:01 (MAGE-A1 KVL/A2) to the previously disclosed TCR, referred to herein as “TCR D1”, TCR expression and target peptide-HLA binding was investigated. TCR D1 is TCR 1388.2b from WO 2018/170338. CD8 T cells isolated from healthy donor PBMCs were retrovirally transduced with MAGE-A1 HLA-A2 restricted TCR 4F7 and TCR D1 as well as a negative control CMV-pp65 HLA-A2 restricted TCR. TCR expression in bulk MAGE-A1 TCR transduced T cells was measured using an anti-mTCR antibody. TCR 4F7 transgenic T cells expressed higher levels of mTCR than TCR D1 transgenic T cells (FIG. 15A). Increased TCR expression of TCR 4F7 was associated with an increased ability of these cells to bind MAGE-A1 KVL/A2 pHLA-tetramer. In CD8 T cells from three donors, all three TCR 4F7 enriched T cell populations bound pHLA-tetramer to a higher level than TCR D1 expressing T cell populations (FIG. 15B). Overall, the level of mTCR expression as well as pHLA-Tetramer binding was significantly higher for TCR 4F7 T cells compared to TCR D1 T cells (FIG. 15C). Superior TCR expression and pHLA tetramer binding by TCR 4F7 transgenic T cells could indicate a greater sensitivity to target peptide HLA complexes, which could be especially valuable when targeting tumor cells expressing lower levels of MAGE-A1. Extending the range of MAGE A1 TCR targetable tumors could increase the patient population eligible for therapy with MAGE A1 TCR T cells.

### DISCUSSION

The inventors aimed to broaden the TCR gene therapeutic treatment options for cancer patients by extending the number of tumor-specific TCRs reactive against different CT genes and restricted by prevalent HLA alleles. As a first step, the most promising CT genes were selected by bioinformatic tools and subsequent HLA peptidomics revealed the naturally processed and presented HLA class I peptides of these genes. In total, 31 peptides presented in the most common HLA alleles were identified by this method and another 8 were included based on literature or prediction. pHLA-tetramer positive CD8⁺ T cells were isolated and the most promising T cell clones were selected based on various effectivity and safety screenings. Subsequently, to determine clinical potential of the identified T cell clones, TCRs were sequenced and transduced into peripheral blood derived CD8⁺ T cells. Several of the 7 TCRs with unique CT specificity and HLA restriction were reactive against multiple different tumor types, including hematopoietic tumors and non-hematopoietic tumors. The reactivity of TCR-6G4 specific for LTQ peptide of MAGE-A1 in HLA-A1 was very effective against MM samples, but limited to non-hematopoietic tumor cells. Advantageously, the inventors identified several potent and unique CT-specific TCRs effectively targeting MAGE-A1, MAGE-A3, MAGE-A6 or MAGE-A9 expressing tumors in the context of HLA-A1, -A2, -A3, -B7, -B35 or -C7. Treatment with MAGE-A1 specific TCR-T cells resulted in total tumor eradication of MAGE-A1 expressing MM cells in an orthotopic xenograft mouse model, demonstrating strong antitumor reactivity of the identified TCRs in vivo.

The identified set of CT-specific TCRs will enlarge the group of cancer patients that can be treated. The targeted HLA alleles; HLA-A*01:01; -A*02:01; -A*03:01; -B*07:02; -B*35:01 and; -C*07:02 have relative allele frequencies in the worldwide population of 17%, 39%, 17%, 13%, 8%, 21% respectively. MAGE-A1, MAGE-A3, MAGE-A6 and MAGE-A9 are expressed in a variety of different tumor types including in melanomas (MAGE-A1: 16-20% primary and 48-51% metastases), non-small cell lung carcinomas (MAGE-A1: 27-46%, MAGE-A3: 38-55%, MAGE-A6: 26%), breast carcinomas (MAGE-A9: 45%, MAGE-A3/A6: 10-15%, MAGE-A1: 6%), ovarian carcinomas (MAGE-A1: 15-54%, MAGE-A3: 36-37%, MAGE-A9: 37%), colon carcinomas and multiple myelomas (MAGE-A1: 12-30%, MAGE-A3: 20-27%) [23]. Based on HLA typing and CT expression approximately 37% of metastasized melanoma patients, 24-39% of non-small cell lung cancer patients, 23-52% of ovarian carcinoma patients and 14-26% of (relapsed) multiple myeloma patients can be treated with this novel set of TCRs.

Advantageously, the novel TCRs identified herein may be exploited as an “off-the-shelf” TCR gene therapy. The importance of proper safety screenings during the selection process of CT-specific TCRs was underlined by two clinical trials using a MAGE-A3 TCR causing fatal toxicity. In the first study, two patients died of neurotoxicity caused by the recognition of MAGE-A12 in the brain [15]. To overcome unwanted recognition of any of the look-a-like MAGE-A family members, the inventors screened their TCRs against a panel of Raji Td with all family members. The TCRs recognizing a MAGE-A family member with an unsafe expression profile were excluded from further analysis. In a second clinical trial, unexpected reactivity against titin resulted in fatal cardiac toxicity of 2 patients [14]. To limit the chance to induce off-target toxicity, the inventors included a CT-negative tumor panel consisting of tumors from different origins and only continued with those T cells identified as safe.

In summary, the inventors used a high-throughput method to identify a novel set of CT-specific TCRs useful for TCR gene therapeutic strategies. This resulted in the identification of MAGE-A1, MAGE-A3, MAGE-A6 and MAGE-A9 specific TCRs targeting peptides in the context of HLA-A1, -A2, -A3, -B7, -B35 or -C7. Advantageously, the identified TCRs can be included in an “off-the-shelf” TCR library that makes it possible to treat cancer patients with prior generated TCR constructs in a personalized and multi-antigen-targeting T-cell therapy.

### List of Abbreviations

B: Burkitt lymphoma
B2M: beta-2 microglobulin
CML: chronic myeloid leukemia
CPM: counts per minute
CT: Cancer Testis
DMEM: Dulbecco's modified eagle medium
E:T: effector target ratio
EBV-LCLs: Epstein-Barr Virus transformed B lymphoblastoid cell lines
ELISA: enzyme-linked immunosorbent assay
FBS: fetal bovine serum
FCF: Flow cytometry Core Facility
GTEx: Genotype-Tissue Expression
HC: heavy chain
HKG: Housekeeping gene
HLA: Human leukocyte antigen
HPLC: high-performance liquid chromatography
HPA: Human Protein Atlas
IMDM: Iscove's modified Dulbecco's medium
IMGT: ImMunoGeneTics
I.V.: intravenous
MACS: magnetic-activated cell sorting
MAGE: melanoma-associated antigen
MAM: mammary carcinoma
MEL: melanoma
MFI: mean fluorescent intensity
MM: multiple myeloma
NEAA: nonessential amino acids
NSG: NOD scid gamma mouse
OST: osteosarcoma
PBMC: peripheral blood mononuclear cells
Pen/Strep: penicillin/streptomycin
PHA: phytohemagglutinin
pHLA: peptide HLA complex
qPCR: Quantitative reverse transcription polymerase chain reaction
TCM: T cell medium
TCR: T cell receptor
Td: transduced

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### Sequences

TABLE 6
TCR CT27.4F7; target peptide KVLEYVIKV (SEQ ID NO 127); target gene MAGE-A1 A*02:01
Sequences for TCR CT27.4F7; target peptide KVLEYVIKV (SEQ ID NO 127); target
gene MAGE-A1 A*02:01.

| SEQ ID | TCR | AA or | |
|---|---|---|---|
| NO | POLYPEPTIDE | NT | SEQUENCE |
| 1 | α CDR1 | AA | DSSSTY |
| 2 | α CDR2 | AA | IFSNMDM |
| 3 | α CDR3 | AA | CAESIDARLMF |
| 4 | β CDR1 | AA | SGDLS |
| 5 | β CDR2 | AA | YYNGEE |
| 6 | β CDR3 | AA | CASSPGGDTQYF |
| 7 | α VJ | AA | MKTFAGFSFLFLWLQLDCMSRGEDVEQSLFLSVREGDSSVINCTYTDS |
| | | | SSTYLYWYKQEPGAGLQLLTYIFSNMDMKQDQRLTVLLNKKDKHLSLRI |
| | | | ADTQTGDSAIYFCAESIDARLMFGDGTQLVVKP |
| 8 | α VJ | NT | ATGAAGACATTTGCTGGATTTTCGTTCCTGTTTTTGTGGCTGCAGCT |
| | | | GGACTGTATGAGTAGAGGAGAGGATGTGGAGCAGAGTCTTTTCCTG |
| | | | AGTGTCCGAGAGGGAGACAGCTCCGTTATAAACTGCACTTACACAG |
| | | | ACAGCTCCTCCACCTACTTATACTGGTATAAGCAAGAACCTGGAGCA |
| | | | GGTCTCCAGTTGCTGACGTATATTTTTTCAAATATGGACATGAAACAA |
| | | | GACCAAAGACTCACTGTTCTATTGAATAAAAAGGATAAACATCTGTCT |
| | | | CTGCGCATTGCAGACACCCAGACTGGGGACTCAGCTATCTACTTCT |
| | | | GTGCAGAGAGTATAGATGCCAGACTCATGTTTGGAGATGGAACTCA |
| | | | GCTGGTGGTGAAGCCC |
| 9 | β VDJ | AA | MGFRLLCCVAFCLLGAGPVDSGVTQTPKHLITATGQRVTLRCSPRSGD |
| | | | LSVYWYQQSLDQGLQFLIQYYNGEERAKGNILERFSAQQFPDLHSELNL |
| | | | SSLELGDSALYFCASSPGGDTQYFGPGTRLTVL |
| 10 | β VDJ | NT | ATGGGCTTCAGGCTCCTCTGCTGTGTGGCCTTTTGTCTCCTGGGAG |
| | | | CAGGCCCAGTGGATTCTGGAGTCACACAAACCCCAAAGCACCTGAT |
| | | | CACAGCAACTGGACAGCGAGTGACGCTGAGATGCTCCCCTAGGTCT |
| | | | GGAGACCTCTCTGTGTACTGGTACCAACAGAGCCTGGACCAGGGCC |
| | | | TCCAGTTCCTCATTCAGTATTATAATGGAGAAGAGAGAGCAAAAGGA |
| | | | AACATTCTTGAACGATTCTCCGCACAACAGTTCCCTGACTTGCACTC |
| | | | TGAACTAAACCTGAGCTCTCTGGAGCTGGGGGACTCAGCTTTGTATT |
| | | | TCTGTGCCAGCAGCCCCGGCGGGGATACGCAGTATTTTGGCCCAG |
| | | | GCACCCGGCTGACAGTGCTC |
| 11 | α VJ | AA | MKTFAGFSFLFLWLQLDCMSRGEDVEQSLFLSVREGDSSVINCTYTDS |
| | and | | SSTYLYWYKQEPGAGLQLLTYIFSNMDMKQDQRLTVLLNKKDKHLSLRI |
| | constant | | ADTQTGDSAIYFCAESIDARLMFGDGTQLVVKPNIQNPDPAVYQLRDSK |
| | | | SSDKSVCLFTDFDSQTNVSQSKDSDVYITDKTVLDMRSMDFKSNSAVA |
| | | | WSNKSDFACANAFNNSIIPEDTFFPSPESSCDVKLVEKSFETDTNLNFQ |
| | | | NLSVIGFRILLLKVAGFNLLMTLRLWSS |
| 12 | α VJ | NT | ATGAAGACATTTGCTGGATTTTCGTTCCTGTTTTTGTGGCTGCAGCT |
| | and | | GGACTGTATGAGTAGAGGAGAGGATGTGGAGCAGAGTCTTTTCCTG |
| | constant | | AGTGTCCGAGAGGGAGACAGCTCCGTTATAAACTGCACTTACACAG |
| | | | ACAGCTCCTCCACCTACTTATACTGGTATAAGCAAGAACCTGGAGCA |
| | | | GGTCTCCAGTTGCTGACGTATATTTTTTCAAATATGGACATGAAACAA |
| | | | GACCAAAGACTCACTGTTCTATTGAATAAAAAGGATAAACATCTGTCT |
| | | | CTGCGCATTGCAGACACCCAGACTGGGGACTCAGCTATCTACTTCT |
| | | | GTGCAGAGAGTATAGATGCCAGACTCATGTTTGGAGATGGAACTCA |
| | | | GCTGGTGGTGAAGCCCAATATCCAGAACCCTGACCCTGCCGTGTAC |
| | | | CAGCTGAGAGACTCTAAATCCAGTGACAAGTCTGTCTGCCTATTCAC |
| | | | CGATTTTGATTCTCAAACAAATGTGTCACAAAGTAAGGATTCTGATGT |
| | | | GTATATCACAGACAAAACTGTGCTAGACATGAGGTCTATGGACTTCA |
| | | | AGAGCAACAGTGCTGTGGCCTGGAGCAACAAATCTGACTTTGCATG |
| | | | TGCAAACGCCTTCAACAACAGCATTATTCCAGAAGACACCTTCTTCC |
| | | | CCAGCCCAGAAAGTTCCTGTGATGTCAAGCTGGTCGAGAAAAGCTTT |
| | | | GAAACAGATACGAACCTAAACTTTCAAAACCTGTCAGTGATTGGGTT |
| | | | CCGAATCCTCCTCCTGAAAGTGGCCGGGTTTAATCTGCTCATGACG |
| | | | CTGCGGTTGTGGTCCAGCTGA |
| 13 | β VDJ | AA | MGFRLLCCVAFCLLGAGPVDSGVTQTPKHLITATGQRVTLRCSPRSGD |
| | and | | LSVYWYQQSLDQGLQFLIQYYNGEERAKGNILERFSAQQFPDLHSELNL |
| | constant | | SSLELGDSALYFCASSPGGDTQYFGPGTRLTVLEDLNKVFPPEVAVFEP |
| | | | SEAEISHTQKATLVCLATGFFPDHVELSWWVNGKEVHSGVSTDPQPLK |
| | | | EQPALNDSRYCLSSRLRVSATFWQNPRNHFRCQVQFYGLSENDEWTQ |
| | | | DRAKPVTQIVSAEAWGRADCGFTSVSYQQGVLSATILYEILLGKATLYAV |
| | | | LVSALVLMAMVKRKDF |
| 14 | β VDJ | NT | ATGGGCTTCAGGCTCCTCTGCTGTGTGGCCTTTTGTCTCCTGGGAG |
| | and | | CAGGCCCAGTGGATTCTGGAGTCACACAAACCCCAAAGCACCTGAT |
| | constant | | CACAGCAACTGGACAGCGAGTGACGCTGAGATGCTCCCCTAGGTCT |
| | | | GGAGACCTCTCTGTGTACTGGTACCAACAGAGCCTGGACCAGGGCC |
| | | | TCCAGTTCCTCATTCAGTATTATAATGGAGAAGAGAGAGCAAAAGGA |
| | | | AACATTCTTGAACGATTCTCCGCACAACAGTTCCCTGACTTGCACTC |
| | | | TGAACTAAACCTGAGCTCTCTGGAGCTGGGGGACTCAGCTTTGTATT |
| | | | TCTGTGCCAGCAGCCCCGGCGGGGATACGCAGTATTTTGGCCCAG |
| | | | GCACCCGGCTGACAGTGCTCGAGGACCTGAACAAGGTGTTCCCACC |
| | | | CGAGGTCGCTGTGTTTGAGCCATCAGAAGCAGAGATCTCCCACACC |
| | | | CAAAAGGCCACACTGGTGTGCCTGGCCACAGGCTTCTTCCCCGACC |
| | | | ACGTGGAGCTGAGCTGGTGGGTGAATGGGAAGGAGGTGCACAGTG |
| | | | GGGTCAGCACAGACCCGCAGCCCCTCAAGGAGCAGCCCGCCCTCA |
| | | | ATGACTCCAGATACTGCCTGAGCAGCCGCCTGAGGGTCTCGGCCAC |
| | | | CTTCTGGCAGAACCCCCGCAACCACTTCCGCTGTCAAGTCCAGTTCT |
| | | | ACGGGCTCTCGGAGAATGACGAGTGGACCCAGGATAGGGCCAAAC |
| | | | CCGTCACCCAGATCGTCAGCGCCGAGGCCTGGGGTAGAGCAGACT |
| | | | GTGGCTTTACCTCGGTGTCCTACCAGCAAGGGGTCCTGTCTGCCAC |
| | | | CATCCTCTATGAGATCCTGCTAGGGAAGGCCACCCTGTATGCTGTG |
| | | | CTGGTCAGCGCCCTTGTGTTGATGGCCATGGTCAAGAGAAAGGATT |
| | | | TCTGA |

TABLE 7
TCR CT31.10C1; target peptide VRFFFPSL (SEQ ID NO 128); target gene MAGE-A1 C*07:02
Sequences for TCR CT31.10C1; target peptide VRFFFPSL (SEQ ID NO 128); target
gene MAGE-A1 C*07:02.

| | | AA | |
|---|---|---|---|
| SEQ ID | TCR | or | |
| NO | POLYPEPTIDE | NT | SEQUENCE |
| 15 | α CDR1 | AA | NSMFDY |
| 16 | α CDR2 | AA | ISISSIKDK |
| 17 | α CDR3 | AA | CAASAQNNDMRF |
| 18 | β CDR1 | AA | SGHDY |
| 19 | β CDR2 | AA | FNNNVP |
| 20 | β CDR3 | AA | CASGTGWDTEAFF |
| 21 | α VJ | AA | MAMLLGASVLILWLQPDWVNSQQKNDDQQVKQNSPSLSVQEGRISILN |
| | | | CDYTNSMFDYFLWYKKYPAEGPTFLISISSIKDKNEDGRFTVFLNKSAKH |
| | | | LSLHIVPSQPGDSAVYFCAASAQNNDMRFGAGTRLTVKP |
| 22 | α VJ | NT | ATGGCCATGCTCCTGGGGGCATCAGTGCTGATTCTGTGGCTTCAGC |
| | | | CAGACTGGGTAAACAGTCAACAGAAGAATGATGACCAGCAAGTTAA |
| | | | GCAAAATTCACCATCCCTGAGCGTCCAGGAAGGAAGAATTTCTATTC |
| | | | TGAACTGTGACTATACTAACAGCATGTTTGATTATTTCCTATGGTACA |
| | | | AAAAATACCCTGCTGAAGGTCCTACATTCCTGATATCTATAAGTTCCA |
| | | | TTAAGGATAAAAATGAAGATGGAAGATTCACTGTCTTCTTAAACAAAA |
| | | | GTGCCAAGCACCTCTCTCTGCACATTGTGCCCTCCCAGCCTGGAGA |
| | | | CTCTGCAGTGTACTTCTGTGCAGCAAGCGCGCAGAACAATGACATG |
| | | | CGCTTTGGAGCAGGGACCAGACTGACAGTAAAACCA |
| 23 | β VDJ | AA | MGSWTLCCVSLCILVAKHTDAGVIQSPRHEVTEMGQEVTLRCKPISGH |
| | | | DYLFWYRQTMMRGLELLIYFNNNVPIDDSGMPEDRFSAKMPNASFSTL |
| | | | KIQPSEPRDSAVYFCASGTGWDTEAFFGQGTRLTVV |
| 24 | β VDJ | NT | ATGGGCTCCTGGACCCTCTGCTGTGTGTCCCTTTGCATCCTGGTAG |
| | | | CAAAGCACACAGATGCTGGAGTTATCCAGTCACCCCGGCACGAGGT |
| | | | GACAGAGATGGGACAAGAAGTGACTCTGAGATGTAAACCAATTTCAG |
| | | | GACACGACTACCTTTTCTGGTACAGACAGACCATGATGCGGGGACT |
| | | | GGAGTTGCTCATTTACTTTAACAACAACGTTCCGATAGATGATTCAG |
| | | | GGATGCCCGAGGATCGATTCTCAGCTAAGATGCCTAATGCATCATTC |
| | | | TCCACTCTGAAGATCCAGCCCTCAGAACCCAGGGACTCAGCTGTGT |
| | | | ACTTCTGTGCCAGCGGAACAGGGTGGGACACTGAAGCTTTCTTTGG |
| | | | ACAAGGCACCAGACTCACAGTTGTA |
| 25 | α VJ and | AA | MAMLLGASVLILWLQPDWVNSQQKNDDQQVKQNSPSLSVQEGRISILN |
| | constant | | CDYTNSMFDYFLWYKKYPAEGPTFLISISSIKDKNEDGRFTVFLNKSAKH |
| | | | LSLHIVPSQPGDSAVYFCAASAQNNDMRFGAGTRLTVKPNIQNPDPAV |
| | | | YQLRDSKSSDKSVCLFTDFDSQTNVSQSKDSDVYITDKTVLDMRSMDF |
| | | | KSNSAVAWSNKSDFACANAFNNSIIPEDTFFPSPESSCDVKLVEKSFET |
| | | | DTNLNFQNLSVIGFRILLLKVAGFNLLMTLRLWSS |
| 26 | α VJ and | NT | ATGGCCATGCTCCTGGGGGCATCAGTGCTGATTCTGTGGCTTCAGC |
| | constant | | CAGACTGGGTAAACAGTCAACAGAAGAATGATGACCAGCAAGTTAA |
| | | | GCAAAATTCACCATCCCTGAGCGTCCAGGAAGGAAGAATTTCTATTC |
| | | | TGAACTGTGACTATACTAACAGCATGTTTGATTATTTCCTATGGTACA |
| | | | AAAAATACCCTGCTGAAGGTCCTACATTCCTGATATCTATAAGTTCCA |
| | | | TTAAGGATAAAAATGAAGATGGAAGATTCACTGTCTTCTTAAACAAAA |
| | | | GTGCCAAGCACCTCTCTCTGCACATTGTGCCCTCCCAGCCTGGAGA |
| | | | CTCTGCAGTGTACTTCTGTGCAGCAAGCGCGCAGAACAATGACATG |
| | | | CGCTTTGGAGCAGGGACCAGACTGACAGTAAAACCAAATATCCAGA |
| | | | ACCCTGACCCTGCCGTGTACCAGCTGAGAGACTCTAAATCCAGTGA |
| | | | CAAGTCTGTCTGCCTATTCACCGATTTTGATTCTCAAACAAATGTGTC |
| | | | ACAAAGTAAGGATTCTGATGTGTATATCACAGACAAAACTGTGCTAG |
| | | | ACATGAGGTCTATGGACTTCAAGAGCAACAGTGCTGTGGCCTGGAG |
| | | | CAACAAATCTGACTTTGCATGTGCAAACGCCTTCAACAACAGCATTA |
| | | | TTCCAGAAGACACCTTCTTCCCCAGCCCAGAAAGTTCCTGTGATGTC |
| | | | AAGCTGGTCGAGAAAAGCTTTGAAACAGATACGAACCTAAACTTTCA |
| | | | AAACCTGTCAGTGATTGGGTTCCGAATCCTCCTCCTGAAAGTGGCC |
| | | | GGGTTTAATCTGCTCATGACGCTGCGGTTGTGGTCCAGCTGA |
| 27 | β VDJ | AA | MGSWTLCCVSLCILVAKHTDAGVIQSPRHEVTEMGQEVTLRCKPISGH |
| | and | | DYLFWYRQTMMRGLELLIYFNNNVPIDDSGMPEDRFSAKMPNASFSTL |
| | constant | | KIQPSEPRDSAVYFCASGTGWDTEAFFGQGTRLTVVEDLNKVFPPEVA |
| | | | VFEPSEAEISHTQKATLVCLATGFFPDHVELSWWVNGKEVHSGVSTDP |
| | | | QPLKEQPALNDSRYCLSSRLRVSATFWQNPRNHFRCQVQFYGLSEND |
| | | | EWTQDRAKPVTQIVSAEAWGRADCGFTSVSYQQGVLSATILYEILLGKA |
| | | | TLYAVLVSALVLMAMVKRKDF |
| 28 | β VDJ | NT | ATGGGCTCCTGGACCCTCTGCTGTGTGTCCCTTTGCATCCTGGTAG |
| | and | | CAAAGCACACAGATGCTGGAGTTATCCAGTCACCCCGGCACGAGGT |
| | constant | | GACAGAGATGGGACAAGAAGTGACTCTGAGATGTAAACCAATTTCAG |
| | | | GACACGACTACCTTTTCTGGTACAGACAGACCATGATGCGGGGACT |
| | | | GGAGTTGCTCATTTACTTTAACAACAACGTTCCGATAGATGATTCAG |
| | | | GGATGCCCGAGGATCGATTCTCAGCTAAGATGCCTAATGCATCATTC |
| | | | TCCACTCTGAAGATCCAGCCCTCAGAACCCAGGGACTCAGCTGTGT |
| | | | ACTTCTGTGCCAGCGGAACAGGGTGGGACACTGAAGCTTTCTTTGG |
| | | | ACAAGGCACCAGACTCACAGTTGTAGAGGACCTGAACAAGGTGTTC |
| | | | CCACCCGAGGTCGCTGTGTTTGAGCCATCAGAAGCAGAGATCTCCC |
| | | | ACACCCAAAAGGCCACACTGGTGTGCCTGGCCACAGGCTTCTTCCC |
| | | | CGACCACGTGGAGCTGAGCTGGTGGGTGAATGGGAAGGAGGTGCA |
| | | | CAGTGGGGTCAGCACAGACCCGCAGCCCCTCAAGGAGCAGCCCGC |
| | | | CCTCAATGACTCCAGATACTGCCTGAGCAGCCGCCTGAGGGTCTCG |
| | | | GCCACCTTCTGGCAGAACCCCCGCAACCACTTCCGCTGTCAAGTCC |
| | | | AGTTCTACGGGCTCTCGGAGAATGACGAGTGGACCCAGGATAGGGC |
| | | | CAAACCCGTCACCCAGATCGTCAGCGCCGAGGCCTGGGGTAGAGC |
| | | | AGACTGTGGCTTTACCTCGGTGTCCTACCAGCAAGGGGTCCTGTCT |
| | | | GCCACCATCCTCTATGAGATCCTGCTAGGGAAGGCCACCCTGTATG |
| | | | CTGTGCTGGTCAGCGCCCTTGTGTTGATGGCCATGGTCAAGAGAAA |
| | | | GGATTTCTGA |

TABLE 8
TCR CT43.2D8; target peptide YVGKEHMFY (SEQ ID NO 129); target gene MAGE-A9 A*01:01
Sequences for TCR CT43.2D8; target peptide YVGKEHMFY (SEQ ID NO 129);
target gene MAGE-A9 A*01:01

| | | AA | |
|---|---|---|---|
| SEQ ID | TCR | or | |
| NO | POLYPEPTIDE | NT | SEQUENCE |
| 29 | α CDR1 | AA | VTNFRS |
| 30 | α CDR2 | AA | LTSSGIE |
| 31 | α CDR3 | AA | CAVEGWDDKIIF |
| 32 | β CDR1 | AA | ENHRY |
| 33 | β CDR2 | AA | SYGVKD |
| 34 | β CDR3 | AA | CAISEGRNEQYF |
| 35 | α VJ | AA | MMKCPQALLAIFWLLLSWVSSEDKVVQSPLSLVVHEGDTVTLNCSYEV |
| | | | TNFRSLLWYKQEKKAPTFLFMLTSSGIEKKSGRLSSILDKKELSSILNITA |
| | | | TQTGDSAIYLCAVEGWDDKIIFGKGTRLHILP |
| 36 | α VJ | NT | ATGATGAAGTGTCCACAGGCTTTACTAGCTATCTTTTGGCTTCTACTG |
| | | | AGCTGGGTGAGCAGTGAAGACAAGGTGGTACAAAGCCCTCTATCTC |
| | | | TGGTTGTCCACGAGGGAGACACCGTAACTCTCAATTGCAGTTATGAA |
| | | | GTGACTAACTTTCGAAGCCTACTATGGTACAAGCAGGAAAAGAAAGC |
| | | | TCCCACATTTCTATTTATGCTAACTTCAAGTGGAATTGAAAAGAAGTC |
| | | | AGGAAGACTAAGTAGCATATTAGATAAGAAAGAACTTTCCAGCATCC |
| | | | TGAACATCACAGCCACCCAGACCGGAGACTCGGCCATCTACCTCTG |
| | | | TGCTGTGGAGGGATGGGATGACAAGATCATCTTTGGAAAAGGGACA |
| | | | CGACTTCATATTCTCCCC |
| 37 | β VDJ | AA | MGTRLFFYVALCLLWTGHMDAGITQSPRHKVTETGTPVTLRCHQTENH |
| | | | RYMYWYRQDPGHGLRLIHYSYGVKDTDKGEVSDGYSVSRSKTEDFLLT |
| | | | LESATSSQTSVYFCAISEGRNEQYFGPGTRLTVT |
| 38 | β VDJ | NT | ATGGGCACAAGGTTGTTCTTCTATGTGGCCCTTTGTCTCCTGTGGAC |
| | | | AGGACACATGGATGCTGGAATCACCCAGAGCCCAAGACACAAGGTC |
| | | | ACAGAGACAGGAACACCAGTGACTCTGAGATGTCATCAGACTGAGA |
| | | | ACCACCGCTATATGTACTGGTATCGACAAGACCCGGGGCATGGGCT |
| | | | GAGGCTGATCCATTACTCATATGGTGTTAAAGATACTGACAAAGGAG |
| | | | AAGTCTCAGATGGCTATAGTGTCTCTAGATCAAAGACAGAGGATTTC |
| | | | CTCCTCACTCTGGAGTCCGCTACCAGCTCCCAGACATCTGTGTACTT |
| | | | CTGTGCCATCAGTGAGGGCCGGAACGAGCAGTACTTCGGGCCGGG |
| | | | CACCAGGCTCACGGTCACA |
| 39 | α VJ | AA | MMKCPQALLAIFWLLLSWVSSEDKVVQSPLSLVVHEGDTVTLNCSYEV |
| | and | | TNFRSLLWYKQEKKAPTFLFMLTSSGIEKKSGRLSSILDKKELSSILNITA |
| | constant | | TQTGDSAIYLCAVEGWDDKIIFGKGTRLHILPNIQNPDPAVYQLRDSKSS |
| | | | DKSVCLFTDFDSQTNVSQSKDSDVYITDKTVLDMRSMDFKSNSAVAWS |
| | | | NKSDFACANAFNNSIIPEDTFFPSPESSCDVKLVEKSFETDTNLNFQNLS |
| | | | VIGFRILLLKVAGFNLLMTLRLWSS |
| 40 | α VJ | NT | ATGATGAAGTGTCCACAGGCTTTACTAGCTATCTTTTGGCTTCTACTG |
| | and | | AGCTGGGTGAGCAGTGAAGACAAGGTGGTACAAAGCCCTCTATCTC |
| | constant | | TGGTTGTCCACGAGGGAGACACCGTAACTCTCAATTGCAGTTATGAA |
| | | | GTGACTAACTTTCGAAGCCTACTATGGTACAAGCAGGAAAAGAAAGC |
| | | | TCCCACATTTCTATTTATGCTAACTTCAAGTGGAATTGAAAAGAAGTC |
| | | | AGGAAGACTAAGTAGCATATTAGATAAGAAAGAACTTTCCAGCATCC |
| | | | TGAACATCACAGCCACCCAGACCGGAGACTCGGCCATCTACCTCTG |
| | | | TGCTGTGGAGGGATGGGATGACAAGATCATCTTTGGAAAAGGGACA |
| | | | CGACTTCATATTCTCCCCAATATCCAGAACCCTGACCCTGCCGTGTA |
| | | | CCAGCTGAGAGACTCTAAATCCAGTGACAAGTCTGTCTGCCTATTCA |
| | | | CCGATTTTGATTCTCAAACAAATGTGTCACAAAGTAAGGATTCTGATG |
| | | | TGTATATCACAGACAAAACTGTGCTAGACATGAGGTCTATGGACTTC |
| | | | AAGAGCAACAGTGCTGTGGCCTGGAGCAACAAATCTGACTTTGCAT |
| | | | GTGCAAACGCCTTCAACAACAGCATTATTCCAGAAGACACCTTCTTC |
| | | | CCCAGCCCAGAAAGTTCCTGTGATGTCAAGCTGGTCGAGAAAAGCT |
| | | | TTGAAACAGATACGAACCTAAACTTTCAAAACCTGTCAGTGATTGGG |
| | | | TTCCGAATCCTCCTCCTGAAAGTGGCCGGGTTTAATCTGCTCATGAC |
| | | | GCTGCGGTTGTGGTCCAGCTGA |
| 41 | β VDJ | AA | MGTRLFFYVALCLLWTGHMDAGITQSPRHKVTETGTPVTLRCHQTENH |
| | and | | RYMYWYRQDPGHGLRLIHYSYGVKDTDKGEVSDGYSVSRSKTEDFLLT |
| | constant | | LESATSSQTSVYFCAISEGRNEQYFGPGTRLTVTEDLNKVFPPEVAVFE |
| | | | PSEAEISHTQKATLVCLATGFFPDHVELSWWWVNGKEVHSGVSTDPQPL |
| | | | KEQPALNDSRYCLSSRLRVSATFWQNPRNHFRCQVQFYGLSENDEWT |
| | | | QDRAKPVTQIVSAEAWGRADCGFTSVSYQQGVLSATILYEILLGKATLY |
| | | | AVLVSALVLMAMVKRKDF |
| 42 | β VDJ | NT | ATGGGCACAAGGTTGTTCTTCTATGTGGCCCTTTGTCTCCTGTGGAC |
| | and | | AGGACACATGGATGCTGGAATCACCCAGAGCCCAAGACACAAGGTC |
| | constant | | ACAGAGACAGGAACACCAGTGACTCTGAGATGTCATCAGACTGAGA |
| | | | ACCACCGCTATATGTACTGGTATCGACAAGACCCGGGGCATGGGCT |
| | | | GAGGCTGATCCATTACTCATATGGTGTTAAAGATACTGACAAAGGAG |
| | | | AAGTCTCAGATGGCTATAGTGTCTCTAGATCAAAGACAGAGGATTTC |
| | | | CTCCTCACTCTGGAGTCCGCTACCAGCTCCCAGACATCTGTGTACTT |
| | | | CTGTGCCATCAGTGAGGGCCGGAACGAGCAGTACTTCGGGCCGGG |
| | | | CACCAGGCTCACGGTCACAGAGGACCTGAACAAGGTGTTCCCACCC |
| | | | GAGGTCGCTGTGTTTGAGCCATCAGAAGCAGAGATCTCCCACACCC |
| | | | AAAAGGCCACACTGGTGTGCCTGGCCACAGGCTTCTTCCCCGACCA |
| | | | CGTGGAGCTGAGCTGGTGGGTGAATGGGAAGGAGGTGCACAGTGG |
| | | | GGTCAGCACAGACCCGCAGCCCCTCAAGGAGCAGCCCGCCCTCAA |
| | | | TGACTCCAGATACTGCCTGAGCAGCCGCCTGAGGGTCTCGGCCACC |
| | | | TTCTGGCAGAACCCCCGCAACCACTTCCGCTGTCAAGTCCAGTTCTA |
| | | | CGGGCTCTCGGAGAATGACGAGTGGACCCAGGATAGGGCCAAACC |
| | | | CGTCACCCAGATCGTCAGCGCCGAGGCCTGGGGTAGAGCAGACTG |
| | | | TGGCTTTACCTCGGTGTCCTACCAGCAAGGGGTCCTGTCTGCCACC |
| | | | ATCCTCTATGAGATCCTGCTAGGGAAGGCCACCCTGTATGCTGTGCT |
| | | | GGTCAGCGCCCTTGTGTTGATGGCCATGGTCAAGAGAAAGGATTTC |
| | | | TGA |

TABLE 9
TCR CT44.6G4; target peptide LTQDLVQEKYLEY (SEQ ID NO 130); target gene MAGE-A1
A*01:01
Sequences for TCR CT44.6G4; target peptide LTQDLVQEKYLEY (SEQ ID NO: 130);
target gene MAGE-A1 A*01:01.

| | | AA | |
|---|---|---|---|
| SEQ ID | TCR | or | |
| NO | POLYPEPTIDE | NT | SEQUENCE |
| 43 | α CDR1 | AA | DSAIYN |
| 44 | α CDR2 | AA | IQSSQRE |
| 45 | α CDR3 | AA | CAVLNQAGTALIF |
| 46 | β CDR1 | AA | SGDLS |
| 47 | β CDR2 | AA | YYNGEE |
| 48 | β CDR3 | AA | CASSVWSVGSTEAFF |
| 49 | α VJ | AA | METLLGLLILWLQLQWVSSKQEVTQIPAALSVPEGENLVLNCSFTDSAIY |
| | | | NLQWFRQDPGKGLTSLLLIQSSQREQTSGRLNASLDKSSGRSTLYIAAS |
| | | | QPGDSATYLCAVLNQAGTALIFGKGTTLSVSS |
| 50 | α VJ | NT | ATGGAGACCCTCTTGGGCCTGCTTATCCTTTGGCTGCAGCTGCAAT |
| | | | GGGTGAGCAGCAAACAGGAGGTGACACAGATTCCTGCAGCTCTGAG |
| | | | TGTCCCAGAAGGAGAAAACTTGGTTCTCAACTGCAGTTTCACTGATA |
| | | | GCGCTATTTACAACCTCCAGTGGTTTAGGCAGGACCCTGGGAAAGG |
| | | | TCTCACATCTCTGTTGCTTATTCAGTCAAGTCAGAGAGAGCAAACAA |
| | | | GTGGAAGACTTAATGCCTCGCTGGATAAATCATCAGGACGTAGTACT |
| | | | TTATACATTGCAGCTTCTCAGCCTGGTGACTCAGCCACCTACCTCTG |
| | | | TGCTGTCCTAAACCAGGCAGGAACTGCTCTGATCTTTGGGAAGGGA |
| | | | ACCACCTTATCAGTGAGTTCC |
| 51 | β VDJ | AA | MGFRLLCCVAFCLLGAGPVDSGVTQTPKHLITATGQRVTLRCSPRSGD |
| | | | LSVYWYQQSLDQGLQFLIQYYNGEERAKGNILERFSAQQFPDLHSELNL |
| | | | SSLELGDSALYFCASSVWSVGSTEAFFGQGTRLTVV |
| 52 | β VDJ | NT | ATGGGCTTCAGGCTCCTCTGCTGTGTGGCCTTTTGTCTCCTGGGAG |
| | | | CAGGCCCAGTGGATTCTGGAGTCACACAAACCCCAAAGCACCTGAT |
| | | | CACAGCAACTGGACAGCGAGTGACGCTGAGATGCTCCCCTAGGTCT |
| | | | GGAGACCTCTCTGTGTACTGGTACCAACAGAGCCTGGACCAGGGCC |
| | | | TCCAGTTCCTCATTCAGTATTATAATGGAGAAGAGAGAGCAAAAGGA |
| | | | AACATTCTTGAACGATTCTCCGCACAACAGTTCCCTGACTTGCACTC |
| | | | TGAACTAAACCTGAGCTCTCTGGAGCTGGGGGACTCAGCTTTGTATT |
| | | | TCTGTGCCAGCAGCGTATGGTCAGTCGGGAGCACTGAAGCTTTCTT |
| | | | TGGACAAGGCACCAGACTCACAGTTGTA |
| 53 | α VJ | AA | METLLGLLILWLQLQWVSSKQEVTQIPAALSVPEGENLVLNCSFTDSAIY |
| | and | | NLQWFRQDPGKGLTSLLLIQSSQREQTSGRLNASLDKSSGRSTLYIAAS |
| | constant | | QPGDSATYLCAVLNQAGTALIFGKGTTLSVSSNIQNPDPAVYQLRDSKS |
| | | | SDKSVCLFTDFDSQTNVSQSKDSDVYITDKTVLDMRSMDFKSNSAVAW |
| | | | SNKSDFACANAFNNSIIPEDTFFPSPESSCDVKLVEKSFETDTNLNFQNL |
| | | | SVIGFRILLLKVAGFNLLMTLRLWSS |
| 54 | α VJ | NT | ATGGAGACCCTCTTGGGCCTGCTTATCCTTTGGCTGCAGCTGCAAT |
| | and | | GGGTGAGCAGCAAACAGGAGGTGACACAGATTCCTGCAGCTCTGAG |
| | constant | | TGTCCCAGAAGGAGAAAACTTGGTTCTCAACTGCAGTTTCACTGATA |
| | | | GCGCTATTTACAACCTCCAGTGGTTTAGGCAGGACCCTGGGAAAGG |
| | | | TCTCACATCTCTGTTGCTTATTCAGTCAAGTCAGAGAGAGCAAACAA |
| | | | GTGGAAGACTTAATGCCTCGCTGGATAAATCATCAGGACGTAGTACT |
| | | | TTATACATTGCAGCTTCTCAGCCTGGTGACTCAGCCACCTACCTCTG |
| | | | TGCTGTCCTAAACCAGGCAGGAACTGCTCTGATCTTTGGGAAGGGA |
| | | | ACCACCTTATCAGTGAGTTCCAATATCCAGAACCCTGACCCTGCCGT |
| | | | GTACCAGCTGAGAGACTCTAAATCCAGTGACAAGTCTGTCTGCCTAT |
| | | | TCACCGATTTTGATTCTCAAACAAATGTGTCACAAAGTAAGGATTCTG |
| | | | ATGTGTATATCACAGACAAAACTGTGCTAGACATGAGGTCTATGGAC |
| | | | TTCAAGAGCAACAGTGCTGTGGCCTGGAGCAACAAATCTGACTTTGC |
| | | | ATGTGCAAACGCCTTCAACAACAGCATTATTCCAGAAGACACCTTCT |
| | | | TCCCCAGCCCAGAAAGTTCCTGTGATGTCAAGCTGGTCGAGAAAAG |
| | | | CTTTGAAACAGATACGAACCTAAACTTTCAAAACCTGTCAGTGATTG |
| | | | GGTTCCGAATCCTCCTCCTGAAAGTGGCCGGGTTTAATCTGCTCATG |
| | | | ACGCTGCGGTTGTGGTCCAGCTGA |
| 55 | β VDJ | AA | MGFRLLCCVAFCLLGAGPVDSGVTQTPKHLITATGQRVTLRCSPRSGD |
| | and | | LSVYWYQQSLDQGLQFLIQYYNGEERAKGNILERFSAQQFPDLHSELNL |
| | constant | | SSLELGDSALYFCASSVWSVGSTEAFFGQGTRLTVVEDLNKVFPPEVA |
| | | | VFEPSEAEISHTQKATLVCLATGFFPDHVELSWWVNGKEVHSGVSTDP |
| | | | QPLKEQPALNDSRYCLSSRLRVSATFWQNPRNHFRCQVQFYGLSEND |
| | | | EWTQDRAKPVTQIVSAEAWGRADCGFTSVSYQQGVLSATILYEILLGKA |
| | | | TLYAVLVSALVLMAMVKRKDF |
| 56 | β VDJ | NT | ATGGGCTTCAGGCTCCTCTGCTGTGTGGCCTTTTGTCTCCTGGGAG |
| | and | | CAGGCCCAGTGGATTCTGGAGTCACACAAACCCCAAAGCACCTGAT |
| | constant | | CACAGCAACTGGACAGCGAGTGACGCTGAGATGCTCCCCTAGGTCT |
| | | | GGAGACCTCTCTGTGTACTGGTACCAACAGAGCCTGGACCAGGGCC |
| | | | TCCAGTTCCTCATTCAGTATTATAATGGAGAAGAGAGAGCAAAAGGA |
| | | | AACATTCTTGAACGATTCTCCGCACAACAGTTCCCTGACTTGCACTC |
| | | | TGAACTAAACCTGAGCTCTCTGGAGCTGGGGGACTCAGCTTTGTATT |
| | | | TCTGTGCCAGCAGCGTATGGTCAGTCGGGAGCACTGAAGCTTTCTT |
| | | | TGGACAAGGCACCAGACTCACAGTTGTAGAGGACCTGAACAAGGTG |
| | | | TTCCCACCCGAGGTCGCTGTGTTTGAGCCATCAGAAGCAGAGATCT |
| | | | CCCACACCCAAAAGGCCACACTGGTGTGCCTGGCCACAGGCTTCTT |
| | | | CCCCGACCACGTGGAGCTGAGCTGGTGGGTGAATGGGAAGGAGGT |
| | | | GCACAGTGGGGTCAGCACAGACCCGCAGCCCCTCAAGGAGCAGCC |
| | | | CGCCCTCAATGACTCCAGATACTGCCTGAGCAGCCGCCTGAGGGTC |
| | | | TCGGCCACCTTCTGGCAGAACCCCCGCAACCACTTCCGCTGTCAAG |
| | | | TCCAGTTCTACGGGCTCTCGGAGAATGACGAGTGGACCCAGGATAG |
| | | | GGCCAAACCCGTCACCCAGATCGTCAGCGCCGAGGCCTGGGGTAG |
| | | | AGCAGACTGTGGCTTTACCTCGGTGTCCTACCAGCAAGGGGTCCTG |
| | | | TCTGCCACCATCCTCTATGAGATCCTGCTAGGGAAGGCCACCCTGT |
| | | | ATGCTGTGCTGGTCAGCGCCCTTGTGTTGATGGCCATGGTCAAGAG |
| | | | AAAGGATTTCTGA |

TABLE 10
TCR CT23.3H4; target peptide SLFRAVITK (SEQ ID NO 131); target gene MAGE-A1 A*03:01
Sequences for TCR CT23.3H4; target peptide SLFRAVITK (SEQ ID NO 131);
target gene MAGE-A1 A*03:01.

| | | AA | |
|---|---|---|---|
| SEQ ID | TCR | or | |
| NO | POLYPEPTIDE | NT | SEQUENCE |
| 57 | α CDR1 | AA | NSASQS |
| 58 | α CDR2 | AA | VYSSGN |
| 59 | α CDR3 | AA | CVVNFNYGQNFVF |
| 60 | β CDR1 | AA | SEHNRL |
| 61 | β CDR2 | AA | FQNEAQ |
| 62 | β CDR3 | AA | CASSPLGVNTEAFF |
| 63 | α VJ | AA | MKSLRVLLVILWLQLSWVWSQRKEVEQDPGPFNVPEGATVAFNCTYSN |
| | | | SASQSFFWYRQDCRKEPKLLMSVYSSGNEDGRFTAQLNRASQYISLLI |
| | | | RDSKLSDSATYLCVVNFNYGQNFVFGPGTRLSVLP |
| 64 | α VJ | NT | ATGAAATCCTTGAGAGTTTTACTAGTGATCCTGTGGCTTCAGTTGAG |
| | | | CTGGGTTTGGAGCCAACGGAAGGAGGTGGAGCAGGATCCTGGACC |
| | | | CTTCAATGTTCCAGAGGGAGCCACTGTCGCTTTCAACTGTACTTACA |
| | | | GCAACAGTGCTTCTCAGTCTTTCTTCTGGTACAGACAGGATTGCAGG |
| | | | AAAGAACCTAAGTTGCTGATGTCCGTATACTCCAGTGGTAATGAAGA |
| | | | TGGAAGGTTTACAGCACAGCTCAATAGAGCCAGCCAGTATATTTCCC |
| | | | TGCTCATCAGAGACTCCAAGCTCAGTGATTCAGCCACCTACCTCTGT |
| | | | GTGGTGAATTTTAACTATGGTCAGAATTTTGTCTTTGGTCCCGGAAC |
| | | | CAGATTGTCCGTGCTGCCC |
| 65 | β VDJ | AA | MGTSLLCWMALCLLGADHADTGVSQDPRHKITKRGQNVTFRCDPISEH |
| | | | NRLYWYRQTLGQGPEFLTYFQNEAQLEKSRLLSDRFSAERPKGSFSTL |
| | | | EIQRTEQGDSAMYLCASSPLGVNTEAFFGQGTRLTVV |
| 66 | β VDJ | NT | ATGGGCACCAGCCTCCTCTGCTGGATGGCCCTGTGTCTCCTGGGGG |
| | | | CAGATCACGCAGATACTGGAGTCTCCCAGGACCCCAGACACAAGAT |
| | | | CACAAAGAGGGGACAGAATGTAACTTTCAGGTGTGATCCAATTTCTG |
| | | | AACACAACCGCCTTTATTGGTACCGACAGACCCTGGGGCAGGGCCC |
| | | | AGAGTTTCTGACTTACTTCCAGAATGAAGCTCAACTAGAAAAATCAA |
| | | | GGCTGCTCAGTGATCGGTTCTCTGCAGAGAGGCCTAAGGGATOTTT |
| | | | CTCCACCTTGGAGATCCAGCGCACAGAGCAGGGGGACTCGGCCAT |
| | | | GTATCTCTGTGCCAGCAGCCCCCTGGGGGTGAACACTGAAGCTTTC |
| | | | TTTGGACAAGGCACCAGACTCACAGTTGTA |
| 67 | α VJ | AA | MKSLRVLLVILWLQLSWVWSQRKEVEQDPGPFNVPEGATVAFNCTYSN |
| | and | | SASQSFFWYRQDCRKEPKLLMSVYSSGNEDGRFTAQLNRASQYISLLI |
| | constant | | RDSKLSDSATYLCVVNFNYGQNFVFGPGTRLSVLPYIQNPDPAVYQLR |
| | | | DSKSSDKSVCLFTDFDSQTNVSQSKDSDVYITDKTVLDMRSMDFKSNS |
| | | | AVAWSNKSDFACANAFNNSIIPEDTFFPSPESSCDVKLVEKSFETDTNL |
| | | | NFQNLSVIGFRILLLKVAGFNLLMTLRLWSS |
| 68 | α VJ | NT | ATGAAATCCTTGAGAGTTTTACTAGTGATCCTGTGGCTTCAGTTGAG |
| | and | | CTGGGTTTGGAGCCAACGGAAGGAGGTGGAGCAGGATCCTGGACC |
| | constant | | CTTCAATGTTCCAGAGGGAGCCACTGTCGCTTTCAACTGTACTTACA |
| | | | GCAACAGTGCTTCTCAGTCTTTCTTCTGGTACAGACAGGATTGCAGG |
| | | | AAAGAACCTAAGTTGCTGATGTCCGTATACTCCAGTGGTAATGAAGA |
| | | | TGGAAGGTTTACAGCACAGCTCAATAGAGCCAGCCAGTATATTTCCC |
| | | | TGCTCATCAGAGACTCCAAGCTCAGTGATTCAGCCACCTACCTCTGT |
| | | | GTGGTGAATTTTAACTATGGTCAGAATTTTGTCTTTGGTCCCGGAAC |
| | | | CAGATTGTCCGTGCTGCCCTATATCCAGAACCCTGACCCTGCCGTG |
| | | | TACCAGCTGAGAGACTCTAAATCCAGTGACAAGTCTGTCTGCCTATT |
| | | | CACCGATTTTGATTCTCAAACAAATGTGTCACAAAGTAAGGATTCTGA |
| | | | TGTGTATATCACAGACAAAACTGTGCTAGACATGAGGTCTATGGACT |
| | | | TCAAGAGCAACAGTGCTGTGGCCTGGAGCAACAAATCTGACTTTGC |
| | | | ATGTGCAAACGCCTTCAACAACAGCATTATTCCAGAAGACACCTTCT |
| | | | TCCCCAGCCCAGAAAGTTCCTGTGATGTCAAGCTGGTCGAGAAAAG |
| | | | CTTTGAAACAGATACGAACCTAAACTTTCAAAACCTGTCAGTGATTG |
| | | | GGTTCCGAATCCTCCTCCTGAAAGTGGCCGGGTTTAATCTGCTCATG |
| | | | ACGCTGCGGTTGTGGTCCAGCTGA |
| 69 | β VDJ | AA | MGTSLLCWMALCLLGADHADTGVSQDPRHKITKRGQNVTFRCDPISEH |
| | and | | NRLYWYRQTLGQGPEFLTYFQNEAQLEKSRLLSDRFSAERPKGSFSTL |
| | constant | | EIQRTEQGDSAMYLCASSPLGVNTEAFFGQGTRLTVVEDLNKVFPPEV |
| | | | AVFEPSEAEISHTQKATLVCLATGFFPDHVELSWWWNGKEVHSGVSTD |
| | | | PQPLKEQPALNDSRYCLSSRLRVSATFWQNPRNHFRCQVQFYGLSEN |
| | | | DEWTQDRAKPVTQIVSAEAWGRADCGFTSVSYQQGVLSATILYEILLGK |
| | | | ATLYAVLVSALVLMAMVKRKDF |
| 70 | β VDJ | NT | ATGGGCACCAGCCTCCTCTGCTGGATGGCCCTGTGTCTCCTGGGGG |
| | and | | CAGATCACGCAGATACTGGAGTCTCCCAGGACCCCAGACACAAGAT |
| | constant | | CACAAAGAGGGGACAGAATGTAACTTTCAGGTGTGATCCAATTTCTG |
| | | | AACACAACCGCCTTTATTGGTACCGACAGACCCTGGGGCAGGGCCC |
| | | | AGAGTTTCTGACTTACTTCCAGAATGAAGCTCAACTAGAAAAATCAA |
| | | | GGCTGCTCAGTGATCGGTTCTCTGCAGAGAGGCCTAAGGGATOTTT |
| | | | CTCCACCTTGGAGATCCAGCGCACAGAGCAGGGGGACTCGGCCAT |
| | | | GTATCTCTGTGCCAGCAGCCCCCTGGGGGTGAACACTGAAGCTTTC |
| | | | TTTGGACAAGGCACCAGACTCACAGTTGTAGAGGACCTGAACAAGG |
| | | | TGTTCCCACCCGAGGTCGCTGTGTTTGAGCCATCAGAAGCAGAGAT |
| | | | CTCCCACACCCAAAAGGCCACACTGGTGTGCCTGGCCACAGGCTTC |
| | | | TTCCCCGACCACGTGGAGCTGAGCTGGTGGGTGAATGGGAAGGAG |
| | | | GTGCACAGTGGGGTCAGCACAGACCCGCAGCCCCTCAAGGAGCAG |
| | | | CCCGCCCTCAATGACTCCAGATACTGCCTGAGCAGCCGCCTGAGGG |
| | | | TCTCGGCCACCTTCTGGCAGAACCCCCGCAACCACTTCCGCTGTCA |
| | | | AGTCCAGTTCTACGGGCTCTCGGAGAATGACGAGTGGACCCAGGAT |
| | | | AGGGCCAAACCCGTCACCCAGATCGTCAGCGCCGAGGCCTGGGGT |
| | | | AGAGCAGACTGTGGCTTTACCTCGGTGTCCTACCAGCAAGGGGTCC |
| | | | TGTCTGCCACCATCCTCTATGAGATCCTGCTAGGGAAGGCCACCCT |
| | | | GTATGCTGTGCTGGTCAGCGCCCTTGTGTTGATGGCCATGGTCAAG |
| | | | AGAAAGGATTTCTGA |

TABLE 11
TCR MRM23.3B2; target peptide SLFRAVITK (SEQ ID NO 131); target gene MAGE-
A1 A*03:01
Sequences for TCR MRM23.3B2; target peptide SLFRAVITK (SEQ ID NO 131);
target gene MAGE-A1 A*03:01.

| | | AA | |
|---|---|---|---|
| SEQ ID | TCR | or | |
| NO | POLYPEPTIDE | NT | SEQUENCE |
| 71 | α CDR1 | AA | VSNAYN |
| 72 | α CDR2 | AA | GSKP |
| 73 | α CDR3 | AA | CAVVHNYGQNFVF |
| 74 | β CDR1 | AA | SEHNRL |
| 75 | β CDR2 | AA | FQNEAQ |
| 76 | β CDR3 | AA | CASSPLASDTGELFF |
| 77 | α VJ | AA | MALQSTLGAVWLGLLLNSLWKVAESKDQVFQPSTVASSEGAVVEIFCN |
| | | | HSVSNAYNFFWYLHFPGCAPRLLVKGSKPSQQGRYNMTYERFSSSLLI |
| | | | LQVREADAAVYYCAVVHNYGQNFVFGPGTRLSVLP |
| 78 | α VJ | NT | ATGGCTTTGCAGAGCACTCTGGGGGCGGTGTGGCTAGGGCTTCTCC |
| | | | TCAACTCTCTCTGGAAGGTTGCAGAAAGCAAGGACCAAGTGTTTCAG |
| | | | CCTTCCACAGTGGCATCTTCAGAGGGAGCTGTGGTGGAAATCTTCT |
| | | | GTAATCACTCTGTGTCCAATGCTTACAACTTCTTCTGGTACCTTCACT |
| | | | TCCCGGGATGTGCACCAAGACTCCTTGTTAAAGGCTCAAAGCCTTCT |
| | | | CAGCAGGGACGATACAACATGACCTATGAACGGTTCTCTTCATCGCT |
| | | | GCTCATCCTCCAGGTGCGGGAGGCAGATGCTGCTGTTTACTACTGT |
| | | | GCTGTGGTTCATAACTATGGTCAGAATTTTGTCTTTGGTCCCGGAAC |
| | | | CAGATTGTCCGTGCTGCCC |
| 79 | β VDJ | AA | MGTSLLCWMALCLLGADHADTGVSQDPRHKITKRGQNVTFRCDPISEH |
| | | | NRLYWYRQTLGQGPEFLTYFQNEAQLEKSRLLSDRFSAERPKGSFSTL |
| | | | EIQRTEQGDSAMYLCASSPLASDTGELFFGEGSRLTVL |
| 80 | β VDJ | NT | ATGGGCACCAGCCTCCTCTGCTGGATGGCCCTGTGTCTCCTGGGGG |
| | | | CAGATCACGCAGATACTGGAGTCTCCCAGGACCCCAGACACAAGAT |
| | | | CACAAAGAGGGGACAGAATGTAACTTTCAGGTGTGATCCAATTTCTG |
| | | | AACACAACCGCCTTTATTGGTACCGACAGACCCTGGGGCAGGGCCC |
| | | | AGAGTTTCTGACTTACTTCCAGAATGAAGCTCAACTAGAAAAATCAA |
| | | | GGCTGCTCAGTGATCGGTTCTCTGCAGAGAGGCCTAAGGGATCTTT |
| | | | CTCCACCTTGGAGATCCAGCGCACAGAGCAGGGGGACTCGGCCAT |
| | | | GTATCTCTGTGCCAGCAGCCCCTTAGCCAGTGACACCGGGGAGCTG |
| | | | TTTTTTGGAGAAGGCTCTAGGCTGACCGTACTG |
| 81 | α VJ | AA | MALQSTLGAVWLGLLLNSLWKVAESKDQVFQPSTVASSEGAVVEIFCN |
| | and | | HSVSNAYNFFWYLHFPGCAPRLLVKGSKPSQQGRYNMTYERFSSSLLI |
| | constant | | LQVREADAAVYYCAVVHNYGQNFVFGPGTRLSVLPYIQNPDPAVYQLR |
| | | | DSKSSDKSVCLFTDFDSQTNVSQSKDSDVYITDKTVLDMRSMDFKSNS |
| | | | AVAWSNKSDFACANAFNNSIIPEDTFFPSPESSCDVKLVEKSFETDTNL |
| | | | NFQNLSVIGFRILLLKVAGFNLLMTLRLWSS |
| 82 | α VJ | NT | ATGGCTTTGCAGAGCACTCTGGGGGCGGTGTGGCTAGGGCTTCTCC |
| | and | | TCAACTCTCTCTGGAAGGTTGCAGAAAGCAAGGACCAAGTGTTTCAG |
| | constant | | CCTTCCACAGTGGCATCTTCAGAGGGAGCTGTGGTGGAAATCTTCT |
| | | | GTAATCACTCTGTGTCCAATGCTTACAACTTCTTCTGGTACCTTCACT |
| | | | TCCCGGGATGTGCACCAAGACTCCTTGTTAAAGGCTCAAAGCCTTCT |
| | | | CAGCAGGGACGATACAACATGACCTATGAACGGTTCTCTTCATCGCT |
| | | | GCTCATCCTCCAGGTGCGGGAGGCAGATGCTGCTGTTTACTACTGT |
| | | | GCTGTGGTTCATAACTATGGTCAGAATTTTGTCTTTGGTCCCGGAAC |
| | | | CAGATTGTCCGTGCTGCCCTATATCCAGAACCCTGACCCTGCCGTG |
| | | | TACCAGCTGAGAGACTCTAAATCCAGTGACAAGTCTGTCTGCCTATT |
| | | | CACCGATTTTGATTCTCAAACAAATGTGTCACAAAGTAAGGATTCTGA |
| | | | TGTGTATATCACAGACAAAACTGTGCTAGACATGAGGTCTATGGACT |
| | | | TCAAGAGCAACAGTGCTGTGGCCTGGAGCAACAAATCTGACTTTGC |
| | | | ATGTGCAAACGCCTTCAACAACAGCATTATTCCAGAAGACACCTTCT |
| | | | TCCCCAGCCCAGAAAGTTCCTGTGATGTCAAGCTGGTCGAGAAAAG |
| | | | CTTTGAAACAGATACGAACCTAAACTTTCAAAACCTGTCAGTGATTG |
| | | | GGTTCCGAATCCTCCTCCTGAAAGTGGCCGGGTTTAATCTGCTCATG |
| | | | ACGCTGCGGTTGTGGTCCAGCTGA |
| 83 | β VDJ | AA | MGTSLLCWMALCLLGADHADTGVSQDPRHKITKRGQNVTFRCDPISEH |
| | and | | NRLYWYRQTLGQGPEFLTYFQNEAQLEKSRLLSDRFSAERPKGSFSTL |
| | constant | | EIQRTEQGDSAMYLCASSPLASDTGELFFGEGSRLTVLEDLNKVFPPEV |
| | | | AVFEPSEAEISHTQKATLVCLATGFFPDHVELSWWWVNGKEVHSGVSTD |
| | | | PQPLKEQPALNDSRYCLSSRLRVSATFWQNPRNHFRCQVQFYGLSEN |
| | | | DEWTQDRAKPVTQIVSAEAWGRADCGFTSVSYQQGVLSATILYEILLGK |
| | | | ATLYAVLVSALVLMAMVKRKDF |
| 84 | β VDJ | NT | ATGGGCACCAGCCTCCTCTGCTGGATGGCCCTGTGTCTCCTGGGGG |
| | and | | CAGATCACGCAGATACTGGAGTCTCCCAGGACCCCAGACACAAGAT |
| | constant | | CACAAAGAGGGGACAGAATGTAACTTTCAGGTGTGATCCAATTTCTG |
| | | | AACACAACCGCCTTTATTGGTACCGACAGACCCTGGGGCAGGGCCC |
| | | | AGAGTTTCTGACTTACTTCCAGAATGAAGCTCAACTAGAAAAATCAA |
| | | | GGCTGCTCAGTGATCGGTTCTCTGCAGAGAGGCCTAAGGGATOTTT |
| | | | CTCCACCTTGGAGATCCAGCGCACAGAGCAGGGGGACTCGGCCAT |
| | | | GTATCTCTGTGCCAGCAGCCCCTTAGCCAGTGACACCGGGGAGCTG |
| | | | TTTTTTGGAGAAGGCTCTAGGCTGACCGTACTGGAGGACCTGAACA |
| | | | AGGTGTTCCCACCCGAGGTCGCTGTGTTTGAGCCATCAGAAGCAGA |
| | | | GATCTCCCACACCCAAAAGGCCACACTGGTGTGCCTGGCCACAGGC |
| | | | TTCTTCCCCGACCACGTGGAGCTGAGCTGGTGGGTGAATGGGAAGG |
| | | | AGGTGCACAGTGGGGTCAGCACAGACCCGCAGCCCCTCAAGGAGC |
| | | | AGCCCGCCCTCAATGACTCCAGATACTGCCTGAGCAGCCGCCTGAG |
| | | | GGTCTCGGCCACCTTCTGGCAGAACCCCCGCAACCACTTCCGCTGT |
| | | | CAAGTCCAGTTCTACGGGCTCTCGGAGAATGACGAGTGGACCCAGG |
| | | | ATAGGGCCAAACCCGTCACCCAGATCGTCAGCGCCGAGGCCTGGG |
| | | | GTAGAGCAGACTGTGGCTTTACCTCGGTGTCCTACCAGCAAGGGGT |
| | | | CCTGTCTGCCACCATCCTCTATGAGATCCTGCTAGGGAAGGCCACC |
| | | | CTGTATGCTGTGCTGGTCAGCGCCCTTGTGTTGATGGCCATGGTCA |
| | | | AGAGAAAGGATTTCTGA |

TABLE 12
TCR CT12.3G2; target peptide RVRFFFPSL (SEQ ID NO 132); target gene MAGE-A1
B*07:02
Sequences for TCR CT12.3G2; target peptide RVRFFFPSL (SEQ ID NO 132);
target gene MAGE-A1 B*07:02

| | | AA | |
|---|---|---|---|
| SEQ ID | TCR | or | |
| NO | POLYPEPTIDE | NT | SEQUENCE |
| 85 | α CDR1 | AA | TRDTTYY |
| 86 | α CDR2 | AA | RNSFDEQN |
| 87 | α CDR3 | AA | CALSEAYSGAGSYQLTF |
| 88 | β CDR1 | AA | SNHLY |
| 89 | β CDR2 | AA | FYNNEI |
| 90 | β CDR3 | AA | CASSEALQPQHF |
| 91 | α VJ | AA | MLTASLLRAVIASICVVSSMAQKVTQAQTEISVVEKEDVTLDCVYETRDT |
| | | | TYYLFWYKQPPSGELVFLIRRNSFDEQNEISGRYSWNFQKSTSSFNFTI |
| | | | TASQVVDSAVYFCALSEAYSGAGSYQLTFGKGTKLSVIP |
| 92 | α VJ | NT | ATGCTGACTGCCAGCCTGTTGAGGGCAGTCATAGCCTCCATCTGTG |
| | | | TTGTATCCAGCATGGCTCAGAAGGTAACTCAAGCGCAGACTGAAATT |
| | | | TCTGTGGTGGAGAAGGAGGATGTGACCTTGGACTGTGTGTATGAAA |
| | | | CCCGTGATACTACTTATTACTTATTCTGGTACAAGCAACCACCAAGT |
| | | | GGAGAATTGGTTTTCCTTATTCGTCGGAACTCTTTTGATGAGCAAAAT |
| | | | GAAATAAGTGGTCGGTATTCTTGGAACTTCCAGAAATCCACCAGTTC |
| | | | CTTCAACTTCACCATCACAGCCTCACAAGTCGTGGACTCAGCAGTAT |
| | | | ACTTCTGTGCTCTGAGTGAGGCATACTCTGGGGCTGGGAGTTACCA |
| | | | ACTCACTTTCGGGAAGGGGACCAAACTCTCGGTCATACCA |
| 93 | β VDJ | AA | MDTWLVCWAIFSLLKAGLTEPEVTQTPSHQVTQMGQEVILRCVPISNHL |
| | | | YFYWYRQILGQKVEFLVSFYNNEISEKSEIFDDQFSVERPDGSNFTLKIR |
| | | | STKLEDSAMYFCASSEALQPQHFGDGTRLSIL |
| 94 | β VDJ | NT | ATGGATACCTGGCTCGTATGCTGGGCAATTTTTAGTCTCTTGAAAGC |
| | | | AGGACTCACAGAACCTGAAGTCACCCAGACTCCCAGCCATCAGGTC |
| | | | ACACAGATGGGACAGGAAGTGATCTTGCGCTGTGTCCCCATCTCTA |
| | | | ATCACTTATACTTCTATTGGTACAGACAAATCTTGGGGCAGAAAGTC |
| | | | GAGTTTCTGGTTTCCTTTTATAATAATGAAATCTCAGAGAAGTCTGAA |
| | | | ATATTCGATGATCAATTCTCAGTTGAAAGGCCTGATGGATCAAATTTC |
| | | | ACTCTGAAGATCCGGTCCACAAAGCTGGAGGACTCAGCCATGTACT |
| | | | TCTGTGCCAGCAGTGAAGCTCTGCAGCCCCAGCATTTTGGTGATGG |
| | | | GACTCGACTCTCCATCCTA |
| 95 | α VJ | AA | MLTASLLRAVIASICVVSSMAQKVTQAQTEISVVEKEDVTLDCVYETRDT |
| | and | | TYYLFWYKQPPSGELVFLIRRNSFDEQNEISGRYSWNFQKSTSSFNFTI |
| | constant | | TASQVVDSAVYFCALSEAYSGAGSYQLTFGKGTKLSVIPNIQNPDPAVY |
| | | | QLRDSKSSDKSVCLFTDFDSQTNVSQSKDSDVYITDKTVLDMRSMDFK |
| | | | SNSAVAWSNKSDFACANAFNNSIIPEDTFFPSPESSCDVKLVEKSFETD |
| | | | TNLNFQNLSVIGFRILLLKVAGFNLLMTLRLWSS |
| 96 | α VJ | NT | ATGCTGACTGCCAGCCTGTTGAGGGCAGTCATAGCCTCCATCTGTG |
| | and | | TTGTATCCAGCATGGCTCAGAAGGTAACTCAAGCGCAGACTGAAATT |
| | constant | | TCTGTGGTGGAGAAGGAGGATGTGACCTTGGACTGTGTGTATGAAA |
| | | | CCCGTGATACTACTTATTACTTATTCTGGTACAAGCAACCACCAAGT |
| | | | GGAGAATTGGTTTTCCTTATTCGTCGGAACTCTTTTGATGAGCAAAAT |
| | | | GAAATAAGTGGTCGGTATTCTTGGAACTTCCAGAAATCCACCAGTTC |
| | | | CTTCAACTTCACCATCACAGCCTCACAAGTCGTGGACTCAGCAGTAT |
| | | | ACTTCTGTGCTCTGAGTGAGGCATACTCTGGGGCTGGGAGTTACCA |
| | | | ACTCACTTTCGGGAAGGGGACCAAACTCTCGGTCATACCAAATATCC |
| | | | AGAACCCTGACCCTGCCGTGTACCAGCTGAGAGACTCTAAATCCAG |
| | | | TGACAAGTCTGTCTGCCTATTCACCGATTTTGATTCTCAAACAAATGT |
| | | | GTCACAAAGTAAGGATTCTGATGTGTATATCACAGACAAAACTGTGC |
| | | | TAGACATGAGGTCTATGGACTTCAAGAGCAACAGTGCTGTGGCCTG |
| | | | GAGCAACAAATCTGACTTTGCATGTGCAAACGCCTTCAACAACAGCA |
| | | | TTATTCCAGAAGACACCTTCTTCCCCAGCCCAGAAAGTTCCTGTGAT |
| | | | GTCAAGCTGGTCGAGAAAAGCTTTGAAACAGATACGAACCTAAACTT |
| | | | TCAAAACCTGTCAGTGATTGGGTTCCGAATCCTCCTCCTGAAAGTGG |
| | | | CCGGGTTTAATCTGCTCATGACGCTGCGGTTGTGGTCCAGCTGA |
| 97 | β VDJ | AA | MDTWLVCWAIFSLLKAGLTEPEVTQTPSHQVTQMGQEVILRCVPISNHL |
| | and | | YFYWYRQILGQKVEFLVSFYNNEISEKSEIFDDQFSVERPDGSNFTLKIR |
| | constant | | STKLEDSAMYFCASSEALQPQHFGDGTRLSILEDLNKVFPPEVAVFEPS |
| | | | EAEISHTQKATLVCLATGFFPDHVELSWWWVNGKEVHSGVSTDPQPLKE |
| | | | QPALNDSRYCLSSRLRVSATFWQNPRNHFRCQVQFYGLSENDEWTQD |
| | | | RAKPVTQIVSAEAWGRADCGFTSVSYQQGVLSATILYEILLGKATLYAVL |
| | | | VSALVLMAMVKRKDF |
| 98 | β VDJ | NT | ATGGATACCTGGCTCGTATGCTGGGCAATTTTTAGTCTCTTGAAAGC |
| | and | | AGGACTCACAGAACCTGAAGTCACCCAGACTCCCAGCCATCAGGTC |
| | constant | | ACACAGATGGGACAGGAAGTGATCTTGCGCTGTGTCCCCATCTCTA |
| | | | ATCACTTATACTTCTATTGGTACAGACAAATCTTGGGGCAGAAAGTC |
| | | | GAGTTTCTGGTTTCCTTTTATAATAATGAAATCTCAGAGAAGTCTGAA |
| | | | ATATTCGATGATCAATTCTCAGTTGAAAGGCCTGATGGATCAAATTTC |
| | | | ACTCTGAAGATCCGGTCCACAAAGCTGGAGGACTCAGCCATGTACT |
| | | | TCTGTGCCAGCAGTGAAGCTCTGCAGCCCCAGCATTTTGGTGATGG |
| | | | GACTCGACTCTCCATCCTAGAGGACCTGAACAAGGTGTTCCCACCC |
| | | | GAGGTCGCTGTGTTTGAGCCATCAGAAGCAGAGATCTCCCACACCC |
| | | | AAAAGGCCACACTGGTGTGCCTGGCCACAGGCTTCTTCCCCGACCA |
| | | | CGTGGAGCTGAGCTGGTGGGTGAATGGGAAGGAGGTGCACAGTGG |
| | | | GGTCAGCACAGACCCGCAGCCCCTCAAGGAGCAGCCCGCCCTCAA |
| | | | TGACTCCAGATACTGCCTGAGCAGCCGCCTGAGGGTCTCGGCCACC |
| | | | TTCTGGCAGAACCCCCGCAACCACTTCCGCTGTCAAGTCCAGTTCTA |
| | | | CGGGCTCTCGGAGAATGACGAGTGGACCCAGGATAGGGCCAAACC |
| | | | CGTCACCCAGATCGTCAGCGCCGAGGCCTGGGGTAGAGCAGACTG |
| | | | TGGCTTTACCTCGGTGTCCTACCAGCAAGGGGTCCTGTCTGCCACC |
| | | | ATCCTCTATGAGATCCTGCTAGGGAAGGCCACCCTGTATGCTGTGCT |
| | | | GGTCAGCGCCCTTGTGTTGATGGCCATGGTCAAGAGAAAGGATTTC |
| | | | TGA |

TABLE 13
TCR CT4.20.2C2; target peptide RVRFFFPSL (SEQ ID NO 132); target gene MAGE-
A1 B*07:02
Sequences for TCR CT4.20.2C2; target peptide RVRFFFPSL (SEQ ID NO 132);
target gene MAGE-A1 B*07:02

| | | AA | |
|---|---|---|---|
| SEQ ID | TCR | or | |
| NO | POLYPEPTIDE | NT | SEQUENCE |
| 99 | α CDR1 | AA | NSASQS |
| 100 | α CDR2 | AA | VYSSGN |
| 101 | α CDR3 | AA | CVVFDNYGQNFVF |
| 102 | β CDR1 | AA | MNHEY |
| 103 | β CDR2 | AA | SMNVEV |
| 104 | β CDR3 | AA | CASRKSTSTYNEQFF |
| 105 | α VJ | AA | MKSLRVLLVILWLQLSWVWSQRKEVEQDPGPFNVPEGATVAFNCTYSN |
| | | | SASQSFFWYRQDCRKEPKLLMSVYSSGNEDGRFTAQLNRASQYISLLI |
| | | | RDSKLSDSATYLCVVFDNYGQNFVFGPGTRLSVLP |
| 106 | α VJ | NT | ATGAAATCCTTGAGAGTTTTACTAGTGATCCTGTGGCTTCAGTTGAG |
| | | | CTGGGTTTGGAGCCAACGGAAGGAGGTGGAGCAGGATCCTGGACC |
| | | | CTTCAATGTTCCAGAGGGAGCCACTGTCGCTTTCAACTGTACTTACA |
| | | | GCAACAGTGCTTCTCAGTCTTTCTTCTGGTACAGACAGGATTGCAGG |
| | | | AAAGAACCTAAGTTGCTGATGTCCGTATACTCCAGTGGTAATGAAGA |
| | | | TGGAAGGTTTACAGCACAGCTCAATAGAGCCAGCCAGTATATTTCCC |
| | | | TGCTCATCAGAGACTCCAAGCTCAGTGATTCAGCCACCTACCTCTGT |
| | | | GTGGTGTTCGATAACTATGGTCAGAATTTTGTCTTTGGTCCCGGAAC |
| | | | CAGATTGTCCGTGCTGCCC |
| 107 | β VDJ | AA | MGPQLLGYVVLCLLGAGPLEAQVTQNPRYLITVTGKKLTVTCSQNMNH |
| | | | EYMSWYRQDPGLGLRQIYYSMNVEVTDKGDVPEGYKVSRKEKRNFPLI |
| | | | LESPSPNQTSLYFCASRKSTSTYNEQFFGPGTRLTVL |
| 108 | β VDJ | NT | ATGGGCCCCCAGCTCCTTGGCTATGTGGTCCTTTGCCTTCTAGGAG |
| | | | CAGGCCCCCTGGAAGCCCAAGTGACCCAGAACCCAAGATACCTCAT |
| | | | CACAGTGACTGGAAAGAAGTTAACAGTGACTTGTTCTCAGAATATGA |
| | | | ACCATGAGTATATGTCCTGGTATCGACAAGACCCAGGGCTGGGCTT |
| | | | AAGGCAGATCTACTATTCAATGAATGTTGAGGTGACTGATAAGGGAG |
| | | | ATGTTCCTGAAGGGTACAAAGTCTCTCGAAAAGAGAAGAGGAATTTC |
| | | | CCCCTGATCCTGGAGTCGCCCAGCCCCAACCAGACCTCTCTGTACT |
| | | | TCTGTGCCAGCCGAAAGTCGACTAGCACCTACAATGAGCAGTTCTTC |
| | | | GGGCCAGGGACACGGCTCACCGTGCTA |
| 109 | α VJ | AA | MKSLRVLLVILWLQLSWVWSQRKEVEQDPGPFNVPEGATVAFNCTYSN |
| | and | | SASQSFFWYRQDCRKEPKLLMSVYSSGNEDGRFTAQLNRASQYISLLI |
| | constant | | RDSKLSDSATYLCVVFDNYGQNFVFGPGTRLSVLPYIQNPDPAVYQLR |
| | | | DSKSSDKSVCLFTDFDSQTNVSQSKDSDVYITDKTVLDMRSMDFKSNS |
| | | | AVAWSNKSDFACANAFNNSIIPEDTFFPSPESSCDVKLVEKSFETDTNL |
| | | | NFQNLSVIGFRILLLKVAGFNLLMTLRLWSS |
| 110 | α VJ | NT | ATGAAATCCTTGAGAGTTTTACTAGTGATCCTGTGGCTTCAGTTGAG |
| | and | | CTGGGTTTGGAGCCAACGGAAGGAGGTGGAGCAGGATCCTGGACC |
| | constant | | CTTCAATGTTCCAGAGGGAGCCACTGTCGCTTTCAACTGTACTTACA |
| | | | GCAACAGTGCTTCTCAGTCTTTCTTCTGGTACAGACAGGATTGCAGG |
| | | | AAAGAACCTAAGTTGCTGATGTCCGTATACTCCAGTGGTAATGAAGA |
| | | | TGGAAGGTTTACAGCACAGCTCAATAGAGCCAGCCAGTATATTTCCC |
| | | | TGCTCATCAGAGACTCCAAGCTCAGTGATTCAGCCACCTACCTCTGT |
| | | | GTGGTGTTCGATAACTATGGTCAGAATTTTGTCTTTGGTCCCGGAAC |
| | | | CAGATTGTCCGTGCTGCCCTATATCCAGAACCCTGACCCTGCCGTG |
| | | | TACCAGCTGAGAGACTCTAAATCCAGTGACAAGTCTGTCTGCCTATT |
| | | | CACCGATTTTGATTCTCAAACAAATGTGTCACAAAGTAAGGATTCTGA |
| | | | TGTGTATATCACAGACAAAACTGTGCTAGACATGAGGTCTATGGACT |
| | | | TCAAGAGCAACAGTGCTGTGGCCTGGAGCAACAAATCTGACTTTGC |
| | | | ATGTGCAAACGCCTTCAACAACAGCATTATTCCAGAAGACACCTTCT |
| | | | TCCCCAGCCCAGAAAGTTCCTGTGATGTCAAGCTGGTCGAGAAAAG |
| | | | CTTTGAAACAGATACGAACCTAAACTTTCAAAACCTGTCAGTGATTG |
| | | | GGTTCCGAATCCTCCTCCTGAAAGTGGCCGGGTTTAATCTGCTCATG |
| | | | ACGCTGCGGTTGTGGTCCAGCTGA |
| 111 | β VDJ | AA | MGPQLLGYVVLCLLGAGPLEAQVTQNPRYLITVTGKKLTVTCSQNMNH |
| | and | | EYMSWYRQDPGLGLRQIYYSMNVEVTDKGDVPEGYKVSRKEKRNFPLI |
| | constant | | LESPSPNQTSLYFCASRKSTSTYNEQFFGPGTRLTVLEDLNKVFPPEVA |
| | | | VFEPSEAEISHTQKATLVCLATGFFPDHVELSWWWVNGKEVHSGVSTDP |
| | | | QPLKEQPALNDSRYCLSSRLRVSATFWQNPRNHFRCQVQFYGLSEND |
| | | | EWTQDRAKPVTQIVSAEAWGRADCGFTSVSYQQGVLSATILYEILLGKA |
| | | | TLYAVLVSALVLMAMVKRKDF |
| 112 | β VDJ | NT | ATGGGCCCCCAGCTCCTTGGCTATGTGGTCCTTTGCCTTCTAGGAG |
| | and | | CAGGCCCCCTGGAAGCCCAAGTGACCCAGAACCCAAGATACCTCAT |
| | constant | | CACAGTGACTGGAAAGAAGTTAACAGTGACTTGTTCTCAGAATATGA |
| | | | ACCATGAGTATATGTCCTGGTATCGACAAGACCCAGGGCTGGGCTT |
| | | | AAGGCAGATCTACTATTCAATGAATGTTGAGGTGACTGATAAGGGAG |
| | | | ATGTTCCTGAAGGGTACAAAGTCTCTCGAAAAGAGAAGAGGAATTTC |
| | | | CCCCTGATCCTGGAGTCGCCCAGCCCCAACCAGACCTCTCTGTACT |
| | | | TCTGTGCCAGCCGAAAGTCGACTAGCACCTACAATGAGCAGTTCTTC |
| | | | GGGCCAGGGACACGGCTCACCGTGCTAGAGGACCTGAACAAGGTG |
| | | | TTCCCACCCGAGGTCGCTGTGTTTGAGCCATCAGAAGCAGAGATCT |
| | | | CCCACACCCAAAAGGCCACACTGGTGTGCCTGGCCACAGGCTTCTT |
| | | | CCCCGACCACGTGGAGCTGAGCTGGTGGGTGAATGGGAAGGAGGT |
| | | | GCACAGTGGGGTCAGCACAGACCCGCAGCCCCTCAAGGAGCAGCC |
| | | | CGCCCTCAATGACTCCAGATACTGCCTGAGCAGCCGCCTGAGGGTC |
| | | | TCGGCCACCTTCTGGCAGAACCCCCGCAACCACTTCCGCTGTCAAG |
| | | | TCCAGTTCTACGGGCTCTCGGAGAATGACGAGTGGACCCAGGATAG |
| | | | GGCCAAACCCGTCACCCAGATCGTCAGCGCCGAGGCCTGGGGTAG |
| | | | AGCAGACTGTGGCTTTACCTCGGTGTCCTACCAGCAAGGGGTCCTG |
| | | | TCTGCCACCATCCTCTATGAGATCCTGCTAGGGAAGGCCACCCTGT |
| | | | ATGCTGTGCTGGTCAGCGCCCTTGTGTTGATGGCCATGGTCAAGAG |
| | | | AAAGGATTTCTGA |

TABLE 14
TCR CT23.2H9; target peptide EVDPIGHLY (SEQ ID NO: 133)/EVDPIGHVY (SEQ ID NO 134);
target gene MAGE-A3/A6 B*35:01
Sequences for TCR CT23.2H9; target peptide EVDPIGHLY (SEQ ID NO: 133)/
EVDPIGHVY (SEQ ID NO 134); target gene MAGE-A3/A6 B*35:01.

| | | AA | |
|---|---|---|---|
| SEQ ID | TCR | or | |
| NO | POLYPEPTIDE | NT | SEQUENCE |
| 113 | α CDR1 | AA | VSNAYN |
| 114 | α CDR2 | AA | GSKP |
| 115 | α CDR3 | AA | CAVPNNYGQNFVF |
| 116 | β CDR1 | AA | MNHNY |
| 117 | β CDR2 | AA | SVGAGI |
| 118 | β CDR3 | AA | CASSYSGQGLYEQYF |
| 119 | α VJ | AA | MALQSTLGAVWLGLLLNSLWKVAESKDQVFQPSTVASSEGAVVEIFCN |
| | | | HSVSNAYNFFWYLHFPGCAPRLLVKGSKPSQQGRYNMTYERFSSSLLI |
| | | | LQVREADAAVYYCAVPNNYGQNFVFGPGTRLSVLP |
| 120 | α VJ | NT | ATGGCTTTGCAGAGCACTCTGGGGGCGGTGTGGCTAGGGCTTCTCC |
| | | | TCAACTCTCTCTGGAAGGTTGCAGAAAGCAAGGACCAAGTGTTTCAG |
| | | | CCTTCCACAGTGGCATCTTCAGAGGGAGCTGTGGTGGAAATCTTCT |
| | | | GTAATCACTCTGTGTCCAATGCTTACAACTTCTTCTGGTACCTTCACT |
| | | | TCCCGGGATGTGCACCAAGACTCCTTGTTAAAGGCTCAAAGCCTTCT |
| | | | CAGCAGGGACGATACAACATGACCTATGAACGGTTCTCTTCATCGCT |
| | | | GCTCATCCTCCAGGTGCGGGAGGCAGATGCTGCTGTTTACTACTGT |
| | | | GCTGTTCCTAATAACTATGGTCAGAATTTTGTCTTTGGTCCCGGAAC |
| | | | CAGATTGTCCGTGCTGCCC |
| 121 | β VDJ | AA | MSISLLCCAAFPLLWAGPVNAGVTQTPKFRILKIGQSMTLQCTQDMNHN |
| | | | YMYWYRQDPGMGLKLIYYSVGAGITDKGEVPNGYNVSRSTTEDFPLRL |
| | | | ELAAPSQTSVYFCASSYSGQGLYEQYFGPGTRLTVT |
| 122 | β VDJ | NT | ATGAGCATCAGCCTCCTGTGCTGTGCAGCCTTTCCTCTCCTGTGGG |
| | | | CAGGTCCAGTGAATGCTGGTGTCACTCAGACCCCAAAATTCCGCAT |
| | | | CCTGAAGATAGGACAGAGCATGACACTGCAGTGTACCCAGGATATG |
| | | | AACCATAACTACATGTACTGGTATCGACAAGACCCAGGCATGGGGC |
| | | | TGAAGCTGATTTATTATTCAGTTGGTGCTGGTATCACTGATAAAGGA |
| | | | GAAGTCCCGAATGGCTACAACGTCTCCAGATCAACCACAGAGGATTT |
| | | | CCCGCTCAGGCTGGAGTTGGCTGCTCCCTCCCAGACATCTGTGTAC |
| | | | TTCTGTGCCAGCAGTTACTCTGGACAGGGTTTATACGAGCAGTACTT |
| | | | CGGGCCGGGCACCAGGCTCACGGTCACA |
| 123 | α VJ | AA | MALQSTLGAVWLGLLLNSLWKVAESKDQVFQPSTVASSEGAVVEIFCN |
| | and | | HSVSNAYNFFWYLHFPGCAPRLLVKGSKPSQQGRYNMTYERFSSSLLI |
| | constant | | LQVREADAAVYYCAVPNNYGQNFVFGPGTRLSVLPYIQNPDPAVYQLR |
| | | | DSKSSDKSVCLFTDFDSQTNVSQSKDSDVYITDKTVLDMRSMDFKSNS |
| | | | AVAWSNKSDFACANAFNNSIIPEDTFFPSPESSCDVKLVEKSFETDTNL |
| | | | NFQNLSVIGFRILLLKVAGFNLLMTLRLWSS |
| 124 | α VJ | NT | ATGGCTTTGCAGAGCACTCTGGGGGCGGTGTGGCTAGGGCTTCTCC |
| | and | | TCAACTCTCTCTGGAAGGTTGCAGAAAGCAAGGACCAAGTGTTTCAG |
| | constant | | CCTTCCACAGTGGCATCTTCAGAGGGAGCTGTGGTGGAAATCTTCT |
| | | | GTAATCACTCTGTGTCCAATGCTTACAACTTCTTCTGGTACCTTCACT |
| | | | TCCCGGGATGTGCACCAAGACTCCTTGTTAAAGGCTCAAAGCCTTCT |
| | | | CAGCAGGGACGATACAACATGACCTATGAACGGTTCTCTTCATCGCT |
| | | | GCTCATCCTCCAGGTGCGGGAGGCAGATGCTGCTGTTTACTACTGT |
| | | | GCTGTTCCTAATAACTATGGTCAGAATTTTGTCTTTGGTCCCGGAAC |
| | | | CAGATTGTCCGTGCTGCCCTATATCCAGAACCCTGACCCTGCCGTG |
| | | | TACCAGCTGAGAGACTCTAAATCCAGTGACAAGTCTGTCTGCCTATT |
| | | | CACCGATTTTGATTCTCAAACAAATGTGTCACAAAGTAAGGATTCTGA |
| | | | TGTGTATATCACAGACAAAACTGTGCTAGACATGAGGTCTATGGACT |
| | | | TCAAGAGCAACAGTGCTGTGGCCTGGAGCAACAAATCTGACTTTGC |
| | | | ATGTGCAAACGCCTTCAACAACAGCATTATTCCAGAAGACACCTTCT |
| | | | TCCCCAGCCCAGAAAGTTCCTGTGATGTCAAGCTGGTCGAGAAAAG |
| | | | CTTTGAAACAGATACGAACCTAAACTTTCAAAACCTGTCAGTGATTG |
| | | | GGTTCCGAATCCTCCTCCTGAAAGTGGCCGGGTTTAATCTGCTCATG |
| | | | ACGCTGCGGTTGTGGTCCAGCTGA |
| 125 | β VDJ | AA | MSISLLCCAAFPLLWAGPVNAGVTQTPKFRILKIGQSMTLQCTQDMNHN |
| | and | | YMYWYRQDPGMGLKLIYYSVGAGITDKGEVPNGYNVSRSTTEDFPLRL |
| | constant | | ELAAPSQTSVYFCASSYSGQGLYEQYFGPGTRLTVTEDLNKVFPPEVA |
| | | | VFEPSEAEISHTQKATLVCLATGFFPDHVELSWWVNGKEVHSGVSTDP |
| | | | QPLKEQPALNDSRYCLSSRLRVSATFWQNPRNHFRCQVQFYGLSEND |
| | | | EWTQDRAKPVTQIVSAEAWGRADCGFTSVSYQQGVLSATILYEILLGKA |
| | | | TLYAVLVSALVLMAMVKRKDF |
| 126 | β VDJ | NT | ATGAGCATCAGCCTCCTGTGCTGTGCAGCCTTTCCTCTCCTGTGGG |
| | and | | CAGGTCCAGTGAATGCTGGTGTCACTCAGACCCCAAAATTCCGCAT |
| | constant | | CCTGAAGATAGGACAGAGCATGACACTGCAGTGTACCCAGGATATG |
| | | | AACCATAACTACATGTACTGGTATCGACAAGACCCAGGCATGGGGC |
| | | | TGAAGCTGATTTATTATTCAGTTGGTGCTGGTATCACTGATAAAGGA |
| | | | GAAGTCCCGAATGGCTACAACGTCTCCAGATCAACCACAGAGGATTT |
| | | | CCCGCTCAGGCTGGAGTTGGCTGCTCCCTCCCAGACATCTGTGTAC |
| | | | TTCTGTGCCAGCAGTTACTCTGGACAGGGTTTATACGAGCAGTACTT |
| | | | CGGGCCGGGCACCAGGCTCACGGTCACAGAGGACCTGAACAAGGT |
| | | | GTTCCCACCCGAGGTCGCTGTGTTTGAGCCATCAGAAGCAGAGATC |
| | | | TCCCACACCCAAAAGGCCACACTGGTGTGCCTGGCCACAGGCTTCT |
| | | | TCCCCGACCACGTGGAGCTGAGCTGGTGGGTGAATGGGAAGGAGG |
| | | | TGCACAGTGGGGTCAGCACAGACCCGCAGCCCCTCAAGGAGCAGC |
| | | | CCGCCCTCAATGACTCCAGATACTGCCTGAGCAGCCGCCTGAGGGT |
| | | | CTCGGCCACCTTCTGGCAGAACCCCCGCAACCACTTCCGCTGTCAA |
| | | | GTCCAGTTCTACGGGCTCTCGGAGAATGACGAGTGGACCCAGGATA |
| | | | GGGCCAAACCCGTCACCCAGATCGTCAGCGCCGAGGCCTGGGGTA |
| | | | GAGCAGACTGTGGCTTTACCTCGGTGTCCTACCAGCAAGGGGTCCT |
| | | | GTCTGCCACCATCCTCTATGAGATCCTGCTAGGGAAGGCCACCCTG |
| | | | TATGCTGTGCTGGTCAGCGCCCTTGTGTTGATGGCCATGGTCAAGA |
| | | | GAAAGGATTTCTGA |

SEQ ID NO: 127
KVLEYVIKV
SEQ ID NO: 128
VRFFFPSL
SEQ ID NO: 129
YVGKEHMFY
SEQ ID NO: 130
LTQDLVQEKYLEY
SEQ ID NO: 131
SLFRAVITK
SEQ ID NO: 132
RVRFFFPSL
SEQ ID NO: 133
EVDPIGHLY
SEQ ID NO: 134
EVDPIGHVY
SEQ ID NO: 135
GAGTCCTTGTTCCGAGCAGT (MAGE-A1, forward)
SEQ ID NO: 136
GGCTCCCTGGCTCGATATTT (MAGE-A1, reverse)
SEQ ID NO: 137
CCTGAGCAACGAGCGACG (MAGE-A3/6, forward)
SEQ ID NO: 138
TCAGAACCTTGCCTCCTCACC (MAGE-A3/6, reverse)
SEQ ID NO: 139
GATCCTGCGCACTACGAGTT (MAGE-A9, forward)
SEQ ID NO: 140
ATGGGTAGCAGATGGGCTCT (MAGE-A9, reverse)
SEQ ID NO: 141
ACTGAACAGTCACCGACGAG (GUSB, forward)
SEQ ID NO: 142
GGAACGCTGCACTTTTTGGT (GUSB, reverse)
SEQ ID NO: 143
GTTTCCGCAACATCTCTCGC (PSMB4, forward)
SEQ ID NO: 144
CATCAATCACCATCTGGCCG (PSMB4, reverse)
SEQ ID NO: 145
TGAGAGGAGACTTCGATGAGAATC (VPS29, forward)
SEQ ID NO: 146
TCTGCAACAGGGCTAAGCTG (VPS29, reverse)
SEQ ID NO: 147
ALIEVGPDHFC
SEQ ID NO: 148
ALKDVEERV
SEQ ID NO: 149
ALKLKVAEL
SEQ ID NO: 150
ATLPPFMCNK
SEQ ID NO: 151
ALREEEEGV
SEQ ID NO: 152
EADPTGHSY
SEQ ID NO: 153
FPSLREAAL
SEQ ID NO: 154
FVYGEPREL
SEQ ID NO: 155
GLLGDNQIMPK
SEQ ID NO: 156
GVYAGREHFV
SEQ ID NO: 157
GVYDGREHTV
SEQ ID NO: 158
IMPKAGLLII
SEQ ID NO: 159
IVLGVILTK
SEQ ID NO: 160
KASEKIFYV
SEQ ID NO: 161
KIWEELSVLEV
SEQ ID NO: 162
KVLEFLAKL
SEQ ID NO: 163
LVFGIELMEV
SEQ ID NO: 164
RCFPVIFGK
SEQ ID NO: 165
RIFPKIMPK
SEQ ID NO: 166
RPADLTRVIM
SEQ ID NO: 167
RVRFFFPSLR
SEQ ID NO: 168
RVRIAYPSLR
SEQ ID NO: 169
SMLGDGHSMPK
SEQ ID NO: 170
SVMGVYVGK
SEQ ID NO: 171
TLDEKVAEL
SEQ ID NO: 172
TQDLVQEKY
SEQ ID NO: 173
VAELVHFLL
SEQ ID NO: 174
VIWEVLNAV
SEQ ID NO: 175
VLGEEQEGV
SEQ ID NO: 176
YPSLREAAL
SEQ ID NO: 177
YVGKEHMF
SEQ ID NO: 178
RVRIAYPSL
SEQ ID NO: 179
KVAELVHFL
SEQ ID NO: 180
KMAELVHFL
SEQ ID NO: 181
ESDPIVAQY
SEQ ID NO: 182
GLYDGREHSV
SEQ ID NO: 183
EVDPIGHXY, wherein X is L or V

### REFERENCES

1. Consortium, G. T., The Genotype-Tissue Expression (GTEx) project. Nat Genet, 2013. 45(6): p. 580-5.
2. Fawcett, D. W., L. V. Leak, and P. M. Heidger, Jr., Electron microscopic observations on the structural components of the blood-testis barrier. J Reprod Fertil Suppl, 1970. 10: p. 105-22.
3. Schaller, J., et al., Lack of HLA-molecules on human spermatozoa and in seminal plasma. Andrologia, 1993. 25(2): p. 77-81.
4. Kowalik, I., et al., Evaluation of HLA expression on gametogenic cells isolated from human testis. Andrologia, 1989. 21(3): p. 237-43.
5. Gotter, J., et al., Medullary epithelial cells of the human thymus express a highly diverse selection of tissue-specific genes colocalized in chromosomal clusters. J Exp Med, 2004. 199(2): p. 155-66.
6. Theobald, M., et al., Tolerance to p53 by A2.1-restricted cytotoxic T lymphocytes. J Exp Med, 1997. 185(5): p. 833-41.
7. Grob, J. J., et al., Safety and immunogenicity of MAGE-A3 cancer immunotherapeutic with dacarbazine in patients with MAGE-A3-positive metastatic cutaneous melanoma: an open phase I/II study with a first assessment of a predictive gene signature. ESMO Open, 2017. 2(5): p. e000203.
8. Vansteenkiste, J. F., et al., Efficacy of the MAGE-A3 cancer immunotherapeutic as adjuvant therapy in patients with resected MAGE-A3-positive non-small-cell lung cancer (MAGRIT): a randomised, double-blind, placebo-controlled, phase 3 trial. Lancet Oncol, 2016. 17(6): p. 822-835.
9. Sadovnikova, E. and H. J. Stauss, Peptide-specific cytotoxic T lymphocytes restricted by nonself major histocompatibility complex class I molecules: reagents for tumor immunotherapy. Proc Natl Acad Sci USA, 1996. 93(23): p. 13114-8.
10. Amir, A. L., et al., PRAME-specific Allo-HLA-restricted T cells with potent antitumor reactivity useful for therapeutic T-cell receptor gene transfer. Clin Cancer Res, 2011. 17(17): p. 5615-25.
11. Gezgin, G., et al., PRAME as a Potential Target for Immunotherapy in Metastatic Uveal Melanoma. JAMA Ophthalmol, 2017. 135(6): p. 541-549.
12. Jahn, L., et al., TCR-based therapy for multiple myeloma and other B-cell malignancies targeting intracellular transcription factor BOB1. Blood, 2017. 129(10): p. 1284-1295.
13. Luk, S. J., et al., PRAME and HLA Class I expression patterns make synovial sarcoma a suitable target for PRAME specific T-cell receptor gene therapy. Oncoimmunology, 2018. 7(12): p. e1507600.
14. Linette, G. P., et al., Cardiovascular toxicity and titin cross-reactivity of affinity-enhanced T cells in myeloma and melanoma. Blood, 2013. 122(6): p. 863-71.
15. Morgan, R. A., et al., Cancer regression and neurological toxicity following anti-MAGE-A3 TCR gene therapy. J Immunother, 2013. 36(2): p. 133-51.
16. Bijen, H. M., et al., Preclinical Strategies to Identify Off-Target Toxicity of High-Affinity TCRs. Molecular Therapy, 2018. 26(5): p. 1206-1214.
17. van der Lee, D. I., et al., Mutated nucleophosmin 1 as immunotherapy target in acute myeloid leukemia. J Clin Invest, 2019. 129(2): p. 774-785.
18. Burrows, S. R., et al., Peptide-MHC class I tetrameric complexes display exquisite ligand specificity. J Immunol, 2000. 165(11): p. 6229-34.
19. van Bergen, C. A., et al., Selective graft-versus-leukemia depends on magnitude and diversity of the alloreactive T cell response. J Clin Invest, 2017. 127(2): p. 517-529.
20. Koning, M. T., et al., ARTISAN PCR: rapid identification of full-length immunoglobulin rearrangements without primer binding bias. Br J Haematol, 2017. 178(6): p. 983-986.
21. Lefranc, M. P., IMGT, the international ImMunoGeneTics database. Nucleic Acids Res, 2003. 31(1): p. 307-10.
22. Meeuwsen, M. H., et al., A broad and systematic approach to identify B cell malignancy-targeting TCRs for multi-antigen-based T cell therapy. Mol Ther, 2022. 30(2): p. 564-578.
23. Weon, J. L. and P. R. Potts, The MAGE protein family and cancer. Curr Opin Cell Biol, 2015. 37: p. 1-8.
24. Zerfas, B. L., M. E. Maresh, and D. J. Trader, The Immunoproteasome: An Emerging Target in Cancer and Autoimmune and Neurological Disorders. J Med Chem, 2020. 63(5): p. 1841-1858.
25. Rock, K. L. and A. L. Goldberg, Degradation of cell proteins and the generation of MHC class I-presented peptides. Annu Rev Immunol, 1999. 17: p. 739-79.
26. Rock, K. L., et al., Inhibitors of the proteasome block the degradation of most cell proteins and the generation of peptides presented on MHC class I molecules. Cell, 1994. 78(5): p. 761-71.
27. Aki, M., et al., Interferon-gamma induces different subunit organizations and functional diversity of proteasomes. J Biochem, 1994. 115(2): p. 257-69.
28. Driscoll, J., et al., MHC-linked LMP gene products specifically alter peptidase activities of the proteasome. Nature, 1993. 365(6443): p. 262-4.
29. Gaczynska, M., K. L. Rock, and A. L. Goldberg, Gamma-interferon and expression of MHC genes regulate peptide hydrolysis by proteasomes. Nature, 1993. 365(6443): p. 264-7.
30. Cameron, B. J., et al., Identification of a Titin-derived HLA-A1-presented peptide as a cross-reactive target for engineered MAGE A3-directed T cells. Sci Transl Med, 2013. 5(197): p. 197ra103.
31. Szomolay, B., et al., Identification of human viral protein-derived ligands recognized by individual MHCI-restricted T-cell receptors. Immunol Cell Biol, 2016. 94(6): p. 573-82.
32. Di Stasi, A., et al., Inducible apoptosis as a safety switch for adoptive cell therapy. N Engl J Med, 2011. 365(18): p. 1673-83.

1. An isolated nucleic acid composition that encodes a melanoma-associated antigen (MAGE) antigen-specific binding protein having a TCR α chain variable (Vα) domain and a TCR β chain variable (Vβ) domain, the composition comprising:
(i) a nucleic acid sequence that encodes a TCR Vα domain comprising a CDR3 amino acid sequence having at least 80% sequence identity to SEQ ID NO: 17, or a functional fragment thereof; and a nucleic acid sequence that encodes a TCR Vβ domain comprising a CDR3 amino acid sequence having at least 80% sequence identity to SEQ ID NO: 20, or a functional fragment thereof, wherein the CDR3 sequences together specifically bind to MAGE-A1; or
(ii) a nucleic acid sequence that encodes a TCR Vα domain comprising a CDR3 amino acid sequence having at least 80% sequence identity to SEQ ID NO:3, or a functional fragment thereof; and a nucleic acid sequence that encodes a TCR Vβ domain comprising a CDR3 amino acid sequence having at least 80% sequence identity to SEQ ID NO:6, or a functional fragment thereof, wherein the CDR3 sequences together specifically bind to MAGE-A1; or
(iii) a nucleic acid sequence that encodes a TCR Vα domain comprising a CDR3 amino acid sequence having at least 80% sequence identity to SEQ ID NO:31, or a functional fragment thereof; and a nucleic acid sequence that encodes a TCR Vβ domain comprising a CDR3 amino acid sequence having at least 80% sequence identity to SEQ ID NO:34, or a functional fragment thereof, wherein the CDR3 sequences together specifically bind to MAGE-A9; or
(iv) a nucleic acid sequence that encodes a TCR Vα domain comprising a CDR3 amino acid sequence having at least 80% sequence identity to SEQ ID NO:45, or a functional fragment thereof; and a nucleic acid sequence that encodes a TCR Vβ domain comprising a CDR3 amino acid sequence having at least 80% sequence identity to SEQ ID NO:48, or a functional fragment thereof, wherein the CDR3 sequences together specifically bind to MAGE-A1; or
(v) a nucleic acid sequence that encodes a TCR Vα domain comprising a CDR3 amino acid sequence having at least 80% sequence identity to SEQ ID NO:59, or a functional fragment thereof; and a nucleic acid sequence that encodes a TCR Vβ domain comprising a CDR3 amino acid sequence having at least 80% sequence identity to SEQ ID NO: 62, or a functional fragment thereof, wherein the CDR3 sequences together specifically bind to MAGE-A1; or
(vi) a nucleic acid sequence that encodes a TCR Vα domain comprising a CDR3 amino acid sequence having at least 80% sequence identity to SEQ ID NO:73, or a functional fragment thereof; and a nucleic acid sequence that encodes a TCR Vβ domain comprising a CDR3 amino acid sequence having at least 80% sequence identity to SEQ ID NO: 76, or a functional fragment thereof, wherein the CDR3 sequences together specifically bind to MAGE-A1; or
(vii) a nucleic acid sequence that encodes a TCR Vα domain comprising a CDR3 amino acid sequence having at least 80% sequence identity to SEQ ID NO:87, or a functional fragment thereof; and a nucleic acid sequence that encodes a TCR Vβ domain comprising a CDR3 amino acid sequence having at least 80% sequence identity to SEQ ID NO: 90, or a functional fragment thereof, wherein the CDR3 sequences together specifically bind to MAGE-A1; or
(viii) a nucleic acid sequence that encodes a TCR Vα domain comprising a CDR3 amino acid sequence having at least 80% sequence identity to SEQ ID NO: 101, or a functional fragment thereof; and a nucleic acid sequence that encodes a TCR Vβ domain comprising a CDR3 amino acid sequence having at least 80% sequence identity to SEQ ID NO: 104, or a functional fragment thereof, wherein the CDR3 sequences together specifically bind to MAGE-A1; or
(ix) a nucleic acid sequence that encodes a TCR Vα domain comprising a CDR3 amino acid sequence having at least 80% sequence identity to SEQ ID NO:115, or a functional fragment thereof; and a nucleic acid sequence that encodes a TCR Vβ domain comprising a CDR3 amino acid sequence having at least 80% sequence identity to SEQ ID NO: 118, or a functional fragment thereof, wherein the CDR3 sequences together specifically bind to MAGE-A3 and/or MAGE-A6.

2. The nucleic acid composition of claim 1, wherein:
(i) the CDR3 of the Vα domain comprises or consists of the amino acid sequence of SEQ ID NO: 17, and the CDR3 of the Vβ domain comprises or consists of the amino acid sequence of SEQ ID NO:20; or
(ii) the CDR3 of the Vα domain comprises or consists of the amino acid sequence of SEQ ID NO: 3, and the CDR3 of the Vβ domain comprises or consists of the amino acid sequence of SEQ ID NO:6; or
(iii) the CDR3 of the Vα domain comprises or consists of the amino acid sequence of SEQ ID NO: 31, and the CDR3 of the Vβ domain comprises or consists of the amino acid sequence of SEQ ID NO:34; or
(iv) the CDR3 of the Vα domain comprises or consists of the amino acid sequence of SEQ ID NO: 45, and the CDR3 of the Vβ domain comprises or consists of the amino acid sequence of SEQ ID NO:48; or
(v) the CDR3 of the Vα domain comprises or consists of the amino acid sequence of SEQ ID NO: 59, and the CDR3 of the Vβ domain comprises or consists of the amino acid sequence of SEQ ID NO:62; or
(vi) the CDR3 of the Vα domain comprises or consists of the amino acid sequence of SEQ ID NO: 73, and the CDR3 of the Vβ domain comprises or consists of the amino acid sequence of SEQ ID NO:76; or
(vii) the CDR3 of the Vα domain comprises or consists of the amino acid sequence of SEQ ID NO: 87, and the CDR3 of the Vβ domain comprises or consists of the amino acid sequence of SEQ ID NO:90; or
(viii) the CDR3 of the Vα domain comprises or consists of the amino acid sequence of SEQ ID NO: 101, and the CDR3 of the Vβ domain comprises or consists of the amino acid sequence of SEQ ID NO: 104; or
(ix) the CDR3 of the Vα domain comprises or consists of the amino acid sequence of SEQ ID NO: 115, and the CDR3 of the Vβ domain comprises or consists of the amino acid sequence of SEQ ID NO:118.

3. The nucleic acid composition of claim 1, wherein:
(i) the Vα domain comprises an amino acid sequence having at least 80% sequence identity to, comprising, or consisting of, SEQ ID NO: 21; and the Vβ domain comprises an amino acid sequence having at least 80% sequence identity to, comprising, or consisting of, SEQ ID NO: 23; or
(ii) the Vα domain comprises an amino acid sequence having at least 80% sequence identity to, comprising, or consisting of, SEQ ID NO: 7; and the Vβ domain comprises an amino acid sequence having at least 80% sequence identity to, comprising, or consisting of, SEQ ID NO: 9; or
(iii) the Vα domain comprises an amino acid sequence having at least 80% sequence identity to, comprising, or consisting of, SEQ ID NO: 35; and the Vβ domain comprises an amino acid sequence having at least 80% sequence identity to, comprising, or consisting of, SEQ ID NO: 37; or
(iv) the Vα domain comprises an amino acid sequence having at least 80% sequence identity to, comprising, or consisting of, SEQ ID NO: 49; and the Vβ domain comprises an amino acid sequence having at least 80% sequence identity to, comprising, or consisting of, SEQ ID NO: 51; or
(v) the Vα domain comprises an amino acid sequence having at least 80% sequence identity to, comprising, or consisting of, SEQ ID NO: 63; and the Vβ domain comprises an amino acid sequence having at least 80% sequence identity to, comprising, or consisting of, SEQ ID NO: 65; or
(vi) the Vα domain comprises an amino acid sequence having at least 80% sequence identity to, comprising, or consisting of, SEQ ID NO: 77; and the Vβ domain comprises an amino acid sequence having at least 80% sequence identity to, comprising, or consisting of, SEQ ID NO: 79; or
(vii) the Vα domain comprises an amino acid sequence having at least 80% sequence identity to, comprising, or consisting of, SEQ ID NO: 91; and the Vβ domain comprises an amino acid sequence having at least 80% sequence identity to, comprising, or consisting of, SEQ ID NO: 93; or
(viii) the Vα domain comprises an amino acid sequence having at least 80% sequence identity to, comprising, or consisting of, SEQ ID NO: 105; and the Vβ domain comprises an amino acid sequence having at least 80% sequence identity to, comprising, or consisting of, SEQ ID NO: 107; or
(ix) the Vα domain comprises an amino acid sequence having at least 80% sequence identity to, comprising, or consisting of, SEQ ID NO: 119; and the Vβ domain comprises an amino acid sequence having at least 80% sequence identity to, comprising, or consisting of, SEQ ID NO: 121.

4. The nucleic acid composition of claim 1, wherein the MAGE antigen comprises an amino acid sequence selected from the group consisting of: VRFFFPSL (SEQ ID NO: 128), KVLEYVIKV (SEQ ID NO: 127), YVGKEHMFY (SEQ ID NO: 129), LTQDLVQEKYLEY (SEQ ID NO: 130), SLFRAVITK (SEQ ID NO: 131), RVRFFFPSL (SEQ ID NO: 132), EVDPIGHLY (SEQ ID NO: 133), EVDPIGHVY (SEQ ID NO: 134) and EVDPIGHXY (SEQ ID NO: 183).

5. The nucleic acid composition of claim 4, wherein the encoded binding protein is capable of specifically binding to a peptide:HLA complex selected from the group consisting of: a VRFFFPSL:HLA-C*07:02 complex (SEQ ID NO: 128), a KVLEYVIKV:HLA-A*02:01 complex (SEQ ID NO: 127), a YVGKEHMFY:HLA-A*01:01 complex (SEQ ID NO: 129), a LTQDLVQEKYLEY:HLA-A*01:01 complex (SEQ ID NO: 130), a SLFRAVITK:HLA-A*03:01 (SEQ ID NO: 131), a RVRFFFPSL:HLA-B*07:02 complex (SEQ ID NO: 132), a EVDPIGHLY:HLA-B*35:01 complex (SEQ ID NO: 133), a EVDPIGHVY:HLA-B*35:01 complex (SEQ ID NO: 134) and EVDPIGHXY:HLA-B*35:01 complex (SEQ ID NO: 183).

6.-7. (canceled)

8. The nucleic acid composition of claim 1, wherein the encoded binding protein comprises a TCR, an antigen binding fragment of a TCR, a chimeric antigen receptor (CAR), or an ImmTAC.

9. (canceled)

10. A vector system comprising a nucleic acid composition according to claim 1.

11. The vector system of claim 10, wherein the vector is a plasmid, a viral vector, or a cosmid, optionally wherein the vector is selected from the group consisting of a retrovirus, lentivirus, adeno-associated virus, adenovirus, vaccinia virus, canary poxvirus, herpes virus, minicircle vector and synthetic DNA or RNA.

12. A modified cell comprising a nucleic acid composition according to claim 1, or a vector system according to claim 10.

13. The modified cell of claim 12, wherein the modified cell is selected from the group consisting of a CD8 T cell, a CD4 T cell, an NK cell, an NK-T cell, a gamma-delta T cell, a hematopoietic stem cell, an inducible pluripotent stem cell, a progenitor cell, a T cell line and a NK-92 cell line.

14. (canceled)

15. A pharmaceutical composition comprising a nucleic acid composition according to claim 1, a vector system according to claim 10, or a modified cell according to claim 12, and a pharmaceutically acceptable excipient, adjuvant, diluent and/or carrier.

16. A method for inducing or enhancing an immune response in human subject diagnosed with a MAGE associated disease or condition, wherein the method comprises administering to the human subject the pharmaceutical composition according to claim 15.

17. A method for stimulating a cell mediated immune response to a target cell population or tissue in a human subject, wherein the method comprises administering to the human subject the pharmaceutical composition according to claim 15.

18. A method for providing anti-tumor immunity to a human subject, wherein the method comprises administering to the human subject the pharmaceutical composition according to claim 15.

19. A method for treating a human subject having a disease or condition associated with an elevated level of HLA-restricted MAGE antigen, wherein the method comprises administering to the human subject the pharmaceutical composition according to claim 15.

20.-21. (canceled)

22. The method according to claim 16, wherein the MAGE associated disease or condition is a hematological malignancy or a solid tumor.

23. The method according to claim 22, wherein the hematological malignancy is selected from the group consisting of: Multiple myeloma, plasma cell leukemia, Amyloidosis (AL), Acute lymphoblastoid leukemia (ALL), Chronic lymphocytic leukemia (CLL), Waldenstrom macroglobulinemia, Acute myeloid leukemia (AML), Myeloid dysplastic syndrome (MDS) and B cell lymphoma, optionally wherein the B cell lymphoma is selected from the group consisting of: Diffuse large B cell lymphoma (DLBCL), High grade B cell lymphoma, Mantel cell lymphoma (MCL), Follicular lymphoma (FL) and Burkitt Lymphoma.

24. (canceled)

25. The method according to claim 22, wherein the solid tumor is selected from the group consisting of: Melanoma, Lung carcinoma, Bladder carcinoma, Ovarian carcinoma, Head and neck carcinoma, Breast carcinoma, Sarcoma, Uveal melanoma and Uterine carcinoma, optionally wherein the solid tumor is selected from the group consisting of: non-small cell lung carcinoma, head and neck squamous cell carcinoma, invasive breast carcinoma and synovial sarcoma.

26. A method of generating a binding protein that is capable of specifically binding to a peptide containing a MAGE antigen and does not bind to a peptide that does not contain the MAGE antigen, comprising contacting a nucleic acid composition according to claim 1 with a cell under conditions in which the nucleic acid composition is incorporated and expressed by the cell.

27. (canceled)

28. An isolated nucleic acid sequence comprising or consisting of the nucleotide sequence of any one of SEQ ID NOs: 8, 10, 12, 14, 22, 24, 26, 28, 36, 38, 40, 42, 50, 52, 54, 56, 64, 66, 68, 70, 78, 80, 82, 84, 92, 94, 96, 98, 106, 108, 110, 112, 120, 122, 124 or 126.

29. An isolated nucleic acid sequence comprising or consisting of the nucleotide sequence of any one of SEQ ID NOs: 8, 10, 12, 14, 22, 24, 26, 28, 36, 38, 40, 42, 50, 52, 54, 56, 64, 66, 68, 70, 78, 80, 82, 84, 92, 94, 96, 98, 106, 108, 110, 112, 120, 122, 124 or 126 for use in therapy.


SEQUENCE LISTING (183 sequences)

SEQ ID NO: 1 - protein, 6 aa, Homo sapiens
DSSSTY

SEQ ID NO: 2 - protein, 7 aa, Homo sapiens
IFSNMDM

SEQ ID NO: 3 - protein, 11 aa, Homo sapiens
CAESIDARLMF

SEQ ID NO: 4 - protein, 5 aa, Homo sapiens
SGDLS

SEQ ID NO: 5 - protein, 6 aa, Homo sapiens
YYNGEE

SEQ ID NO: 6 - protein, 12 aa, Homo sapiens
CASSPGGDTQYF

SEQ ID NO: 7 - protein, 130 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAESIDARLMFGDGTQLVVKP

SEQ ID NO: 8 - DNA, 390 bp, Homo sapiens
  source 1..390: mol_type = genomic DNA
atgaagacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagagagtatagatgccagactcatgtttggagatggaactcagctggtggtgaagccc

SEQ ID NO: 9 - protein, 130 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSPGGDTQYFGPGTRLTVL

SEQ ID NO: 10 - DNA, 390 bp, Homo sapiens
  source 1..390: mol_type = genomic DNA
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagccccggcggggatacgcagtattttggcccaggcacccggctgacagtgctc

SEQ ID NO: 11 - protein, 271 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAESIDARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 12 - DNA, 816 bp, Homo sapiens
  source 1..816: mol_type = genomic DNA
atgaagacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagagagtatagatgccagactcatgtttggagatggaactcagctggtggtgaagcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 13 - protein, 307 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSPGGDTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 14 - DNA, 924 bp, Homo sapiens
  source 1..924: mol_type = genomic DNA
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagccccggcggggatacgcagtattttggcccaggcacccggctgacagtgctcgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 15 - protein, 6 aa, Homo sapiens
NSMFDY

SEQ ID NO: 16 - protein, 9 aa, Homo sapiens
ISISSIKDK

SEQ ID NO: 17 - protein, 12 aa, Homo sapiens
CAASAQNNDMRF

SEQ ID NO: 18 - protein, 5 aa, Homo sapiens
SGHDY

SEQ ID NO: 19 - protein, 6 aa, Homo sapiens
FNNNVP

SEQ ID NO: 20 - protein, 13 aa, Homo sapiens
CASGTGWDTEAFF

SEQ ID NO: 21 - protein, 137 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASAQNNDMRFGAGTRLTVKP

SEQ ID NO: 22 - DNA, 411 bp, Homo sapiens
  source 1..411: mol_type = genomic DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagcgcgcagaacaatgacatgcgctttggagcagggaccagactgacagtaaaacca

SEQ ID NO: 23 - protein, 132 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASGTGWDTEAFFGQGTRLTVV

SEQ ID NO: 24 - DNA, 396 bp, Homo sapiens
  source 1..396: mol_type = genomic DNA
atgggctcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcggaacagggtgggacactgaagctttctttggacaaggcaccagactcacagttgta

SEQ ID NO: 25 - protein, 278 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASAQNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 26 - DNA, 837 bp, Homo sapiens
  source 1..837: mol_type = genomic DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagcgcgcagaacaatgacatgcgctttggagcagggaccagactgacagtaaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 27 - protein, 309 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASGTGWDTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 28 - DNA, 930 bp, Homo sapiens
  source 1..930: mol_type = genomic DNA
atgggctcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcggaacagggtgggacactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 29 - protein, 6 aa, Homo sapiens
VTNFRS

SEQ ID NO: 30 - protein, 7 aa, Homo sapiens
LTSSGIE

SEQ ID NO: 31 - protein, 12 aa, Homo sapiens
CAVEGWDDKIIF

SEQ ID NO: 32 - protein, 5 aa, Homo sapiens
ENHRY

SEQ ID NO: 33 - protein, 6 aa, Homo sapiens
SYGVKD

SEQ ID NO: 34 - protein, 12 aa, Homo sapiens
CAISEGRNEQYF

SEQ ID NO: 35 - protein, 131 aa, Homo sapiens
MMKCPQALLAIFWLLLSWVSSEDKVVQSPLSLVVHEGDTVTLNCSYEVTNFRSLLWYKQEKKAPTFLFMLTSSGIEKKSGRLSSILDKKELSSILNITATQTGDSAIYLCAVEGWDDKIIFGKGTRLHILP

SEQ ID NO: 36 - DNA, 393 bp, Homo sapiens
  source 1..393: mol_type = genomic DNA
atgatgaagtgtccacaggctttactagctatcttttggcttctactgagctgggtgagcagtgaagacaaggtggtacaaagccctctatctctggttgtccacgagggagacaccgtaactctcaattgcagttatgaagtgactaactttcgaagcctactatggtacaagcaggaaaagaaagctcccacatttctatttatgctaacttcaagtggaattgaaaagaagtcaggaagactaagtagcatattagataagaaagaactttccagcatcctgaacatcacagccacccagaccggagactcggccatctacctctgtgctgtggagggatgggatgacaagatcatctttggaaaagggacacgacttcatattctcccc

SEQ ID NO: 37 - protein, 130 aa, Homo sapiens
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISEGRNEQYFGPGTRLTVT

SEQ ID NO: 38 - DNA, 390 bp, Homo sapiens
  source 1..390: mol_type = genomic DNA
atgggcacaaggttgttcttctatgtggccctttgtctcctgtggacaggacacatggatgctggaatcacccagagcccaagacacaaggtcacagagacaggaacaccagtgactctgagatgtcatcagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcagtgagggccggaacgagcagtacttcgggccgggcaccaggctcacggtcaca

SEQ ID NO: 39 - protein, 272 aa, Homo sapiens
MMKCPQALLAIFWLLLSWVSSEDKVVQSPLSLVVHEGDTVTLNCSYEVTNFRSLLWYKQEKKAPTFLFMLTSSGIEKKSGRLSSILDKKELSSILNITATQTGDSAIYLCAVEGWDDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 40 - DNA, 819 bp, Homo sapiens
  source 1..819: mol_type = genomic DNA
atgatgaagtgtccacaggctttactagctatcttttggcttctactgagctgggtgagcagtgaagacaaggtggtacaaagccctctatctctggttgtccacgagggagacaccgtaactctcaattgcagttatgaagtgactaactttcgaagcctactatggtacaagcaggaaaagaaagctcccacatttctatttatgctaacttcaagtggaattgaaaagaagtcaggaagactaagtagcatattagataagaaagaactttccagcatcctgaacatcacagccacccagaccggagactcggccatctacctctgtgctgtggagggatgggatgacaagatcatctttggaaaagggacacgacttcatattctccccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 41 - protein, 307 aa, Homo sapiens
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISEGRNEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 42 - DNA, 924 bp, Homo sapiens
  source 1..924: mol_type = genomic DNA
atgggcacaaggttgttcttctatgtggccctttgtctcctgtggacaggacacatggatgctggaatcacccagagcccaagacacaaggtcacagagacaggaacaccagtgactctgagatgtcatcagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcagtgagggccggaacgagcagtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 43 - protein, 6 aa, Homo sapiens
DSAIYN

SEQ ID NO: 44 - protein, 7 aa, Homo sapiens
IQSSQRE

SEQ ID NO: 45 - protein, 13 aa, Homo sapiens
CAVLNQAGTALIF

SEQ ID NO: 46 - protein, 5 aa, Homo sapiens
SGDLS

SEQ ID NO: 47 - protein, 6 aa, Homo sapiens
YYNGEE

SEQ ID NO: 48 - protein, 15 aa, Homo sapiens
CASSVWSVGSTEAFF

SEQ ID NO: 49 - protein, 131 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVLNQAGTALIFGKGTTLSVSS

SEQ ID NO: 50 - DNA, 393 bp, Homo sapiens
  source 1..393: mol_type = genomic DNA
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtcctaaaccaggcaggaactgctctgatctttgggaagggaaccaccttatcagtgagttcc

SEQ ID NO: 51 - protein, 133 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVWSVGSTEAFFGQGTRLTVV

SEQ ID NO: 52 - DNA, 399 bp, Homo sapiens
  source 1..399: mol_type = genomic DNA
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtatggtcagtcgggagcactgaagctttctttggacaaggcaccagactcacagttgta

SEQ ID NO: 53 - protein, 272 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVLNQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 54 - DNA, 819 bp, Homo sapiens
  source 1..819: mol_type = genomic DNA
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtcctaaaccaggcaggaactgctctgatctttgggaagggaaccaccttatcagtgagttccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 55 - protein, 310 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVWSVGSTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 56 - DNA, 933 bp, Homo sapiens
  source 1..933: mol_type = genomic DNA
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtatggtcagtcgggagcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 57 - protein, 6 aa, Homo sapiens
NSASQS

SEQ ID NO: 58 - protein, 6 aa, Homo sapiens
VYSSGN

SEQ ID NO: 59 - protein, 13 aa, Homo sapiens
CVVNFNYGQNFVF

SEQ ID NO: 60 - protein, 6 aa, Homo sapiens
SEHNRL

SEQ ID NO: 61 - protein, 6 aa, Homo sapiens
FQNEAQ

SEQ ID NO: 62 - protein, 14 aa, Homo sapiens
CASSPLGVNTEAFF

SEQ ID NO: 63 - protein, 131 aa, Homo sapiens
MKSLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNFNYGQNFVFGPGTRLSVLP

SEQ ID NO: 64 - DNA, 393 bp, Homo sapiens
  source 1..393: mol_type = genomic DNA
atgaaatccttgagagttttactagtgatcctgtggcttcagttgagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgaattttaactatggtcagaattttgtctttggtcccggaaccagattgtccgtgctgccc

SEQ ID NO: 65 - protein, 133 aa, Homo sapiens
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSPLGVNTEAFFGQGTRLTVV

SEQ ID NO: 66 - DNA, 399 bp, Homo sapiens
  source 1..399: mol_type = genomic DNA
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagccccctgggggtgaacactgaagctttctttggacaaggcaccagactcacagttgta

SEQ ID NO: 67 - protein, 272 aa, Homo sapiens
MKSLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNFNYGQNFVFGPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 68 - DNA, 819 bp, Homo sapiens
  source 1..819: mol_type = genomic DNA
atgaaatccttgagagttttactagtgatcctgtggcttcagttgagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgaattttaactatggtcagaattttgtctttggtcccggaaccagattgtccgtgctgccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 69 - protein, 310 aa, Homo sapiens
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSPLGVNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 70 - DNA, 933 bp, Homo sapiens
  source 1..933: mol_type = genomic DNA
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagccccctgggggtgaacactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 71 - protein, 6 aa, Homo sapiens
VSNAYN

SEQ ID NO: 72 - protein, 4 aa, Homo sapiens
GSKP

SEQ ID NO: 73 - protein, 13 aa, Homo sapiens
CAVVHNYGQNFVF

SEQ ID NO: 74 - protein, 6 aa, Homo sapiens
SEHNRL

SEQ ID NO: 75 - protein, 6 aa, Homo sapiens
FQNEAQ

SEQ ID NO: 76 - protein, 15 aa, Homo sapiens
CASSPLASDTGELFF

SEQ ID NO: 77 - protein, 131 aa, Homo sapiens
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVVHNYGQNFVFGPGTRLSVLP

SEQ ID NO: 78 - DNA, 393 bp, Homo sapiens
  source 1..393: mol_type = genomic DNA
atggctttgcagagcactctgggggcggtgtggctagggcttctcctcaactctctctggaaggttgcagaaagcaaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgtggttcataactatggtcagaattttgtctttggtcccggaaccagattgtccgtgctgccc

SEQ ID NO: 79 - protein, 134 aa, Homo sapiens
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSPLASDTGELFFGEGSRLTVL

SEQ ID NO: 80 - DNA, 402 bp, Homo sapiens
  source 1..402: mol_type = genomic DNA
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagccccttagccagtgacaccggggagctgttttttggagaaggctctaggctgaccgtactg

SEQ ID NO: 81 - protein, 272 aa, Homo sapiens
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVVHNYGQNFVFGPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 82 - DNA, 819 bp, Homo sapiens
  source 1..819: mol_type = genomic DNA
atggctttgcagagcactctgggggcggtgtggctagggcttctcctcaactctctctggaaggttgcagaaagcaaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgtggttcataactatggtcagaattttgtctttggtcccggaaccagattgtccgtgctgccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 83 - protein, 311 aa, Homo sapiens
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSPLASDTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 84 - DNA, 936 bp, Homo sapiens
  source 1..936: mol_type = genomic DNA
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagccccttagccagtgacaccggggagctgttttttggagaaggctctaggctgaccgtactggaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 85 - protein, 7 aa, Homo sapiens
TRDTTYY

SEQ ID NO: 86 - protein, 8 aa, Homo sapiens
RNSFDEQN

SEQ ID NO: 87 - protein, 17 aa, Homo sapiens
CALSEAYSGAGSYQLTF

SEQ ID NO: 88 - protein, 5 aa, Homo sapiens
SNHLY

SEQ ID NO: 89 - protein, 6 aa, Homo sapiens
FYNNEI

SEQ ID NO: 90 - protein, 12 aa, Homo sapiens
CASSEALQPQHF

SEQ ID NO: 91 - protein, 138 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEAYSGAGSYQLTFGKGTKLSVIP

SEQ ID NO: 92 - DNA, 414 bp, Homo sapiens
  source 1..414: mol_type = genomic DNA
atgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgaggcatactctggggctgggagttaccaactcactttcgggaaggggaccaaactctcggtcatacca

SEQ ID NO: 93 - protein, 131 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEALQPQHFGDGTRLSIL

SEQ ID NO: 94 - DNA, 393 bp, Homo sapiens
  source 1..393: mol_type = genomic DNA
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgaagctctgcagccccagcattttggtgatgggactcgactctccatccta

SEQ ID NO: 95 - protein, 279 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEAYSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 96 - DNA, 840 bp, Homo sapiens
  source 1..840: mol_type = genomic DNA
atgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgaggcatactctggggctgggagttaccaactcactttcgggaaggggaccaaactctcggtcataccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 97 - protein, 308 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEALQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 98 - DNA, 927 bp, Homo sapiens
  source 1..927: mol_type = genomic DNA
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgaagctctgcagccccagcattttggtgatgggactcgactctccatcctagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 99 - protein, 6 aa, Homo sapiens
NSASQS

SEQ ID NO: 100 - protein, 6 aa, Homo sapiens
VYSSGN

SEQ ID NO: 101 - protein, 13 aa, Homo sapiens
CVVFDNYGQNFVF

SEQ ID NO: 102 - protein, 5 aa, Homo sapiens
MNHEY

SEQ ID NO: 103 - protein, 6 aa, Homo sapiens
SMNVEV

SEQ ID NO: 104 - protein, 15 aa, Homo sapiens
CASRKSTSTYNEQFF

SEQ ID NO: 105 - protein, 131 aa, Homo sapiens
MKSLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVFDNYGQNFVFGPGTRLSVLP

SEQ ID NO: 106 - DNA, 393 bp, Homo sapiens
  source 1..393: mol_type = genomic DNA
atgaaatccttgagagttttactagtgatcctgtggcttcagttgagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgttcgataactatggtcagaattttgtctttggtcccggaaccagattgtccgtgctgccc

SEQ ID NO: 107 - protein, 133 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASRKSTSTYNEQFFGPGTRLTVL

SEQ ID NO: 108 - DNA, 399 bp, Homo sapiens
  source 1..399: mol_type = genomic DNA
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagccgaaagtcgactagcacctacaatgagcagttcttcgggccagggacacggctcaccgtgcta

SEQ ID NO: 109 - protein, 272 aa, Homo sapiens
MKSLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVFDNYGQNFVFGPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 110 - DNA, 819 bp, Homo sapiens
  source 1..819: mol_type = genomic DNA
atgaaatccttgagagttttactagtgatcctgtggcttcagttgagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgttcgataactatggtcagaattttgtctttggtcccggaaccagattgtccgtgctgccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 111 - protein, 310 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASRKSTSTYNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 112 - DNA, 933 bp, Homo sapiens
  source 1..933: mol_type = genomic DNA
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagccgaaagtcgactagcacctacaatgagcagttcttcgggccagggacacggctcaccgtgctagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 113 - protein, 6 aa, Homo sapiens
VSNAYN

SEQ ID NO: 114 - protein, 4 aa, Homo sapiens
GSKP

SEQ ID NO: 115 - protein, 13 aa, Homo sapiens
CAVPNNYGQNFVF

SEQ ID NO: 116 - protein, 5 aa, Homo sapiens
MNHNY

SEQ ID NO: 117 - protein, 6 aa, Homo sapiens
SVGAGI

SEQ ID NO: 118 - protein, 15 aa, Homo sapiens
CASSYSGQGLYEQYF

SEQ ID NO: 119 - protein, 131 aa, Homo sapiens
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVPNNYGQNFVFGPGTRLSVLP

SEQ ID NO: 120 - DNA, 393 bp, Homo sapiens
  source 1..393: mol_type = genomic DNA
atggctttgcagagcactctgggggcggtgtggctagggcttctcctcaactctctctggaaggttgcagaaagcaaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgttcctaataactatggtcagaattttgtctttggtcccggaaccagattgtccgtgctgccc

SEQ ID NO: 121 - protein, 133 aa, Homo sapiens
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYSGQGLYEQYFGPGTRLTVT

SEQ ID NO: 122 - DNA, 399 bp, Homo sapiens
  source 1..399: mol_type = genomic DNA
atgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagttactctggacagggtttatacgagcagtacttcgggccgggcaccaggctcacggtcaca

SEQ ID NO: 123 - protein, 272 aa, Homo sapiens
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVPNNYGQNFVFGPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 124 - DNA, 819 bp, Homo sapiens
  source 1..819: mol_type = genomic DNA
atggctttgcagagcactctgggggcggtgtggctagggcttctcctcaactctctctggaaggttgcagaaagcaaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgttcctaataactatggtcagaattttgtctttggtcccggaaccagattgtccgtgctgccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 125 - protein, 310 aa, Homo sapiens
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYSGQGLYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 126 - DNA, 933 bp, Homo sapiens
  source 1..933: mol_type = genomic DNA
atgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagttactctggacagggtttatacgagcagtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 127 - protein, 9 aa, Homo sapiens
KVLEYVIKV

SEQ ID NO: 128 - protein, 8 aa, Homo sapiens
VRFFFPSL

SEQ ID NO: 129 - protein, 9 aa, Homo sapiens
YVGKEHMFY

SEQ ID NO: 130 - protein, 13 aa, Homo sapiens
LTQDLVQEKYLEY

SEQ ID NO: 131 - protein, 9 aa, Homo sapiens
SLFRAVITK

SEQ ID NO: 132 - protein, 9 aa, Homo sapiens
RVRFFFPSL

SEQ ID NO: 133 - protein, 9 aa, Homo sapiens
EVDPIGHLY

SEQ ID NO: 134 - protein, 9 aa, Homo sapiens
EVDPIGHVY

SEQ ID NO: 135 - DNA, 20 bp, synthetic construct
  source 1..20: mol_type = other DNA; note = MAGE-A1 forward Primer
gagtccttgttccgagcagt

SEQ ID NO: 136 - DNA, 20 bp, synthetic construct
  source 1..20: mol_type = other DNA; note = MAGE-A1 reverse primer
ggctccctggctcgatattt

SEQ ID NO: 137 - DNA, 18 bp, synthetic construct
  source 1..18: mol_type = other DNA; note = MAGE-A3/A6 forward primer
cctgagcaacgagcgacg

SEQ ID NO: 138 - DNA, 21 bp, synthetic construct
  source 1..21: mol_type = other DNA; note = MAGE-A3/A6 reverse primer
tcagaaccttgcctcctcacc

SEQ ID NO: 139 - DNA, 20 bp, synthetic construct
  source 1..20: mol_type = other DNA; note = MAGE-A9 forward primer
gatcctgcgcactacgagtt

SEQ ID NO: 140 - DNA, 20 bp, synthetic construct
  source 1..20: mol_type = other DNA; note = MAGE-A9 reverse primer
atgggtagcagatgggctct

SEQ ID NO: 141 - DNA, 20 bp, synthetic construct
  source 1..20: mol_type = other DNA; note = GUSB forward primer
actgaacagtcaccgacgag

SEQ ID NO: 142 - DNA, 20 bp, synthetic construct
  source 1..20: mol_type = other DNA; note = GUSB reverse primer
ggaacgctgcactttttggt

SEQ ID NO: 143 - DNA, 20 bp, synthetic construct
  source 1..20: mol_type = other DNA; note = PSMB4 forward primer
gtttccgcaacatctctcgc

SEQ ID NO: 144 - DNA, 20 bp, synthetic construct
  source 1..20: mol_type = other DNA; note = PSMB4 reverse primer
catcaatcaccatctggccg

SEQ ID NO: 145 - DNA, 24 bp, synthetic construct
  source 1..24: mol_type = other DNA; note = VPS29 forward primer
tgagaggagacttcgatgagaatc

SEQ ID NO: 146 - DNA, 20 bp, synthetic construct
  source 1..20: mol_type = other DNA; note = VPS29 reverse primer
tctgcaacagggctaagctg

SEQ ID NO: 147 - protein, 11 aa, Homo sapiens
ALIEVGPDHFC

SEQ ID NO: 148 - protein, 9 aa, Homo sapiens
ALKDVEERV

SEQ ID NO: 149 - protein, 9 aa, Homo sapiens
ALKLKVAEL

SEQ ID NO: 150 - protein, 10 aa, Homo sapiens
ATLPPFMCNK

SEQ ID NO: 151 - protein, 9 aa, Homo sapiens
ALREEEEGV

SEQ ID NO: 152 - protein, 9 aa, Homo sapiens
EADPTGHSY

SEQ ID NO: 153 - protein, 9 aa, Homo sapiens
FPSLREAAL

SEQ ID NO: 154 - protein, 9 aa, Homo sapiens
FVYGEPREL

SEQ ID NO: 155 - protein, 11 aa, Homo sapiens
GLLGDNQIMPK

SEQ ID NO: 156 - protein, 10 aa, Homo sapiens
GVYAGREHFV

SEQ ID NO: 157 - protein, 10 aa, Homo sapiens
GVYDGREHTV

SEQ ID NO: 158 - protein, 10 aa, Homo sapiens
IMPKAGLLII

SEQ ID NO: 159 - protein, 9 aa, Homo sapiens
IVLGVILTK

SEQ ID NO: 160 - protein, 9 aa, Homo sapiens
KASEKIFYV

SEQ ID NO: 161 - protein, 11 aa, Homo sapiens
KIWEELSVLEV

SEQ ID NO: 162 - protein, 9 aa, Homo sapiens
KVLEFLAKL

SEQ ID NO: 163 - protein, 10 aa, Homo sapiens
LVFGIELMEV

SEQ ID NO: 164 - protein, 9 aa, Homo sapiens
RCFPVIFGK

SEQ ID NO: 165 - protein, 9 aa, Homo sapiens
RIFPKIMPK

SEQ ID NO: 166 - protein, 10 aa, Homo sapiens
RPADLTRVIM

SEQ ID NO: 167 - protein, 10 aa, Homo sapiens
RVRFFFPSLR

SEQ ID NO: 168 - protein, 10 aa, Homo sapiens
RVRIAYPSLR

SEQ ID NO: 169 - protein, 11 aa, Homo sapiens
SMLGDGHSMPK

SEQ ID NO: 170 - protein, 9 aa, Homo sapiens
SVMGVYVGK

SEQ ID NO: 171 - protein, 9 aa, Homo sapiens
TLDEKVAEL

SEQ ID NO: 172 - protein, 9 aa, Homo sapiens
TQDLVQEKY

SEQ ID NO: 173 - protein, 9 aa, Homo sapiens
VAELVHFLL

SEQ ID NO: 174 - protein, 9 aa, Homo sapiens
VIWEVLNAV

SEQ ID NO: 175 - protein, 9 aa, Homo sapiens
VLGEEQEGV

SEQ ID NO: 176 - protein, 9 aa, Homo sapiens
YPSLREAAL

SEQ ID NO: 177 - protein, 8 aa, Homo sapiens
YVGKEHMF

SEQ ID NO: 178 - protein, 9 aa, Homo sapiens
RVRIAYPSL

SEQ ID NO: 179 - protein, 9 aa, Homo sapiens
KVAELVHFL

SEQ ID NO: 180 - protein, 9 aa, Homo sapiens
KMAELVHFL

SEQ ID NO: 181 - protein, 9 aa, Homo sapiens
ESDPIVAQY

SEQ ID NO: 182 - protein, 10 aa, Homo sapiens
GLYDGREHSV

SEQ ID NO: 183 - protein, 9 aa, Homo sapiens
  VARIANT 8: note = L or V
EVDPIGHXY